(19) United States
(12) Patent Application Publication
(10) Pub. No.: US 2025/0145599 A1
(43) Pub. Date: May 8, 2025

(54) CERTAIN CHEMICAL ENTITIES, COMPOSITIONS, AND METHODS

(71) Applicant: Retune Pharma Inc., New York, NY (US)

(72) Inventors: Andrew I. MCDONALD, New York, NY (US); Shawn QIAN, New York, NY (US)

(21) Appl. No.: 18/835,167

(22) PCT Filed: Feb. 3, 2023
(86) PCT No.: PCT/US2023/061911

§ 371 Date: Aug. 1, 2024

Related U.S. Application Data
(60) Provisional application No. 63/306,944, filed on Feb. 4, 2022.
(60) Provisional application No. 63/419,988, filed on Oct. 27, 2022.

(51) Int. Cl. C07D 413/14; A61K 31/4709; A61K 31/496; A61K 31/517; A61K 31/519; A61K 31/5377; C07D 401/04; C07D 401/14; C07D 403/04; C07D 405/14; C07D 487/04

(52) U.S. Cl. CPC C07D 413/14; A61K 31/4709; A61K 31/496; A61K 31/517; A61K 31/519; A61K 31/5377; C07D 401/04; C07D 401/14; C07D 403/04; C07D 405/14; C07D 487/04

(57) ABSTRACT

Provided herein are inhibitors of IGF-1R, pharmaceutical compositions comprising said inhibitory compounds, and methods for using said IGF-1R inhibitory compounds for the treatment of disease.

### CROSS-REFERENCE TO RELATED APPLICATION

This application claims the benefit of U.S. Patent Application No. 63/306,944, filed on Feb. 4, 2022; and U.S. Patent Application No. 63/419,988, filed on Oct. 27, 2022, each of which is hereby incorporated by reference in its entirety.

### BACKGROUND

Impaired regulation of IGF-1R has been linked to aberrant cell division, loss of apoptotic regulation, chromosomal instability, and increased incidence of cancer. Accordingly, therapies that target IGF-1R activity are desired for use in the treatment of cancer, autoimmune disorders, and other disorders characterized by aberrant IGF-1R pathway signaling.

### BRIEF SUMMARY OF THE INVENTION

Provided herein are inhibitors of IGF-1R, pharmaceutical compositions comprising said inhibitory compounds, and methods for using said inhibitory compounds for the treatment of disease.

One embodiment provides a compound, or a pharmaceutically acceptable salt or solvate thereof, having the structure of Formula (I):

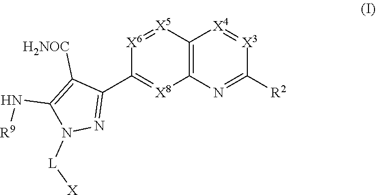

wherein,
   X is optionally substituted alkyl, optionally substituted carbocyclyl, optionally substituted carbocyclylalkyl, optionally substituted aryl, optionally substituted aralkyl, optionally substituted heteroaryl, optionally substituted heteroaralkyl, optionally substituted heterocyclyl, or optionally substituted heterocyclylalkyl;
   L is a bond, or optionally substituted carbocyclyl, optionally substituted carbocyclylalkyl, optionally substituted heterocyclyl, or optionally substituted heterocyclylalkyl;
   R² is optionally substituted carbocyclyl, optionally substituted aryl, optionally substituted heteroaryl, or optionally substituted heterocyclyl; wherein the optional substitution of the optionally substituted carbocyclyl, optionally substituted aryl, optionally substituted heteroaryl, or optionally substituted heterocyclyl is selected from the group consisting of cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted aryloxy, optionally substituted heteroaryloxy, optionally substituted (heterocyclyl)-O—, optionally substituted C1-C6 alkyl, optionally substituted C2-C6 alkynyl, optionally substituted carbocyclyl, optionally substituted C2-C6 alkenyl, optionally substituted aryl, optionally substituted heteroaryl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—N(R¹⁰)₂, or —NR¹¹SO₂—N(R¹⁰)₂;
X³ is N or C—R³;
X⁴ is N or C—R⁴;
X⁵ is N or C—R⁵;
X⁶ is N or C—R⁶;
X⁸ is N or C—R⁸;
R³, R⁴, R⁵, R⁶, and R⁸ are independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C2-C6 alkynyl, optionally substituted carbocyclyl, optionally substituted C2-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl;
R¹² is H or optionally substituted C1-C6 alkyl;
R⁹ is selected from hydrogen, optionally substituted C1-C6 alkyl, optionally substituted C3-C8 carbocyclyl, or optionally substituted C4-C10 carbocyclylalkyl; wherein either R⁹ and L, or R⁹ and X, may be joined together with any intervening atoms to form an optionally substituted heterocyclyl ring.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is selected from cancer, autoimmune disease, or thyroid eye disease.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.
“Amino” refers to the —NH₂ radical.
“Cyano” refers to the —CN radical.
“Nitro” refers to the —NO₂ radical.
“Oxa” refers to the —O-radical.
“Oxo” refers to the ═O radical.
“Thioxo” refers to the ═S radical
“Imino” refers to the ═N—H radical.
“Oximo” refers to the ═N—OH radical.
“Hydrazino” refers to the ═N—NH₂ radical.
“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF₃ group.
“Alkoxy” refers to a radical bonded through an oxygen atom of the formula —O-alkyl, where alkyl is an alkyl chain as defined above.
“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR₂, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₈ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R³, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).
“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.
“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.
“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.
“Aralkynyl” refers to a radical of the formula —R^{c}-aryl, where R^{c} is an alkynylene chain as defined above. The aryl part of the aralkynyl radical is optionally substituted as described above for an aryl group. The alkynylene chain part of the aralkynyl radical is optionally substituted as defined above for an alkynylene chain.
“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.
“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.
“Carbocyclylalkyl” refers to a radical of the formula —R^{c}-carbocyclyl where R^{c} is an alkylene chain as defined above. The alkylene chain and the carbocyclyl radical is optionally substituted as defined above.
“Carbocyclylalkynyl” refers to a radical of the formula —R^{c}-carbocyclyl where R^{c} is an alkynylene chain as defined above. The alkynylene chain and the carbocyclyl radical is optionally substituted as defined above.
“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.
“Halo” or “halogen” refers to bromo, chloro, fluoro or iodo substituents.
“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.
“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.
“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.
“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.
“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.
“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.
“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7] cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.
“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.
“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.
“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.
“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or(S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

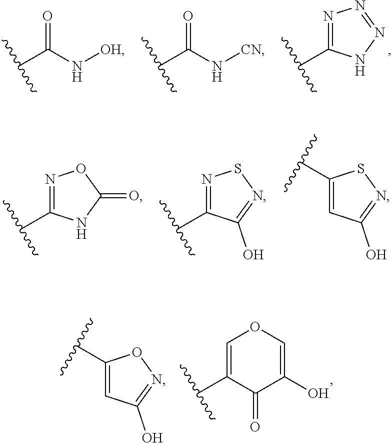

and the like.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

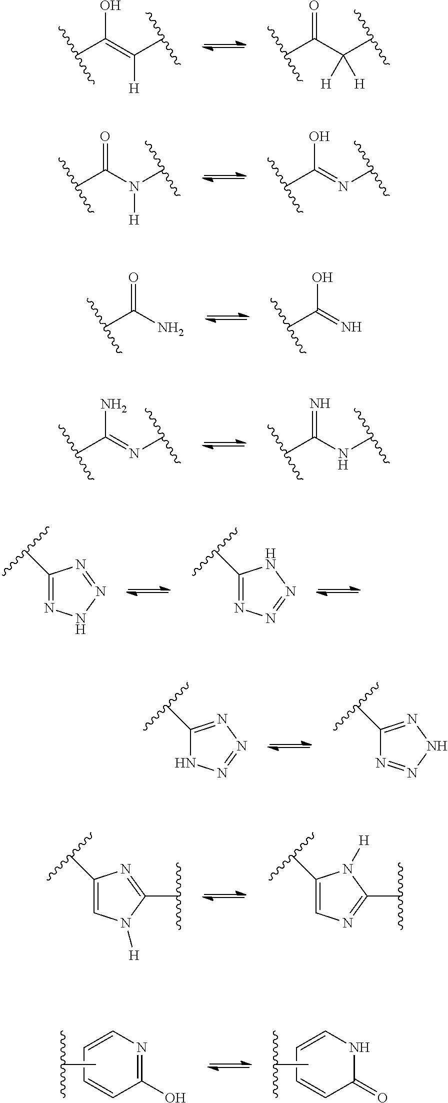

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

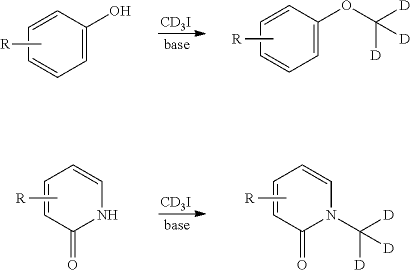

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

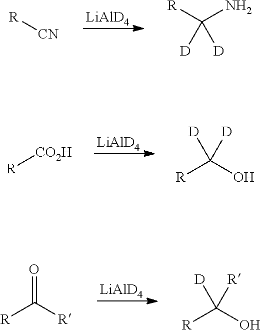

Deuterium gas and palladium catalyst are employed to reduce unsaturated carbon-carbon linkages and to perform a reductive substitution of aryl carbon-halogen bonds as illustrated, by way of example only, in the reaction schemes below.

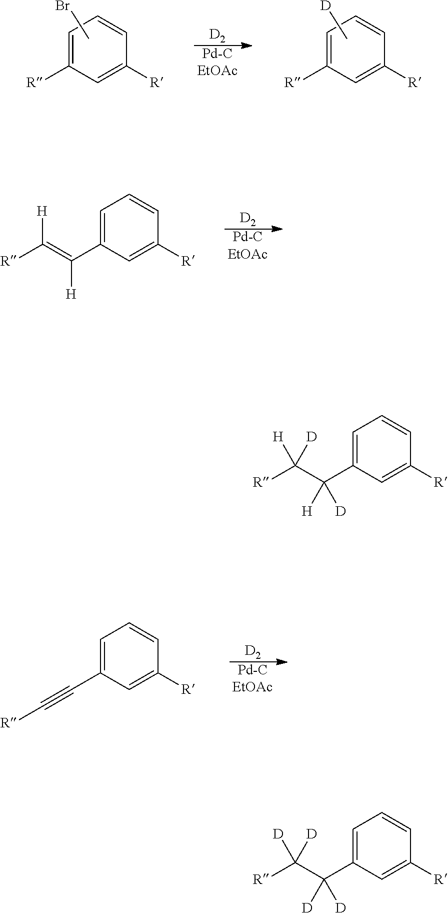

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the IGF-1R inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### IGF-1R Tyrosine Kinase

The type 1 insulin-like growth factor receptor (IGF-1R) is a transmembrane class II receptor tyrosine kinase (RTK), belonging to the insulin receptor family, that plays crucial roles in differentiation, cell growth and cell survival. Signaling through IGF-1R is the principal pathway responsible for somatic growth in fetal mammals, while somatic growth in postnatal animals is achieved through the synergistic interaction of growth hormone (GH) and Insulin-like growth factors (IGF1 and IGF2). IGF-1R expression is widespread among many different cell types. Granulated cytoplasmic protein expression appears ubiquitous in human cells and IGF-1R endocytosis and trafficking to specific subcellular locations during signaling defines the nature of particular signaling responses that are critical during normal and pathological cellular processes. Dysregulation of IGF-1R signaling and function has been implicated in human disorders, including cancers and growth retardation during development. IGF1 signaling continues to have anabolic effects during adulthood and this signaling pathway additionally affects the ageing process. Specific developmental functions for IGF-1R, such as regional-specific regulation of axon growth in medial areas of the forebrain including the hippocampus and cingulate cortex, have also been elucidated.

IGF-1R has been shown to play critical roles in cell transformation events. It is highly overexpressed in a diverse array of malignant tissues where it functions as an anti-apoptotic agent by enhancing cell survival. Elevated IGF-1R expression has been implicated in transformative roles in cancers of breast, ovarian, prostate, colon, and lung tissues as well as in rhabdomyosarcomas, melanomas, and gliomas.

The IGF-1R gene is located on chromosome 15q26.3. The IGF-1R gene contains 21 exons and spans about 100 kb. The promoter region of IGF-1R contains numerous potential SP1 and AP2 binding sites as well as a thyroid response element, but no TATA or CCAAT elements. It is expressed as multiple mRNA transcripts, the most abundant of which is 12 kb, followed by several shorter transcripts of 7 kb and 6.4 kb. In the 12 kb IGF-1R mRNA transcript, 1 kb is 5′-UTR, 4 kb is coding sequence and 7 kb is 3′-UTR. The protein product of this gene is the Insulin-like Growth Factor 1 (IGF-1) Receptor. An alternate human IGF-1R mRNA transcript can be expressed in which a three base pair (CAG) deletion results in the substitution of Arg for Thr898Gly899 eight residues upstream from the start of the transmembrane region of IGF-1R. This CAG-isoform shows reduced internalization and enhanced signaling properties compared to the CAG+ isoform.

Transcriptional regulation of IGF-1R is controlled by a complex interaction involving DNA-binding and non-DNA-binding transcription factors. Stimulatory nuclear proteins including zinc-finger protein Sp1, EWS-WT1, E2F1, Krüppel-like factor-6 (KLF6), and high-mobility group A1 (HMGA1) promote IGF-1R expression. A number of tumor suppressors, including the breast cancer gene-1 (BRCA1), p53, the Wilm's tumor protein-1 (WT1) and the von Hippel-Lindau gene (VHL) are also regulate the IGF-1R locus. Loss-of-function of tumor suppressors can derepress IGF-1R expression thereby leading to increased IGF signaling. This impaired regulation of IGF-1R has been linked to aberrant cell division, loss of apoptotic regulation, chromosomal instability, and increased incidence of cancer. The p53 gene, the most frequently mutated gene in human cancer, functions as a nuclear transcription factor that blocks cell cycle progression and induces apoptosis. Wild-type p53 serves to suppress transcriptional activation of the IGF-1R promoter, whereas mutant p53 can have a stimulatory effect on IGF-1R promoter activity. Due to a central role of insulin-like growth factor signaling in cell cycle progression and cell transformation, derepression of the IGF-1R promoter constitutes an important paradigm for tumorigenesis.

After translation, IGF-1R is 1,367-amino acid receptor precursor, including a 30-residue signal peptide, which is removed during translocation of the nascent polypeptide chain. Cleavage of the precursor generates alpha and beta subunits. Two alpha subunits and two beta subunits make up the IGF-1 receptor. Both the α and β subunits are synthesized from a single mRNA precursor. The precursor is then glycosylated, proteolytically cleaved, and crosslinked by cysteine bonds to form a functional transmembrane αβ chain. After transport to the plasma membrane, the two α chains are located extracellularly, while the β subunits span the membrane and conduct intracellular signal transduction upon ligand stimulation. The ectodomains of IGF-1R have an arrangement of two homologous domains (L1 and L2) separated by a Furin-like cysteine rich region. Each L domain (L1 spanning residues 1-150 and L2 spanning residues 300-460) consists of five and a half leucine-rich repeats and are members of the leucine-rich repeat superfamily. The C-terminal half of their ectodomains consists of three fibronectin type 3 repeats, and an insert domain which contains the α-β cleavage site. There is a single transmembrane sequence (residues 906-929) in IGF-1R. The cytoplasmic portion of IGF-1R consists of a tyrosine kinase catalytic domain flanked by a juxtamembrane and C-tail region, the sites of binding of various signaling molecules. The cytoplasmic domain containing the tyrosine kinase domain (residues 930-1337) spans 408 amino acid residues.

The major feature which separates IGF-1R and its related family members from most other receptor tyrosine kinase families is that they exist on the cell surface as constitutive disulfide-linked dimers and require domain rearrangements rather than receptor oligomerization for cell signaling. Recent studies on signal transduction suggest that ligand-triggered structural changes in the extracellular domain followed by transmembrane domains closure and dimerization lead to trans-autophosphorylation and kinase activity in the intracellular segments of IGF-1R. Ligand binding leads to conformational changes bringing the most distal of the fibronectin type 3 repeats in close proximity to each other followed by dimerization of transmembrane segments inside the lipid bilayer. In its basal state, one of the three tyrosines in the activation loop (A-loop), Tyr1162, is bound in the active site but cannot be phosphorylated in cis as part of the A-loop interferes with the ATP binding site and the catalytic Asp 1150 is not positioned properly to coordinate MgATP. Upon activation, autophosphorylation of Tyr1162, Tyr1158 and Tyr1163 occurs in trans by the kinase domain of the second monomer. Therefore, in the basal state, Tyr1162 competes with the neighboring β-chain, for binding to the active site, but is not cis-phosphorylated because of steric constraints that prevent simultaneous binding of Tyr1162 and MgATP. Autophosphorylation of the three tyrosines in the A-loop, leads to a dramatic change in configuration thereby activating the kinase domain.

Three ligands have been identified as mediating signaling through IGF-1R. These are Insulin Like Growth Factor (IGF1), Insulin Like Growth Factor 2 (IGF2) and insulin. IGF-1R binds its endogenous ligands with the following order of affinity: IGF1 with highest affinity, IGF2 with lower affinity, and insulin with weak affinity. The biological activities of IGF1 and IGF2 are modulated by a family of six IGF-binding proteins. These binding proteins regulate the transport and bioavailability of IGFs and as well as competing with IGFs for binding to IGF-1R. Two ligand-binding sites are present in the extracellular portion of each αβ dimer of IGF-1R. The IGF-1R extracellular domain is autoinhibitory and ligand binding releases this autoinhibition and brings the TM domains together to allow autophosphorylation and subsequent kinase domain activation. IGF2 is a primary growth factor required for early development whereas IGF1 is required for achieving maximal growth. Postnatally, IGF1 is mainly secreted by the liver in response to stimulation from GH, but can also be expressed by other cell types. IGF1 regulates normal physiology and is known to promote cancer progression by inhibiting apoptosis and stimulating cell proliferation. Unlike most growth factors, whose bioactivities are regulated primarily through their release from secretory granules, serum concentrations of both IGF1 and IGF2 in the circulation and tissues far exceed those needed for maximal cellular stimulation. Over 99% of the circulating IGFs are bound to IGFBPs, with most forming a 150-kDa complex with IGFBP-3 and the acid-labile subunit (ALS). This complex prolongs the serum half-life of IGF1 from about 10 minutes to 15 hours and helps to tightly regulate IGF bioavailability at the cellular level. Because the IGF binding affinity for IGFBPs is greater than that for IGF-1R, IGFBPs competitively inhibit IGF/IGF-1R binding and signaling. Local proteases can cleave IGFBPs into fragments with lower binding affinities, thereby releasing IGF for IGF-1R binding.

In leukemias and malignant solid tumors, the IGF pathway is subverted in numerous ways during cellular transformation and tumor metastasis. Genetic risk factors including those at influence the expression of IGF-1R, IGF1, IGF2 and IGFBPs contribute to the risk of developing tumors. As previously mentioned, the expression of IGF-1R is tightly regulated and is often derepressed due to loss of activity of various tumor suppressor pathways. Another type of indirect involvement of the IGF pathway in cancer progression deals with interactions between the IGF pathway and other hormones. Estrogens in breast cancer and androgens in prostate cancer have been shown to enhance IGF-1R signaling. IGF signaling also has a direct contribution to cancer progression in that the pathways activated involve both enhanced cell survival and proliferation, as well as the ability to escape from cell cycle arrests and apoptotic mechanisms that normally function to abort such aberrant cells.

### IGF-1R Activation and Intracellular Signaling Pathways

The lifecycle of a human cell is tightly regulated by intra- and extracellular signals, that together control cellular proliferation, senescence, and apoptosis. When the sum of growth stimulatory and inhibitory signals favors proliferation, the cell enters mitosis. For instance, circulating IGF1 and IGF2 bind to IGF-1R and trigger signal transduction cascades that leads to increased proliferation and enhanced survival of IGF-responsive cells. Such signaling is central to the processes of oncogenesis and involves downstream effector mechanisms to mediate the effect of signal transduction initiation.

Ligand binding to IGF-1R activates the receptor kinase, leading to receptor autophosphorylation, and tyrosine phosphorylation of multiple substrates. These substrates include the insulin-receptor substrates (IRS1/2), Src homology and Collagen (Shc) adaptor proteins and 14-3-3 proteins. Phosphorylation of IRS1 and IRS2 proteins lead to the activation of two main signaling pathways: the PI3K-AKT/PKB pathway and the Ras-MAPK pathway.

Activation of the MAPK pathway results in increased cellular proliferation, whereas activation of the PI3K pathway inhibits apoptosis and stimulates protein synthesis. Phosphorylated IRS1 can activate the 85 kDa regulatory subunit of PI3K (PIK3R1), leading to activation of several downstream substrates, including protein AKT/PKB. AKT phosphorylation can enhance protein synthesis through mTOR activation and triggers the antiapoptotic effects of IGF-1R through phosphorylation and inactivation of BAD (a pro-apoptotic member of the BCL2 family). In an alternative pathway for activation of the PI3K pathway, a different regulatory subunit of PI3K (PIK3R3) binds through its SH2 domain with IGR1R and the Insulin Receptor (INSR) in a kinase-dependent manner, providing a means through which these two receptors can modulate the PI3K pathway.

In parallel to PI3K-driven signaling, recruitment of Grb2/SOS by phosphorylated IRS1 or phosphorylated Shc family members leads to recruitment of Ras and activation of the ras-MAPK pathway. The ras/MAPK pathway has many demonstrated points of involvement in mediating mitogenic, differentiation and migratory signals. The mitogenic activity of IGF-1R is mediated through the Ras and PI3K-AKT pathways and results in the upregulation of cyclin DI and its binding partner CDK4. This leads to the phosphorylation of retinoblastoma protein, the release of E2F transcription factor, and expression of downstream target genes like cyclin E (a crucial regulator of entry into S phase). Other pathways involving cellular proliferation as also regulated by IGF-1R activation. IGF-1R pathway activation has been shown to downregulate cell cycle suppressors p27^{kip1}, p57^{kip2}, and PTEN.

In addition to these two main signaling pathways (PI3K-AKT/PKB and Ras-MAPK), IGF-1R also signals through the Janus kinase/signal transducer and activator of transcription pathway (JAK/STAT). Phosphorylation of JAK proteins can lead to phosphorylation and subsequent activation of signal transducers and activators of transcription (STAT) proteins. The JAK/STAT pathway activates gene transcription and may be responsible for the transforming activity. The particular activation of STAT3 has been demonstrated to be regularly involved in the transforming activity of IGF-1R. JNK kinases have also been shown to be activated by IGF-1R.

Further integration of signal transduction pathways is evidenced by the multiple ways in which the IGF-1R and epidermal growth factor receptor (EGFR) pathways interact. IGF-1R and EGFR directly associate with each other and can heterodimerize. IGF-1R and EGFR can also mediate the availability of ligands for each other. Indirect interactions between the IGF-1R and EGFR pathways involve utilization of shared G protein coupled receptors or other downstream signaling molecules.

It was once thought that when cell surface receptor tyrosine kinases are internalized, their signal transduction is terminated. However, it is now generally accepted that internalized receptors, including IGF-1R, may signal from endosomal and intracellular membrane compartments. In addition, they may also regulate gene transcription by translocating to the nucleus. Although the details are not all clear regarding the mechanisms which determine the subcellular localization of IGF-1R or its compartmentalization with other signaling proteins, it has been suggested that intracellular IGF-1R trafficking is regulated in a cell type-specific way and that cell-specific signals may influence the recruitment and activation of effector proteins. Therefore, cell-specific IGF-1R trafficking, compartmentalization and subcellular location may define how cells respond to extracellular stimuli.

### IGF-1R Inhibitors

Forced overexpression of IGF-1R results in the malignant transformation of cultured cells and elevated levels of IGF-1R are observed in a variety of human tumor types. Downregulation of IGF-1R levels can reverse the transformed phenotype of tumor cells and may render them sensitive to apoptosis in vivo.

Several kinase inhibitors and blocking monoclonal antibodies that inhibit ligand binding and signal transduction have been developed and been tested. Examples of human monoclonal antibodies that bind to IGF-1R include: cixutumumab, ganitumab, teprotumumab, figitumumab, dalotuzumab, and R1507. Several clinical trials, including one involving subjects with metastatic pancreatic cancer, demonstrated that ganitumab was largely ineffective at improving survival rates. Teprotumumab, sold under the brand name Tepezza, is another human monoclonal antibody that binds to IGF-1R. Tepezza has been approved for the treatment of thyroid eye disease (TED), an autoimmune disorder characterized by proptosis. For this condition, Tepezza has been shown to decrease inflammation, thereby preventing muscle and fat tissue remodeling, and thereby leading to prevention of tissue expansion behind the eye. Although Tepezza has been shown to be effective in treating TED, Phase I trials of teprotumumab in treating malignancies demonstrated little effectiveness. The fact that these monoclonal antibody inhibitors of IGF-1R have been largely unsuccessful in clinical trials could potentially be related to how IGF-1R internalization, subcellular location and signaling are controlled in normal and cancer cells.

Renewed attention to potential small molecule inhibitors of IGF-1R is reasonable given the clinical trial failures of antibodies targeted to IGF-1R as cancer therapeutics. One such small molecule inhibitor is OSI-906, a dual IGF-1R/INSR kinase inhibitor. OSI-906 potently and selectively inhibits autophosphorylation of both human IGF-1R and IR, displays in vitro antiproliferative effects in a variety of tumor cell lines and shows robust in vivo anti-tumor efficacy in an IGF-1R-driven xenograft model when administered orally once daily. Unfortunately, a Phase 3 study to test the effectiveness of OSI-906 (Linsitinib) in treating adrenocortical carcinoma resulted in a conclusion that Linsitinib did not increase overall survival. Effective therapeutic targeting of IGF-1R that results in improved cancer survival rates is currently a great unmet need.

### IGF-1R Kinase Inhibitory Compounds

In one aspect, provided herein is an IGF-1R inhibitory compound.

One embodiment provides a compound, or a pharmaceutically acceptable salt or solvate thereof, having the structure of Formula (I):

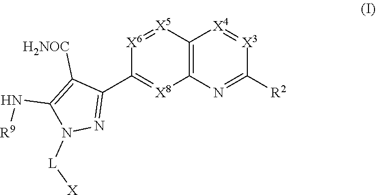

wherein,
   X is optionally substituted alkyl, optionally substituted carbocyclyl, optionally substituted carbocyclylalkyl, optionally substituted aryl, optionally substituted aralkyl, optionally substituted heteroaryl, optionally substituted heteroaralkyl, optionally substituted heterocyclyl, or optionally substituted heterocyclylalkyl;
   L is a bond, or optionally substituted carbocyclyl, optionally substituted carbocyclylalkyl, optionally substituted heterocyclyl, or optionally substituted heterocyclylalkyl;
   R² is optionally substituted carbocyclyl, optionally substituted aryl, optionally substituted heteroaryl, or optionally substituted heterocyclyl; wherein the optional substitution of the optionally substituted carbocyclyl, optionally substituted aryl, optionally substituted heteroaryl, or optionally substituted heterocyclyl is selected from the group consisting of cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted aryloxy, optionally substituted heteroaryloxy, optionally substituted (heterocyclyl)-O—, optionally substituted C1-C6 alkyl, optionally substituted C2-C6 alkynyl, optionally substituted carbocyclyl, optionally substituted C2-C6 alkenyl, optionally substituted aryl, optionally substituted heteroaryl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—N(R¹⁰)₂, or —NR¹¹SO₂—N(R¹⁰)₂;
   X³ is N or C—R³;
   X⁴ is N or C—R⁴;
   X⁵ is N or C—R⁵;
   X⁶ is N or C—R⁶;
   X⁸ is N or C—R⁸;
   R³, R⁴, R⁵, R⁶, and R⁸ are independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C2-C6 alkynyl, optionally substituted carbocyclyl, optionally substituted C2-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—N(R¹⁰)₂, and —NR¹¹SO₂—N(R¹⁰)₂;
   each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
   each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl;
   R¹² is H or optionally substituted C1-C6 alkyl;
   R⁹ is selected from hydrogen, optionally substituted C1-C6 alkyl, optionally substituted C3-C8 carbocyclyl, or optionally substituted C4-C10 carbocyclylalkyl; wherein either R⁹ and L, or R⁹ and X, may be joined together with any intervening atoms to form an optionally substituted heterocyclyl ring.

Another embodiment provides the compound of Formula (I), or pharmaceutically acceptable salt or solvate thereof, wherein X³ is N, and X⁴ is C—R⁴.

Another embodiment provides the compound of Formula (I), or pharmaceutically acceptable salt or solvate thereof, wherein X³ is C—R³, and X⁴ is N.

Another embodiment provides the compound of Formula (I), or pharmaceutically acceptable salt or solvate thereof, wherein X³ is C—R³, and X⁴ is C—R⁴.

Another embodiment provides the compound of Formula (I), or pharmaceutically acceptable salt or solvate thereof, wherein L is a bond. Another embodiment provides the compound of Formula (I), or pharmaceutically acceptable salt or solvate thereof, wherein X is optionally substituted C3-C7 cycloalkyl. Another embodiment provides the compound of Formula (I), or pharmaceutically acceptable salt or solvate thereof, wherein X is optionally substituted C4 cycloalkyl. Another embodiment provides the compound of Formula (I), or pharmaceutically acceptable salt or solvate thereof, wherein X is optionally substituted heterocyclyl. Another embodiment provides the compound of Formula (I), or pharmaceutically acceptable salt or solvate thereof, wherein X is optionally substituted piperidine or pyrrolidine. Another embodiment provides the compound of Formula (I), or pharmaceutically acceptable salt or solvate thereof, wherein X is optionally substituted piperidin-4-yl. Another embodiment provides the compound of Formula (I), or pharmaceutically acceptable salt or solvate thereof, wherein X is optionally substituted C1-C8 alkyl.

Another embodiment provides the compound of Formula (I), or pharmaceutically acceptable salt or solvate thereof, wherein L is an optionally substituted cycloalkyl. Another embodiment provides the compound of Formula (I), or pharmaceutically acceptable salt or solvate thereof, wherein X is optionally substituted C3-C7 cycloalkyl. Another embodiment provides the compound of Formula (I), or pharmaceutically acceptable salt or solvate thereof, wherein X is optionally substituted heterocyclyl. Another embodiment provides the compound of Formula (I), or pharmaceutically acceptable salt or solvate thereof, wherein X is optionally substituted C1-C8 alkyl. Another embodiment provides the compound of Formula (I), or pharmaceutically acceptable salt or solvate thereof, wherein X is optionally substituted C4-C10 cycloalkylalkyl. Another embodiment provides the compound of Formula (I), or pharmaceutically acceptable salt or solvate thereof, wherein X is optionally substituted heterocyclylalkyl.

Another embodiment provides the compound of Formula (I), or pharmaceutically acceptable salt or solvate thereof, wherein R³ is H.

Another embodiment provides the compound of Formula (I), or pharmaceutically acceptable salt or solvate thereof, wherein R⁴ is H. Another embodiment provides the compound of Formula (I), or pharmaceutically acceptable salt or solvate thereof, wherein R⁴ is optionally substituted C1-C4 alkoxy.

Another embodiment provides the compound of Formula (I), or pharmaceutically acceptable salt or solvate thereof, wherein X⁸ is N.

Another embodiment provides the compound of Formula (I), or pharmaceutically acceptable salt or solvate thereof, wherein X⁸ is C—R⁸. Another embodiment provides the compound, or pharmaceutically acceptable salt or solvate thereof, wherein R⁸ is H. Another embodiment provides the compound, or pharmaceutically acceptable salt or solvate thereof, wherein R⁸ is halogen. Another embodiment provides the compound, or pharmaceutically acceptable salt or solvate thereof, wherein R⁸ is F.

Another embodiment provides the compound of Formula (I), or pharmaceutically acceptable salt or solvate thereof, wherein R⁹ is H.

Another embodiment provides the compound of Formula (I), or pharmaceutically acceptable salt or solvate thereof, wherein R² is optionally substituted aryl.

Another embodiment provides the compound of Formula (I), or pharmaceutically acceptable salt or solvate thereof, wherein R² is optionally substituted phenyl. Another embodiment provides the compound, or pharmaceutically acceptable salt or solvate thereof, wherein R² is phenyl substituted with at least one halogen. Another embodiment provides the compound, or pharmaceutically acceptable salt or solvate thereof, wherein R² is 2-fluorophenyl.

Another embodiment provides the compound of Formula (I), or pharmaceutically acceptable salt or solvate thereof, wherein R² is optionally substituted heteroaryl. Another embodiment provides the compound of Formula (I), or pharmaceutically acceptable salt or solvate thereof, wherein R² is optionally substituted pyridine.

Another embodiment provides the compound of Formula (I), or pharmaceutically acceptable salt or solvate thereof, wherein X⁶ is N.

Another embodiment provides the compound of Formula (I), or pharmaceutically acceptable salt or solvate thereof, wherein X⁶ is C—R⁶. Another embodiment provides the compound, or pharmaceutically acceptable salt or solvate thereof, wherein R⁶ is H.

Another embodiment provides the compound of Formula (I), or pharmaceutically acceptable salt or solvate thereof, wherein X⁵ is N.

Another embodiment provides the compound of Formula (I), or pharmaceutically acceptable salt or solvate thereof, wherein X⁵ is C—R⁵. Another embodiment provides the compound, or pharmaceutically acceptable salt or solvate thereof, wherein R⁵ is H.

Another embodiment provides the compound of Formula (I), or pharmaceutically acceptable salt or solvate thereof, wherein X⁵ is C—R⁵ and X⁶ is C—R⁶. Another embodiment provides the compound of Formula (I), or pharmaceutically acceptable salt or solvate thereof, wherein X⁵ is C-Hand X⁶ is C—H.

Another embodiment provides the compound of Formula (I), or pharmaceutically acceptable salt or solvate thereof, wherein X⁵ is C—R⁵, X⁶ is C—R⁶ and X⁸ is C—R⁸. Another embodiment provides the compound of Formula (I), or pharmaceutically acceptable salt or solvate thereof, wherein X⁵ is C—H, X⁶ is C—H, and X⁸ is C—F.

Another embodiment provides the compound of Formula (I), or pharmaceutically acceptable salt or solvate thereof, wherein X³ is C—R³, and X⁴ is C—R⁴. Another embodiment provides the compound of Formula (I), or pharmaceutically acceptable salt or solvate thereof, wherein X³ is C—H, and X⁴ is C—R⁴, wherein R⁴ is optionally substituted C1-C4 alkoxy. Another embodiment provides the compound of Formula (I), or pharmaceutically acceptable salt or solvate thereof, wherein X³ is C—H, and X⁴ is C—OCH₃.

Another embodiment provides the compound of Formula (I), or pharmaceutically acceptable salt or solvate thereof, wherein X³ is C—R³, X⁴ is C—R⁴, X⁵ is C—R⁵, X⁶ is C—R⁶ and X⁸ is C—R⁸. Another embodiment provides the compound of Formula (I), or pharmaceutically acceptable salt or solvate thereof, wherein X³ is C—H, X⁴ is C—OCH₃, X⁵ is C—H, X⁶ is C—H, and X⁸ is C—F.

Another embodiment provides the compound of Formula (I), or pharmaceutically acceptable salt or solvate thereof, wherein R² is phenyl substituted with at least one halogen, X³ is C—R³, X⁴ is C—R⁴, X⁵ is C—R⁵, X⁶ is C—R⁶ and X⁸ is C—R⁸. Another embodiment provides the compound of Formula (I), or pharmaceutically acceptable salt or solvate thereof, wherein R² is phenyl substituted with at least one halogen, X³ is C—H, X⁴ is C—OCH₃, X⁵ is C—H, X⁶ is C—H, and X⁸ is C—F. Another embodiment provides the compound, or pharmaceutically acceptable salt or solvate thereof, wherein R² is 2-fluorophenyl.

Another embodiment provides the compound of Formula (I), or pharmaceutically acceptable salt or solvate thereof, wherein -L-X is

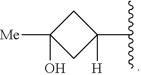 [SMILES: *C1CC(C)(O)C1]

One embodiment provides an IGF-1R inhibitory compound, or a pharmaceutically acceptable salt or solvate thereof, having a structure presented in Table 1A.

TABLE 1A

| Chemical | | |
|---|---|---|
| Synthesis | | |
| Example | Structure | Chemical Name |
| 1 | 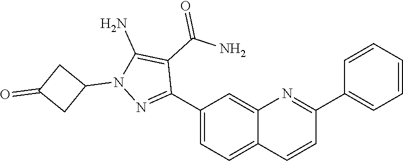 | 5-amino-1-(3-oxocyclobutyl)-3- (2-phenylquinolin-7-yl)-1H- pyrazole-4-carboxamide |
| 2 | 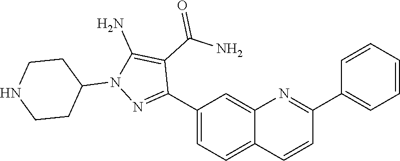 [SMILES: NC(=O)c1c(-c2ccc3ccc(-c4ccccc4)nc3c2)nn(C2CCNCC2)c1N] | 5-amino-3-(2-phenylquinolin-7- yl)-1-(piperidin-4-yl)-1H- pyrazole-4-carboxamide |
| 3 | 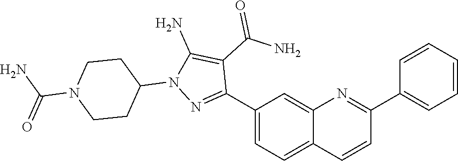 [SMILES: NC(=O)c1c(-c2ccc3ccc(-c4ccccc4)nc3c2)nn(C2CCN(C(N)=O)CC2)c1N] | 4-(5-amino-4-carbamoyl-3-(2- phenylquinolin-7-yl)-1H- pyrazol-1-yl)piperidine-1- carboxamide |
| 4 | 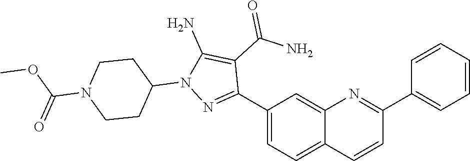 [SMILES: COC(=O)N1CCC(n2nc(-c3ccc4ccc(-c5ccccc5)nc4c3)c(C(N)=O)c2N)CC1] | methyl 4-(5-amino-4- carbamoyl-3-(2-phenylquinolin- 7-yl)-1H-pyrazol-1- yl)piperidine-1-carboxylate |
| 5 | 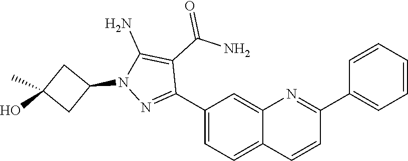 [SMILES: C[C@]1(O)C[C@@H](n2nc(-c3ccc4ccc(-c5ccccc5)nc4c3)c(C(N)=O)c2N)C1] | 5-amino-1-((1s,3s)-3-hydroxy- 3-methylcyclobutyl)-3-(2- phenylquinolin-7-yl)-1H- pyrazole-4-carboxamide |
| 6 | 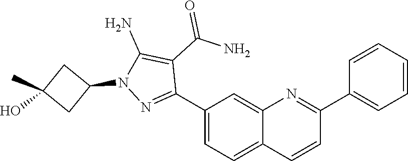 [SMILES: C[C@]1(O)C[C@H](n2nc(-c3ccc4ccc(-c5ccccc5)nc4c3)c(C(N)=O)c2N)C1] | 5-amino-1-((1r,3r)-3-hydroxy- 3-methylcyclobutyl)-3-(2- phenylquinolin-7-yl)-1H- pyrazole-4-carboxamide |
| 7 | 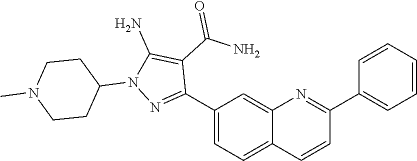 [SMILES: CN1CCC(n2nc(-c3ccc4ccc(-c5ccccc5)nc4c3)c(C(N)=O)c2N)CC1] | 5-amino-1-(1-methylpiperidin- 4-yl)-3-(2-phenylquinolin-7-yl)- 1H-pyrazole-4-carboxamide |
| 8 | 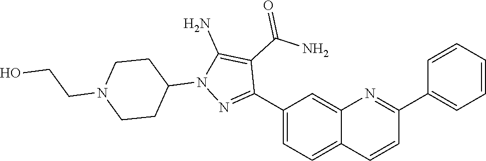 [SMILES: NC(=O)c1c(-c2ccc3ccc(-c4ccccc4)nc3c2)nn(C2CCN(CCO)CC2)c1N] | 5-amino-1-(1-(2- hydroxyethyl)piperidin-4-yl)-3- (2-phenylquinolin-7-yl)-1H- pyrazole-4-carboxamide |
| 9 | 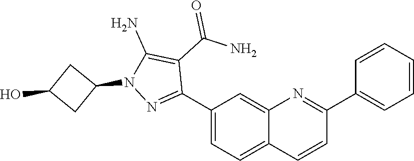 [SMILES: NC(=O)c1c(-c2ccc3ccc(-c4ccccc4)nc3c2)nn([C@H]2C[C@@H](O)C2)c1N] | 5-amino-1-((1s,3s)-3- hydroxycyclobutyl)-3-(2- phenylquinolin-7-yl)-1H- pyrazole-4-carboxamide |
| 10 | 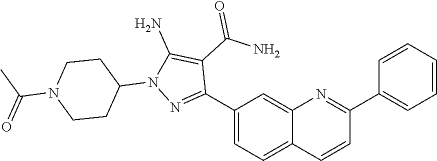 [SMILES: CC(=O)N1CCC(n2nc(-c3ccc4ccc(-c5ccccc5)nc4c3)c(C(N)=O)c2N)CC1] | 1-(1-acetylpiperidin-4-yl)-5- amino-3-(2-phenylquinolin-7- yl)-1H-pyrazole-4-carboxamide |
| 11 | 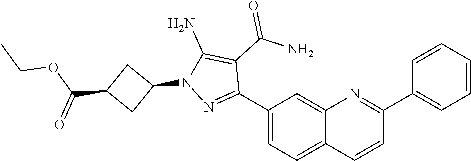 [SMILES: CCOC(=O)[C@H]1C[C@@H](n2nc(-c3ccc4ccc(-c5ccccc5)nc4c3)c(C(N)=O)c2N)C1] | ethyl (1s,3s)-3-(5-amino-4- carbamoyl-3-(2-phenylquinolin- 7-yl)-1H-pyrazol-1- yl)cyclobutane-1-carboxylate |
| 12 | 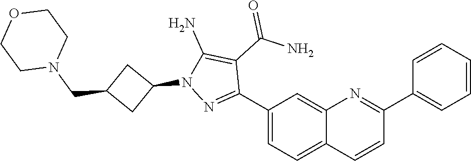 [SMILES: NC(=O)c1c(-c2ccc3ccc(-c4ccccc4)nc3c2)nn([C@H]2C[C@@H](CN3CCOCC3)C2)c1N] | 5-amino-1-((1s,3s)-3- (morpholinomethyl)cyclobutyl)- 3-(2-phenylquinolin-7-yl)-1H- pyrazole-4-carboxamide |
| 13 | 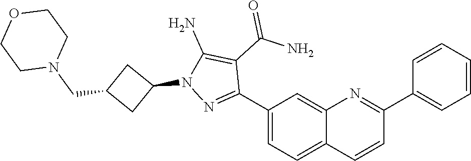 [SMILES: NC(=O)c1c(-c2ccc3ccc(-c4ccccc4)nc3c2)nn([C@H]2C[C@H](CN3CCOCC3)C2)c1N] | 5-amino-1-((1r,3r)-3- (morpholinomethyl)cyclobutyl)- 3-(2-phenylquinolin-7-yl)-1H- pyrazole-4-carboxamide |
| 14 | 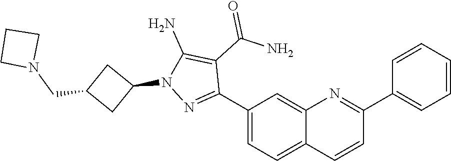 [SMILES: NC(=O)c1c(-c2ccc3ccc(-c4ccccc4)nc3c2)nn([C@H]2C[C@H](CN3CCC3)C2)c1N] | 5-amino-1-((1r,3r)-3-(azetidin- 1-ylmethyl)cyclobutyl)-3-(2- phenylquinolin-7-yl)-1H- pyrazole-4-carboxamide |
| 15 | 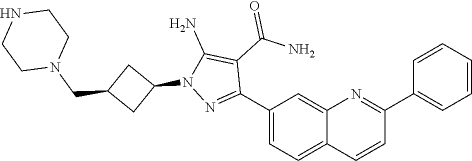 [SMILES: NC(=O)c1c(-c2ccc3ccc(-c4ccccc4)nc3c2)nn([C@H]2C[C@@H](CN3CCNCC3)C2)c1N] | 5-amino-3-(2-phenylquinolin-7- yl)-1-((1s,3s)-3-(piperazin-1- ylmethyl)cyclobutyl)-1H- pyrazole-4-carboxamide |
| 16 | 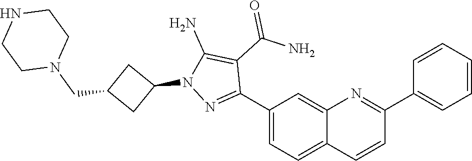 [SMILES: NC(=O)c1c(-c2ccc3ccc(-c4ccccc4)nc3c2)nn([C@H]2C[C@H](CN3CCNCC3)C2)c1N] | 5-amino-3-(2-phenylquinolin-7- yl)-1-((1r,3r)-3-(piperazin-1- ylmethyl)cyclobutyl)-1H- pyrazole-4-carboxamide |
| 17 | 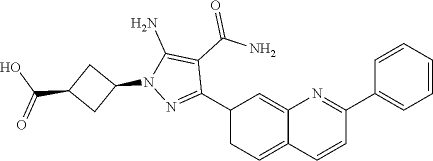 [SMILES: NC(=O)c1c(C2C=c3nc(-c4ccccc4)ccc3=CC2)nn([C@H]2C[C@@H](C(=O)O)C2)c1N] | (1s,3s)-3-(5-amino-4- carbamoyl-3-(2-phenylquinolin- 7-yl)-1H-pyrazol-1- yl)cyclobutane-1-carboxylic acid |
| 18 | 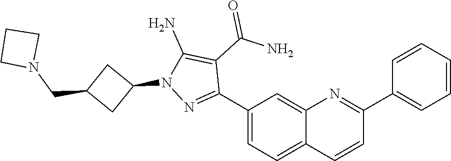 [SMILES: NC(=O)c1c(-c2ccc3ccc(-c4ccccc4)nc3c2)nn([C@H]2C[C@@H](CN3CCC3)C2)c1N] | 5-amino-1-((1s,3s)-3-(azetidin- 1-ylmethyl)cyclobutyl)-3-(2- phenylquinolin-7-yl)-1H- pyrazole-4-carboxamide |
| 19 | 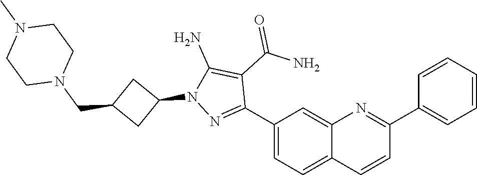 [SMILES: CN1CCN(C[C@H]2C[C@@H](n3nc(-c4ccc5ccc(-c6ccccc6)nc5c4)c(C(N)=O)c3N)C2)CC1] | 5-amino-1-((1s,3s)-3-((4- methylpiperazin-1- yl)methyl)cyclobutyl)-3-(2- phenylquinolin-7-yl)-1H- pyrazole-4-carboxamide |
| 20 | 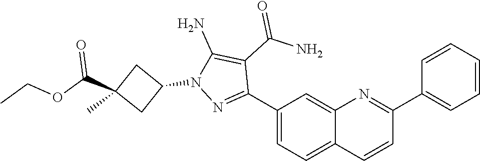 [SMILES: CCOC(=O)[C@]1(C)C[C@@H](n2nc(-c3ccc4ccc(-c5ccccc5)nc4c3)c(C(N)=O)c2N)C1] | ethyl (1r,3r)-3-(5-amino-4- carbamoyl-3-(2-phenylquinolin- 7-yl)-1H-pyrazol-1-yl)-1- methylcyclobutane-1- carboxylate |
| 21 | 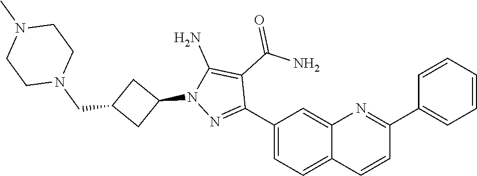 [SMILES: CN1CCN(C[C@H]2C[C@H](n3nc(-c4ccc5ccc(-c6ccccc6)nc5c4)c(C(N)=O)c3N)C2)CC1] | 5-amino-1-((1r,3r)-3-((4- methylpiperazin-1- yl)methyl)cyclobutyl)-3-(2- phenylquinolin-7-yl)-1H- pyrazole-4-carboxamide |
| 22 | 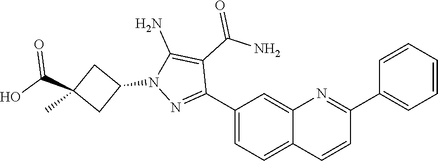 [SMILES: C[C@]1(C(=O)O)C[C@H](n2nc(-c3ccc4ccc(-c5ccccc5)nc4c3)c(C(N)=O)c2N)C1] | (1r,3r)-3-(5-amino-4- carbamoyl-3-(2-phenylquinolin- 7-yl)-1H-pyrazol-1-yl)-1- methylcyclobutane-1-carboxylic acid |
| 23 | 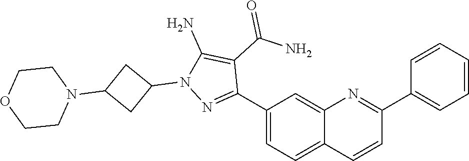 [SMILES: NC(=O)c1c(-c2ccc3ccc(-c4ccccc4)nc3c2)nn(C2CC(N3CCOCC3)C2)c1N] | 5-amino-1-(3- morpholinocyclobutyl)-3-(2- phenylquinolin-7-yl)-1H- pyrazole-4-carboxamide |
| 24 | 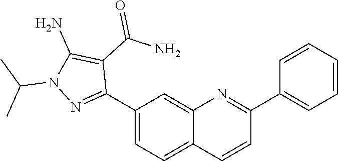 [SMILES: CC(C)n1nc(-c2ccc3ccc(-c4ccccc4)nc3c2)c(C(N)=O)c1N] | 5-amino-1-isopropyl-3-(2- phenylquinolin-7-yl)-1H- pyrazole-4-carboxamide |
| 25 | 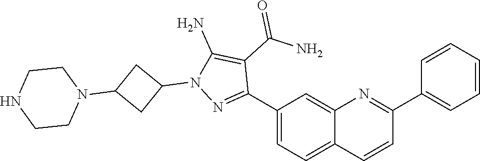 [SMILES: NC(=O)c1c(-c2ccc3ccc(-c4ccccc4)nc3c2)nn(C2CC(N3CCNCC3)C2)c1N] | 5-amino-3-(2-phenylquinolin-7- yl)-1-(3-(piperazin-1- yl)cyclobutyl)-1H-pyrazole-4- carboxamide |
| 26 | 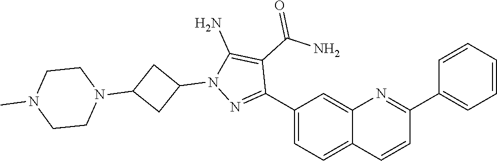 [SMILES: CN1CCN(C2CC(n3nc(-c4ccc5ccc(-c6ccccc6)nc5c4)c(C(N)=O)c3N)C2)CC1] | 5-amino-1-(3-(4- methylpiperazin-1- yl)cyclobutyl)-3-(2- phenylquinolin-7-yl)-1H- pyrazole-4-carboxamide |
| 27 | 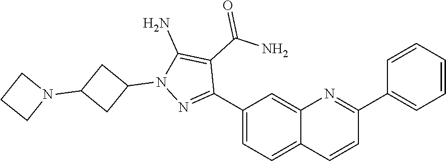 [SMILES: NC(=O)c1c(-c2ccc3ccc(-c4ccccc4)nc3c2)nn(C2CC(N3CCC3)C2)c1N] | 5-amino-1-(3-(azetidin-1- yl)cyclobutyl)-3-(2- phenylquinolin-7-yl)-1H- pyrazole-4-carboxamide |
| 28 | 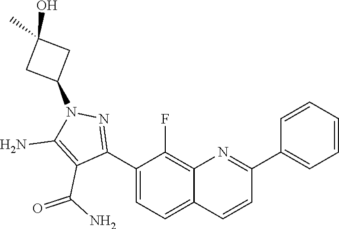 [SMILES: C[C@]1(O)C[C@@H](n2nc(-c3ccc4ccc(-c5ccccc5)nc4c3F)c(C(N)=O)c2N)C1] | 5-amino-3-(8-fluoro-2- phenylquinolin-7-yl)-1-((1s,3s)- 3-hydroxy-3-methylcyclobutyl)- 1H-pyrazole-4-carboxamide |
| 29 | 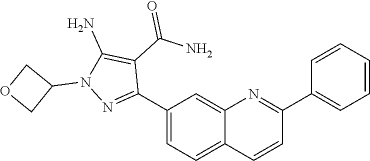 [SMILES: NC(=O)c1c(-c2ccc3ccc(-c4ccccc4)nc3c2)nn(C2COC2)c1N] | 5-amino-1-(oxetan-3-yl)-3-(2- phenylquinolin-7-yl)-1H- pyrazole-4-carboxamide |
| 30 | 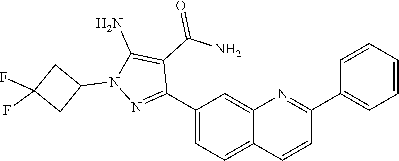 [SMILES: NC(=O)c1c(-c2ccc3ccc(-c4ccccc4)nc3c2)nn(C2CC(F)(F)C2)c1N] | 5-amino-1-(3,3- difluorocyclobutyl)-3-(2- phenylquinolin-7-yl)-1H- pyrazole-4-carboxamide |
| 31 | 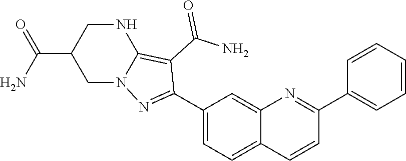 [SMILES: NC(=O)c1c(-c2ccc3ccc(-c4ccccc4)nc3c2)nn2c1NCC(C(N)=O)C2] | 2-(2-phenylquinolin-7-yl)- 4,5,6,7-tetrahydropyrazolo[1,5- a]pyrimidine-3,6- dicarboxamide |
| 32 | 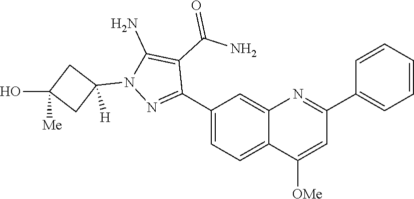 [SMILES: COc1cc(-c2ccccc2)nc2cc(-c3nn([C@H]4C[C@@](C)(O)C4)c(N)c3C(N)=O)ccc12] | 5-amino-1-((1s,3s)-3-hydroxy- 3-methylcyclobutyl)-3-(4- methoxy-2-phenylquinolin-7- yl)-1H-pyrazole-4-carboxamide |
| 33 | 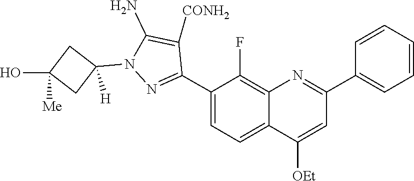 [SMILES: CCOc1cc(-c2ccccc2)nc2c(F)c(-c3nn([C@H]4C[C@@](C)(O)C4)c(N)c3C(N)=O)ccc12] | 5-amino-3-(4-ethoxy-8-fluoro- 2-phenylquinolin-7-yl)-1- ((1s,3s)-3-hydroxy-3- methylcyclobutyl)-1H-pyrazole- 4-carboxamide |
| 34 | 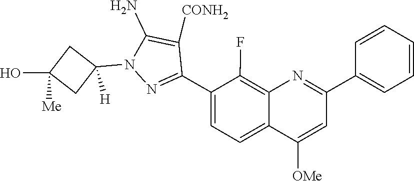 [SMILES: COc1cc(-c2ccccc2)nc2c(F)c(-c3nn([C@H]4C[C@@](C)(O)C4)c(N)c3C(N)=O)ccc12] | 5-amino-3-(8-fluoro-4- methoxy-2-phenylquinolin-7- yl)-1-((1s,3s)-3-hydroxy-3- methylcyclobutyl)-1H-pyrazole- 4-carboxamide |
| 35 | 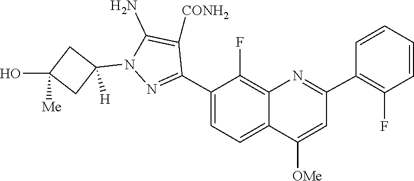 [SMILES: COc1cc(-c2ccccc2F)nc2c(F)c(-c3nn([C@H]4C[C@@](C)(O)C4)c(N)c3C(N)=O)ccc12] | 5-amino-3-(8-fluoro-2-(2- fluorophenyl)-4- methoxyquinolin-7-yl)-1- ((1s,3s)-3-hydroxy-3- methylcyclobutyl)-1H-pyrazole- 4-carboxamide |
| 36 | 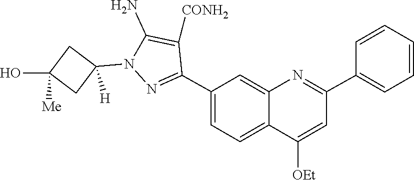 [SMILES: CCOc1cc(-c2ccccc2)nc2cc(-c3nn([C@H]4C[C@@](C)(O)C4)c(N)c3C(N)=O)ccc12] | 5-amino-3-(4-ethoxy-2- phenylquinolin-7-yl)-1-((1s,3s)- 3-hydroxy-3-methylcyclobutyl)- 1H-pyrazole-4-carboxamide |
| 37 | 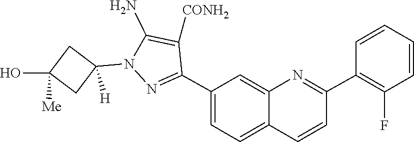 [SMILES: C[C@]1(O)C[C@@H](n2nc(-c3ccc4ccc(-c5ccccc5F)nc4c3)c(C(N)=O)c2N)C1] | 5-amino-3-(2-(2- fluorophenyl)quinolin-7-yl)-1- ((1s,3s)-3-hydroxy-3- methylcyclobutyl)-1H-pyrazole- 4-carboxamide |
| 38 | 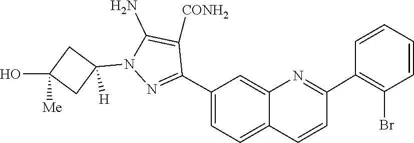 [SMILES: C[C@]1(O)C[C@@H](n2nc(-c3ccc4ccc(-c5ccccc5Br)nc4c3)c(C(N)=O)c2N)C1] | 5-amino-3-(2-(2- bromophenyl)quinolin-7-yl)-1- ((1s,3s)-3-hydroxy-3- methylcyclobutyl)-1H-pyrazole- 4-carboxamide |
| 39 | 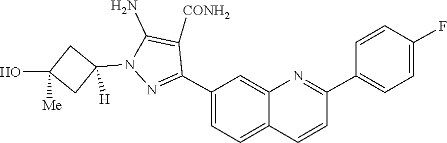 [SMILES: C[C@]1(O)C[C@@H](n2nc(-c3ccc4ccc(-c5ccc(F)cc5)nc4c3)c(C(N)=O)c2N)C1] | 5-amino-3-(2-(4- fluorophenyl)quinolin-7-yl)-1- ((1s,3s)-3-hydroxy-3- methylcyclobutyl)-1H-pyrazole- 4-carboxamide |
| 40 | 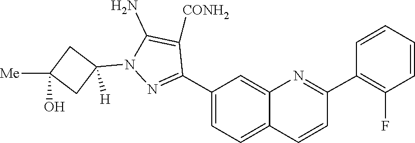 [SMILES: C[C@]1(O)C[C@H](n2nc(-c3ccc4ccc(-c5ccccc5F)nc4c3)c(C(N)=O)c2N)C1] | 5-amino-3-(2-(2- fluorophenyl)quinolin-7-yl)-1- ((1r,3r)-3-hydroxy-3- methylcyclobutyl)-1H-pyrazole- 4-carboxamide |
| 41 | 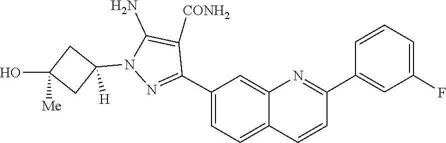 [SMILES: C[C@]1(O)C[C@@H](n2nc(-c3ccc4ccc(-c5cccc(F)c5)nc4c3)c(C(N)=O)c2N)C1] | 5-amino-3-(2-(3- fluorophenyl)quinolin-7-yl)-1- ((1s,3s)-3-hydroxy-3- methylcyclobutyl)-1H-pyrazole- 4-carboxamide |
| 42 | 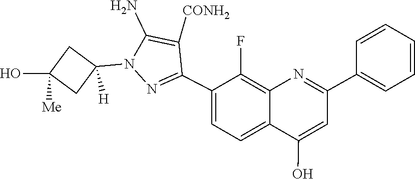 [SMILES: C[C@]1(O)C[C@@H](n2nc(-c3ccc4c(O)cc(-c5ccccc5)nc4c3F)c(C(N)=O)c2N)C1] | 5-amino-3-(8-fluoro-4-hydroxy- 2-phenylquinolin-7-yl)-1- ((1s,3s)-3-hydroxy-3- methylcyclobutyl)-1H-pyrazole- 4-carboxamide |
| 43 | 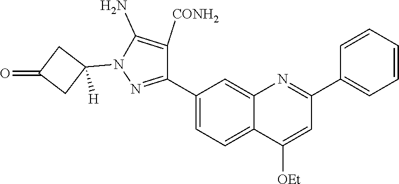 | 5-amino-3-(4-ethoxy-2- phenylquinolin-7-yl)-1-(3- oxocyclobutyl)-1H-pyrazole-4- carboxamide |
| 44 | 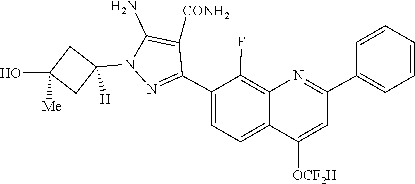 | 5-amino-3-(4- (difluoromethoxy)-8-fluoro-2- phenylquinolin-7-yl)-1-((1s,3s)- 3-hydroxy-3-methylcyclobutyl)- 1H-pyrazole-4-carboxamide |
| 45 | 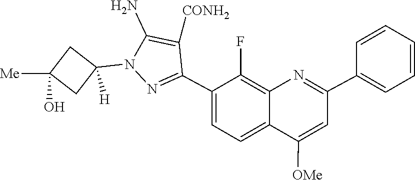 [SMILES: COc1cc(-c2ccccc2)nc2c(F)c(-c3nn([C@H]4C[C@](C)(O)C4)c(N)c3C(N)=O)ccc12] | 5-amino-3-(8-fluoro-4- methoxy-2-phenylquinolin-7- yl)-1-((1r,3r)-3-hydroxy-3- methylcyclobutyl)-1H-pyrazole- 4-carboxamide |
| 46 | 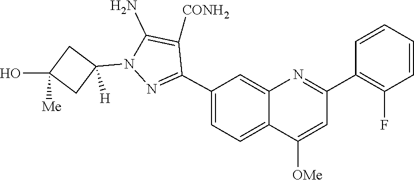 [SMILES: COc1cc(-c2ccccc2F)nc2cc(-c3nn([C@H]4C[C@@](C)(O)C4)c(N)c3C(N)=O)ccc12] | 5-amino-3-(2-(2-fluorophenyl)- 4-methoxyquinolin-7-yl)-1- ((1s,3s)-3-hydroxy-3- methylcyclobutyl)-1H-pyrazole- 4-carboxamide |
| 47 | 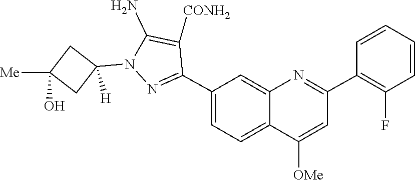 [SMILES: COc1cc(-c2ccccc2F)nc2cc(-c3nn([C@H]4C[C@](C)(O)C4)c(N)c3C(N)=O)ccc12] | 5-amino-3-(2-(2-fluorophenyl)- 4-methoxyquinolin-7-yl)-1- ((1r,3r)-3-hydroxy-3- methylcyclobutyl)-1H-pyrazole- 4-carboxamide |
| 48 | 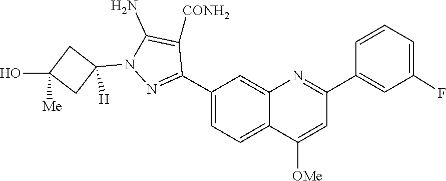 [SMILES: COc1cc(-c2cccc(F)c2)nc2cc(-c3nn([C@H]4C[C@@](C)(O)C4)c(N)c3C(N)=O)ccc12] | 5-amino-3-(2-(3-fluorophenyl)- 4-methoxyquinolin-7-yl)-1- ((1s,3s)-3-hydroxy-3- methylcyclobutyl)-1H-pyrazole- 4-carboxamide |
| 49 | 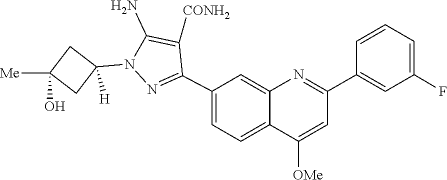 [SMILES: COc1cc(-c2cccc(F)c2)nc2cc(-c3nn([C@H]4C[C@](C)(O)C4)c(N)c3C(N)=O)ccc12] | 5-amino-3-(2-(3-fluorophenyl)- 4-methoxyquinolin-7-yl)-1- ((1r,3r)-3-hydroxy-3- methylcyclobutyl)-1H-pyrazole- 4-carboxamide |
| 50 | 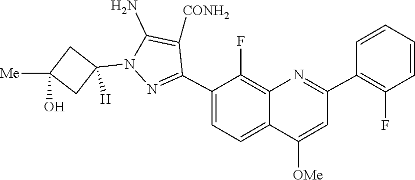 [SMILES: COc1cc(-c2ccccc2F)nc2c(F)c(-c3nn([C@H]4C[C@](C)(O)C4)c(N)c3C(N)=O)ccc12] | 5-amino-3-(8-fluoro-2-(2- fluorophenyl)-4- methoxyquinolin-7-yl)-1- ((1r,3r)-3-hydroxy-3- methylcyclobutyl)-1H-pyrazole- 4-carboxamide |
| 51 | 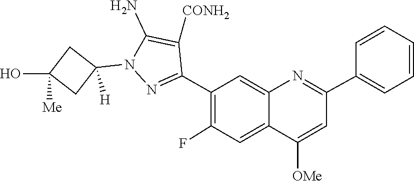 [SMILES: COc1cc(-c2ccccc2)nc2cc(-c3nn([C@H]4C[C@@](C)(O)C4)c(N)c3C(N)=O)c(F)cc12] | 5-amino-3-(6-fluoro-4- methoxy-2-phenylquinolin-7- yl)-1-((1s,3s)-3-hydroxy-3- methylcyclobutyl)-1H-pyrazole- 4-carboxamide |
| 52 | 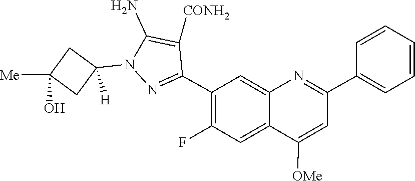 [SMILES: COc1cc(-c2ccccc2)nc2cc(-c3nn([C@H]4C[C@](C)(O)C4)c(N)c3C(N)=O)c(F)cc12] | 5-amino-3-(6-fluoro-4- methoxy-2-phenylquinolin-7- yl)-1-((1r,3r)-3-hydroxy-3- methylcyclobutyl)-1H-pyrazole- 4-carboxamide |
| 53 | 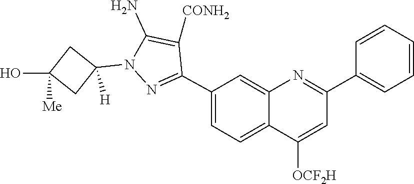 | 5-amino-3-(4- (difluoromethoxy)-2- phenylquinolin-7-yl)-1-((1s,3s)- 3-hydroxy-3-methylcyclobutyl)- 1H-pyrazole-4-carboxamide |
| 54 | 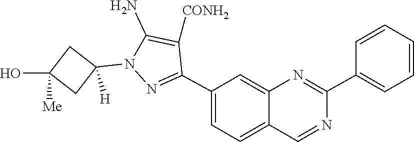 [SMILES: C[C@]1(O)C[C@@H](n2nc(-c3ccc4cnc(-c5ccccc5)nc4c3)c(C(N)=O)c2N)C1] | 5-amino-1-((1s,3s)-3-hydroxy- 3-methylcyclobutyl)-3-(2- phenylquinazolin-7-yl)-1H- pyrazole-4-carboxamide |
| 55 | 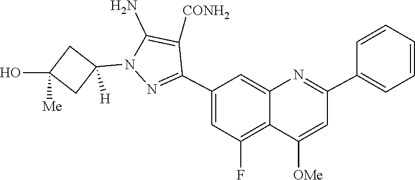 [SMILES: COc1cc(-c2ccccc2)nc2cc(-c3nn([C@H]4C[C@@](C)(O)C4)c(N)c3C(N)=O)cc(F)c12] | 5-amino-3-(5-fluoro-4- methoxy-2-phenylquinolin-7- yl)-1-((1s,3s)-3-hydroxy-3- methylcyclobutyl)-1H- pyrazole-4-carboxamide |
| 56 | 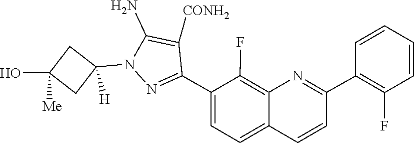 [SMILES: C[C@]1(O)C[C@@H](n2nc(-c3ccc4ccc(-c5ccccc5F)nc4c3F)c(C(N)=O)c2N)C1] | 5-amino-3-(8-fluoro-2-(2- fluorophenyl)quinolin-7-yl)-1- ((1s,3s)-3-hydroxy-3- methylcyclobutyl)-1H- pyrazole-4-carboxamide |
| 57 | 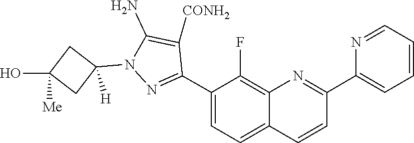 [SMILES: C[C@]1(O)C[C@@H](n2nc(-c3ccc4ccc(-c5ccccn5)nc4c3F)c(C(N)=O)c2N)C1] | 5-amino-3-(8-fluoro-2-(pyridin- 2-yl)quinolin-7-yl)-1-((1s,3s)-3- hydroxy-3-methylcyclobutyl)- 1H-pyrazole-4-carboxamide |
| 58 | 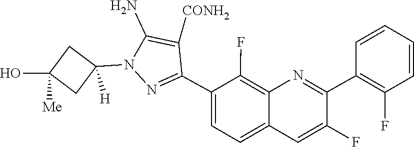 [SMILES: C[C@]1(O)C[C@@H](n2nc(-c3ccc4cc(F)c(-c5ccccc5F)nc4c3F)c(C(N)=O)c2N)C1] | 5-amino-3-(3,8-difluoro-2-(2- fluorophenyl)quinolin-7-yl)-1- ((1s,3s)-3-hydroxy-3- methylcyclobutyl)-1H- pyrazole-4-carboxamide |
| 59 | 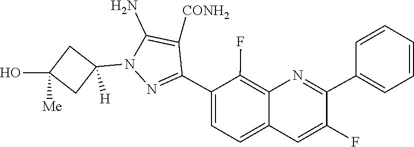 [SMILES: C[C@]1(O)C[C@@H](n2nc(-c3ccc4cc(F)c(-c5ccccc5)nc4c3F)c(C(N)=O)c2N)C1] | 5-amino-3-(3,8-difluoro-2- phenylquinolin-7-yl)-1-((1s,3s)- 3-hydroxy-3-methylcyclobutyl)- 1H-pyrazole-4-carboxamide |
| 60 | 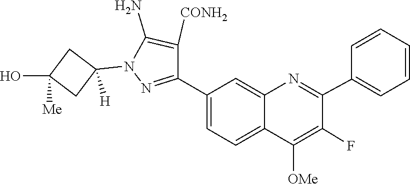 | 5-amino-3-(3-fluoro-4- methoxy-2-phenylquinolin-7- yl)-1-((1s,3s)-3-hydroxy-3- methylcyclobutyl)-1H- pyrazole-4-carboxamide |
| 61 | 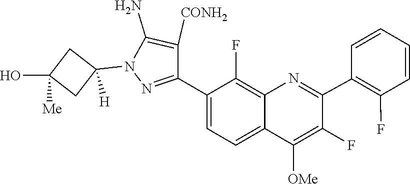 | 5-amino-3-(3,8-difluoro-2-(2- fluorophenyl)-4- methoxyquinolin-7-yl)-1- ((1s,3s)-3-hydroxy-3- methylcyclobutyl)-1H- pyrazole-4-carboxamide |
| 62 | 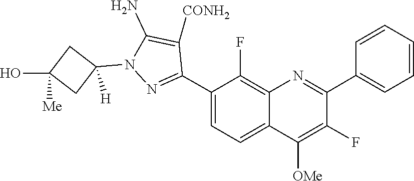 [SMILES: COc1c(F)c(-c2ccccc2)nc2c(F)c(-c3nn([C@H]4C[C@@](C)(O)C4)c(N)c3C(N)=O)ccc12] | 5-amino-3-(3,8-difluoro-4- methoxy-2-phenylquinolin-7- yl)-1-((1s,3s)-3-hydroxy-3- methylcyclobutyl)-1H- pyrazole-4-carboxamide |
| 63 | 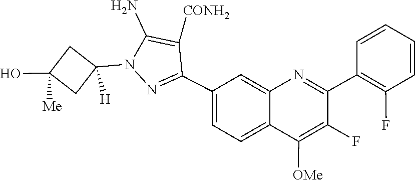 [SMILES: COc1c(F)c(-c2ccccc2F)nc2cc(-c3nn([C@H]4C[C@@](C)(O)C4)c(N)c3C(N)=O)ccc12] | 5-amino-3-(3-fluoro-2-(2- fluorophenyl)-4- methoxyquinolin-7-yl)-1- ((1s,3s)-3-hydroxy-3- methylcyclobutyl)-1H- pyrazole-4-carboxamide |
| 64 | 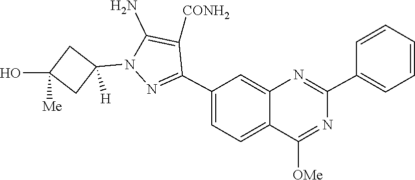 [SMILES: COc1nc(-c2ccccc2)nc2cc(-c3nn([C@H]4C[C@@](C)(O)C4)c(N)c3C(N)=O)ccc12] | 5-amino-1-((1s,3s)-3-hydroxy- 3-methylcyclobutyl)-3-(4- methoxy-2-phenylquinazolin-7- yl)-1H-pyrazole-4-carboxamide |

Another embodiment provides an IGF-1R inhibitory compound, or a pharmaceutically acceptable salt or solvate thereof, as provided in Table 1B.

TABLE 1B

5-amino-3-(8-chloro-2-phenylquinolin-7-yl)-1-
((1s,3s)-3-hydroxy-3-methylcyclobutyl)-1H-
pyrazole-4-carboxamide
5-amino-3-(8-cyano-2-phenylquinolin-7-yl)-1-
((1s,3s)-3-hydroxy-3-methylcyclobutyl)-1H-
pyrazole-4-carboxamide
5-amino-3-(4-ethoxy-8-fluoro-2-(2-
fluorophenyl)quinolin-7-yl)-1-((1s,3s)-3-hydroxy-3-
methylcyclobutyl)-1H-pyrazole-4-carboxamide
5-amino-3-(4-ethoxy-2-(2-fluorophenyl)quinolin-
7-yl)-1-((1s,3s)-3-hydroxy-3-
methylcyclobutyl)-1H-pyrazole-4-carboxamide
5-amino-3-(8-fluoro-2-phenylquinolin-7-yl)-1-(3-
(morpholinomethyl)cyclobutyl)-1H-
pyrazole-4-carboxamide
5-amino-3-(8-fluoro-4-methoxy-2-
phenylquinolin-7-yl)-1-(3-
(morpholinomethyl)cyclobutyl)-
1H-pyrazole-4-carboxamide
5-amino-3-(4-ethoxy-8-fluoro-2-phenylquinolin-
7-yl)-1-(3-(morpholinomethyl)cyclobutyl)-
1H-pyrazole-4-carboxamide
5-amino-3-(4-ethoxy-8-fluoro-2-
(2-fluorophenyl)quinolin-7-yl)-1-(3-
(morpholinomethyl)cyclobutyl)-
1H-pyrazole-4-carboxamide
5-amino-3-(8-fluoro-2-(2-fluorophenyl)-
4-methoxyquinolin-7-yl)-1-(3-
(morpholinomethyl)cyclobutyl)-
1H-pyrazole-4-carboxamide
5-amino-3-(2-(2-fluorophenyl)-4-
methoxyquinolin-7-yl)-1-(3-
(morpholinomethyl)cyclobutyl)-1H-
pyrazole-4-carboxamide
5-amino-3-(4-ethoxy-2-(2-fluorophenyl)quinolin-
7-yl)-1-(3-(morpholinomethyl)cyclobutyl)-
1H-pyrazole-4-carboxamide
5-amino-3-(8-fluoro-2-(2-fluorophenyl)quinolin-
7-yl)-1-(3-(morpholinomethyl)cyclobutyl)-
1H-pyrazole-4-carboxamide
5-amino-3-(4-ethoxy-8-fluoro-2-(2-
fluorophenyl)quinolin-7-yl)-1-
(3-morpholinocyclobutyl)-
1H-pyrazole-4-carboxamide
5-amino-3-(8-fluoro-2-(2-fluorophenyl)-
4-methoxyquinolin-7-yl)-1-(3-
morpholinocyclobutyl)-1H-pyrazole-4-carboxamide
5-amino-3-(2-(2-fluorophenyl)-4-methoxyquinolin-
7-yl)-1-(3-morpholinocyclobutyl)-1H-
pyrazole-4-carboxamide
5-amino-3-(4-ethoxy-2-(2-fluorophenyl)quinolin-
7-yl)-1-(3-morpholinocyclobutyl)-1H-
pyrazole-4-carboxamide
5-amino-3-(8-fluoro-2-(2-fluorophenyl)quinolin-
7-yl)-1-(3-morpholinocyclobutyl)-1H-
pyrazole-4-carboxamide
5-amino-3-(2-(2-fluorophenyl)quinolin-7-yl)-1-(3-
morpholinocyclobutyl)-1H-pyrazole-4-
carboxamide
5-amino-3-(2-(2-fluorophenyl)quinolin-7-yl)-1-(3-
(morpholinomethyl)cyclobutyl)-1H-
pyrazole-4-carboxamide
5-amino-3-(8-fluoro-2-(3-fluoropyridin-2-yl)-4-
methoxyquinolin-7-yl)-1-((1s,3s)-3-hydroxy-
3-methylcyclobutyl)-1H-pyrazole-4-carboxamide
5-amino-3-(8-fluoro-2-(3-fluoropyridin-2-
yl)quinolin-7-yl)-1-((1s,3s)-3-hydroxy-3-
methylcyclobutyl)-1H-pyrazole-4-carboxamide
5-amino-3-(2-(3-fluoropyridin-2-yl)-4-
methoxyquinolin-7-yl)-1-((1s,3s)-3-hydroxy-3-
methylcyclobutyl)-1H-pyrazole-4-carboxamide
5-amino-3-(3,8-difluoro-2-(3-fluoropyridin-2-yl)-
4-methoxyquinolin-7-yl)-1-((1s,3s)-3-
hydroxy-3-methylcyclobutyl)-1H-pyrazole-4-carboxamide
5-amino-3-(3,8-difluoro-4-methoxy-2-(pyridin-
2-yl)quinolin-7-yl)-1-((1s,3s)-3-hydroxy-3-
methylcyclobutyl)-1H-pyrazole-4-carboxamide
5-amino-3-(8-fluoro-4-methoxy-2-(pyridin-2-
yl)quinolin-7-yl)-1-((1s,3s)-3-hydroxy-3-
methylcyclobutyl)-1H-pyrazole-4-carboxamide
5-amino-1-((1s,3s)-3-hydroxy-3-methylcyclobutyl)-
3-(4-methoxy-2-(pyridin-2-yl)quinolin-
7-yl)-1H-pyrazole-4-carboxamide
5-amino-3-(3-fluoro-4-methoxy-2-(pyridin-2-
yl)quinolin-7-yl)-1-((1s,3s)-3-hydroxy-3-
methylcyclobutyl)-1H-pyrazole-4-carboxamide
5-amino-3-(3-fluoro-2-(3-fluoropyridin-2-yl)-4-
methoxyquinolin-7-yl)-1-((1s,3s)-3-hydroxy-
3-methylcyclobutyl)-1H-pyrazole-4-carboxamide

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the IGF-1R inhibitory compound described herein is administered as a pure chemical. In other embodiments, the IGF-1R inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one IGF-1R inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the IGF-1R inhibitory compound as described by Formula (I), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1A or 1B, or a pharmaceutically acceptable salt or solvate thereof.

One embodiment provides a method of preparing a pharmaceutical composition comprising mixing a compound of Table 1A or 1B, or a pharmaceutically acceptable salt or solvate thereof, and a pharmaceutically acceptable carrier.

In certain embodiments, the IGF-1R inhibitory compound as described by Table 1A or 1B, or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the IGF-1R inhibitory compound as described by Formula (I) or Table 1A or 1B, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one IGF-1R inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a pharmaceutical composition comprising a compound of Formula (I), or a pharmaceutically acceptable salt or solvate thereof, and a pharmaceutically acceptable excipient for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (I), or a pharmaceutically acceptable salt or solvate thereof. In some embodiments is provided a method of treating cancer, in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Formula (I), or a pharmaceutically acceptable salt or solvate thereof, for use in a method of treatment of autoimmune disease.

One embodiment provides a pharmaceutical composition comprising a compound of Formula (I), or a pharmaceutically acceptable salt or solvate thereof, and a pharmaceutically acceptable excipient for use in a method of treatment of autoimmune disease.

One embodiment provides a use of a compound of Formula (I), or a pharmaceutically acceptable salt or solvate thereof, in the manufacture of a medicament for the treatment of autoimmune disease.

In some embodiments is provided a method of treating autoimmune disease, in a patient in need thereof, comprising administering to the patient a compound of Formula (I), or a pharmaceutically acceptable salt or solvate thereof. In some embodiments is provided a method of treating autoimmune disease, in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Formula (I), or a pharmaceutically acceptable salt or solvate thereof, for use in a method of treatment of thyroid eye disease.

One embodiment provides a pharmaceutical composition comprising a compound of Formula (I), or a pharmaceutically acceptable salt or solvate thereof, and a pharmaceutically acceptable excipient for use in a method of treatment of thyroid eye disease.

One embodiment provides a use of a compound of Formula (I), or a pharmaceutically acceptable salt or solvate thereof, in the manufacture of a medicament for the treatment of thyroid eye disease.

In some embodiments is provided a method of treating thyroid eye disease, in a patient in need thereof, comprising administering to the patient a compound of Formula (I), or a pharmaceutically acceptable salt or solvate thereof. In some embodiments is provided a method of treating thyroid eye disease, in a patient in need thereof, comprising administering to the patient a pharmaceutical composition comprising a compound of Formula (I), or a pharmaceutically acceptable salt or solvate thereof, and a pharmaceutically acceptable excipient.

One embodiment provides a compound of Table 1A or 1B, or a pharmaceutically acceptable salt or solvate thereof, for use in a method of treatment of the human or animal body.

One embodiment provides a compound of Table 1A or 1B, or a pharmaceutically acceptable salt or solvate thereof, for use in a method of treatment of cancer or neoplastic disease.

One embodiment provides a pharmaceutical composition comprising a compound of Table 1A or 1B, or a pharmaceutically acceptable salt or solvate thereof, and a pharmaceutically acceptable excipient for use in a method of treatment of cancer or neoplastic disease.

One embodiment provides a use of a compound of Table 1A or 1B, or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Table 1A or 1B, or a pharmaceutically acceptable salt or solvate thereof. In some embodiments is provided a method of treating cancer, in a patient in need thereof, comprising administering to the patient a pharmaceutical composition comprising a compound of Table 1A or 1B, or a pharmaceutically acceptable salt or solvate thereof, and a pharmaceutically acceptable excipient.

One embodiment provides a compound of Table 1A or 1B, or a pharmaceutically acceptable salt or solvate thereof, for use in a method of treatment of autoimmune disease.

One embodiment provides a pharmaceutical composition comprising a compound of Table 1A or 1B, or a pharmaceutically acceptable salt or solvate thereof, and a pharmaceutically acceptable excipient for use in a method of treatment of autoimmune disease.

One embodiment provides a use of a compound of Table 1A or 1B, or a pharmaceutically acceptable salt or solvate thereof, in the manufacture of a medicament for the treatment of autoimmune disease.

In some embodiments is provided a method of treating autoimmune disease, in a patient in need thereof, comprising administering to the patient a compound of Table 1A or 1B, or a pharmaceutically acceptable salt or solvate thereof. In some embodiments is provided a method of treating autoimmune disease, in a patient in need thereof, comprising administering to the patient a pharmaceutical composition comprising a compound of Table 1A or 1B, or a pharmaceutically acceptable salt or solvate thereof, and a pharmaceutically acceptable excipient.

One embodiment provides a compound of Table 1A or 1B, or a pharmaceutically acceptable salt or solvate thereof, for use in a method of treatment of thyroid eye disease.

One embodiment provides a pharmaceutical composition comprising a compound of Table 1A or 1B, or a pharmaceutically acceptable salt or solvate thereof, and a pharmaceutically acceptable excipient for use in a method of treatment of thyroid eye disease.

One embodiment provides a use of a compound of Table 1A or 1B, or a pharmaceutically acceptable salt or solvate thereof, in the manufacture of a medicament for the treatment of thyroid eye disease.

In some embodiments is provided a method of treating thyroid eye disease, in a patient in need thereof, comprising administering to the patient a compound of Table 1A or 1B, or a pharmaceutically acceptable salt or solvate thereof. In some embodiments is provided a method of treating thyroid eye disease, in a patient in need thereof, comprising administering to the patient a pharmaceutical composition comprising a compound of Table 1A or 1B, or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

One embodiment provides a method of inhibiting IGF-1R enzyme comprising contacting the IGF-1R enzyme with a compound of Formula (I) or Table 1A or 1B. Another embodiment provides the method of inhibiting IGF-1R enzyme, wherein the IGF-1R enzyme is contacted in an in vivo setting. Another embodiment provides the method of inhibiting an IGF-1R enzyme, wherein the IGF-1R enzyme is contacted in an in vitro setting.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the IGF-1R inhibitory compounds disclosed herein are synthesized according to the following examples. As used below, and throughout the description of the invention, the following abbreviations, unless otherwise indicated, shall be understood to have the following meanings:
° C. degrees Celsius
δ_{H} chemical shift in parts per million downfield from tetramethylsilane
DCM dichloromethane (CH₂Cl₂)
DMF dimethylformamide
DMSO dimethylsulfoxide
EA ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
S singlet (spectral)
t triplet (spectral)
T temperature
TFA trifluoroacetic acid
THF tetrahydrofuran

### Example 1: Preparation of 5-amino-1-(3-oxocyclobutyl)-3-(2-phenylquinolin-7-yl)-1H-pyrazole-4-carboxamide

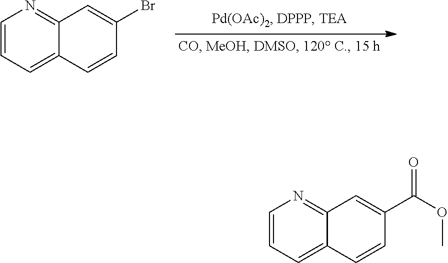

To a solution of 7-bromoquinoline (20 g, 96.6 mmol, 1.0 eq), DPPP (8.0 g, 19.3 mmol, 0.2 eq) and Pd(OAc)₂ (2.1 g, 9.7 mmol, 0.1 eq) in DMSO/MeOH (300 mL/300 mL) was added TEA (40 mL, 289.8 mmol. 3.0 eq). The mixture was stirred at 120° C. for 15 h under CO (5 atm), then concentrated in vacuum. The resulting residue was purified by column chromatography on silica gel (PE/EA=5/1, v/v) to afford methyl quinoline-7-carboxylate (16.4 g, 91.1%) as a yellow solid. LRMS (M+H⁺) m/z calculated 188.1, found 188.0.

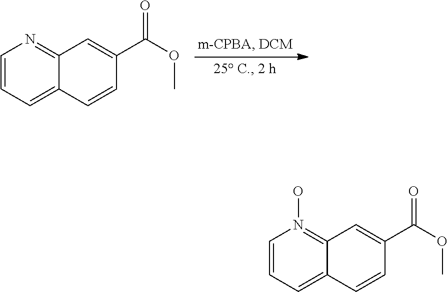

To a stirred solution of methyl quinoline-7-carboxylate (16.4 g, 87.7 mmol, 1.0 eq) in DCM (300 mL) was added m-CPBA (22.7 g, 131.6 mmol, 1.5 eq) at 25° C. The mixture was stirred at 25° C. for 2 h, then poured into ice, adjusted to pH 13 with addition of saturated Na₂CO₃ aqueous solution, and extracted with DCM (500 mL×3). The combined organic layers were dried over sodium sulfate and concentrated in vacuum to afford 7-(methoxycarbonyl) quinoline 1-oxide (17.5 g, 98.3%) as a yellow oil. LRMS (M+H⁺) m/z calculated 204.1, found 204.1.

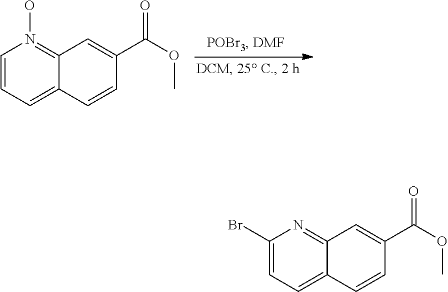

To a solution of 7-(methoxycarbonyl) quinoline 1-oxide (17.5 g, 86.2 mmol, 1.0 eq) in DCM (500 mL) were added POBr₃ (32.1 g, 112.1 mmol, 1.3 eq) and DMF (3.3 mL, 43.1 mmol, 0.5 eq) at −78° C. The mixture was stirred at 25° C. for 2 h, then poured into ice, adjusted to pH 13 with addition of saturated Na₂CO₃ aqueous solution, and extracted with DCM (500 mL×3). The combined organic layers were dried over anhydrous sodium sulfate and concentrated in vacuum. The residue was purified by column chromatography on silica gel (PE/EA=5/1, v/v) to afford methyl 2-bromoquinoline-7-carboxylate (17.2 g, 75.4%) as a yellow solid. LRMS (M+H⁺) m/z calculated 266.0, found 266.0. ¹H NMR (DMSO-d₆, 400 MHz) δ 8.50 (s, 1H), 8.45 (d, 1H), 8.12-8.21 (m, 2H), 8.86 (d, 1H), 3.95 (s, 3H).

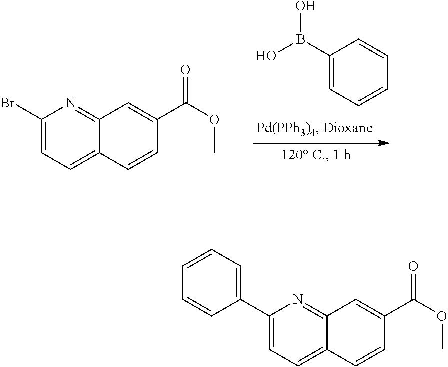 [SMILES: COC(=O)c1ccc2ccc(Br)nc2c1 ; OB(O)c1ccccc1 ; COC(=O)c1ccc2ccc(-c3ccccc3)nc2c1]

To a solution of methyl 2-bromoquinoline-7-carboxylate (17.2 g, 64.7 mmol, 1.0 eq) in dioxane (300 mL) were added phenylboronic acid (15.8 g, 129.3 mmol, 2.00 eq), Pd(PPh₃)₄ (7.5 g, 6.4 mmol, 0.1 eq). The mixture was stirred at 120° C. for 1 h, then concentrated in vacuum and diluted with water (100 mL), and extracted with EtOAc (150 mL×2). The combined organic layers were washed with brine (150 mL), dried over anhydrous sodium sulfate and concentrated in vacuum. The residue was purified by column chromatography on silica gel (PE/EA=5/1, v/v) to afford methyl 2-phenylquinoline-7-carboxylate (8.8 g, 50.2%) as a white solid. LRMS (M+H⁺) m/z calculated 264.1, found 264.1.

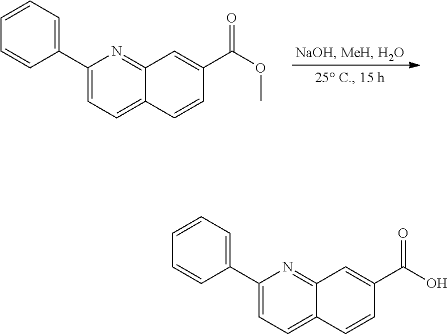 [SMILES: COC(=O)c1ccc2ccc(-c3ccccc3)nc2c1 ; O=C(O)c1ccc2ccc(-c3ccccc3)nc2c1]

To a solution of methyl 2-phenylquinoline-7-carboxylate (8.8 g, 33.5 mmol, 1.0 eq) in MeOH (100 mL) and H₂O (30 mL) was added NaOH (2.0 g, 50.2 mmol, 1.5 eq). The mixture was stirred at 80° C. for 15 h, then concentrated in vacuum and diluted with water (60 mL). 37% HCl was added to adjust to pH 2. The resulting mixture was stirred for 5 min, filtrated and concentrated in vacuum to afford 2-phenylquinoline-7-carboxylic acid (7.8 g, 93.9%) as a white solid. LRMS (M+H⁺) m/z calculated 250.1, found 250.0.

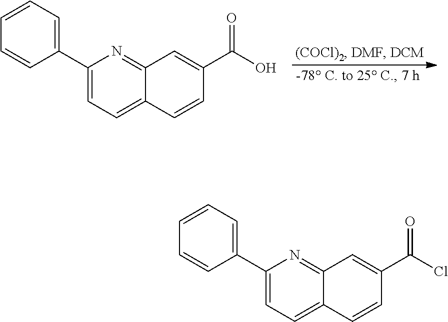 [SMILES: O=C(O)c1ccc2ccc(-c3ccccc3)nc2c1 ; O=C(Cl)c1ccc2ccc(-c3ccccc3)nc2c1]

To a solution of 2-phenylquinoline-7-carboxylic acid (2.1 g, 8.4 mmol, 1.0 eq) in DCM (100 mL) were added (COCl)₂ (3.6 mL, 42.1 mmol, 5.0 eq) and DMF (5 drops) at −78° C. The mixture was stirred at rt for 7 h, then concentrated in vacuum to afford 2-phenylquinoline-7-carbonyl chloride as a yellow solid (2.6 g, ca 100.0%). LRMS (M+H⁺) m/z calculated 264.1, found 264.1 in MeOH.

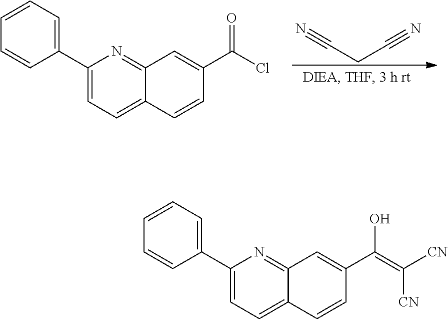 [SMILES: O=C(Cl)c1ccc2ccc(-c3ccccc3)nc2c1 ; N#CCC#N ; N#CC(C#N)=C(O)c1ccc2ccc(-c3ccccc3)nc2c1]

To a solution of 2-phenylquinoline-7-carbonyl chloride (1.0 g, 3.7 mmol, 1.0 eq) in THF (100 mL) were added malononitrile (247.1 mg, 3.70 mmol, 1.0 eq) and DIEA (1.8 mL, 11.20 mmol, 3.0 eq) at ice bath. The mixture was stirred at rt for 3 h, then concentrated and diluted with water (20 mL). The resulting mixture was extracted with EtOAc (50 mL×2). The combined organic layers were washed with brine (50 mL), dried over anhydrous sodium sulfate, and concentrated in vacuum. The resulting residue was purified by column chromatography on silica gel (DCM/MeOH=50/1, v/v) to afford 2-(hydroxy(2-phenylquinolin-7-yl)methylene)malononitrile as a yellow oil (600 mg, 67.5%). LRMS (M+H⁺) m/z calculated 298.1, found 298.0.

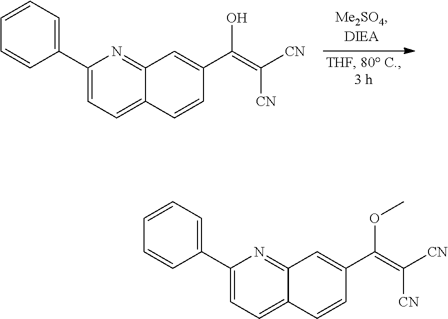 [SMILES: N#CC(C#N)=C(O)c1ccc2ccc(-c3ccccc3)nc2c1 ; COC(=C(C#N)C#N)c1ccc2ccc(-c3ccccc3)nc2c1]

To a solution of 2-(hydroxy (2-phenylquinolin-7-yl)methylene)malononitrile (500 mg, 1.70 mmol, 1.0 eq) in THF (30 mL) were added Me₂SO₄ (0.3 mL, 3.4 mmol, 2.0 eq) and DIEA (0.6 mL, 3.4 mmol, 2.0 eq) at rt. The mixture was stirred at 80° C. for 3 h, then concentrated in vacuum, diluted with water (20 mL), and extracted with EtOAc (50 mL×2). The combined organic layers were washed with brine (50 mL), dried over anhydrous sodium sulfate and concentrated in vacuum. The residue was purified by column chromatography on silica gel (PE/EA=1/1, v/v) to afford 2-(methoxy(2-phenylquinolin-7-yl)methylene)malononitrile as a yellow oil (450 mg, 94.2%). LRMS (M+H⁺) m/z calculated 312.1, found 312.1.

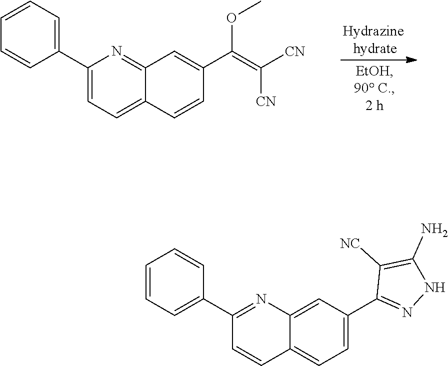 [SMILES: COC(=C(C#N)C#N)c1ccc2ccc(-c3ccccc3)nc2c1 ; N#Cc1c(-c2ccc3ccc(-c4ccccc4)nc3c2)n[nH]c1N]

To a solution of 2-(methoxy(2-phenylquinolin-7-yl)methylene)malononitrile (450 mg, 1.80 mmol, 1.0 eq) in EtOH (30 mL) was added Hydrazine hydrate (0.9 mL, 18.00 mmol, 10.0 eq). The mixture was stirred at 90° C. for 2 h, then concentrated in vacuum, and diluted with water (20 mL). The resulting mixture was stirred for 5 min, and filtrated. The solide was dried to afford 5-amino-3-(2-phenylquinolin-7-yl)-1H-pyrazole-4-carbonitrile as a white solid (456 mg, 100.0%). LRMS (M+H⁺) m/z calculated 312.1, found 312.1.

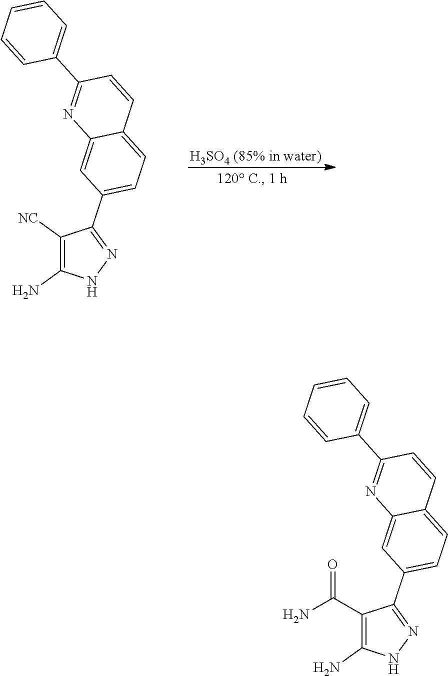 [SMILES: N#Cc1c(-c2ccc3ccc(-c4ccccc4)nc3c2)n[nH]c1N ; "H3SO4 (85% in water)" ; NC(=O)c1c(-c2ccc3ccc(-c4ccccc4)nc3c2)n[nH]c1N]

A solution of 5-amino-3-(2-phenylquinolin-7-yl)-1H-pyrazole-4-carbonitrile (150 mg, 0.48 mmol, 1.0 eq) in H₃PO₄ (10 mL) was stirred at 120° C. for 1 h. The reaction mixture was diluted with water (20 mL). Na₂CO₃ was added to adjust the pH to 12˜13. The mixture was extracted with EtOAc (50 mL×2). The combined organic layers were washed with brine (50 mL) and dried over anhydrous sodium sulfate, and concentrated to afford 5-amino-3-(2-phenylquinolin-7-yl)-1H-pyrazole-4-carboxamide as a white solid (132 mg, 88.0%). LRMS (M+H⁺) m/z calculated 330.1, found 330.1.

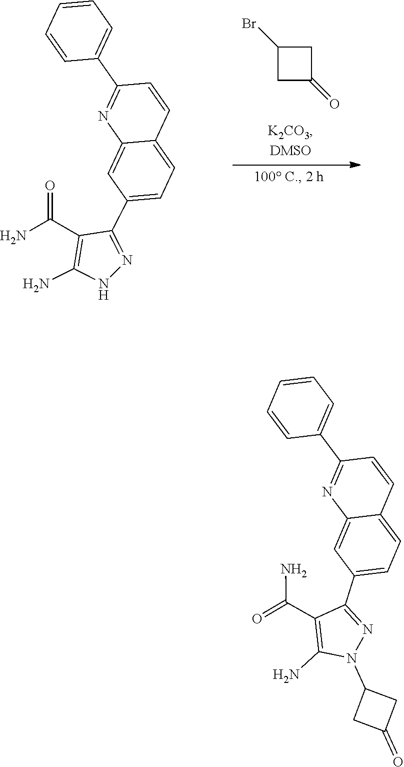 [SMILES: NC(=O)c1c(-c2ccc3ccc(-c4ccccc4)nc3c2)n[nH]c1N ; O=C1CC(Br)C1 ; NC(=O)c1c(-c2ccc3ccc(-c4ccccc4)nc3c2)nn(C2CC(=O)C2)c1N]

To a solution of 5-amino-3-(2-phenylquinolin-7-yl)-1H-pyrazole-4-carboxamide (60 mg, 0.18 mmol, 1.0 eq) in DMSO (20 mL) were added 3-bromocyclobutan-1-one (1.7 mg, 0.72 mmol, 4.0 eq) and K₂CO₃ (75 mg, 0.54 mmol, 3.0 eq). The mixture was stirred at 100° C. for 2 h, then diluted with water (50 mL) and extracted with EtOAc (50 mL×3). The combined organic layers were washed with brine (30 mL×2), and concentrated in vacuum. The resulting residue was purified by Prep-HPLC to afford 5-amino-1-(3-oxocyclobutyl)-3-(2-phenylquinolin-7-yl)-1H-pyrazole-4-carboxamide as a white solid (1.0 mg, 1.6%). LRMS (M+H⁺) m/z calculated 398.2, found 398.0. ¹H NMR (400 MHz, DMSO) δ 8.61 (s, 1H), 8.40 (d, 1H), 8.29 (d, 2H), 8.04-8.10 (m, 2H), 7.86 (d, 1H), 7.49-7.58 (m, 3H), 4.26-4.28 (m, 1H), 2.60-2.67 (m, 2H), 2.32-2.34 (m, 2H).

### Example 2: Preparation of 5-amino-3-(2-phenylquinolin-7-yl)-1-(piperidin-4-yl)-1H-pyrazole-4-carboxamide

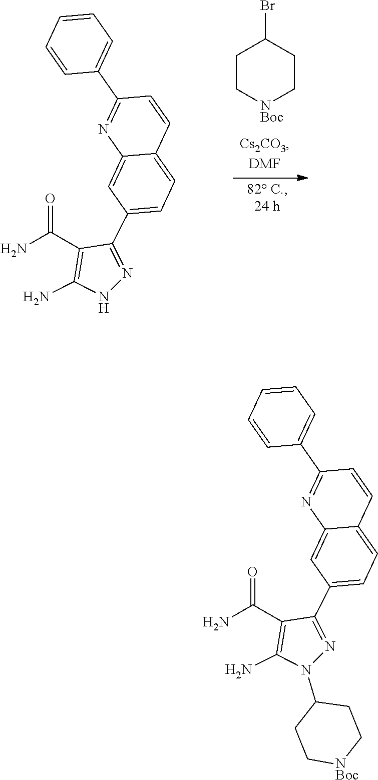 [SMILES: NC(=O)c1c(-c2ccc3ccc(-c4ccccc4)nc3c2)n[nH]c1N ; CC(C)(C)OC(=O)N1CCC(Br)CC1 ; CC(C)(C)OC(=O)N1CCC(n2nc(-c3ccc4ccc(-c5ccccc5)nc4c3)c(C(N)=O)c2N)CC1]

To a solution of 5-amino-3-(2-phenylquinolin-7-yl)-1H-pyrazole-4-carboxamide (260 mg, 0.8 mmol, 1.0 eq) in DMF (20 mL) were added tert-butyl 4-bromopiperidine-1-carboxylate (1.3 g, 4.8 mmol. 6.0 eq) and Cs₂CO₃ (770.5 mg, 2.4 mmol, 3.0 eq). The mixture was stirred at 80° C. for 24 h, then diluted with water (20 mL), extracted with EtOAc (50 mL×2). The combined organic layers were washed with brine (50 mL), dried over anhydrous sodium sulfate and concentrated in vacuum. The resulting residue was purified by column chromatography on silica gel (PE/EA=1/1, v/v) to afford tert-butyl 4-(5-amino-4-carbamoyl-3-(2-phenylquinolin-7-yl)-1H-pyrazol-1-yl) piperidine-1-carboxylate as a yellow oil (240 mg, 94.2%), LRMS (M+H⁺) m/z calculated 513.3, found 513.3.

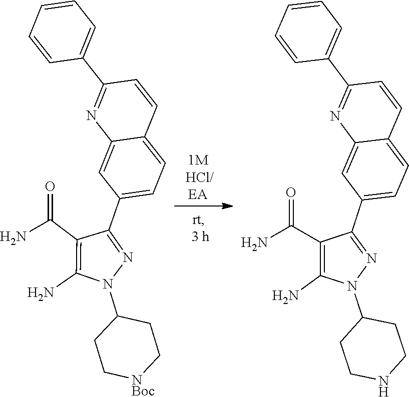

To a solution of tert-butyl 4-(5-amino-4-carbamoyl-3-(2-phenylquinolin-7-yl)-1H-pyrazol-1-yl) piperidine-1-carboxylate (220 mg, 0.5 mmol, 1.0 eq) in DCM (5 mL) was added IM HCl/EA (5 mL). The mixture was stirred at rt for 3 h, then concentrated, The resulting residue was purified by Prep-HPLC to afford 5-amino-3-(2-phenylquinolin-7-yl)-1-(piperidin-4-yl)-1H-pyrazole-4-carboxamide (2.1 mg, 1.2%) as a white solid, LRMS (M+H⁺) m/z calculated 413.2, found 413.2, ¹H NMR (400 MHz, DMSO) δ 8.49 (d, 1H), 8.28-8.48 (m, 4H), 8.16-8.19 (m, 2H), 8.04 (d, 1H), 7.76 (dd, 1H), 7.51-7.59 (m, 4H), 6.33 (s, 2H), 4.31-4.35 (m, 1H), 3.23 (d, 2H), 2.78 (t, 2H), 1.92-2.21 (m, 4H).

### Example 3: Preparation of 4-(5-amino-4-carbamoyl-3-(2-phenylquinolin-7-yl)-1H-pyrazol-1-yl) piperidine-1-carboxamide

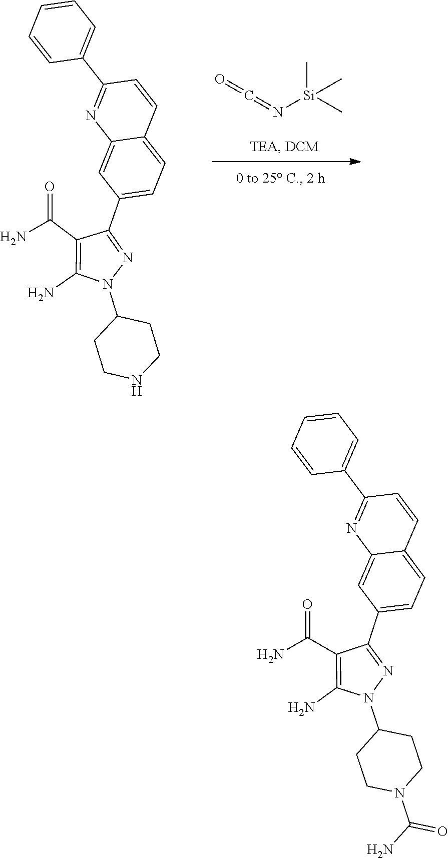

To a solution of 5-amino-3-(2-phenylquinolin-7-yl)-1-(piperidin-4-yl)-1H-pyrazole-4-carboxamide (37 mg, 0.09 mmol, 1.0 eq) in DCM (5 mL) were added TEA (0.1 mL, 0.72 mmol, 8.0 eq) and isocyanatotrimethylsilane (20.7 mg, 0.18 mmol, 2.0 eq) at 0° C. The reaction mixture was stirred at rt for 16 h, then partitioned between saturated NaHCO₃ (20 mL) and DCM (20 mL). The aqueous layer was extracted with DCM (20 mL). The combined organic layers were washed with brine (10 mL), dried with anhydrous sodium sulfate, filtered and concentrated in vacuum. The resulting residue was purified by Prep-HPLC to afford 4-(5-amino-4-carbamoyl-3-(2-phenylquinolin-7-yl)-1H-pyrazol-1-yl) piperidine-1-carboxamide (9.6 mg, 23.4%) as a white solid. LRMS (M+H⁺) m/z calculated 456.2, found 456.1. ¹H NMR (DMSO-d₆, 400 MHz) δ 8.56 (d, 1H), 8.28-8.30 (m, 2H), 8.23 (s, 1H), 8.19 (d, 1H), 8.06 (d, 1H), 7.80 (dd, 1H), 7.53-7.61 (m, 3H), 4.34-4.39 (m, 1H), 4.07-4.11 (m, 2H), 2.77-2.85 (m, 2H), 1.83-1.89 (m, 4H).

### Example 4: Preparation of methyl 4-(5-amino-4-carbamoyl-3-(2-phenylquinolin-7-yl)-1H-pyrazol-1-yl) piperidine-1-carboxylate

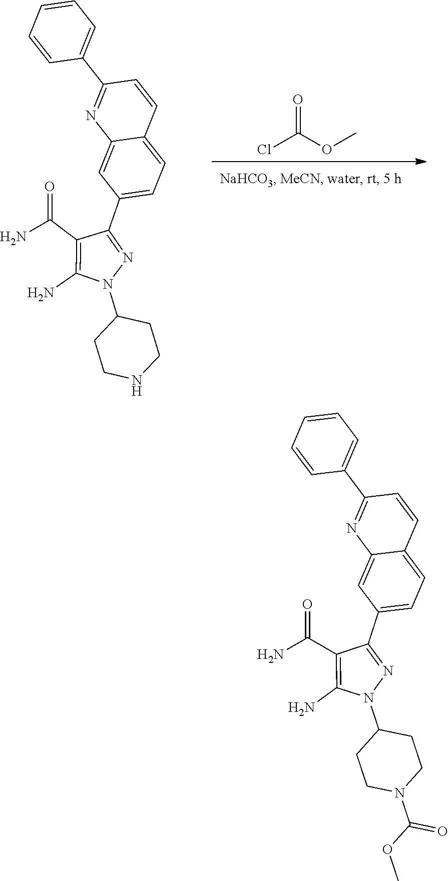

To a solution of 5-amino-3-(2-phenylquinolin-7-yl)-1-(piperidin-4-yl)-1H-pyrazole-4-carboxamide (70 mg, 0.2 mmol, 1.0 eq) and NaHCO₃ (44.7 mg, 0.5 mmol, 3.0 eq) in MeCN (6.0 mL) and H₂O (2.0 mL) were added methyl carbonochloridate (18.9 mg, 0.2 mmol. 1.0 eq). The mixture was stirred at rt for 5 h, then quenched by H₂O (20.0 mL) and extracted with DCM/MeOH (10/1, 30 mL×3). The combined organic layers were dried over anhydrous sodium sulfate and concentrated in vacuum. The resulting residue was purified by Prep-HPLC to afford methyl 4-(5-amino-4-carbamoyl-3-(2-phenylquinolin-7-yl)-1H-pyrazol-1-yl) piperidine-1-carboxylate as a white solid (17.0 mg. 21.3%). LRMS (M+H⁺) m/z calculated 471.2, found 471.1. ¹H NMR (DMSO-d₆, 400 MHz) δ 8.48 (d, 1H), 8.29-8.31 (m, 2H), 8.17 (d, 2H), 8.03 (d, 1H), 7.76 (d, 1H), 7.51-7.59 (m, 3H), 6.35 (s, 2H), 4.38-4.22 (m, 1H), 4.11-4.13 (m, 2H), 3.65 (s, 3H), 2.94-2.95 (m, 2H), 1.86-1.91 (m, 4H).

### Example 5: Preparation of 5-amino-1-((1s,3s)-3-hydroxy-3-methylcyclobutyl)-3-(2-phenylquinolin-7-yl)-1H-pyrazole-4-carboxamide

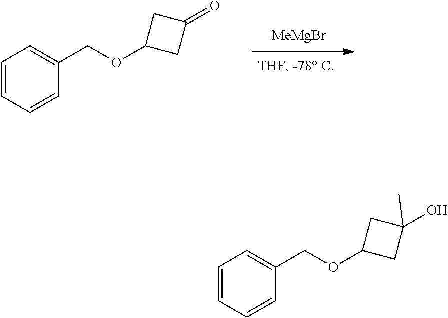

MeMgBr (3 M, 85.2 mL, 255.7 mmol, 1.5 eq) was added dropwise to a solution of 3-(benzyloxy)cyclobutanone (30 g, 170.5 mmol, 1.0 eq) in THF (300 mL) at −78° C. The mixture was stirred at −78° C. for 1 h, then quenched by aqueous NH₄CI solution (500 mL). The aqueous layer was extracted with ethyl acetate (150 mL×3). The combined organic layers were washed with brine (300 mL), dried with anhydrous sodium sulfate and concentrated in vacuum. The resulting residue was purified by column chromatography on silica gel (PE/EA=1/1, v/v) to give 3-(benzyloxy)-1-methylcyclobutan-1-ol (30.3 g, 92.6%) as a yellow oil. ¹H NMR (DMSO-d₆, 400 MHz) δ 7.27-7.36 (m, 5H), 4.97 (s, 1H), 4.34 (s, 2H), 3.64-3.68 (m, 1H), 2.22-2.28 (m, 2H), 1.91-1.96 (m, 2H), 1.15 (s, 3H).

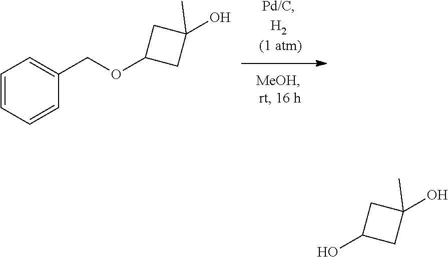

A mixture of 3-benzyloxy-1-methyl-cyclobutanol (30.3 g, 157.8 mmol, 1.0 eq) and Pd/C (10 wt %, 10 g) in MeOH (500 mL) was stirred under hydrogen (1 atm) at rt for 16 h, then filtered and concentrated in vacuum to afford 1-methylcyclobutane-1,3-diol (16 g, ca 100%) as a yellow oil. ¹H NMR (DMSO-d₆, 400 MHz) δ 4.87 (d, 1H), 4.81 (s, 1H), 3.69-3.71 (m, 1H), 2.16-2.22 (m, 2H), 1.84-1.89 (m, 2H), 1.13 (s, 3H).

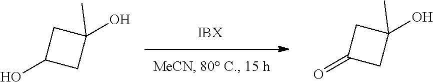

A mixture of 1-methylcyclobutane-1,3-diol (16 g, 156.9 mmol, 1.0 eq) and IBX (87.8 g, 313.7 mmol, 2.0 eq) in MeCN (200 mL) was stirred at 80° C. for 15 h, then filtered and concentrated in vacuum to afford 3-(benzyloxy)cyclobutanone (10 g, 64.1%) as a yellow oil. ¹H NMR (DMSO-d₆, 400 MHz) δ 5.49 (s, 1H), 2.98 (s, 4H), 1.48 (s, 3H).

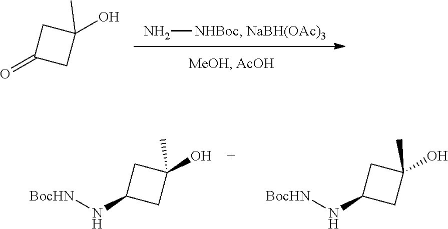

To a stirred solution of 3-hydroxy-3-methylcyclobutan-1-one (10 g, 100.0 mmol, 1.0 eq) in MeOH (200 mL) at rt were added BocNHNH₂ (15.8 g, 120.0 mmol, 1.2 eq) and AcOH (0.5 mL, 8.3 mmol, 0.1 eq). The reaction mixture was stirred at rt for 3 h, then NaBH₃CN (12.6 g, 200.0 mol, 2.0 eq) was addedrt. The reaction mixture was stirred at rt for 2 h, then stirred at 80° C. for 18 h, cooled to rt and concentrated in vacuum. The resulting residue was diluted with EtOAc (200 mL), washed with water (200 mL) and brine (200 mL). The organic layer was dried over anhydrous Sodium sulfate and concentrated in vacuum. The resulting residue was purified by Prep-HPLC to afford tert-butyl 2-((1s,3s)-3-hydroxy-3-methylcyclobutyl)hydrazine-1-carboxylate (4.8 g, 22.2%) and tert-butyl 2-((1r,3r)-3-hydroxy-3-methylcyclobutyl)hydrazine-1-carboxylate (3.6 g, 16.6%) as a colorless oil. tert-butyl 2-((1s,3s)-3-hydroxy-3-methylcyclobutyl)hydrazine-1-carboxylate: ¹H NMR (DMSO-d₆, 400 MHz) δ 8.15 (s, 1H), 4.78 (s, 1H), 4.32-4.34 (m, 1H), 2.95-3.01 (m, 1H), 1.94-1.99 (m, 2H), 1.79-1.84 (m, 2H), 1.38 (s, 9H), 1.15 (s, 3H). LRMS (M+H⁺) m/z calculated 217.1, found 217.1. tert-butyl 2-((1r,3r)-3-hydroxy-3-methylcyclobutyl)hydrazine-1-carboxylate: ¹H NMR (DMSO-d₆, 400 MHz) δ 8.17 (s, 1H), 4.67 (s, 1H), 4.35-4.37 (m, 1H), 3.44-3.46 (m, 1H), 1.93-1.99 (m, 2H), 1.67-1.76 (m, 2H), 1.38 (s, 9H), 1.25 (s, 3H). LRMS (M+H⁺) m/z calculated 217.1, found 217.1.

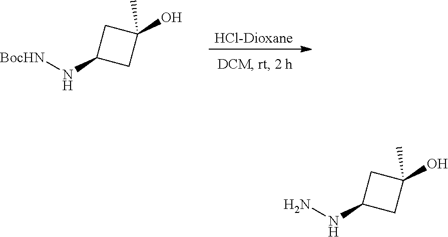

To a stirring solution of tert-butyl 2-((1s,3s)-3-hydroxy-3-methylcyclobutyl)hydrazine-1-carboxylate (1.5 g, 6.9 mmol, 1.0 eq) in DCM (10 mL) was added HCl in Dioxane (4 N, 10 mL). The reaction was stirred at 30° C. for 30 min, then the mixture was concentrated in vacuum to afford (1s,3s)-3-hydrazineyl-1-methylcyclobutan-1-ol (1.2 g, ca 100%) as a white solid. LRMS (M+H⁺) m/z calculated 117.1, found 117.1.

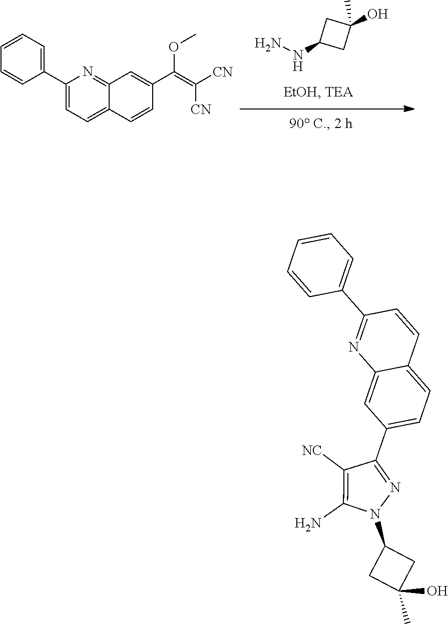

To a solution of 2-(methoxy(2-phenylquinolin-7-yl)methylene)malononitrile (2.1 g, 6.9 mmol, 1.0 eq) and (1s,3s)-3-hydrazineyl-1-methylcyclobutan-1-ol (1.2 g, 10.3 mmol, 1.5 eq) in EtOH (50 mL) was added TEA (7.6 mL, 55.2 mmol, 8.0 eq) at rt. The reaction mixture was stirred at 90° C. for 2 h, then the mixture was concentrated in vacuum and purified by column chromatography on silica gel (PE/EA=1/1, v/v) to afford 5-amino-1-((1s,3s)-3-hydroxy-3-methylcyclobutyl)-3-(2-phenylquinolin-7-yl)-1H-pyrazole-4-carbonitrile (1.7 g, 62.9%) as a yellow solid. LRMS (M+H⁺) m/z calculated 395.2, found 396.3.

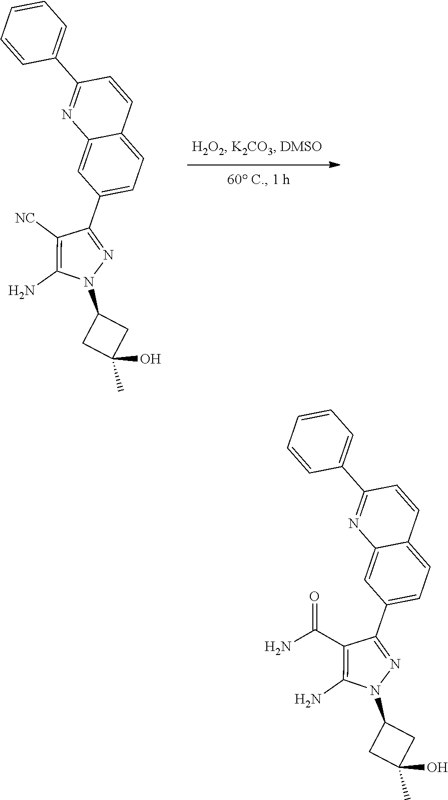 [SMILES: C[C@]1(O)C[C@@H](n2nc(-c3ccc4ccc(-c5ccccc5)nc4c3)c(C#N)c2N)C1 ; C[C@]1(O)C[C@@H](n2nc(-c3ccc4ccc(-c5ccccc5)nc4c3)c(C(N)=O)c2N)C1]

To a stirred solution of 5-amino-1-((1s,3s)-3-hydroxy-3-methylcyclobutyl)-3-(2-phenylquinolin-7-yl)-1H-pyrazole-4-carbonitrile (1.7 g, 4.3 mmol, 1.0 eq) and K₂CO₃ (1.8 g, 12.9 mmol, 3.0 eq) in DMSO (30 mL) at rt was added H₂O₂ (30%, 9.8 g, 86.1 mmol, 20.0 eq). After addition was completed, the reaction mixture was stirred at 60° C. for 1 h. Water (80 mL) was added and the mixture was extracted with EtOAc (200 mL). The organic layer was washed with brine (100 mL), dried with anhydrous Sodium sulfate, and purified by Prep-HPLC to give 5-amino-1-((1s,3s)-3-hydroxy-3-methylcyclobutyl)-3-(2-phenylquinolin-7-yl)-1H-pyrazole-4-carboxamide (1050.2 mg, 61.8%) as a white solid. LRMS (M+H⁺) m/z calculated 414.2, found 414.1. ¹H NMR (DMSO-d₆, 400 MHz) δ 8.50 (d, 1H), 8.30 (d, 2H), 8.21 (s, 1H), 8.18 (d, 1H), 8.05 (d, 1H), 7.78 (dd, 1H), 7.49-7.59 (m, 3H), 6.31 (brs, 2H), 5.19 (brs, 1H), 4.42-4.51 (m, 1H), 2.59-2.65 (m, 2H), 2.36-2.42 (m, 2H), 1.35 (s, 3H).

### Example 6: Preparation of 5-amino-1-((1r,3r)-3-hydroxy-3-methylcyclobutyl)-3-(2-phenylquinolin-7-yl)-1H-pyrazole-4-carboxamide

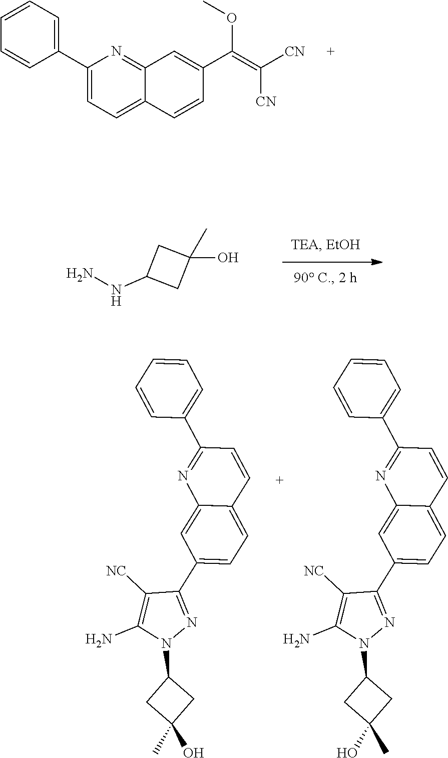

To a solution of 2-(methoxy(2-phenylquinolin-7-yl)methylene)malononitrile (268.1 mg, 0.86 mmol, 1.0 eq) and 3-hydrazineyl-1-methylcyclobutan-1-ol (150 mg, 1.3 mmol, 1.5 eq) in EtOH (50 mL) was added TEA (1.0 mL, 6.9 mmol, 8.0 eq) at rt. The reaction mixture was stirred at 90° C. for 2 h, then concentrated in vacuum. The resulting residue was purified by reverse chromatography, eluted with (MeCN in H₂O, from 10% to 70%) to afford 5-amino-1-((1s,3s)-3-hydroxy-3-methylcyclobutyl)-3-(2-phenylquinolin-7-yl)-1H-pyrazole-4-carbonitrile (55 mg, 16.2%) and 5-amino-1-((1r,3r)-3-hydroxy-3-methylcyclobutyl)-3-(2-phenylquinolin-7-yl)-1H-pyrazole-4-carbonitrile (60 mg, 17.6%) as a yellow solid. They were confirmed by NOESY. 5-amino-1-((1s,3s)-3-hydroxy-3-methylcyclobutyl)-3-(2-phenylquinolin-7-yl)-1H-pyrazole-4-carbonitrile, ¹H NMR (DMSO-d₆, 400 MHz) δ 8.54 (s, 1H), 8.48 (d, 1H), 8.28-8.31 (m, 2H), 8.17 (d, 1H), 8.09 (s, 2H), 7.49-7.59 (m, 3H), 6.80 (s, 2H), 5.28 (s, 1H), 4.45-4.50 (m, 1H), 2.60-2.66 (m, 2H), 2.39-2.44 (m, 2H), 1.34 (s, 3H). LRMS (M+H⁺) m/z calculated 396.2, found 396.3. 5-amino-1-((1r,3r)-3-hydroxy-3-methylcyclobutyl)-3-(2-phenylquinolin-7-yl)-1H-pyrazole-4-carbonitrile, ¹H NMR (DMSO-d₆, 400 MHz) δ 8.53 (s, 1H), 8.48 (d, 1H), 8.28-8.31 (m, 2H), 8.17 (d, 1H), 8.09 (s, 2H), 7.50-7.60 (m, 3H), 6.77 (s, 2H), 5.03 (s, 1H), 4.94-4.99 (m, 1H), 2.54-2.58 (m, 2H), 2.43-2.49 (m, 2H), 1.41 (s, 3H). LRMS (M+H⁺) m/z calculated 396.2, found 396.3.

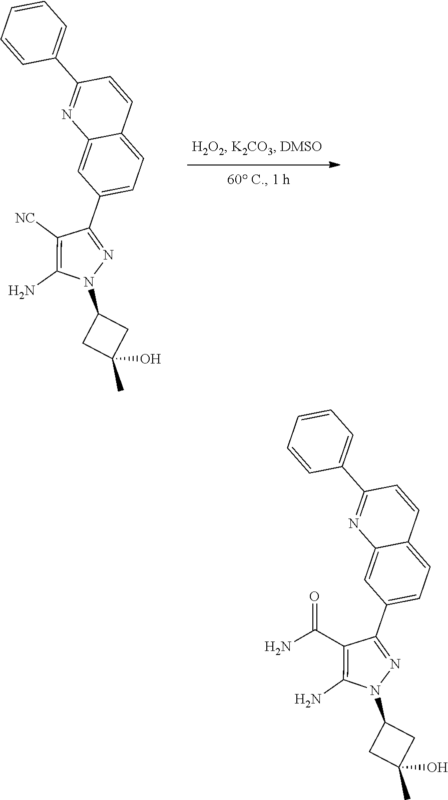 [SMILES: C[C@]1(O)C[C@H](n2nc(-c3ccc4ccc(-c5ccccc5)nc4c3)c(C#N)c2N)C1 ; C[C@]1(O)C[C@H](n2nc(-c3ccc4ccc(-c5ccccc5)nc4c3)c(C(N)=O)c2N)C1]

To a stirred solution of 5-amino-1-((1r,3r)-3-hydroxy-3-methylcyclobutyl)-3-(2-phenylquinolin-7-yl)-1H-pyrazole-4-carbonitrile (60 mg, 0.15 mmol, 1.0 eq) and K₂CO₃ (62.9 mg, 0.46 mmol, 3.0 eq) in DMSO (30 mL) at rt was added H₂O₂ (30%, 344.3 mg, 3.0 mmol, 20.0 eq). After addition was completed, the reaction mixture was stirred at 60° C. for 2 h. Water (20 mL) was added and the mixture was extracted with EtOAc (50 mL×3). The combined organic layers were washed with brine (30 mL), dried with anhydrous sodium sulfate and concentrated in vacuum. The resulting residue was purified by Prep-HPLC to afford 5-amino-1-((1r,3r)-3-hydroxy-3-methylcyclobutyl)-3-(2-phenylquinolin-7-yl)-1H-pyrazole-4-carboxamide (5.7 mg, 9.5%) as a white solid. LRMS (M+H⁺) m/z calculated 414.2, found 414.1. ¹H NMR (DMSO-d₆, 400 MHz) δ 8.49 (d, 1H), 8.29-8.32 (m, 2H), 8.16-8.20 (m, 2H), 8.05 (d, 1H), 7.78 (dd, 1H), 7.49-7.59 (m, 3H), 6.28 (s, 2H), 4.93-4.97 (m, 1H), 2.54-2.56 (m, 2H), 2.39-2.45 (m, 2H), 1.36 (s, 3H).

### Example 7: Preparation of 5-amino-3-(2-phenylquinolin-7-yl)-1-(piperidin-4-yl)-1H-pyrazole-4-carboxamide

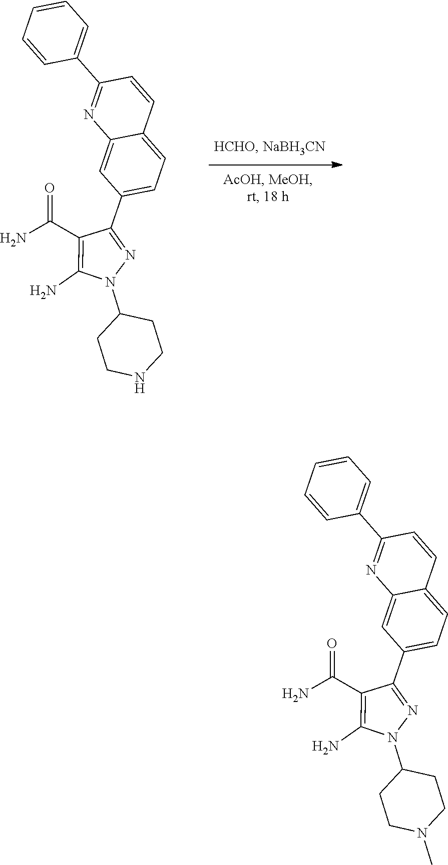 [SMILES: NC(=O)c1c(-c2ccc3ccc(-c4ccccc4)nc3c2)nn(C2CCNCC2)c1N ; CN1CCC(n2nc(-c3ccc4ccc(-c5ccccc5)nc4c3)c(C(N)=O)c2N)CC1]

To a solution of 5-amino-3-(2-phenylquinolin-7-yl)-1-(piperidin-4-yl)-1H-pyrazole-4-carboxamide (20 mg, 0.05 mmol, 1.0 eq) in MeOH (10 mL) were added HCHO (37%, 0.24 mL, 0.05 mmol. 1.0 eq), AcOH (1 drop) and NaBH₃CN (9.5 mg, 0.15 mmol, 3.0 eq). The mixture was stirred at rt for 18 h, then diluted with water (10 mL). Na₂CO₃ was added to adjust to pH 10˜11. The mixture was extracted with EtOAc (20 mL×2). The combined organic layers were washed with brine (50 mL), dried over sodium sulfate and concentrated in vacuum. The resulting residue was purified by Prep-HPLC to afford 5-amino-1-(1-methylpiperidin-4-yl)-3-(2-phenylquinolin-7-yl)-1H-pyrazole-4-carboxamide (1.9 mg, 9.2%) as a white solid. LCMS (M+H⁺) m/z calculated 427.2, found 427.3, ¹H NMR (400 MHz, CD₃OD) δ 8.40 (d, 1H), 8.01 (d, 1H), 8.05 (d, 2H), 8.01 (d, 2H) 7.75 (d, 1H), 7.40-7.45 (m, 3H), 4.58 (s, 3H), 3.18-3.22 (m, 2H), 2.47 (s, 3H), 2.31-2.35 (m, 2H), 2.04 (d, 2H).

### Example 8: Preparation of 5-amino-1-(1-(2-hydroxyethyl) piperidin-4-yl)-3-(2-phenylquinolin-7-yl)-1H-pyrazole-4-carboxamide

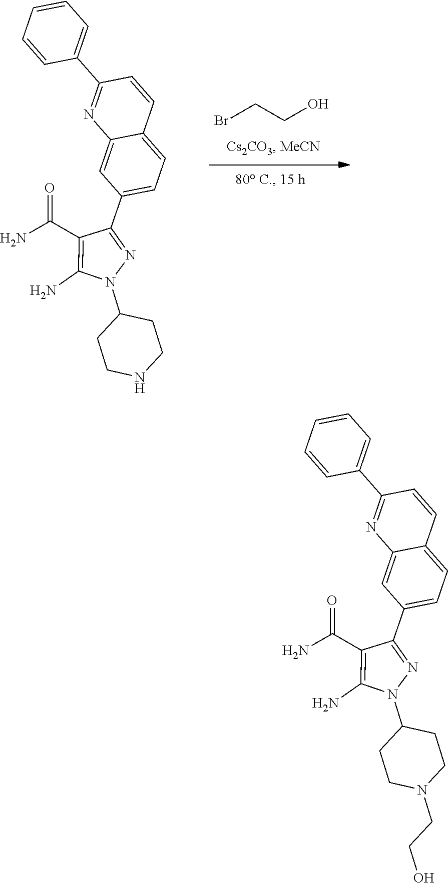

To a solution of 5-amino-3-(2-phenylquinolin-7-yl)-1-(piperidin-4-yl)-1H-pyrazole-4-carboxamide (20 mg, 0.049 mmol, 1.0 eq) in MeCN (8 mL) were added 2-bromoethan-1-ol (8 mg, 0.064 mmol, 1.3 eq) and Cs₂CO₃ (32 mg, 0.098 mmol. 2 eq). The mixture was stirred at 80° C. for 15 h, then diluted with water (50 mL), and extracted with EtOAc (20 mL×2). The combined organic layers were washed with water (20 mL) and brine (20 mL), dried over anhydrous sodium sulfate and concentrated in vacuum. The resulting residue was purified by Prep-HPLC to afford 5-amino-1-(1-(2-hydroxyethyl) piperidin-4-yl)-3-(2-phenylquinolin-7-yl)-1H-pyrazole-4-carboxamide as a white solid (2.7 mg, 12.2%). LRMS (M+H⁺) m/z calculated 457.2, found 457.1. ¹H NMR (DMSO-d₆, 400 MHz) δ 8.48 (d, 1H), 8.28-8.30 (m, 2H), 8.15-8.18 (m, 2H), 8.03 (d, 1H), 7.57-7.78 (dd, 1H), 7.49-7.59 (m, 3H), 6.30 (s, 2H), 4.39 (s, 1H), 4.13-4.18 (m, 1H), 3.51 (s, 2H), 3.00 (d, 2H), 2.44 (t, 2H), 1.99-2.17 (m, 4H), 1.80-1.83 (m, 2H).

### Example 9: Preparation of 5-amino-1-((1s,3s)-3-hydroxycyclobutyl)-3-(2-phenylquinolin-7-yl)-1H-pyrazole-4-carboxamide and 5-amino-1-((1r,3r)-3-hydroxycyclobutyl)-3-(2-phenylquinolin-7-yl)-1H-pyrazole-4-carboxamide

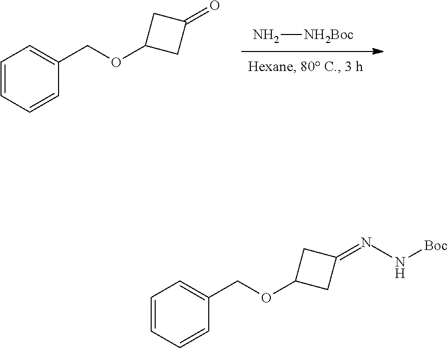

To a solution of 3-(benzyloxy)cyclobutan-1-one (2.0 g, 11.40 mmol, 1.0 eq) in hexane (100 mL) was added NH₂—NH₂Boc (1.5 g, 11.40 mmol, 1.0 eq). The mixture was stirred at 80° C. for 3 h, then concentrated in vacuum. The resulting residue was purified by column chromatography on silica gel (PE/EA=5/1, v/v) to afford tert-butyl 2-(3-(benzyloxy)cyclobutylidene)hydrazine-1-carboxylate as a yellow oil (2.4 g, 90.2%), LRMS (M+H⁺) m/z calculated 291.2, found 291.1

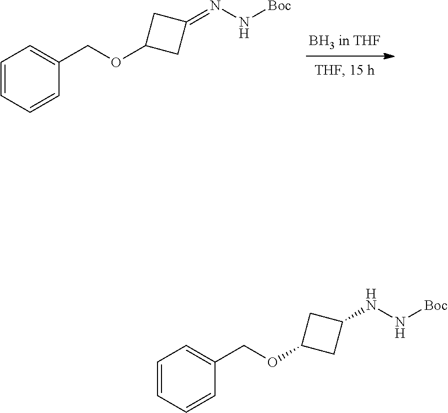 [SMILES: CC(C)(C)OC(=O)NN=C1CC(OCc2ccccc2)C1 ; CC(C)(C)OC(=O)NN[C@H]1C[C@@H](OCc2ccccc2)C1]

To a solution of tert-butyl 2-(3-(benzyloxy)cyclobutylidene)hydrazine-1-carboxylate (2.0 g, 6.9 mmol, 1.0 eq) in THF (100 mL) was added BH₃ (1 M, 22 mL, 22.00 mmol, 3.0 eq) under ice bath. The mixture was stirred at rt for 15 h, then quenched with saturated aqueous NH₄Cl, extracted with EtOAc (200 mL×2). The combined organic layers were washed with brine (200 mL), concentrated in vacuum. The resulting residue was purified by column chromatography on silica gel (PE/EA=5/1, v/v) to afford tert-butyl 2-((1s,3s)-3-(benzyloxy)cyclobutyl)hydrazine-1-carboxylate (2.1 g, ca 100%) as a colorless oil, LRMS (M+H⁺) m/z calculated 293.2, found 293.1.

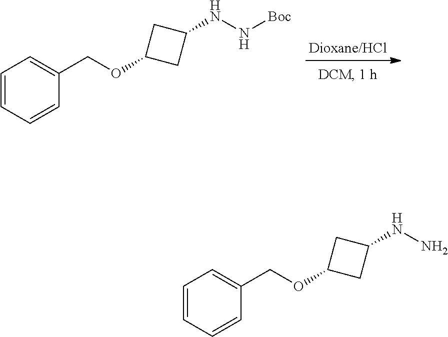 [SMILES: CC(C)(C)OC(=O)NN[C@H]1C[C@@H](OCc2ccccc2)C1 ; NN[C@H]1C[C@@H](OCc2ccccc2)C1]

To a stirred solution of tert-butyl 2-((1s,3s)-3-(benzyloxy)cyclobutyl)hydrazine-1-carboxylate (2.4 g, 10.30 mmol, 1.0 eq) in DCM (80 mL) was added 4 N HCl/Dioxane (50 mL). The reaction was stirred at 30° C. for 30 min, then concentrated in vacuum to afford ((1s,3s)-3-(benzyloxy)cyclobutyl)hydrazine (2.1 g, ca 100.0%). LRMS (M+H⁺) m/z calculated 193.1, found 193.0.

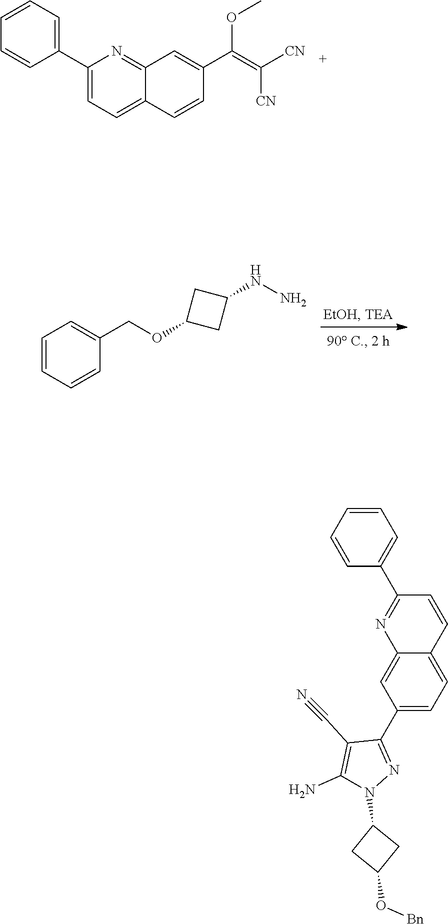 [SMILES: COC(=C(C#N)C#N)c1ccc2ccc(-c3ccccc3)nc2c1 ; NN[C@H]1C[C@@H](OCc2ccccc2)C1 ; N#Cc1c(-c2ccc3ccc(-c4ccccc4)nc3c2)nn([C@H]2C[C@@H](OCc3ccccc3)C2)c1N]

To a solution of ((1s,3s)-3-(benzyloxy)cyclobutyl)hydrazine (500 mg, 2.60 mmol, 1.0 eq) in EtOH (30 mL) were added 2-(methoxy(2-phenylquinolin-7-yl)methylene)malononitrile (1.2 g, 3.9 mmol, 1.5 eq) and TEA (5.4 mL, 39.1 mmol, 15.0 eq). The mixture was stirred at 90° C. for 2 h, then concentrated, diluted with water (20 mL), extracted with EtOAc (50 mL×2). The combined organic layers were washed with brine (50 mL), dried over anhydrous sodium sulfate and concentrated in vacuum. The resulting residue was purified by column chromatography on silica gel (PE/EA=1/1, v/v) to afford 5-amino-1-((1s,3s)-3-(benzyloxy)cyclobutyl)-3-(2-phenylquinolin-7-yl)-1H-pyrazole-4-carbonitrile (450 mg, 36.8%) as a yellow solid. LRMS (M+H⁺) m/z calculated 472.2, found 472.2.

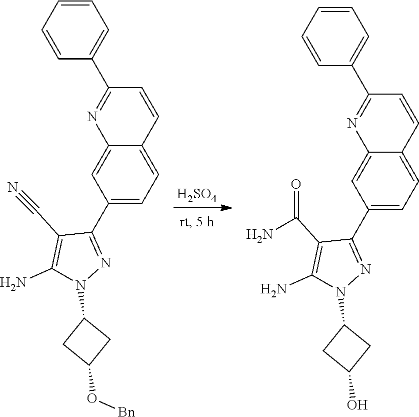

A solution of 5-amino-1-((1s,3s)-3-(benzyloxy)cyclobutyl)-3-(2-phenylquinolin-7-yl)-1H-pyrazole-4-carbonitrile (450 mg, 0.95 mmol, 1.0 eq) in H₂SO₄ (10 mL) was stirred at rt for 5 h, then poured into ice. Na₂CO₃ was added to adjust to pH=12˜13. The aqueous layer was concentrated in vacuum and The resulting residue was triturated with DCM/MeOH (1/1, 300 mL) and filtered. The organic layer was concentrated in vacuum, and the resulting residue was purified by Prep-HPLC to afford 5-amino-1-((1s,3s)-3-hydroxycyclobutyl)-3-(2-phenylquinolin-7-yl)-1H-pyrazole-4-carboxamide as a white solid (250 mg, 65.8%), LRMS (M+H⁺) m/z calculated 400.2, found 400.3. ¹H NMR (400 MHz, DMSO-d₆) δ 8.49 (d, 1H), 8.40-8.33 (m, 2H), 8.22 (d, 1H), 8.17 (d, 1H), 8.05 (d, 1H), 7.83 (dd, 1H), 7.49-7.59 (m, 3H), 6.26 (s, 2H), 4.33-4.49 (m, 2H), 2.57-2.76 (m, 4H).

### Example 10: Preparation of 1-(1-acetylpiperidin-4-yl)-5-amino-3-(2-phenylquinolin-7-yl)-1H-pyrazole-4-carboxamide

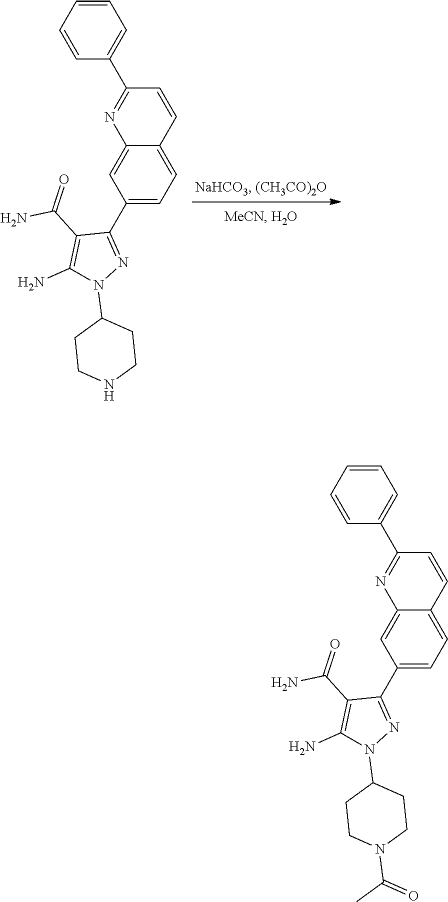 [SMILES: NC(=O)c1c(-c2ccc3ccc(-c4ccccc4)nc3c2)nn(C2CCNCC2)c1N ; CC(=O)N1CCC(n2nc(-c3ccc4ccc(-c5ccccc5)nc4c3)c(C(N)=O)c2N)CC1]

To a solution of 5-amino-3-(2-phenylquinolin-7-yl)-1-(piperidin-4-yl)-1H-pyrazole-4-carboxamide (25 mg, 0.06 mmol, 1.0 eq) in MeCN/H₂O (1/1, 5 mL) were added NaHCO₃ (15 mg, 0.18 mmol. 3.0 eq) and (CH₃CO)₂O (0.1 mL, 0.09 mmol, 1.5 eq). The mixture was stirred at rt for 3 h, then concentrated in vacuum and diluted with water (10 mL), and extracted with EtOAc (20 mL×2). The combined organic layers were washed with brine (50 mL) and dried over anhydrous sodium sulfate, concentrated in vacuum. The resulting residue was purified by Prep-HPLC to afford 1-(1-acetylpiperidin-4-yl)-5-amino-3-(2-phenylquinolin-7-yl)-1H-pyrazole-4-carboxamide (22 mg, 78.5%) as a white solid. LRMS (M+H⁺) m/z calculated 455.2, found 455.1. ¹H NMR (400 MHz, DMSO-d₆) δ 8.48 (d, 1H), 8.29 (d, 2H), 8.15-8.19 (m, 2H), 8.03 (d, 1H), 7.76 (d, 1H), 7.51-7.59 (m, 3H), 6.35 (s, 2H), 4.42-4.54 (m, 2H), 3.95-3.99 (m, 1H), 3.17-3.33 (m, 1H), 2.50-2.71 (m, 1H), 1.98 (s, 3H), 1.75-1.96 (m, 4H).

### Example 11: Preparation of ethyl (1s,3s)-3-(5-amino-4-carbamoyl-3-(2-phenylquinolin-7-yl)-1H-pyrazol-1-yl)cyclobutane-1-carboxylate

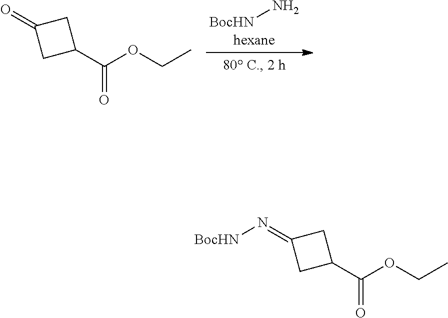

To a solution of ethyl 3-oxocyclobutane-1-carboxylate (20.0 g, 140.8 mmol, 1.0 eq) in hexane (200 mL) was added tert-butyl hydrazinecarboxylate (22.3 g, 169.0 mmol, 1.2 eq) at rt. The mixture was stirred at 80° C. for 2 h, then concentrated in vacuum. The resulting residue was purified by column chromatography on silica gel (PE/EA=2/1, v/v) to afford tert-butyl 2-(3-(ethoxycarbonyl)cyclobutylidene)hydrazine-1-carboxylate (32 g, 88.9%) as a white solid. LRMS (M+H⁺) m/z calculated 257.1, found 257.0.

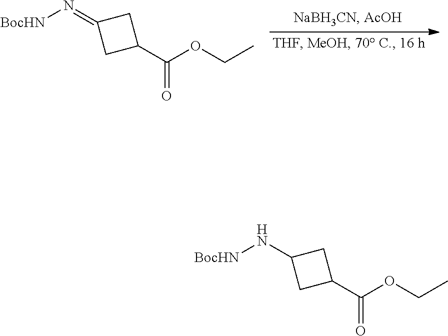 [SMILES: CCOC(=O)C1CC(=NNC(=O)OC(C)(C)C)C1 ; CCOC(=O)C1CC(NNC(=O)OC(C)(C)C)C1]

To a stirred solution of tert-butyl 2-(3-(ethoxycarbonyl)cyclobutylidene)hydrazine-1-carboxylate (27 g 105.5 mmol, 1.0 eq) and NaBH₃CN (13.3 g, 210.9 mmol, 2.0 eq) in MeOH (100 mL)/THF (200 mL) was added AcOH (3 mL, 50 mmol, 0.5 eq) at rt. The reaction mixture was stirred at 70° C. for 18, then cooled to rt and concentrated in vacuum. The resulting residue was diluted with EtOAc (200 mL), washed with water (200 mL) and brine (200 mL), dried over anhydrous sodium sulfate, and concentrated in vacuum. The resulting residue was purified by column chromatography on silica gel (DCM/MeOH=40:1, v/v) to afford tert-butyl 2-(3-(ethoxycarbonyl)cyclobutyl)hydrazine-1-carboxylate (27.3 g, ca 100%) as a white solid. LRMS (M+H⁺) m/z calculated 259.2, found 259.1

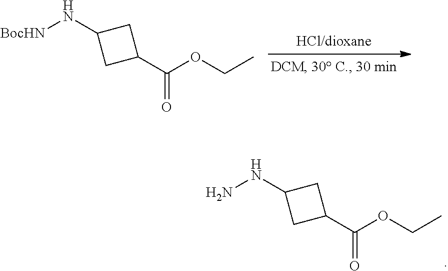 [SMILES: CCOC(=O)C1CC(NNC(=O)OC(C)(C)C)C1 ; CCOC(=O)C1CC(NN)C1]

To a stirred solution of tert-butyl 2-(3-(ethoxycarbonyl)cyclobutyl)hydrazine-1-carboxylate (27.3 g, 105.8 mmol, 1.0 eq) in DCM (80 mL) was added HCl in dioxane (4 N, 30 mL). The reaction was stirred at 30° C. for 30 min, then concentrated in vacuum to afford ethyl 3-hydrazineylcyclobutane-1-carboxylate as a white solid (23.1 g, ca 100%). LRMS (M+H⁺) m/z calculated 159.1, found 159.0.

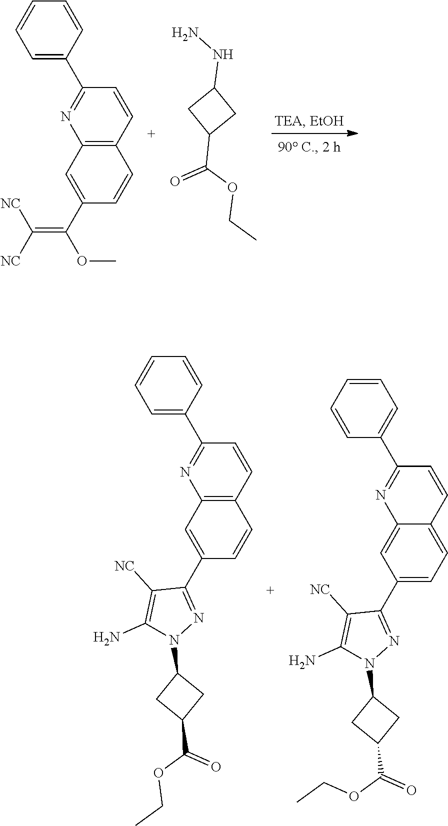

To a solution of 2-(methoxy(2-phenylquinolin-7-yl)methylene)malononitrile (2 g, 6.4 mmol, 1.0 eq) and ethyl 3-hydrazineylcyclobutane-1-carboxylate (1.5 g, 9.6 mmol, 1.5 eq) in EtOH (30 mL) was added TEA (7.1 mL, 51.4 mmol, 8.0 eq) at rt. The reaction mixture was stirred at 90° C. for 2 h, then concentrated in vacuum. The resulting residue was purified by silica gel chromatography column (PE/EA=2/1, v/v) to afford to afford ethyl (1s,3s)-3-(5-amino-4-cyano-3-(2-phenylquinolin-7-yl)-1H-pyrazol-1-yl)cyclobutane-1-carboxylate (900 mg, 32.1%) and ethyl (1r,3s)-3-(5-amino-4-cyano-3-(2-phenylquinolin-7-yl)-1H-pyrazol-1-yl)cyclobutane-1-carboxylate (830 mg, 29.6%) as a white solid. Ethyl (1s,3s)-3-(5-amino-4-cyano-3-(2-phenylquinolin-7-yl)-1H-pyrazol-1-yl)cyclobutane-1-carboxylate: ¹H NMR (DMSO-d₆, 400 MHz) δ 8.53 (s, 1H), 8.49 (d, 1H), 8.30 (d, 2H), 8.18 (d, 1H), 8.05-8.10 (m, 2H), 7.50-7.59 (m, 3H), 6.85 (s, 2H), 4.77-4.81 (m, 1H), 4.13 (q, 2H), 2.95-2.99 (m, 1H), 2.73-2.80 (m, 2H), 2.64-2.69 (m, 2H), 1.22 (t, 3H). LRMS (M+H⁺) m/z calculated 438.2, found 438.1. Ethyl (1r,3s)-3-(5-amino-4-cyano-3-(2-phenylquinolin-7-yl)-1H-pyrazol-1-yl)cyclobutane-1-carboxylate: ¹H NMR (DMSO-d₆, 400 MHz) δ 8.56 (s, 1H), 8.49 (d, 1H), 8.30 (d, 2H), 8.18 (d, 1H), 8.07-8.13 (m, 2H), 7.51-7.60 (m, 3H), 6.85 (s, 2H), 5.02-5.06 (m, 1H), 4.15 (q, 2H), 3.23-3.28 (m, 1H), 2.84-2.91 (m, 2H), 2.61-2.68 (m, 2H), 1.25 (t, 3H). LRMS (M+H⁺) m/z calculated 438.2, found 438.1.

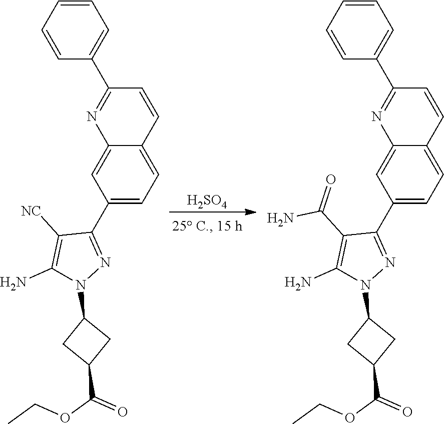

A solution of ethyl (1s,3s)-3-(5-amino-4-cyano-3-(2-phenylquinolin-7-yl)-1H-pyrazol-1-yl)cyclobutane-1-carboxylate (25 mg, 0.057 mmol, 1.0 eq) in conc. H₂SO₄ (3 mL) was stirred at 25° C. for 15 h, then adjusted to pH 8 with saturated sodium carbonate solution, extracted with EtOAc (30 mL×2). The combined organic layers were washed with brine (20 mL), dried over anhydrous sodium sulfate, and concentrated in vacuum. The resulting residue was purified by Prep-HPLC to afford ethyl (1s,3s)-3-(5-amino-4-carbamoyl-3-(2-phenylquinolin-7-yl)-1H-pyrazol-1-yl)cyclobutane-1-carboxylate as a white solid (8.5 mg, 32.6%). LRMS (M+H⁺) m/z calculated 456.2, found 456.1. ¹H NMR (DMSO-d₆, 400 MHz) δ 8.50 (d, 1H), 8.29-8.31 (m, 2H), 8.17-8.20 (m, 2H), 8.06 (d, 1H), 7.75-7.78 (dd, 1H), 7.50-7.59 (m, 3H), 6.31 (s, 2H), 4.76-4.80 (m, 1H), 4.06-4.12 (m, 2H), 2.94-2.98 (m, 1H), 2.70-2.77 (m, 2H), 2.58-2.65 (m, 2H), 1.23 (s, 3H).

### Example 12: Preparation of 5-amino-1-((1s,3s)-3-(morpholinomethyl)cyclobutyl)-3-(2-phenylquinolin-7-yl)-1H-pyrazole-4-carboxamide

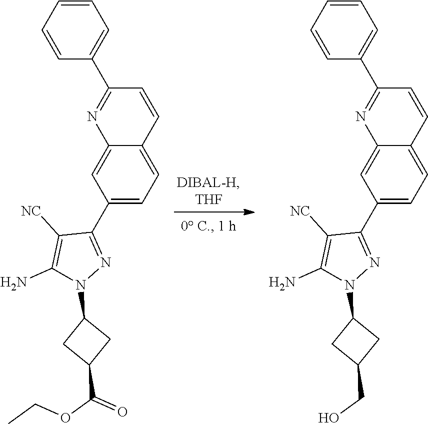 [SMILES: CCOC(=O)[C@H]1C[C@@H](n2nc(-c3ccc4ccc(-c5ccccc5)nc4c3)c(C#N)c2N)C1 ; N#Cc1c(-c2ccc3ccc(-c4ccccc4)nc3c2)nn([C@H]2C[C@@H](CO)C2)c1N]

To a stirred solution of ethyl (1s,3s)-3-(5-amino-4-cyano-3-(2-phenylquinolin-7-yl)-1H-pyrazol-1-yl)cyclobutane-1-carboxylate (2.0 g, 4.6 mmol, 1.0 eq) in anhydrous THF (20 mL) was added DIBAL-H in hexane (1 N, 9.2 mL, 9.2 mmol, 2.0 eq) under ice bath. The reaction was stirred for 2 h at 0° C., then quenched by saturated NH₄Cl aqueous solution (20.0 mL), and extracted by EtOAc (50 mL×3). The combined organic layers were dried over sodium sulfate and concentrated in vacuum. The resulting residue was purified by column chromatography on silica gel (PE/EtOAc=1/1, v/v) to afford 5-amino-1-((1s,3s)-3-(hydroxymethyl)cyclobutyl)-3-(2-phenylquinolin-7-yl)-1H-pyrazole-4-carbonitrile as a light yellow oil (600 mg, 33.3%). LRMS (M+H⁺) m/z calculated 396.2, found 396.1.

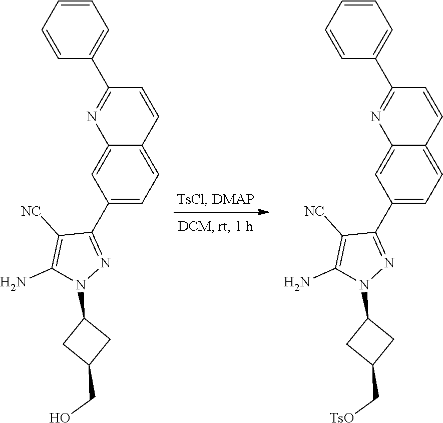 [SMILES: N#Cc1c(-c2ccc3ccc(-c4ccccc4)nc3c2)nn([C@H]2C[C@@H](CO)C2)c1N ; Cc1ccc(S(=O)(=O)OC[C@H]2C[C@@H](n3nc(-c4ccc5ccc(-c6ccccc6)nc5c4)c(C#N)c3N)C2)cc1]

To a stirred solution of 5-amino-1-((1s,3s)-3-(hydroxymethyl)cyclobutyl)-3-(2-phenylquinolin-7-yl)-1H-pyrazole-4-carbonitrile (600 mg, 1.5 mmol, 1.0 eq) and DMAP (366.6 mg, 3.0 mmol, 2.0 eq) in DCM (30 mL) was added TsCl (438.6 mg, 2.3 mmol, 1.5 eq) in portions. The reaction mixture was stirred at 35° C. for 1 h, then quenched by H₂O (20.0 mL) and extracted by EtOAc (50 mL×3). The combined organic layers were dried over sodium sulfate and concentrated in vacuum. The resulting residue was purified by column chromatography on silica gel (PE/EtOAc=2/1, v/v) to afford ((1s,3s)-3-(5-amino-4-cyano-3-(2-phenylquinolin-7-yl)-1H-pyrazol-1-yl)cyclobutyl)methyl 4-methylbenzenesulfonate as a light yellow solid (800 mg, 95.9%). LRMS (M+H⁺) m/z calculated 550.2, found 550.3.

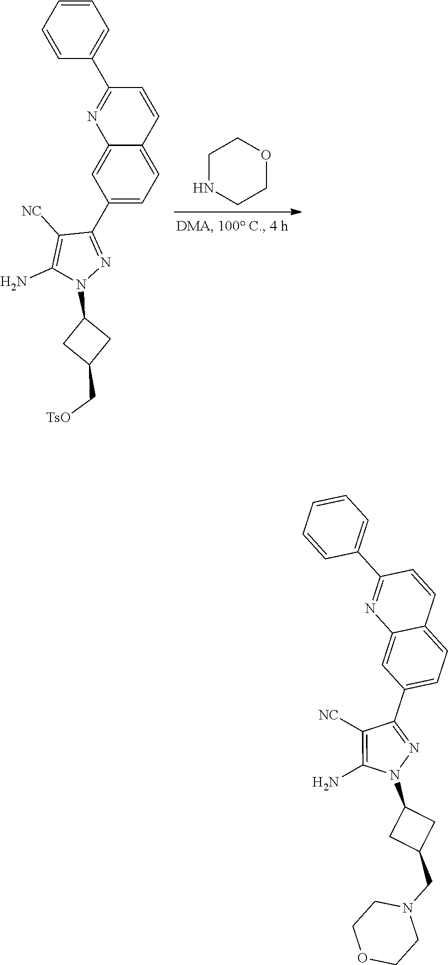 [SMILES: Cc1ccc(S(=O)(=O)OC[C@H]2C[C@@H](n3nc(-c4ccc5ccc(-c6ccccc6)nc5c4)c(C#N)c3N)C2)cc1 ; C1COCCN1 ; N#Cc1c(-c2ccc3ccc(-c4ccccc4)nc3c2)nn([C@H]2C[C@@H](CN3CCOCC3)C2)c1N]

The solution of ((1s,3s)-3-(5-amino-4-cyano-3-(2-phenylquinolin-7-yl)-1H-pyrazol-1-yl)cyclobutyl)methyl 4-methylbenzenesulfonate (800 mg, 1.5 mmol, 1.0 eq) and morpholine (652.5 mg, 7.5 mmol, 5.0 eq) in DMA (10.0 mL) was stirred at 100° C. for 4 h, then quenched by H₂O (100.0 mL) and extracted by DCM/MeOH (10/1, 50 mL×3). The combined organic layers were dried over sodium sulfate and concentrated in vacuum. The resulting residue was purified by column chromatography on silica gel (DCM/MeOH=10/1, v/v) to afford 5-amino-1-((1s,3s)-3-(morpholinomethyl)cyclobutyl)-3-(2-phenylquinolin-7-yl)-1H-pyrazole-4-carbonitrile as a light yellow oil (600 mg, 88.6%). LRMS (M+H⁺) m/z calculated 465.2, found 465.1.

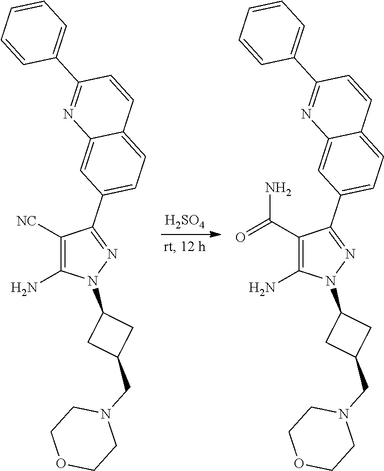

The mixture of 5-amino-1-((1s,3s)-3-(morpholinomethyl)cyclobutyl)-3-(2-phenylquinolin-7-yl)-1H-pyrazole-4-carbonitrile (400 mg, 0.9 mmol, 1.0 eq) in H₂SO₄ (1 mL) was stirred at rt for 12 h, then quenched by H₂O (20.0 mL) and the mixture was adjusted to pH 8.0 by adding saturated Na₂CO₃ aqueous solution, then extracted by DCM:MeOH (10:1, 50 mL×3). The combined organic layers were dried over sodium sulfate and concentrated in vacuum. The resulting residue was purified by reverse chromatography, eluted with MeCN in H₂O (10% to 70%) to afford 5-amino-1-((1s,3s)-3-(morpholinomethyl)cyclobutyl)-3-(2-phenylquinolin-7-yl)-1H-pyrazole-4-carboxamide as a white solid (301.5 mg, 72.5%). LRMS (M+H⁺) m/z calculated 483.2, found 483.1. ¹H NMR (MeOD-d₄, 400 MHz) δ 9.22 (d, 1H), 8.72 (s, 1H), 8.17-8.44 (m, 5H), 7.77-7.81 (m, 3H), 4.03-4.07 (m 2H), 3.83-4.03 (m, 3H), 3.41-3.50 (m, 5H), 3.18-3.30 (m, 3H), 2.70-2.82 (m, 2H), 2.62 (s, 1H).

### Example 13: Preparation of 5-amino-1-((1r,3r)-3-(morpholinomethyl)cyclobutyl)-3-(2-phenylquinolin-7-yl)-1H-pyrazole-4-carboxamide

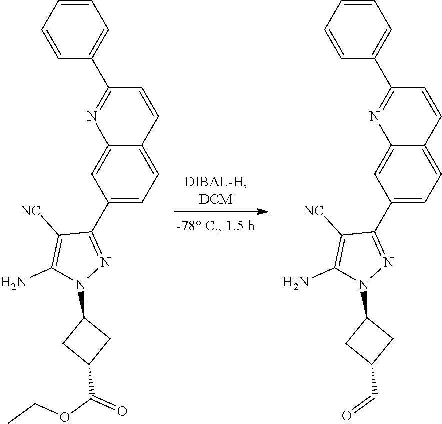 [SMILES: CCOC(=O)[C@H]1C[C@H](n2nc(-c3ccc4ccc(-c5ccccc5)nc4c3)c(C#N)c2N)C1 ; N#Cc1c(-c2ccc3ccc(-c4ccccc4)nc3c2)nn([C@H]2C[C@H](C=O)C2)c1N]

To a solution of ethyl (1r,3r)-3-(5-amino-4-cyano-3-(2-phenylquinolin-7-yl)-1H-pyrazol-1-yl)cyclobutane-1-carboxylate (530 mg, 1.2 mmol, 1.0 eq) in DCM (15 mL), was added DIBAL-H (1 M, 1.5 mL, 1.5 mmol, 1.2 eq) dropwise at −78° C. over a period of 10 min under N₂. The reaction mixture was stirred at −70° C. for 30 min, then quenched with water (5 mL) at 0° C., filtered. The combined organic layers were washed with brine (15 mL), dried over Sodium sulfate, filtered and concentrated in vacuum to give 5-amino-1-((1r,3r)-3-formylcyclobutyl)-3-(2-phenylquinolin-7-yl)-1H-pyrazole-4-carbonitrile (471 mg, ca 100%) as white solid. LRMS (M+H⁺) m/z calculated 394.2, found 394.1.

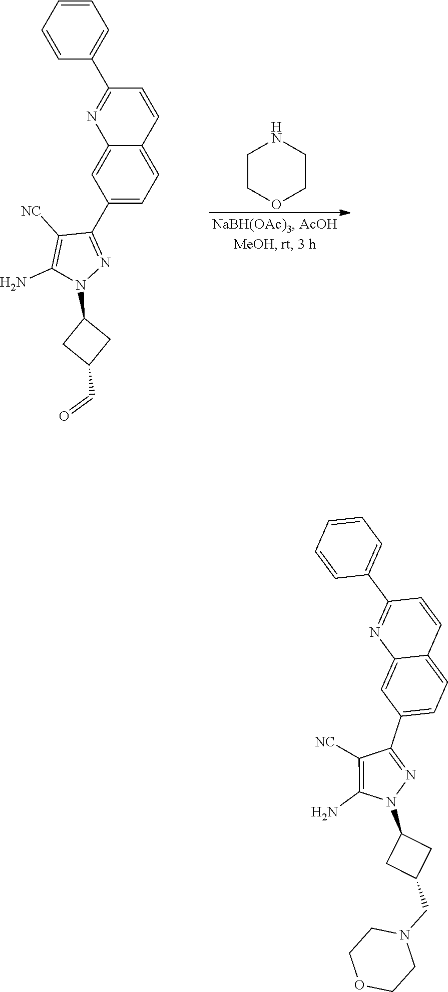 [SMILES: N#Cc1c(-c2ccc3ccc(-c4ccccc4)nc3c2)nn([C@H]2C[C@H](C=O)C2)c1N ; C1COCCN1 ; N#Cc1c(-c2ccc3ccc(-c4ccccc4)nc3c2)nn([C@H]2C[C@H](CN3CCOCC3)C2)c1N]

To a solution of 5-amino-1-((1r,3r)-3-formylcyclobutyl)-3-(2-phenylquinolin-7-yl)-1H-pyrazole-4-carbonitrile (200 mg, 0.51 mmol, 1.0 eq) in MeOH (20 mL) were added NaBH(OAc)₃ (161.8 mg, 0.76 mmol, 1.5 eq), AcOH (15.3 mg, 0.25 mmol, 0.5 eq) and morpholine (66.4 mg, 0.76 mmol, 1.5 eq) and the mixture was stirred at 20° C. for 3 h, then quenched with water and MeOH was removed in vacuum. The resulting residue was diluted with H₂O (20 mL), extracted with EtOAc (50 mL) and the combined organic layers were dried over anhydrous sodium sulfate and concentrated in vacuum. The resulting residue was purified by column chromatography on silica gel (PE/EA=1/1, v/v) to obtain 5-amino-1-((1r,3r)-3-(morpholinomethyl)cyclobutyl)-3-(2-phenylquinolin-7-yl)-1H-pyrazole-4-carbonitrile (150 mg, 63.5%) as a white solid.

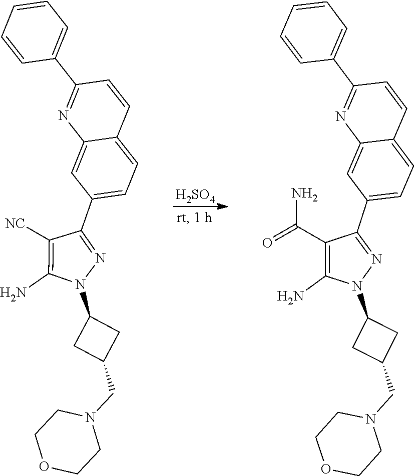

5-Amino-1-((1r,3r)-3-(morpholinomethyl)cyclobutyl)-3-(2-phenylquinolin-7-yl)-1H-pyrazole-4-carbonitrile (150 mg, 0.32 mmol, 1.0 eq) was added to 98% sulfuric acid (3 mL) at rt. The mixture was stirred for 1 h, then slowly poured into ice. The mixture was adjusted to pH 7 with saturated NaHCO₃ aqueous solution, and extracted with ethyl acetate (30 mL×3). The combined organic layers were washed with brine (30 mL), dried over anhydrous sodium sulfate and concentrated in vacuum. The resulting residue was purified by Prep-HPLC to afford 5-amino-1-((1r,3r)-3-(morpholinomethyl)cyclobutyl)-3-(2-phenylquinolin-7-yl)-1H-pyrazole-4-carboxamide (60.2 mg, 38.7%) as a white solid. LRMS (M+H⁺) m/z calculated 483.2, found 483.1. ¹H NMR (DMSO-d₆, 400 MHz) δ 8.50 (d, 1H), 8.29-8.32 (m, 2H), 8.22 (s, 1H), 8.18 (d, 1H), 8.06 (d, 1H), 7.80 (dd, 1H), 7.52-7.60 (m, 3H), 6.28 (s, 2H), 4.96-5.00 (m, 1H), 3.58 (t, 4H), 2.63-2.68 (m, 2H), 2.50-2.52 (m, 3H), 2.36-2.39 (m, 4H), 2.16 (t, 2H).

### Example 14: Preparation of 5-amino-1-((1r,3r)-3-(azetidin-1-ylmethyl)cyclobutyl)-3-(2-phenylquinolin-7-yl)-1H-pyrazole-4-carboxamide

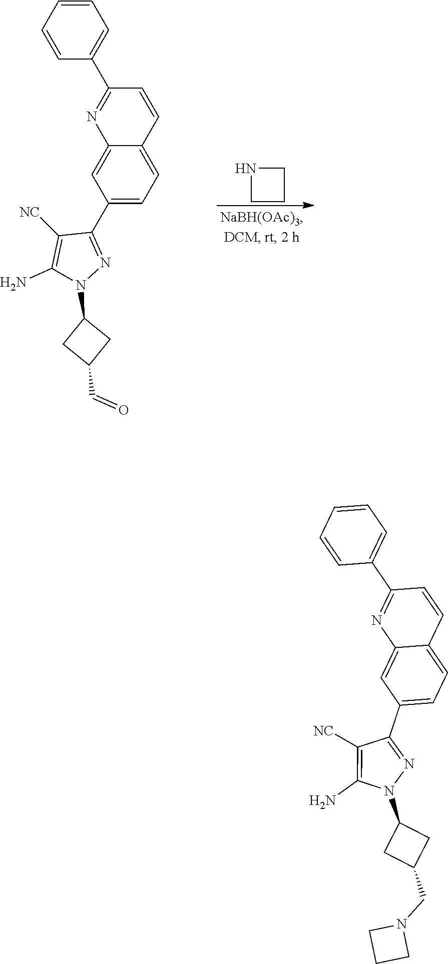

To a solution of 5-amino-1-((1r,3r)-3-formylcyclobutyl)-3-(2-phenylquinolin-7-yl)-1H-pyrazole-4-carbonitrile (70 mg, 0.18 mmol, 1.0 eq) in DCM (20 mL) were added NaBH(OAc)₃ (56.6 mg, 0.27 mmol, 1.5 eq), AcOH (5.3 mg, 0.09 mmol, 0.5 eq) and azetidine (15.2 mg, 0.27 mmol, 1.5 eq). The mixture was stirred at 20° C. for 3 h, then quenched with water and concentrated in vacuum. The resulting residue was diluted with H₂O (20 mL), extracted with EtOAc (50 mL). The combined organic layers were washed with brine (20 mL), dried over anhydrous sodium sulfate, filtered and concentrated in vacuum. The resulting residue was purified by column chromatography on silica gel (PE/EA=1/1, v/v) to obtain 5-amino-1-((1r,3r)-3-(azetidin-1-ylmethyl)cyclobutyl)-3-(2-phenylquinolin-7-yl)-1H-pyrazole-4-carbonitrile (20 mg, 63.5%) as a yellow solid. LRMS (M+H⁺) m/z calculated 435.2, found 435.1.

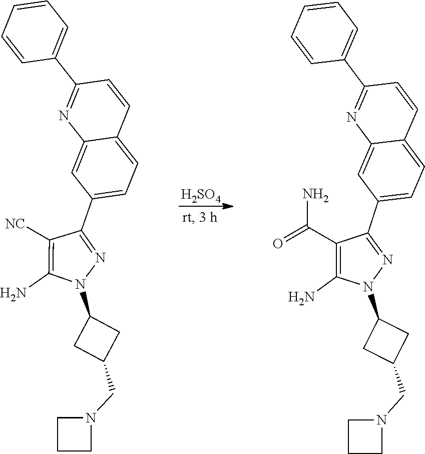

5-Amino-1-((1r,3r)-3-(azetidin-1-ylmethyl)cyclobutyl)-3-(2-phenylquinolin-7-yl)-1H-pyrazole-4-carbonitrile (20 mg, 0.32 mmol, 1.0 eq) was added to 98% sulfuric acid (3 mL) and the mixture was stirred for 3 h at rt, then slowly poured into ice and adjusted to pH 7 with the addition of saturated NaHCO₃ (aq). The mixture was extracted with ethyl acetate (30 mL×3). The combined organic layers were washed with brine (30 mL), dried over anhydrous sodium sulfate and concentrated in vacuum. The resulting residue was purified by Prep-HPLC to afford 5-amino-1-((1r,3r)-3-(morpholinomethyl)cyclobutyl)-3-(2-phenylquinolin-7-yl)-1H-pyrazole-4-carboxamide (60.2 mg, 38.7%) as a white solid. LRMS (M+H⁺) m/z calculated 453.2, found 453.1. ¹H NMR (CD₃OD, 400 MHz) δ 8.46 (d, 1H), 8.32 (s, 1H), 8.16 (d, 2H), 8.06 (dd, 2H), 7.76-7.80 (m, 1H), 7.50-7.58 (m, 3H), 4.86-4.90 (m, 1H), 3.31-3.35 (m, 5H), 2.70-2.80 (m, 4H), 2.11-2.28 (m, 4H).

### Example 15: Preparation of 5-amino-3-(2-phenylquinolin-7-yl)-1-((1s,3s)-3-(piperazin-1-ylmethyl)cyclobutyl)-1H-pyrazole-4-carboxamide

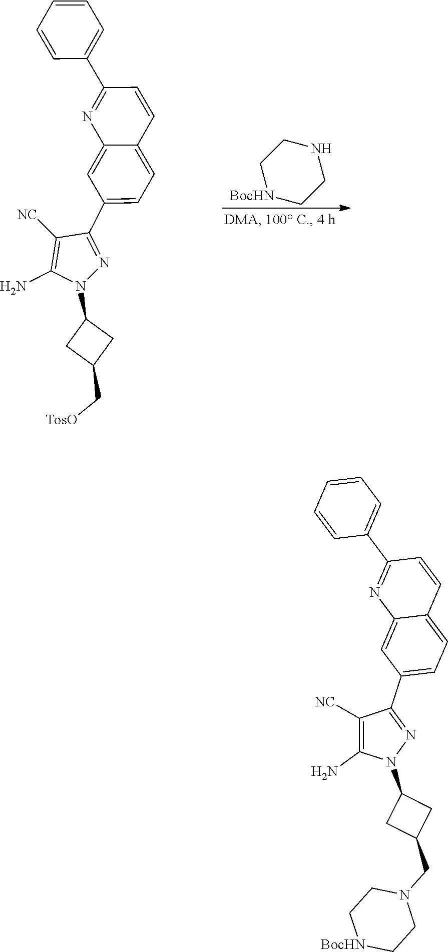

The solution of ((1s,3s)-3-(5-amino-4-cyano-3-(2-phenylquinolin-7-yl)-1H-pyrazol-1-yl)cyclobutyl)methyl 4-methylbenzenesulfonate (40 mg, 0.07 mmol, 1.0 eq), tert-butyl piperazine-1-carboxylate (651.0 mg, 3.5 mmol, 5.0 eq) in DMA (5.0 mL) was stirred at 100° C. for 4 h, then quenched by H₂O (50.0 mL) and extracted by DCM:MeOH (10:1, 20 mL×3). The combined organic layers were dried over sodium sulfate and concentrated in vacuum. The resulting residue was purified by column chromatography on silica gel (DCM/MeOH=10/1, v/v) to afford tert-butyl 4-(((1s,3s)-3-(5-amino-4-cyano-3-(2-phenylquinolin-7-yl)-1H-pyrazol-1-yl)cyclobutyl)methyl)piperazine-1-carboxylate as a light yellow oil (20 mg, 48.8%). LRMS (M+H⁺) m/z calculated 564.3, found 564.4.

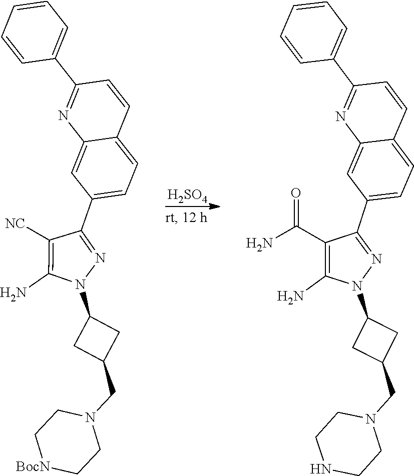

The mixture of tert-butyl 4-(((1s,3s)-3-(5-amino-4-cyano-3-(2-phenylquinolin-7-yl)-1H-pyrazol-1-yl)cyclobutyl)methyl)piperazine-1-carboxylate (20 mg, 0.04 mmol, 1.0 eq) in conc. H₂SO₄ (1.0 mL) was stirred at rt for 12 h, then quenched by H₂O (20.0 mL). Then adjusted to pH 8.0 with the addition of saturated Na₂CO₃ aqueous solution. The mixture was extracted by DCM/MeOH (10/1, 50 mL×3). The combined organic layers were dried over sodium sulfate and concentrated in vacuum. The resulting residue was purified by Prep-HPLC to afford 5-amino-3-(2-phenylquinolin-7-yl)-1-((1s,3s)-3-(piperazin-1-ylmethyl)cyclobutyl)-1H-pyrazole-4-carboxamide as a white solid (1.9 mg, 11.2%). LRMS (M+H⁺) m/z calculated 482.3, found 482.2. ¹H NMR (DMSO-d₆, 400 MHz) δ 8.50 (d, 1H), 8.31 (d, 2H), 8.17-8.19 (m, 2H), 8.06 (d, 1H), 7.77 (d, 1H), 7.52-7.59 (m, 3H), 6.26 (s, 2H), 4.66-4-70 (m, 1H), 2.67-2.72 (m, 4H), 2.39-2.50 (m, 3H), 2.19-2.31 (m, 5H), 2.18-2.19 (m, 3H).

### Example 16: Preparation of 5-amino-3-(2-phenylquinolin-7-yl)-1-((1r,3r)-3-(piperazin-1-ylmethyl)cyclobutyl)-1H-pyrazole-4-carboxamide

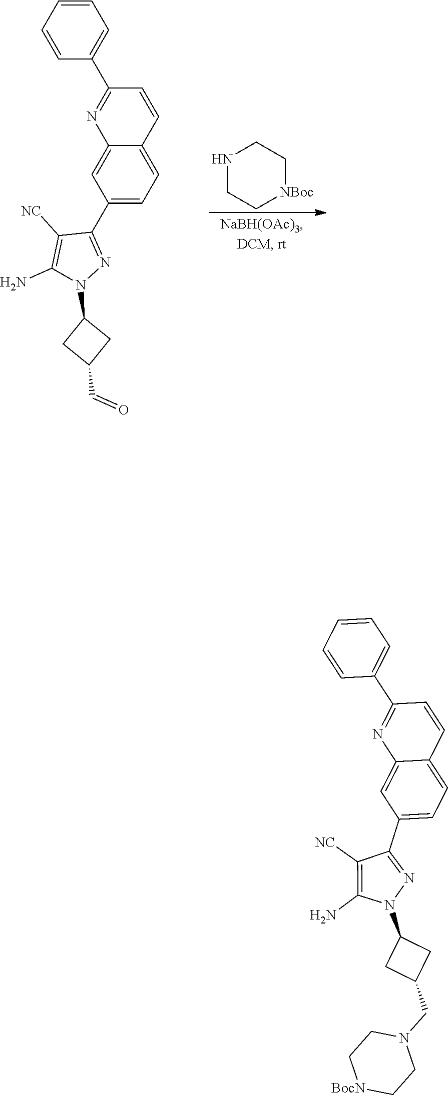 [SMILES: N#Cc1c(-c2ccc3ccc(-c4ccccc4)nc3c2)nn([C@H]2C[C@H](C=O)C2)c1N ; CC(C)(C)OC(=O)N1CCNCC1 ; CC(C)(C)OC(=O)N1CCN(C[C@H]2C[C@H](n3nc(-c4ccc5ccc(-c6ccccc6)nc5c4)c(C#N)c3N)C2)CC1]

To a solution of 5-amino-1-((1r,3r)-3-formylcyclobutyl)-3-(2-phenylquinolin-7-yl)-1H-pyrazole-4-carbonitrile (140 mg, 0.36 mmol, 1.0 eq) in DCM (20 mL) were added NaBH(OAc)₃ (113.2 mg, 0.53 mmol, 1.5 eq), AcOH (10.7 mg, 0.18 mmol, 0.5 eq) and tert-butyl piperazine-1-carboxylate (99.4 mg, 0.53 mmol, 1.5 eq). The mixture was stirred at 20° C. for 3 h, then quenched with water and concentrated in vacuum. The resulting residue was diluted with H₂O (20 mL), extracted with EtOAc (50 mL). The combined organic layers were washed with brine (20 mL), dried over anhydrous sodium sulfate and concentrated in vacuum. The resulting residue was purified by column chromatography (SiO₂, PE/EA=1/1, v/v) to obtain tert-butyl 4-(((1r,3r)-3-(5-amino-4-cyano-3-(2-phenylquinolin-7-yl)-1H-pyrazol-1-yl)cyclobutyl)methyl)piperazine-1-carboxylate (30 mg, 15.0%) as a yellow solid. LRMS (M+H⁺) m/z calculated 564.3, found 564.2.

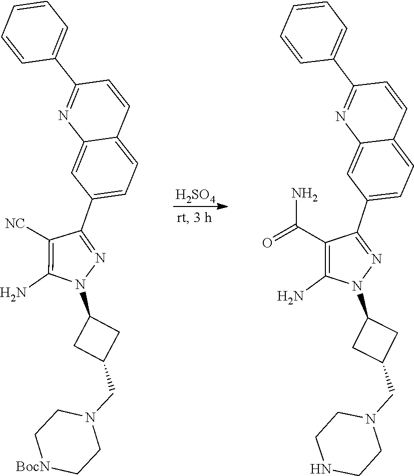

Tert-butyl 4-(((1r,3r)-3-(5-amino-4-cyano-3-(2-phenylquinolin-7-yl)-1H-pyrazol-1-yl)cyclobutyl)methyl)piperazine-1-carboxylate (30 mg, 0.053 mmol, 1.0 eq) was added to conc. H₂SO₄ (3 mL) at rt and the mixture was stirred for 3 h, then slowly poured into ice and adjusted to pH 7 with the addition of saturated NaHCO₃ (aq). The mixture was extracted with ethyl acetate (30 mL×3). The combined organic layers were washed with brine (30 mL), dried over anhydrous sodium sulfate and concentrated in vacuum. The resulting residue was purified by Prep-HPLC to afford 5-amino-3-(2-phenylquinolin-7-yl)-1-((1r,3r)-3-(piperazin-1-ylmethyl)cyclobutyl)-1H-pyrazole-4-carboxamide (6.3 mg, 24.6%) as a white solid. LRMS (M+H⁺) m/z calculated 482.3, found 482.2. ¹H NMR (CD₃OD, 400 MHz) δ 8.79 (d, 1H), 8.46 (s, 1H), 8.21 (d, 2H), 8.14-8.17 (m, 2H), 8.00 (d, 1H), 7.64-7.66 (m, 3H), 4.86-4.95 (m, 1H), 3.47 (brs, 4H), 3.24-3.31 (m, 4H), 2.86-2.91 (m, 3H), 2.44-2.47 (m, 2H).

### Example 17: Preparation of (1s,3s)-3-(5-amino-4-carbamoyl-3-(2-phenylquinolin-7-yl)-1H-pyrazol-1-yl)cyclobutane-1-carboxylic acid

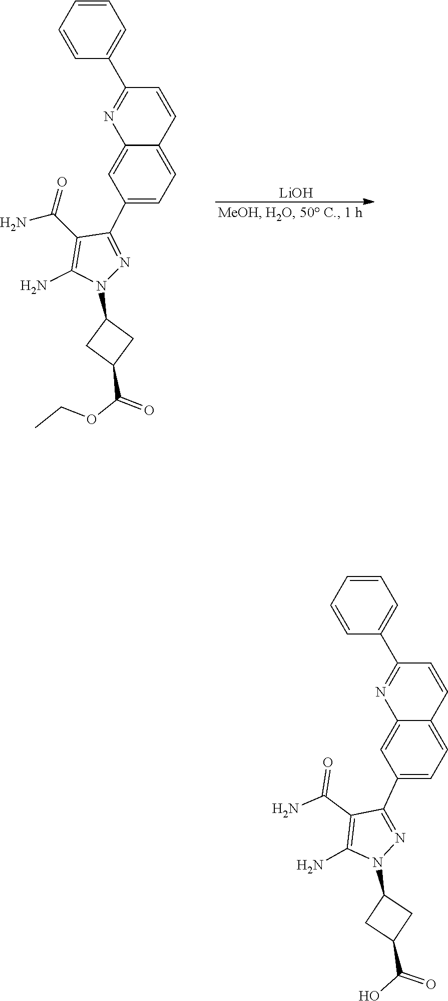 [SMILES: CCOC(=O)[C@H]1C[C@@H](n2nc(-c3ccc4ccc(-c5ccccc5)nc4c3)c(C(N)=O)c2N)C1 ; "LiOH" ; NC(=O)c1c(-c2ccc3ccc(-c4ccccc4)nc3c2)nn([C@H]2C[C@@H](C(=O)O)C2)c1N]

To a solution of ethyl (1s,3s)-3-(5-amino-4-carbamoyl-3-(2-phenylquinolin-7-yl)-1H-pyrazol-1-yl)cyclobutane-1-carboxylate (70 mg, 0.15 mmol, 1.0 eq) in MeOH/H₂O (9 mL/3 mL) was added LiOH (10.8 mg, 0.45 mmol, 3.0 eq). The mixture was stirred at 50° C. for 1 h, then concentrated in vacuum. The mixture was adjusted to pH 5 with 37% HCl, the solid was filtered and further purified by Prep-HPLC to afford (1s,3s)-3-(5-amino-4-carbamoyl-3-(2-phenylquinolin-7-yl)-1H-pyrazol-1-yl)cyclobutane-1-carboxylic acid as a white solid (24.0 mg, 36.5%). LCMS (M+H⁺) m/z calculated 428.2, found 428.1. ¹H NMR (DMSO-d₆, 400 MHz) δ 8.49 (d, 1H), 8.29-8.31 (m, 2H), 8.16-8.19 (m, 2H), 8.05 (d, 1H), 7.76-7.78 (dd, 1H), 7.51-7.58 (m, 3H), 6.37 (s, 2H), 4.71-4.75 (m, 1H), 2.69-2.78 (m, 3H), 2.54-2.57 (m, 2H).

### Example 18: Preparation of 5-amino-1-((1s,3s)-3-(azetidin-1-ylmethyl)cyclobutyl)-3-(2-phenylquinolin-7-yl)-1H-pyrazole-4-carboxamide

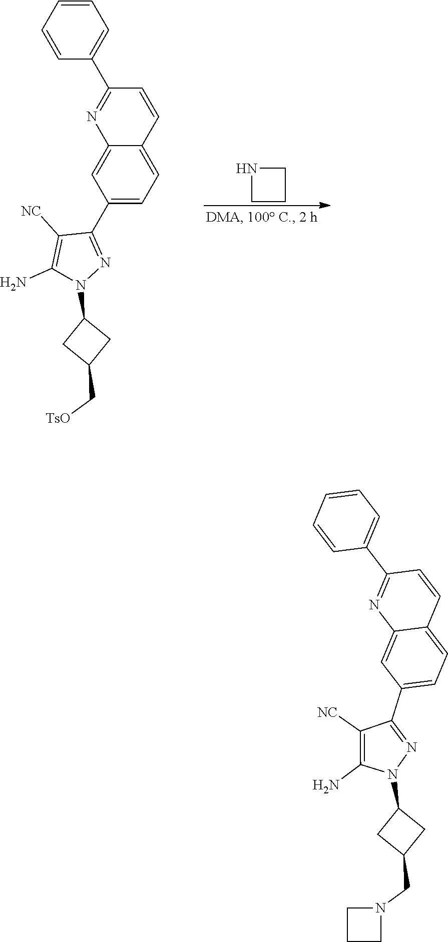

A solution of ((1s,3s)-3-(5-amino-4-cyano-3-(2-phenylquinolin-7-yl)-1H-pyrazol-1-yl)cyclobutyl)methyl 4-methylbenzenesulfonate (140 mg, 0.27 mmol, 1.0 eq) and azetidine (145 mg, 2.7 mmol, 10.0 eq) in DMA (5.0 mL) was stirred at 100° C. for 4 h, then quenched by H₂O (20.0 mL) and extracted by DCM/MeOH (10/1, 20 mL×3). The combined organic layers were dried over sodium sulfate and concentrated in vacuum. The resulting residue was purified by column chromatography on silica gel (DCM/MeOH=10/1, v/v) to afford 5-amino-1-((1s,3s)-3-(azetidin-1-ylmethyl)cyclobutyl)-3-(2-phenylquinolin-7-yl)-1H-pyrazole-4-carbonitrile as a yellow solid (50 mg, 45.2%). LRMS (M+H⁺) m/z calculated 435.2, found 435.1.

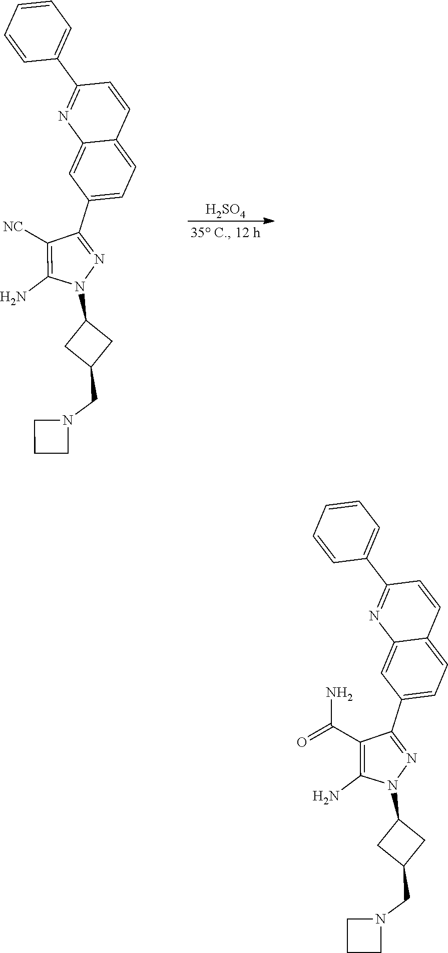

The mixture of 5-amino-1-((1s,3s)-3-(azetidin-1-ylmethyl)cyclobutyl)-3-(2-phenylquinolin-7-yl)-1H-pyrazole-4-carbonitrile (20 mg, 0.04 mmol, 1.0 eq) in H₂SO₄ (2.0 mL) was stirred at rt for 12 h, then quenched by H₂O (20.0 mL) and adjusted to pH=8.0 with the addition of saturated aqueous Na₂CO₃ solution. The mixture was extracted by DCM/MeOH (10/1, 30 mL×3). The combined organic layers were dried over sodium sulfate and concentrated in vacuum. The resulting residue was purified by reverse chromatography, eluted with MeCN in H₂O from 10% to 80% to afford 5-amino-1-((1s,3s)-3-(azetidin-1-ylmethyl)cyclobutyl)-3-(2-phenylquinolin-7-yl)-1H-pyrazole-4-carboxamide as a white solid (1.1 mg, 5.2%). LRMS (M+H⁺) m/z calculated 453.2, found 453.2. ¹H NMR (DMSO-d₆, 400 MHz) δ 8.50 (d, 1H), 8.29-8.31 (m, 2H), 8.17-8.20 (m, 2H), 8.07 (d, 1H), 7.77 (d, 1H), 7.52-7.59 (m, 3H), 6.27 (s, 2H), 4.67-4.71 (m, 1H), 2.67-2.68 (m, 1H), 2.33-2.38 (m, 6H), 2.21-2.25 (m, 3H), 1.98-2.08 (m, 3H).

### Example 19: Preparation of 5-amino-1-((1s,3s)-3-((4-methylpiperazin-1-yl)methyl)cyclobutyl)-3-(2-phenylquinolin-7-yl)-1H-pyrazole-4-carboxamide

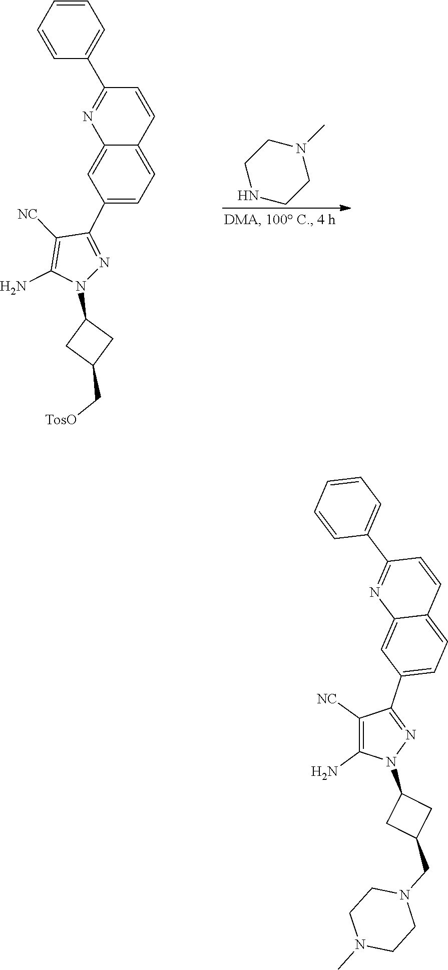

A solution of ((1s,3s)-3-(5-amino-4-cyano-3-(2-phenylquinolin-7-yl)-1H-pyrazol-1-yl)cyclobutyl)methyl 4-methylbenzenesulfonate (130 mg, 0.24 mmol, 1.0 eq) and 1-methylpiperazine (118.4 mg, 1.2 mmol, 5.0 eq) in DMA (5.0 mL) was stirred at 100° C. for 4 h, then quenched by H₂O (50.0 mL) and extracted by DCM/MeOH (10/1, 20 mL×3). The combined organic layers were dried over sodium sulfate and concentrated in vacuum. The resulting residue was purified by column chromatography on silica gel (DCM/MeOH=10/1, v/v) to afford 5-amino-1-((1s,3s)-3-((4-methylpiperazin-1-yl)methyl)cyclobutyl)-3-(2-phenylquinolin-7-yl)-1H-pyrazole-4-carbonitrile as a light yellow oil (90 mg, 79.6%). LRMS (M+H⁺) m/z calculated 478.3, found 478.4.

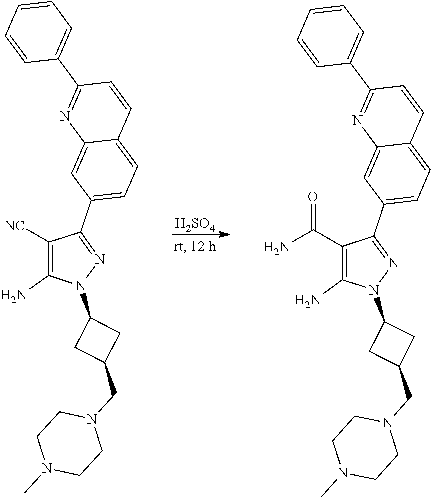

The mixture of 5-amino-1-((1s,3s)-3-((4-methylpiperazin-1-yl)methyl)cyclobutyl)-3-(2-phenylquinolin-7-yl)-1H-pyrazole-4-carbonitrile (40 mg, 0.08 mmol, 1.0 eq) in conc. H₂SO₄ (1.0 mL) was stirred at rt for 12 h, then quenched by H₂O (20.0 mL) and the mixture was adjusted to pH 8.0 with the addition of saturated Na₂CO₃ aqueous solution. The mixture was extracted by DCM:MeOH (10:1, 50 mL×3). The combined organic layers were dried over sodium sulfate and concentrated in vacuum. The resulting residue was purified by Prep-HPLC to afford 5-amino-1-((1s,3s)-3-((4-methylpiperazin-1-yl)methyl)cyclobutyl)-3-(2-phenylquinolin-7-yl)-1H-pyrazole-4-carboxamide as a white solid (21.7 mg, 52.3%). LRMS (M+H⁺) m/z calculated 496.3, found 496.3. ¹H NMR (DMSO-d₆, 400 MHz) δ 8.50 (d, 1H), 8.30-8.32 (m, 2H), 8.17-8.19 (m, 2H), 8.06 (d, 1H), 7.77 (d, 1H), 7.52-7.59 (m, 3H), 6.26 (s, 2H), 4.69-4.71 (m, 1H), 2.49-2.51 (m, 3H), 2.36-2.42 (m, 7H), 2.13-2.35 (m, 5H), 2.14 (s, 3H).

### Example 20: Preparation of ethyl (1r,3r)-3-(5-amino-4-carbamoyl-3-(2-phenylquinolin-7-yl)-1H-pyrazol-1-yl)-1-methylcyclobutane-1-carboxylate

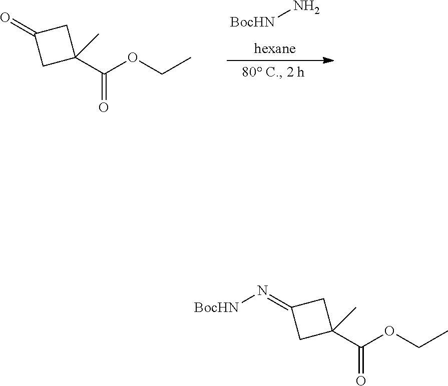 [SMILES: CCOC(=O)C1(C)CC(=O)C1 ; CC(C)(C)OC(=O)NN ; CCOC(=O)C1(C)CC(=NNC(=O)OC(C)(C)C)C1]

To the solution of ethyl 1-methyl-3-oxocyclobutane-1-carboxylate (5.0 g, 32.1 mmol, 1.0 eq) in hexane (80 mL) was added tert-butyl hydrazinecarboxylate (5.1 g, 38.4 mmol, 1.2 eq) at rt. The mixture was stirred at 80° C. for 2 h, then concentrated in vacuum. The resulting residue was purified by column chromatography on silica gel (PE/EA=2/1, v/v) to afford tert-butyl 2-(3-(ethoxycarbonyl)-3-methylcyclobutylidene)hydrazine-1-carboxylate (5 g, 58.1%) as a white solid. LRMS (M+H⁺) m/z calculated 271.2, found 271.1.

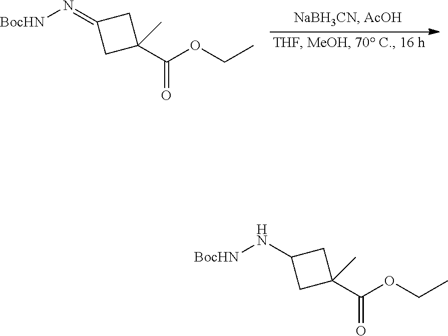 [SMILES: CCOC(=O)C1(C)CC(=NNC(=O)OC(C)(C)C)C1 ; CCOC(=O)C1(C)CC(NNC(=O)OC(C)(C)C)C1]

To a stirred solution of tert-butyl 2-(3-(ethoxycarbonyl)-3-methylcyclobutylidene)hydrazine-1-carboxylate (5 g 18.4 mmol, 1.0 eq) and NaBH₃CN (2.3 g, 36.7 mmol, 2.0 eq) in MeOH (50 mL)/THF (50 mL) was added AcOH (0.6 mL, 9.2 mmol, 0.5 eq) at rt. The reaction mixture was stirred at 70° C. for 16 h, then cooled to rt and concentrated in vacuum. The resulting residue was diluted with EtOAc (200 mL), washed with water (200 mL) and brine (200 mL), dried over anhydrous sodium sulfate, and concentrated in vacuum. The resulting residue was purified by column chromatography on silica gel (DCM/MeOH=40:1, v/v) to afford tert-butyl 2-(3-(ethoxycarbonyl)-3-methylcyclobutyl)hydrazine-1-carboxylate (4.9 g, ca 100%) as a white solid. LRMS (M+H⁺) m/z calculated 273.2, found 273.1

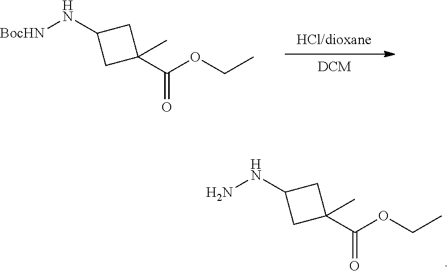 [SMILES: CCOC(=O)C1(C)CC(NNC(=O)OC(C)(C)C)C1 ; CCOC(=O)C1(C)CC(NN)C1]

To a stirred solution of tert-butyl 2-(3-(ethoxycarbonyl)-3-methylcyclobutyl)hydrazine-1-carboxylate (4.9 g, 18.0 mmol, 1.0 eq) in DCM (30 mL) was added 4 N HCl/Dioxane (30 mL). The reaction was stirred at 30° C. for 30 min, then concentrated in vacuum to afford ethyl 3-hydrazineyl-1-methylcyclobutane-1-carboxylate (4 g, ca 100%) as a white solid. LRMS (M+H⁺) m/z calculated 173.1, found 173.0.

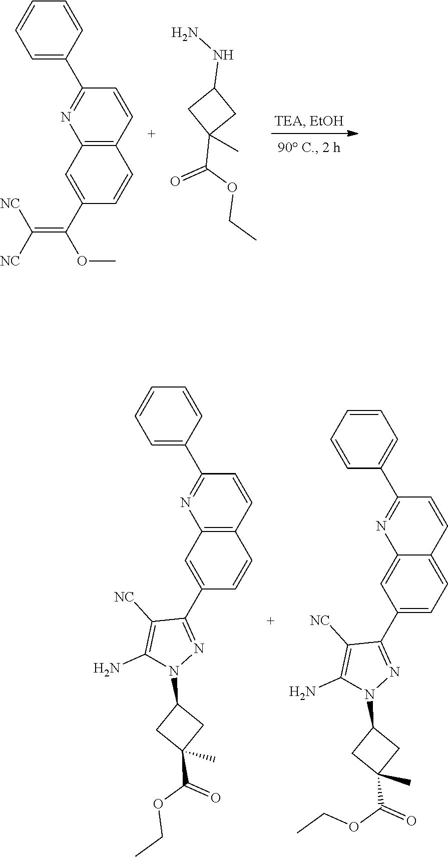

To a solution of 2-(methoxy(2-phenylquinolin-7-yl)methylene)malononitrile (3.0 g, 9.7 mmol, 1.0 eq) and ethyl 3-hydrazineyl-1-methylcyclobutane-1-carboxylate (2.5 g, 14.5 mmol, 1.5 eq) in EtOH (30 mL) was added TEA (10.7 mL, 77.5 mmol, 8.0 eq) at rt. The reaction mixture was stirred at 90° C. for 2 hours, then concentrated in vacuum. The resulting residue was purified by column chromatography on silica gel (PE/EA=2/1, v/v) to afford ethyl (1s,3s)-3-(5-amino-4-cyano-3-(2-phenylquinolin-7-yl)-1H-pyrazol-1-yl)-1-methylcyclobutane-1-carboxylate (45 mg, 1.1%) and ethyl (1r,3r)-3-(5-amino-4-cyano-3-(2-phenylquinolin-7-yl)-1H-pyrazol-1-yl)-1-methylcyclobutane-1-carboxylate (600 mg, 13.9%) as a white solid. Ethyl (1s,3s)-3-(5-amino-4-cyano-3-(2-phenylquinolin-7-yl)-1H-pyrazol-1-yl)-1-methylcyclobutane-1-carboxylate: 1H NMR (DMSO-d₆, 400 MHz) δ 8.54 (s, 1H), 8.50 (d, 1H), 8.30-8.32 (m, 2H), 8.19 (d, 1H), 8.05-8.12 (m, 2H), 7.51-7.61 (m, 3H), 6.88 (s, 2H), 4.99-5.04 (m, 1H), 4.16 (q, 2H), 2.93-2.98 (m, 2H), 2.31-2.37 (m, 2H), 1.48 (s, 3H), 1.26 (t, 3H). LRMS (M+H⁺) m/z calculated 452.2, found 452.1. Ethyl (1r,3r)-3-(5-amino-4-cyano-3-(2-phenylquinolin-7-yl)-1H-pyrazol-1-yl)-1-methylcyclobutane-1-carboxylate: ¹H NMR (DMSO-d₆, 400 MHz) δ 8.56 (s, 1H), 8.50 (d, 1H), 8.29-8.32 (m, 2H), 8.19 (d, 1H), 8.11-8.12 (m, 2H), 7.53-7.61 (m, 3H), 6.85 (s, 2H), 4.88-4.93 (m, 1H), 4.18 (q, 2H), 2.86-2.93 (m, 2H), 2.55-2.61 (m, 2H), 1.50 (s, 3H), 1.26 (t, 3H). LRMS (M+H⁺) m/z calculated 452.2, found 452.1.

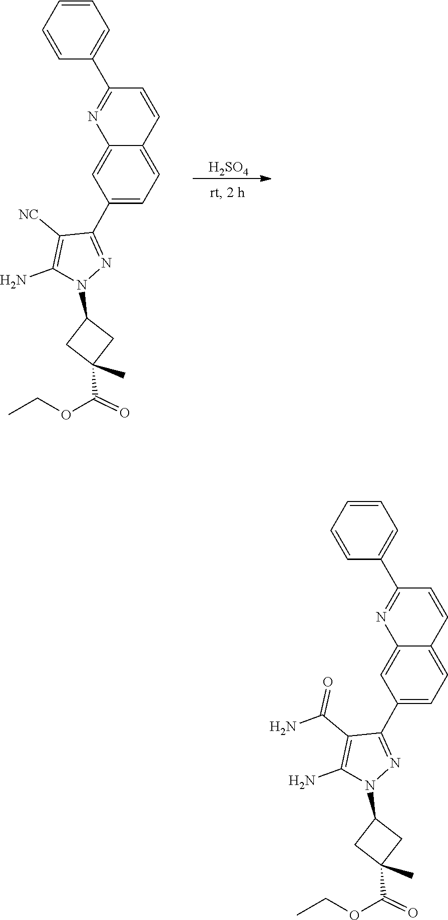

Ethyl (1r,3r)-3-(5-amino-4-cyano-3-(2-phenylquinolin-7-yl)-1H-pyrazol-1-yl)-1-methylcyclobutane-1-carboxylate (100 mg, 0.22 mmol, 1.0 eq) was added to conc. H₂SO₄ (5 mL) at rt, and the mixture was stirred for 2 h, then slowly poured into ice and adjusted to pH 7 with the addition of saturated NaHCO₃ solution, and extracted with ethyl acetate (30 mL×3). The combined organic layers were washed with brine (30 mL), dried over anhydrous sodiumsulfate, and concentrated in vacuum. The resulting residue was purified by Prep-HPLC to afford ethyl (1r,3r)-3-(5-amino-4-carbamoyl-3-(2-phenylquinolin-7-yl)-1H-pyrazol-1-yl)-1-methylcyclobutane-1-carboxylate (23.8 mg, 23.1%) as a white solid. LRMS (M+H⁺) m/z calculated 470.2, found 470.2. ¹H NMR (DMSO-d₆, 400 MHz) δ 8.50 (d, 1H), 8.30 (d, 2H), 8.22 (s, 1H), 8.18 (d, 1H), 8.06 (d, 1H), 7.79 (dd, 1H), 7.51-7.59 (m, 3H), 6.31 (s, 2H), 4.85-4.90 (m, 1H), 4.17 (q, 2H), 2.83-2.89 (m, 2H), 2.52-2.58 (m, 2H), 1.44 (s, 3H), 1.26 (t, 3H).

### Example 21: Preparation of 5-amino-1-((1r,3r)-3-((4-methylpiperazin-1-yl)methyl)cyclobutyl)-3-(2-phenylquinolin-7-yl)-1H-pyrazole-4-carboxamide

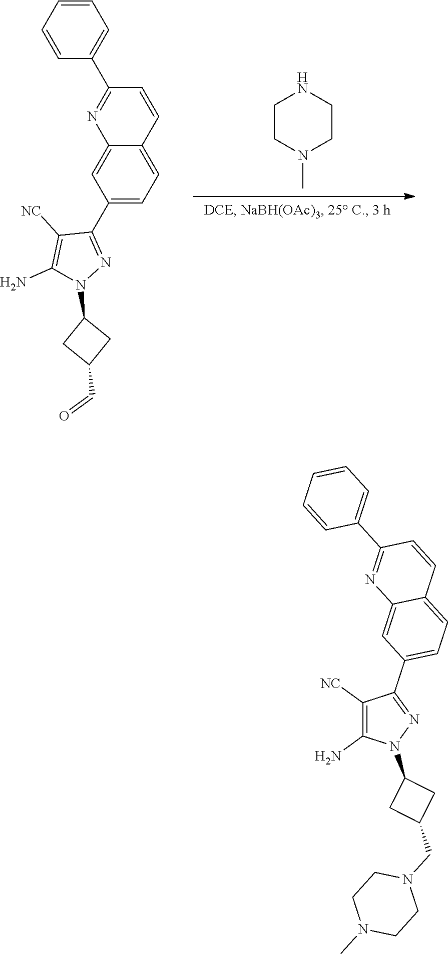

To a solution of 5-amino-1-((1r,3r)-3-formylcyclobutyl)-3-(2-phenylquinolin-7-yl)-1H-pyrazole-4-carbonitrile (160 mg, 0.41 mmol, 1.0 eq) and 1-methylpiperazine (48.8 mg, 0.49 mmol, 1.2 eq) in DCE (35 mL) was added NaBH(OAc)₃ (172 mg, 0.81 mmol. 2.0 eq). The mixture was stirred at 25° C. for 3 h, then concentrated in vacuum. The resulting residue was purified by column chromatography on silica gel (PE/EA=20/1, v/v) to afford 5-amino-1-((1r,3r)-3-((4-methylpiperazin-1-yl)methyl)cyclobutyl)-3-(2-phenylquinolin-7-yl)-1H-pyrazole-4-carbonitrile as a yellow solid (110 mg, 56.6%). LRMS (M+H⁺) m/z calculated 474.3, found 474.2.

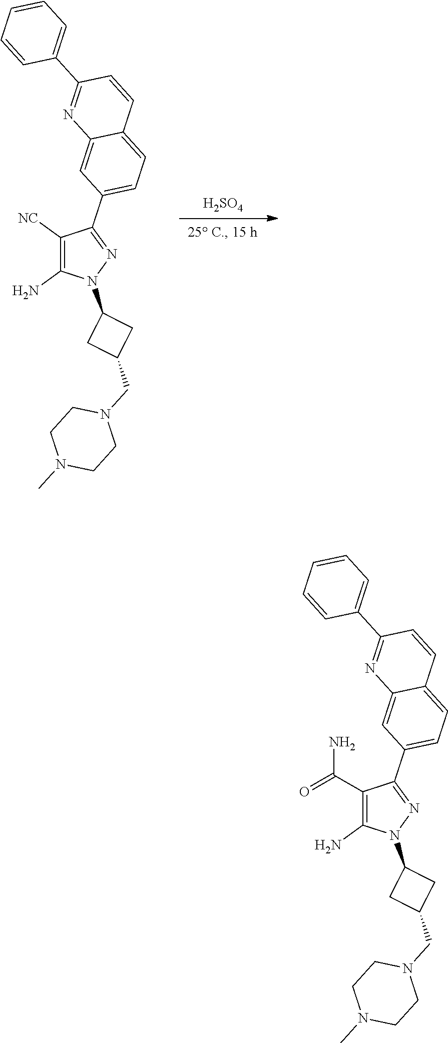

A solution of 5-amino-1-((1r,3r)-3-((4-methylpiperazin-1-yl)methyl)cyclobutyl)-3-(2-phenylquinolin-7-yl)-1H-pyrazole-4-carbonitrile (110 mg, 0.23 mmol, 1.0 eq) in conc. H₂SO₄ (3 mL) was stirred at 25° C. for 15 h, then adjusted to pH=8 with the addition of aqueous saturated sodium carbonate solution. The mixture was extracted with EtOAc (30 mL×2). The combined organic layers were washed with brine (20 mL), dried over anhydrous sodium sulfate, and concentrated in vacuum. The resulting residue was purified by Prep-HPLC to afford to 5-amino-1-((1r,3r)-3-((4-methylpiperazin-1-yl)methyl)cyclobutyl)-3-(2-phenylquinolin-7-yl)-1H-pyrazole-4-carboxamide as a white solid (26.4 mg, 23.2%). LRMS (M+H⁺) m/z calculated 496.3, found 496.3. ¹H NMR (DMSO-d₆, 400 MHz) δ 8.49 (d, 1H), 8.29-8.31, (m, 2H), 8.16-8.21, (m, 2H), 8.05 (d, 1H), 7.79 (dd, 1H), 7.49-7.59 (m, 3H), 6.26 (s, 2H), 4.94-4.98 (m, 1H), 2.63-2.65 (m, 2H), 2.48-2.49, (m, 4H), 2.24-2.37 (m, 7H), 2.11-2.15 (m, 5H).

### Example 22: Preparation of (1r,3r)-3-(5-amino-4-carbamoyl-3-(2-phenylquinolin-7-yl)-1H-pyrazol-1-yl)-1-methylcyclobutane-1-carboxylic acid

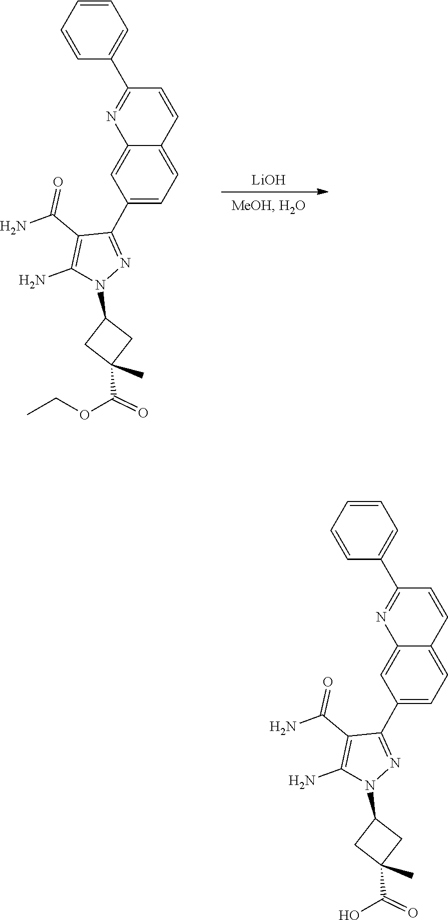 [SMILES: CCOC(=O)[C@]1(C)C[C@@H](n2nc(-c3ccc4ccc(-c5ccccc5)nc4c3)c(C(N)=O)c2N)C1 ; "LiOH" ; C[C@]1(C(=O)O)C[C@H](n2nc(-c3ccc4ccc(-c5ccccc5)nc4c3)c(C(N)=O)c2N)C1]

A solution of ethyl (1r,3r)-3-(5-amino-4-carbamoyl-3-(2-phenylquinolin-7-yl)-1H-pyrazol-1-yl)-1-methylcyclobutane-1-carboxylate (22 mg, 0.046 mmol, 1.0 eq) and LiOH (1.5 mg, 0.07 mmol, 1.5 eq) in MeOH/H₂O (20 mL/5 mL) was stirred at 30° C. for 5 h, then adjusted to pH 5 with 37% HCl and concentrated in vacuum. The resulting residue was purified by Prep-HPLC to afford (1r,3r)-3-(5-amino-4-carbamoyl-3-(2-phenylquinolin-7-yl)-1H-pyrazol-1-yl)-1-methylcyclobutane-1-carboxylic acid (14.4 mg, 70.0%) as a yellow solid. LRMS (M+H⁺) m/z calculated 442.2, found 442.1. ¹H NMR (DMSO-d₆, 400 MHz) δ 8.50 (d, 1H), 8.30 (d, 2H), 8.21 (s, 1H), 8.17 (d, 1H), 8.06 (d, 1H), 7.79 (dd, 1H), 7.49-7.60 (m, 3H), 6.28 (s, 2H), 4.82-4.87 (m, 1H), 2.74-2.80 (m, 2H), 2.27-2.32 (m, 2H), 1.30 (s, 3H).

### Example 23: Preparation of 5-amino-1-(3-morpholinocyclobutyl)-3-(2-phenylquinolin-7-yl)-1H-pyrazole-4-carboxamide

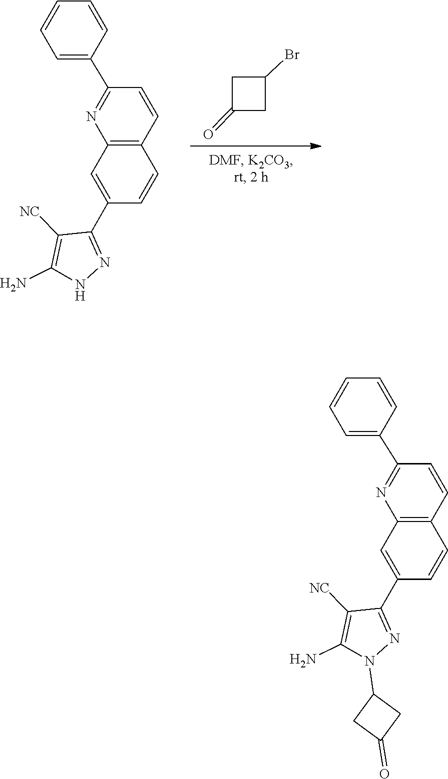 [SMILES: N#Cc1c(-c2ccc3ccc(-c4ccccc4)nc3c2)n[nH]c1N ; O=C1CC(Br)C1 ; N#Cc1c(-c2ccc3ccc(-c4ccccc4)nc3c2)nn(C2CC(=O)C2)c1N]

To a stirred mixture of 5-amino-3-(2-phenylquinolin-7-yl)-1H-pyrazole-4-carbonitrile (400 mg, 1.3 mmol, 1.0 eq) and K₂CO₃ (107.6 mg, 0.8 mmol, 0.6 eq) in DMF (5.0 mL) was added 3-bromocyclobutan-1-one (211.6 mg, 1.4 mmol, 1.1 eq) under ice bath. The mixture was stirred at rt for 4 h, then quenched by H₂O (20.0 mL) and extracted by EtOAc (30 mL×3). The combined organic layers were dried over sodium sulfate and concentrated in vacuum. The resulting residue was purified by column chromatography on silica gel (PE/EtOAc=2/1, v/v) to afford 5-amino-1-(3-oxocyclobutyl)-3-(2-phenylquinolin-7-yl)-1H-pyrazole-4-carbonitrile as a light yellow oil (200 mg, 41.1%). LRMS (M+H⁺) m/z calculated 380.1, found 380.1.

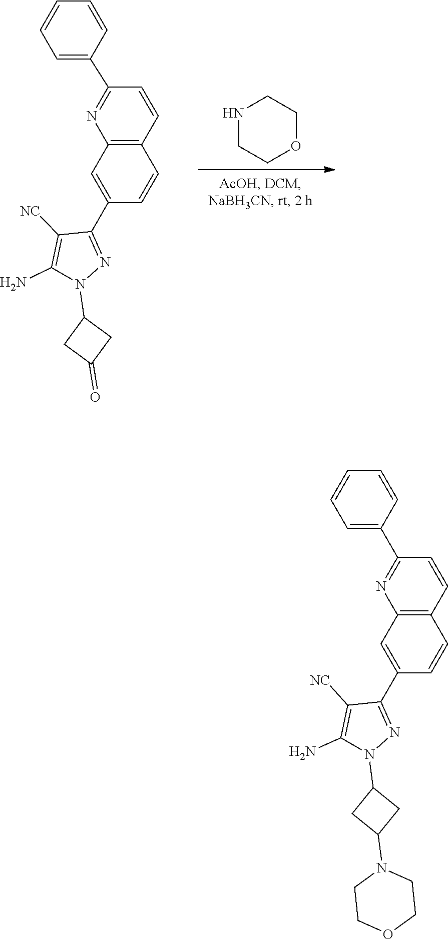 [SMILES: N#Cc1c(-c2ccc3ccc(-c4ccccc4)nc3c2)nn(C2CC(=O)C2)c1N ; C1COCCN1 ; N#Cc1c(-c2ccc3ccc(-c4ccccc4)nc3c2)nn(C2CC(N3CCOCC3)C2)c1N]

To a stirred solution of 5-amino-1-(3-oxocyclobutyl)-3-(2-phenylquinolin-7-yl)-1H-pyrazole-4-carbonitrile (200 mg, 0.5 mmol, 1.0 eq), AcOH (45.0 mg, 0.8 mmol, 1.5 eq) and morpholine (130.5 mg, 1.5 mmol, 3.0 eq) in DCM (5 mL) was added NaBH₃CN (64 mg, 1.0 mmol, 2.0 eq). The reaction was stirred at rt for 4 h, then quenched by H₂O (20.0 mL) and the mixture was extracted by DCM/MeOH (10/1, 30 mL×3). The combined organic layers were dried over sodium sulfate and concentrated in vacuum. The resulting residue was purified by column chromatography on silica gel (DCM/MeOH=10/1, v/v) to afford 5-amino-1-(3-morpholinocyclobutyl)-3-(2-phenylquinolin-7-yl)-1H-pyrazole-4-carbonitrile as a light yellow solid (100 mg, 42.2%). LRMS (M+H⁺) m/z calculated 451.2, found 451.2.

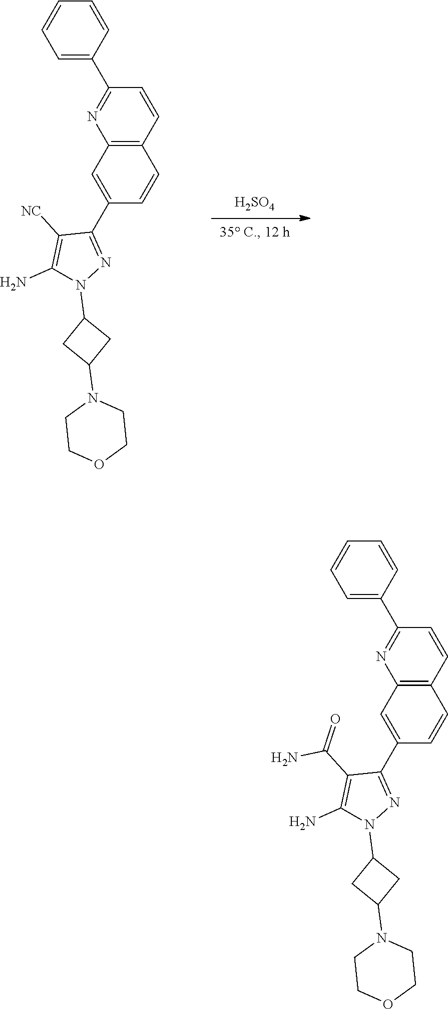 [SMILES: N#Cc1c(-c2ccc3ccc(-c4ccccc4)nc3c2)nn(C2CC(N3CCOCC3)C2)c1N ; "H2SO4" ; NC(=O)c1c(-c2ccc3ccc(-c4ccccc4)nc3c2)nn(C2CC(N3CCOCC3)C2)c1N]

The mixture of 5-amino-1-(3-morpholinocyclobutyl)-3-(2-phenylquinolin-7-yl)-1H-pyrazole-4-carbonitrile (100 mg, 0.2 mmol, 1.0 eq) in conc. H₂SO₄ (1.0 mL) was stirred at rt for 12 h, then quenched with H₂O (20.0 mL) and the mixture was adjusted to pH 8.0 with the addition of saturated Na₂CO₃ aqueous solution. The mixture was extracted by DCM/MeOH (10/1, 20 mL×3). The combined organic layers were dried over sodium sulfate and concentrated in vacuum. The resulting residue was purified by Prep-HPLC to afford 5-amino-1-(3-morpholinocyclobutyl)-3-(2-phenylquinolin-7-yl)-1H-pyrazole-4-carboxamide as a white solid (61.4 mg, 59.0%). LRMS (M+H⁺) m/z calculated 469.2, found 469.1. ¹H NMR (DMSO-d₆, 400 MHz) δ 8.50 (d, 1H), 8.30-8.31 (m, 2H), 8.17-8.19 (m, 2H), 8.06 (d, 1H), 7.77 (m, 1H), 7.52-7.60 (m, 3H), 6.30-6.32 (s, 2H), 4.55-4.59 (m, 1H), 3.58 (d, 4H), 2.50-2.51 (m, 3H), 2.30-2.39 (m, 6H).

### Example 24: Preparation of 5-amino-1-isopropyl-3-(2-phenylquinolin-7-yl)-1H-pyrazole-4-carboxamide

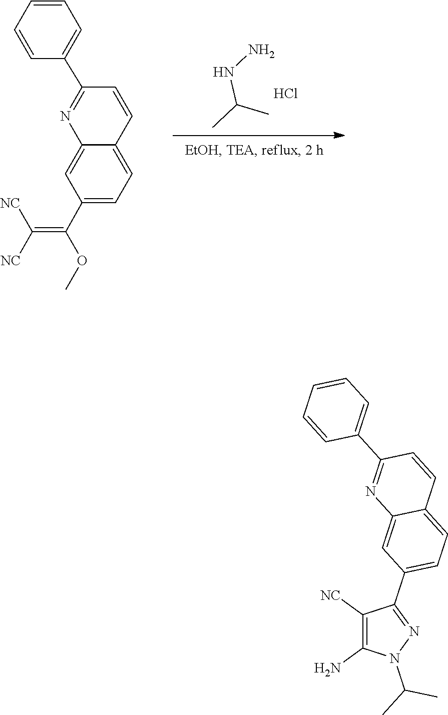

To a stirred solution of 2-(methoxy(2-phenylquinolin-7-yl)methylene)malononitrile (550 mg, 1.8 mmol, 1.0 eq) and TEA (545.4 mg, 5.4 mmol, 3.0 eq) in EtOH (10.0 mL) was added isopropylhydrazine hydrochloride (396 mg, 3.6 mmol. 2.0 eq). The mixture was stirred under reflux for 2 h, then quenched by H₂O (20.0 mL) and extracted with DCM/MeOH (10/1, 30 mL×3). The combined organic layers were concentrated in vacuum. The resulting residue was purified by column chromatography on silica gel (DCM/MeOH=10/1, v/v) to afford 5-amino-1-isopropyl-3-(2-phenylquinolin-7-yl)-1H-pyrazole-4-carbonitrile as a light yellow solid (450.0 mg. 72.1%). LRMS (M+H⁺) m/z calculated 354.2, found 354.2.

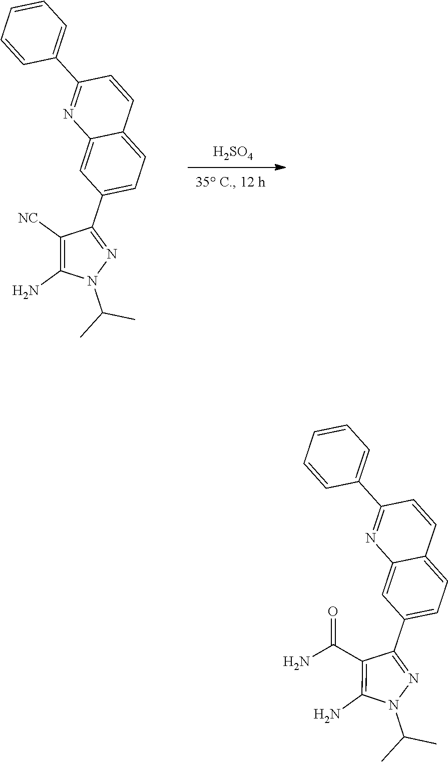 [SMILES: CC(C)n1nc(-c2ccc3ccc(-c4ccccc4)nc3c2)c(C#N)c1N ; "H2SO4" ; CC(C)n1nc(-c2ccc3ccc(-c4ccccc4)nc3c2)c(C(N)=O)c1N]

The mixture of 5-amino-1-isopropyl-3-(2-phenylquinolin-7-yl)-1H-pyrazole-4-carbonitrile (200 mg, 0.57 mmol, 1.0 eq) in H₂SO₄ (1 mL) was stirred at rt for 12 h, then quenched by H₂O (20.0 mL) and the mixture was adjusted to pH8.0 with the addition of saturated Na₂CO₃ aqueous solution, then extracted by DCM/MeOH (10/1, 30 mL×3). The combined organic layers were dried over sodium sulfate and concentrated in vacuum. The resulting residue was purified by Prep-HPLC to afford 5-amino-1-isopropyl-3-(2-phenylquinolin-7-yl)-1H-pyrazole-4-carboxamide as a white solid (94.4 mg, 45.0%). LRMS (M+H⁺) m/z calculated 372.2, found 372.1. ¹H NMR (DMSO-d₆, 400 MHz) δ 8.48-8.50 (d, 1H), 8.23 (d, 2H), 8.16-8.18 (m, 2H), 8.04 (d, 1H), 7.77 (d, 1H), 7.51-7.59 (m, 3H), 6.28 (s, 2H), 4.52-4.55 (m, 1H), 1.38-1.40 (m, 6H).

### Example 25: Preparation of 5-amino-3-(2-phenylquinolin-7-yl)-1-(3-(piperazin-1-yl)cyclobutyl)-1H-pyrazole-4-carboxamide

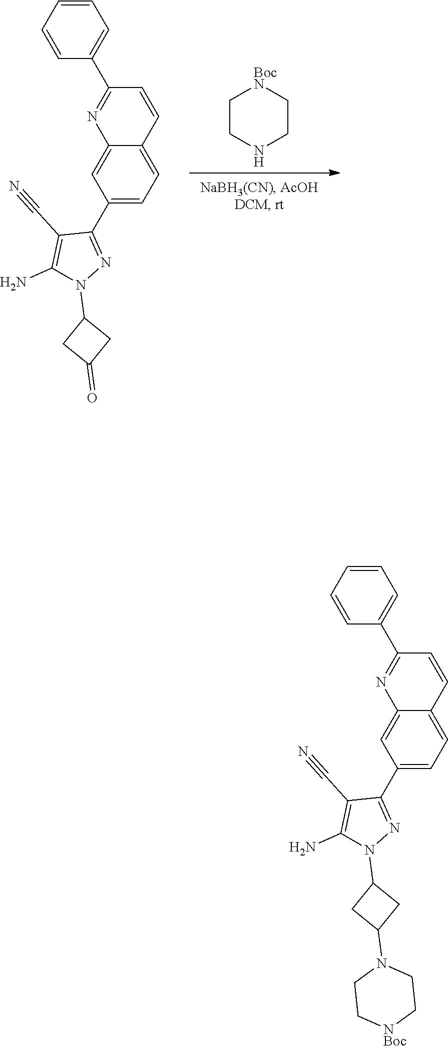 [SMILES: N#Cc1c(-c2ccc3ccc(-c4ccccc4)nc3c2)nn(C2CC(=O)C2)c1N ; CC(C)(C)OC(=O)N1CCNCC1 ; CC(C)(C)OC(=O)N1CCN(C2CC(n3nc(-c4ccc5ccc(-c6ccccc6)nc5c4)c(C#N)c3N)C2)CC1]

To a solution of 5-amino-1-(3-oxocyclobutyl)-3-(2-phenylquinolin-7-yl)-1H-pyrazole-4-carbonitrile (100 mg, 0.26 mmol, 1.0 eq) in DCM (20 mL) were added NaBH₃CN (24.9 mg, 0.40 mmol, 1.5 eq), AcOH (23.7 mg, 0.40 mmol, 1.5 eq) and tert-butyl piperazine-1-carboxylate (147.2 mg, 0.79 mmol, 3.0 eq). The mixture was stirred at 20° C. for 3 h, then quenched with water, and concentrated in vacuum. The resulting residue was diluted with H₂O (20 mL), and extracted with EtOAc (50 mL). The combined organic layers were washed with brine (20 mL), dried over anhydrous sodium sulfate and concentrated in vacuum. The resulting residue was purified by column chromatography (SiO₂, PE/EA=1/1, v/v) to obtain tert-butyl 4-(3-(5-amino-4-cyano-3-(2-phenylquinolin-7-yl)-1H-pyrazol-1-yl)cyclobutyl)piperazine-1-carboxylate (60 mg, 41.3%) as a yellow solid. LRMS (M+H⁺) m/z calculated 550.3, found 550.2.

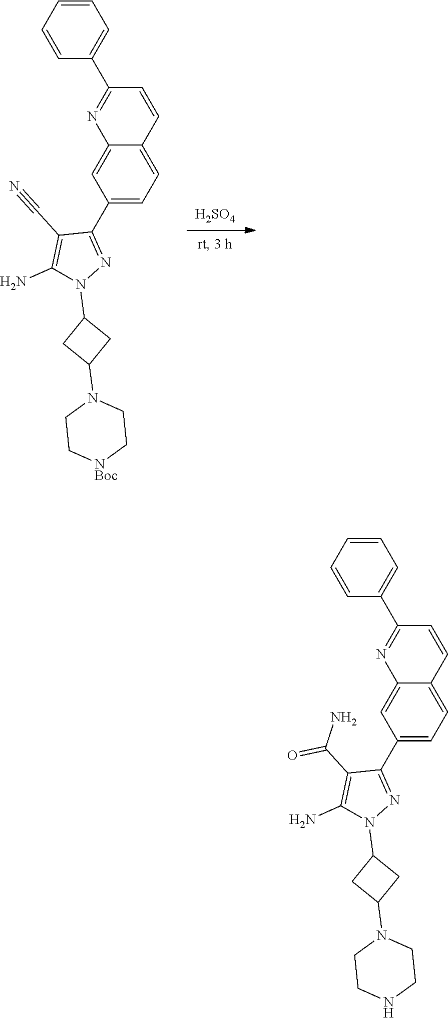

Tert-butyl 4-(3-(5-amino-4-cyano-3-(2-phenylquinolin-7-yl)-1H-pyrazol-1-yl)cyclobutyl)piperazine-1-carboxylate (60 mg, 0.11 mmol, 1.0 eq) was added to conc. H₂SO₄ (5 mL) at rt. The mixture was stirred for 3 h at rt, then slowly poured into ice and adjusted to pH 7 with the addition of saturated NaHCO₃ (aq) and extracted with ethyl acetate (30 mL×3). The combined organic layers were washed with brine (30 mL), dried over anhydrous sodium sulfate, and concentrated in vacuum. The resulting residue was purified by Prep-HPLC to afford 5-amino-3-(2-phenylquinolin-7-yl)-1-(3-(piperazin-1-yl)cyclobutyl)-1H-pyrazole-4-carboxamide (21.9 mg, 43.1%) as a white solid. LRMS (M+H⁺) m/z calculated 468.2, found 468.1. ¹H NMR (DMSO-d₆, 400 MHz) δ 8.50 (d, 1H), 8.31 (d, 2H), 8.19 (s, 1H), 8.18 (d, 1H), 8.05 (d, 1H), 7.77 (dd, 1H), 7.49-7.59 (m, 3H), 6.31 (s, 2H), 4.53-4.57 (m, 1H), 2.63-2.68 (m, 4H), 2.33-2.49 (m, 5H), 2.19-2.22 (m, 4H).

### Example 26: Preparation of 5-amino-1-(3-(4-methylpiperazin-1-yl)cyclobutyl)-3-(2-phenylquinolin-7-yl)-1H-pyrazole-4-carboxamide

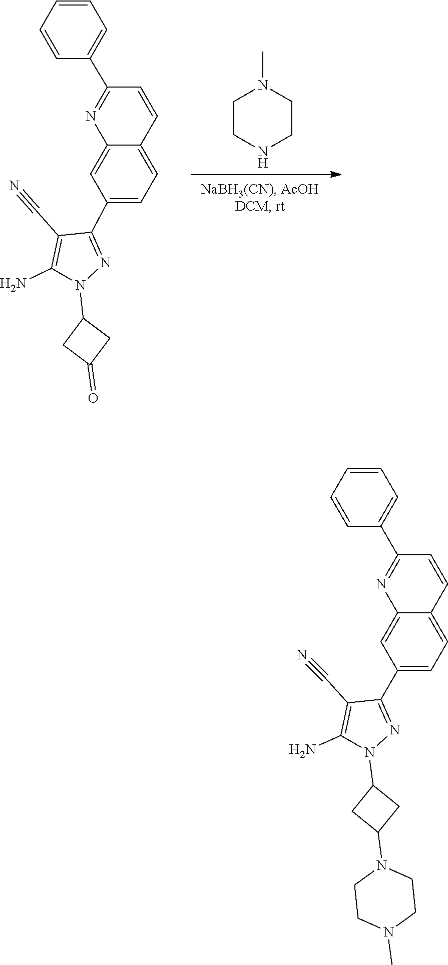

To a solution of 5-amino-1-(3-oxocyclobutyl)-3-(2-phenylquinolin-7-yl)-1H-pyrazole-4-carbonitrile (100 mg, 0.26 mmol, 1.0 eq) in DCM (20 mL) were added NaBH₃CN (24.9 mg, 0.40 mmol, 1.5 eq), AcOH (23.7 mg, 0.40 mmol, 1.5 eq) and 1-methylpiperazine (79.1 mg, 0.79 mmol, 3.0 eq). The mixture was stirred at 30° C. for 3 h, then quenched with water and concentrated in vacuum. The resulting residue was diluted with H₂O (20 mL), and extracted with EtOAc (50 mL×3). The combined organic layers were washed with brine (20 mL), dried over anhydrous sodium sulfate and concentrated in vacuum. The resulting residue was purified by column chromatography on silica gel (SiO₂, PE/EA=1/1, v/v) to obtain 5-amino-1-(3-(4-methylpiperazin-1-yl)cyclobutyl)-3-(2-phenylquinolin-7-yl)-1H-pyrazole-4-carbonitrile (70 mg, 57.3%) as a yellow solid. LRMS (M+H⁺) m/z calculated 464.2, found 464.1.

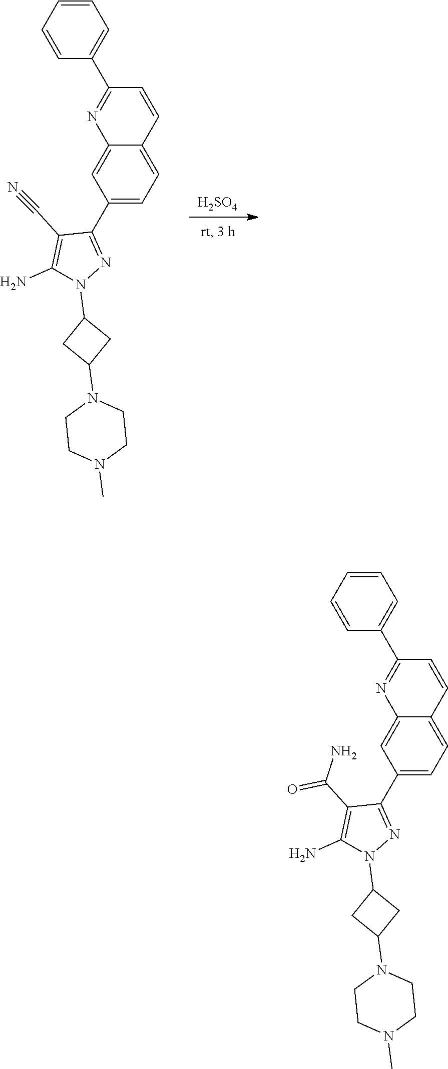

5-Amino-1-(3-(4-methylpiperazin-1-yl)cyclobutyl)-3-(2-phenylquinolin-7-yl)-1H-pyrazole-4-carbonitrile (60 mg, 0.11 mmol, 1.0 eq) was added to conc. H₂SO₄ (5 mL) and the mixture was stirred for 3 h at rt, then slowly poured into ice and adjusted to pH 7 with saturated NaHCO₃ (aq), and extracted with ethyl acetate (30 mL×3). The combined organic layers were washed with brine (30 mL), dried over anhydrous sodium sulfate and concentrated in vacuum. The resulting residue was purified by Prep-HPLC to afford 5-amino-1-(3-(4-methylpiperazin-1-yl)cyclobutyl)-3-(2-phenylquinolin-7-yl)-1H-pyrazole-4-carboxamide (12.6 mg, 43.1%) as a white solid. LRMS (M+H⁺) m/z calculated 482.3, found 482.2. ¹H NMR (DMSO-d₆, 400 MHz) δ 8.50 (d, 1H), 8.30 (d, 2H), 8.19 (s, 1H), 8.18 (d, 1H), 8.04 (d, 1H), 7.77 (dd, 1H), 7.49-7.59 (m, 3H), 6.31 (s, 2H), 4.51-4.60 (m, 1H), 2.50-2.52 (m, 2H), 2.31-2.41 (m, 11H), 2.14 (s, 3H).

### Example 27: Preparation of 5-amino-1-(3-(azetidin-1-yl)cyclobutyl)-3-(2-phenylquinolin-7-yl)-1H-pyrazole-4-carboxamide

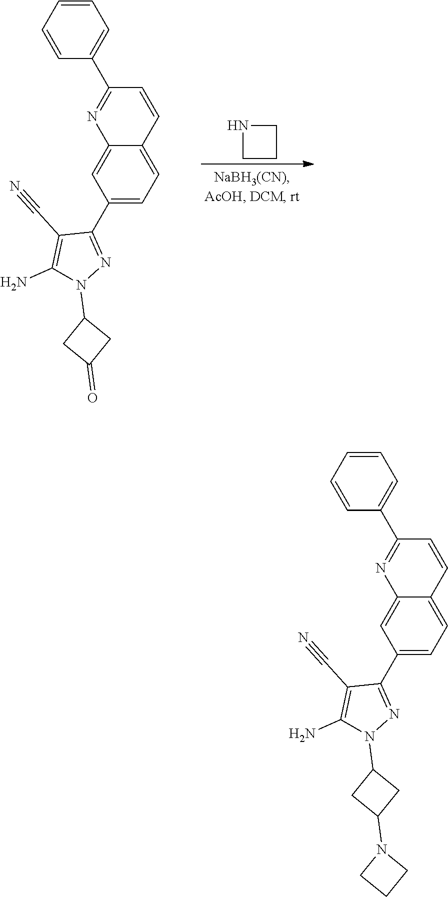

To a stirred solution of 5-amino-1-(3-oxocyclobutyl)-3-(2-phenylquinolin-7-yl)-1H-pyrazole-4-carbonitrile (200 mg, 0.5 mmol, 1.0 eq), AcOH (45.0 mg, 0.8 mmol, 1.5 eq) and azetidine (85.5 mg, 1.5 mmol, 3.0 eq) in DCM (5 mL) was added NaBH₃CN (64 mg, 1.0 mmol, 2.0 eq). The reaction was stirred at rt for 4 h, then quenched by H₂O (20.0 mL), and the mixture was extracted by DCM/MeOH (10/1, 30 mL×3). The combined organic layers were dried over sodium sulfate and concentrated in vacuum. The resulting residue was purified by column chromatography on silica gel (DCM/MeOH=10/1, v/v) to afford 5-amino-1-(3-(azetidin-1-yl) cyclobutyl)-3-(2-phenylquinolin-7-yl)-1H-pyrazole-4-carbonitrile as a light yellow oil (50 mg, 22.5%). LRMS (M+H⁺) m/z calculated 421.2, found 421.2.

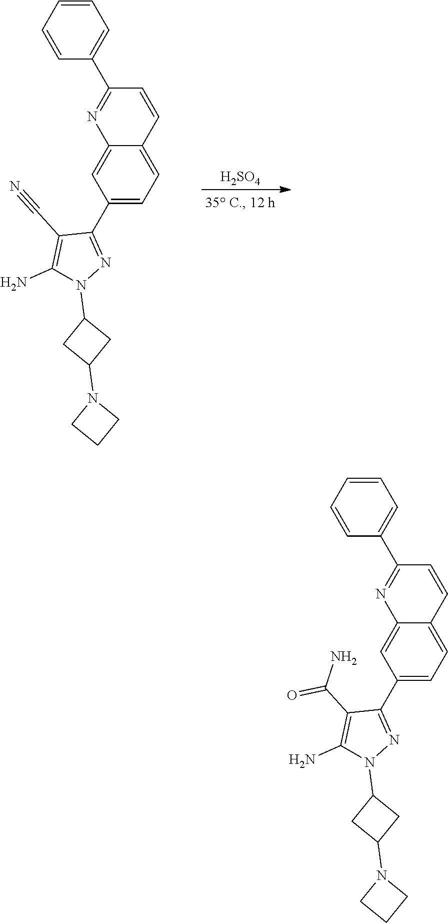

The mixture of 5-amino-1-(3-(azetidin-1-yl)cyclobutyl)-3-(2-phenylquinolin-7-yl)-1H-pyrazole-4-carbonitrile (50 mg, 0.1 mmol, 1.0 eq) in conc. H₂SO₄ (1.0 mL) was stirred at rt for 12 h, then quenched by H₂O (20.0 mL) and the mixture was adjusted to pH 8.0 with the addition of saturated Na₂CO₃ aqueous solution. The mixture was extracted by DCM/MeOH (10/1.20 mL×3). The combined organic layers were dried over sodium sulfate and concentrated in vacuum. The resulting residue was purified by Prep-HPLC to afford 5-amino-1-(3-(azetidin-1-yl)cyclobutyl)-3-(2-phenylquinolin-7-yl)-1H-pyrazole-4-carboxamide as a white solid (3.6 mg, 6.9%). LRMS (M+H⁺) m/z calculated 439.2, found 439.1. ¹H NMR (DMSO-d₆, 400 MHz) δ 8.51 (d, 1H), 8.29-8.31 (m, 2H), 8.17-8.20 (m, 2H), 8.06 (d, 1H), 7.78 (dd, 1H), 7.52-7.59 (m, 3H), 6.30 (s, 2H), 4.50-4.55 (m, 1H), 3.27-3.32 (m, 3H), 3.18-3.19 (m, 1H), 2.67-2.68 (m, 1H), 2.33-2.46 (m, 4H), 1.93-1.97 (m, 2H).

### Example 28: Preparation of 5-amino-3-(8-fluoro-2-phenylquinolin-7-yl)-1-((1s,3s)-3-hydroxy-3-methylcyclobutyl)-1H-pyrazole-4-carboxamide

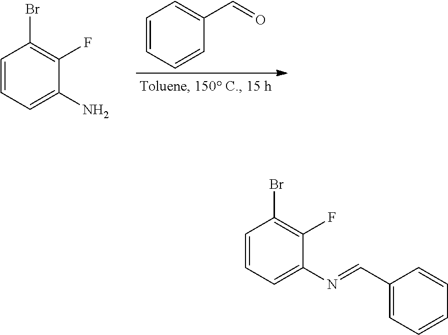 [SMILES: Nc1cccc(Br)c1F ; O=Cc1ccccc1 ; Fc1c(Br)cccc1/N=C/c1ccccc1]

A solution of 3-bromo-2-fluoroaniline (10 g, 52.9 mmol, 1.0 eq) and benzaldehyde (257 g, 2.40 mol, 1.2 eq) in toluene (1.5 L) was stirred at 150° C. for 16 h. The reaction mixture was concentrated in vacuum to dryness to give N-(3-bromo-2-fluorophenyl)-1-phenylmethanimine (7.5 g, 51.3%) as a yellow oil, which was used for the next step without further purification. LRMS (M+H⁺) m/z calculated 278.0, found 278.0. ¹H NMR (DMSO-d₆, 400 MHz) δ 8.68 (s, 1H), 7.95-7.98 (m, 2H), 7.52-7.62 (m, 4H), 7.32 (t, 1H), 7.20 (t, 1H).

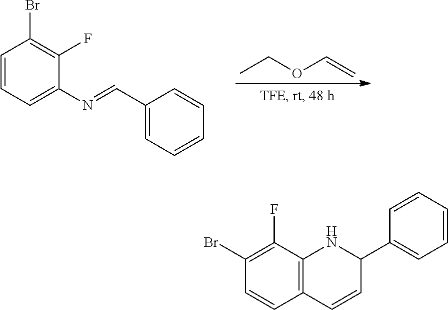

A solution of N-(3-bromo-2-fluorophenyl)-1-phenylmethanimine (7 g, 25.2 mmol, 1.0 eq) and ethoxyethene (9.1 g, 125.9 mol, 5.0 eq) in TFE (100 mL) was stirred at 35° C. for 16 h. The reaction mixture was concentrated in vacuum and purified by column chromatography on silica gel (PE/EA=10/1, v/v) to afford 7-bromo-8-fluoro-2-phenyl-1,2-dihydroquinoline (2.8 g, 37.8%) as a yellow solid. LRMS (M+H⁺) m/z calculated 304.0, found 304.0.

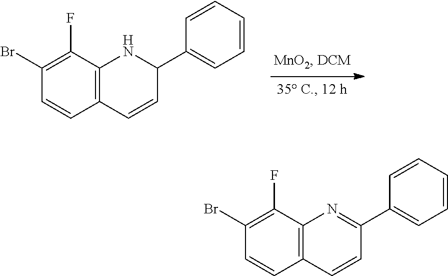 [SMILES: Fc1c(Br)ccc2c1NC(c1ccccc1)C=C2 ; Fc1c(Br)ccc2ccc(-c3ccccc3)nc12]

A suspension of 7-bromo-8-fluoro-2-phenyl-1,2-dihydroquinoline (2.8 g, 9.5 mmol, 1.0 eq) and MnO₂ (16.5 g, 190.1 mol, 5.0 eq) in DCM (50 mL) was stirred at 35° C. for 12 h. The reaction mixture was filtered, the filtrate was concentrated in vacuum and purified by column chromatography on silica gel (PE/EA=10/1, v/v) to afford 7-bromo-8-fluoro-2-phenylquinoline (1.2 g, 42.8%) as a yellow solid. LRMS (M+H⁺) m/z calculated 302.0, found 302.0.

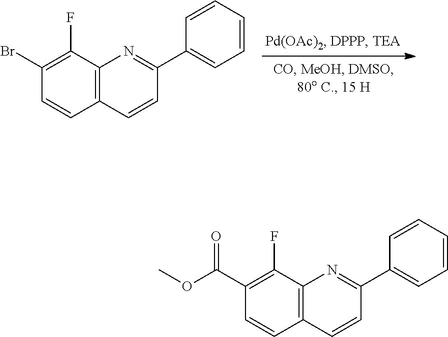 [SMILES: Fc1c(Br)ccc2ccc(-c3ccccc3)nc12 ; COC(=O)c1ccc2ccc(-c3ccccc3)nc2c1F]

To a solution of 7-bromo-8-fluoro-2-phenylquinoline (500 mg, 1.7 mmol, 1.0 eq), DPPP (136.4 mg, 0.33 mmol, 0.2 eq) and Pd(OAc)₂ (37.1 mg, 0.16 mmol, 0.1 eq) in DMSO/MeOH (300 mL/300 mL) was added TEA (40 mL, 289.8 mmol. 3.0 eq). The mixture was stirred at 80° C. for 15 h under CO (1 atm), then concentrated in vacuum. The resulting residue was purified by column chromatography on silica gel (PE/EA=5/1, v/v) to afford methyl 8-fluoro-2-phenylquinoline-7-carboxylate (100 mg, 21.5%) as a yellow oil. LRMS (M+H⁺) m/z calculated 282.1, found 282.1.

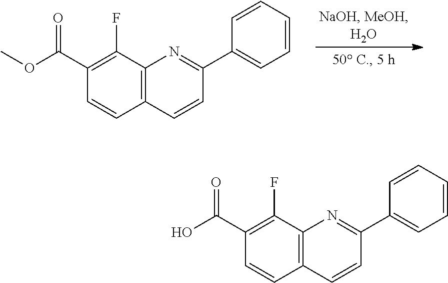 [SMILES: COC(=O)c1ccc2ccc(-c3ccccc3)nc2c1F ; O=C(O)c1ccc2ccc(-c3ccccc3)nc2c1F]

To a solution of methyl 8-fluoro-2-phenylquinoline-7-carboxylate (100 mg, 0.36 mmol, 1.0 eq) in MeOH (30 mL) and H₂O (5 mL) was added NaOH (21.4 mg, 0.53 mmol, 1.5 eq). The mixture was stirred at 50° C. for 5 h, then concentrated in vacuum and diluted with water (20 mL), 37% HCl was added to adjust to pH 2. The resulting mixture was stirred for 5 min, filtrated and concentrated in vacuum to afford 8-fluoro-2-phenylquinoline-7-carboxylic acid (80 mg, 84.2%) as a white solid, LRMS (M+H⁺) m/z calculated 268.1, found 268.1.

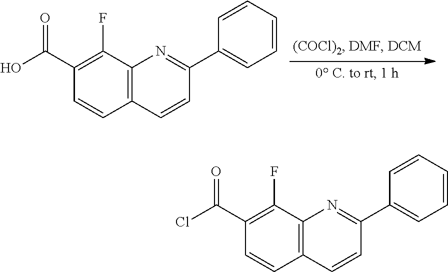 [SMILES: O=C(O)c1ccc2ccc(-c3ccccc3)nc2c1F ; O=C(Cl)c1ccc2ccc(-c3ccccc3)nc2c1F]

To a solution of 8-fluoro-2-phenylquinoline-7-carboxylic acid (80 mg, 0.30 mmol, 1.0 eq) in DCM (10 mL) were added (COCl)₂ (0.13 mL, 1.5 mmol, 5.0 eq) and DMF (1 drop) at −78° C. The mixture was stirred at rt for 1 h, then concentrated in vacuum to afford 8-fluoro-2-phenylquinoline-7-carbonyl chloride as a yellow solid (95 mg, ca 100.0%), LRMS (M+H⁺) m/z calculated 282.1. found 282.1 in MeOH.

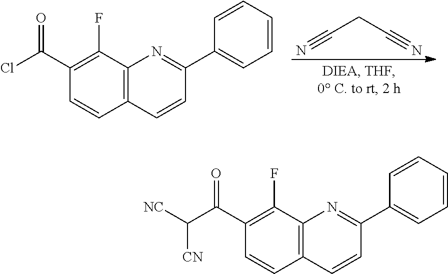

To a solution of 8-fluoro-2-phenylquinoline-7-carbonyl chloride (95 mg, 0.33 mmol, 1.0 eq) in THF (10 mL) were added malononitrile (22.0 mg, 0.33 mmol, 1.0 eq) and DIEA (0.2 mL, 1.0 mmol, 3.0 eq) at ice bath. The mixture was stirred at rt for 3 h, then concentrated in vacuum, diluted with water (20 mL). The resulting mixture was extracted with EtOAc (50 mL×2). The combined organic layers were washed with brine (50 mL), dried over anhydrous sodium sulfate and concentrated in vacuum. The resulting residue was purified by column chromatography on silica gel (DCM/MeOH=50/1, v/v) to afford 2-(8-fluoro-2-phenylquinoline-7-carbonyl)malononitrile as a yellow oil (60 mg, 54.5%), LRMS (M+H⁺) m/z calculated 316.1, found 316.1.

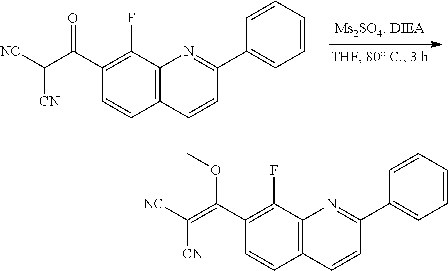

To a solution of 2-(8-fluoro-2-phenylquinoline-7-carbonyl)malononitrile (60 mg, 0.19 mmol, 1.0 eq) in THF (10 mL) were added Me₂SO₄ (48.0 mg, 0.38 mmol, 2.0 eq) and DIEA (49.1 mg, 0.38 mmol, 2.0 eq) at rt. The mixture was stirred at 80° C. for 3 h, then concentrated in vacuum and diluted with water (20 mL), extracted with EtOAc (50 mL×2). The combined organic layers were washed with brine (50 mL), dried over anhydrous sodium sulfate and concentrated in vacuum. The resulting residue was purified by column chromatography on silica gel (PE/EA=1/1, v/v) to afford 2-((8-fluoro-2-phenylquinolin-7-yl)(methoxy)methylene)malononitrile as a yellow oil (40 mg, 63.5%), LRMS (M+H⁺) m/z calculated 330.1, found 330.1.

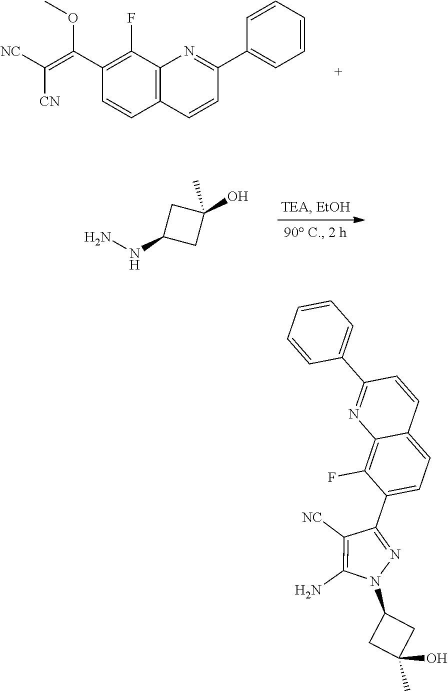

To a solution of 2-((8-fluoro-2-phenylquinolin-7-yl)(methoxy)methylene)malononitrile (40 mg, 0.12 mmol, 1.0 eq) and (1s,3s)-3-hydrazineyl-1-methylcyclobutan-1-ol (21.2 mg, 0.18 mmol, 1.5 eq) in EtOH (20 mL) were added TEA (0.2 mL, 0.97 mmol, 8.0 eq) at rt. The reaction mixture was stirred at 90° C. for 2 h then the mixture was concentrated in vacuum and purified by column chromatography on silica gel (PE/EA=1/1, v/v) to afford 5-amino-3-(8-fluoro-2-phenylquinolin-7-yl)-1-((1s,3s)-3-hydroxy-3-methylcyclobutyl)-1H-pyrazole-4-carbonitrile (30 mg, 60.0%) as a yellow solid. LRMS (M+H⁺) m/z calculated 414.2, found 414.2.

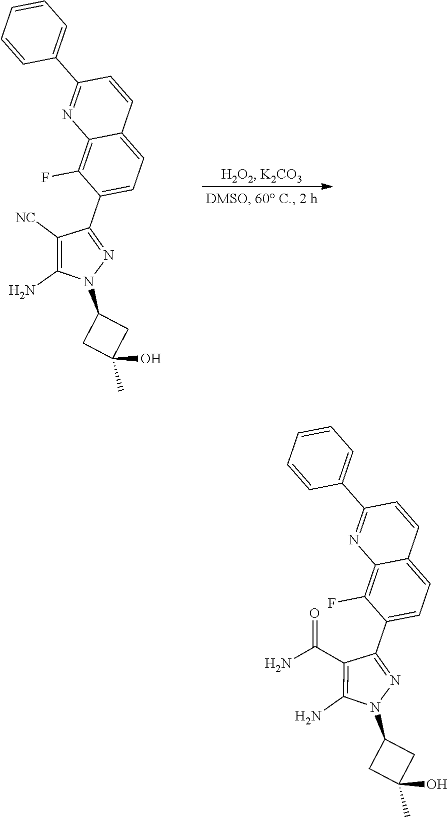 [SMILES: C[C@]1(O)C[C@@H](n2nc(-c3ccc4ccc(-c5ccccc5)nc4c3F)c(C#N)c2N)C1 ; C[C@]1(O)C[C@@H](n2nc(-c3ccc4ccc(-c5ccccc5)nc4c3F)c(C(N)=O)c2N)C1]

To a stirred solution of 5-amino-3-(8-fluoro-2-phenylquinolin-7-yl)-1-((1s,3s)-3-hydroxy-3-methylcyclobutyl)-1H-pyrazole-4-carbonitrile (30 mg, 0.07 mmol, 1.0 eq) and K₂CO₃ (30.1 mg, 0.22 mmol, 3.0 eq) in DMSO (10 mL) at rt was added H₂O₂ (30%, 164.7 mg, 1.5 mmol, 20.0 eq). After addition was completed, the reaction mixture was stirred at 60° C. for 1 h. Water (20 mL) was added and the mixture was extracted with EtOAc (50 mL). The organic layer was washed with brine (100 mL), dried with anhydrous sodium sulfate, and purified by Prep-HPLC to give 5-amino-3-(8-fluoro-2-phenylquinolin-7-yl)-1-((1s,3s)-3-hydroxy-3-methylcyclobutyl)-1H-pyrazole-4-carboxamide (5.4 mg, 17.2%) as a white solid. LRMS (M+H⁺) m/z calculated 432.2, found 432.1. ¹H NMR (DMSO-d₆, 400 MHz) δ 8.57 (d, 1H), 8.27-8.33 (m, 3H), 7.89 (d, 1H), 7.51-7.63 (m, 4H), 6.32 (s, 2H), 5.18 (s, 1H), 4.45-4.50 (m, 1H), 2.55-2.61 (m, 2H), 2.36-2.41 (m, 2H), 1.34 (s, 3H).

### Example 29: Preparation of 5-amino-1-(oxetan-3-yl)-3-(2-phenylquinolin-7-yl)-1H-pyrazole-4-carboxamide

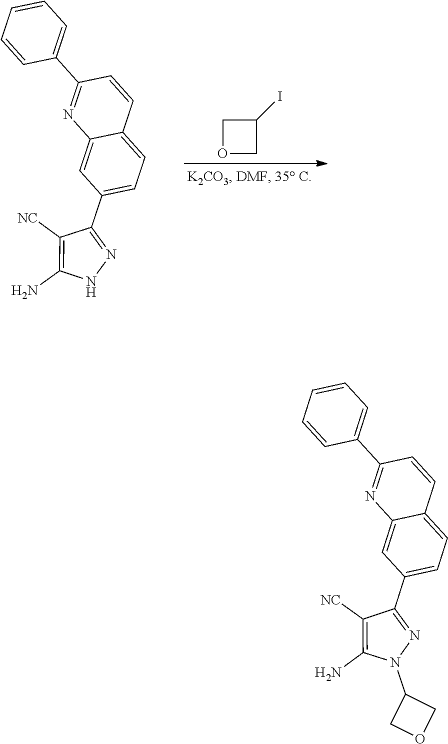 [SMILES: N#Cc1c(-c2ccc3ccc(-c4ccccc4)nc3c2)n[nH]c1N ; IC1COC1 ; N#Cc1c(-c2ccc3ccc(-c4ccccc4)nc3c2)nn(C2COC2)c1N]

To a stirred mixture of 5-amino-3-(2-phenylquinolin-7-yl)-1H-pyrazole-4-carbonitrile (400 mg, 1.3 mmol, 1.0 eq) and K₂CO₃ (107.6 mg, 0.8 mmol, 0.6 eq) in DMF (5.0 mL) was added 3-iodooxetane (260.3 mg, 1.4 mmol, 1.1 eq) at 0° C. The mixture was stirred at 35° C. for 4 h, then quenched by H₂O (20.0 mL) and extracted by EtOAc (30 mL×3). The combined organic layers were dried over sodium sulfate and concentrated in vacuum. The resulting residue was purified by column chromatography on silica gel (PE/EA=2/1, v/v) to afford 5-amino-1-(oxetan-3-yl)-3-(2-phenylquinolin-7-yl)-1H-pyrazole-4-carbonitrile as a yellow oil (220 mg, 46.6%). LRMS (M+H⁺) m/z calculated 368.1, found 368.1.

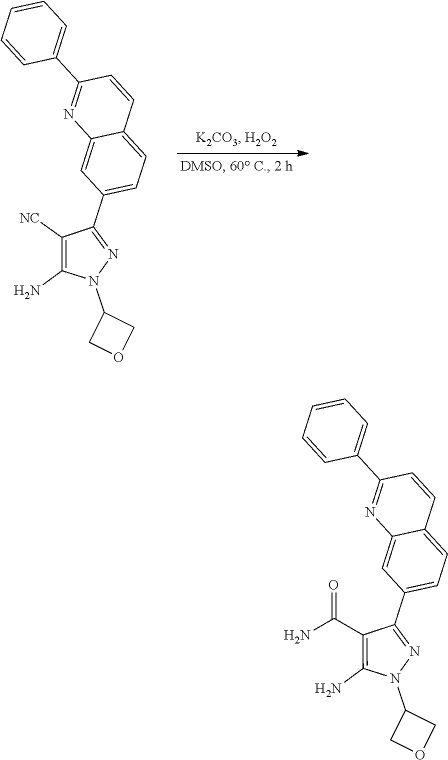 [SMILES: N#Cc1c(-c2ccc3ccc(-c4ccccc4)nc3c2)nn(C2COC2)c1N ; NC(=O)c1c(-c2ccc3ccc(-c4ccccc4)nc3c2)nn(C2COC2)c1N]

To a stirred solution of 5-amino-1-(oxetan-3-yl)-3-(2-phenylquinolin-7-yl)-1H-pyrazole-4-carbonitrile (30 mg, 0.22 mmol, 1.0 eq) and K₂CO₃ (90.2 mg, 0.65 mmol, 3.0 eq) in DMSO (10 mL) at rt was added H₂O₂ (30%, 494.1 mg, 4.4 mmol, 20.0 eq). After addition was completed, the reaction mixture was stirred at 60° C. for 2 h. Water (20 mL) was added and the mixture was extracted with EtOAc (50 mL). The combined organic layers were washed with brine (100 mL), dried over anhydrous Sodium sulfate, and purified by Prep-HPLC to give 5-amino-1-(oxetan-3-yl)-3-(2-phenylquinolin-7-yl)-1H-pyrazole-4-carboxamide (15.4 mg, 18.3%) as a white solid. LRMS (M+H⁺) m/z calculated 386.2, found 386.0. ¹H NMR (DMSO-d₆, 400 MHz) δ 8.51 (d, 1H), 8.29-8.32 (m, 2H), 8.25 (s, 1H), 8.19 (d, 1H), 8.07 (d, 1H), 7.82 (dd, 1H), 7.50-7.60 (m, 3H), 6.35 (s, 2H), 6.05 (brs, 1H), 5.58-5.62 (m, 1H), 5.01 (t, 2H), 4.88 (t, 2H).

### Example 30: Preparation of 5-amino-1-(3,3-difluorocyclobutyl)-3-(2-phenylquinolin-7-yl)-1H-pyrazole-4-carboxamide

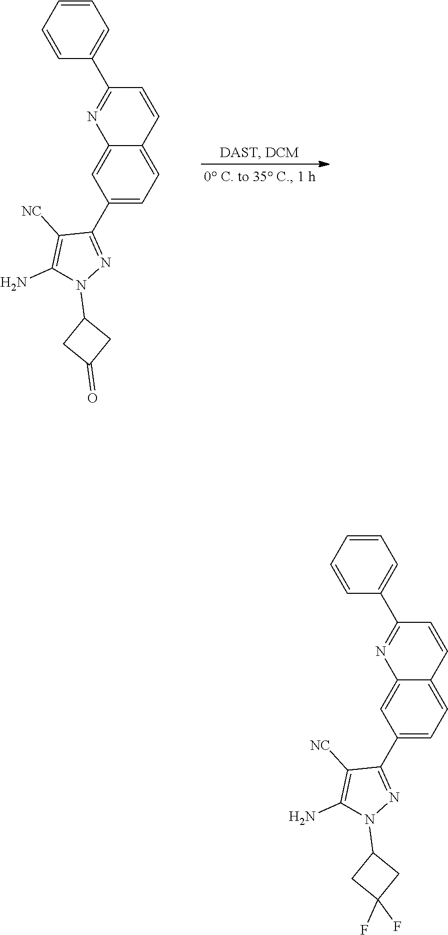

5-Amino-1-(3-oxocyclobutyl)-3-(2-phenylquinolin-7-yl)-1H-pyrazole-4-carbonitrile (250 mg, 0.66 mmol, 1.0 eq) was dissolved in DCM (30 mL) and cooled to 0° C. DAST (531.0 mg, 3.3 mmol, 5.0 eq) was added drop wise and the reaction mixture was stirred at 35° C. for 1 h. The reaction mixture was quenched with water, neutralized by saturated aqueous sodium bicarbonate solution to pH 7 and extracted with DCM (30 mL×3). The organic layers were washed with brine (50 mL), dried over sodium sulfate and concentrated to afford a crude residue. The crude residue was purified by column chromatography on silica gel (PE/EA=5/1, v/v) to afford 5-amino-1-(3,3-difluorocyclobutyl)-3-(2-phenylquinolin-7-yl)-1H-pyrazole-4-carbonitrile (160 mg, 60.6%) as a yellow solid. LRMS (M+H⁺) m/z calculated 402.1, found 402.0.

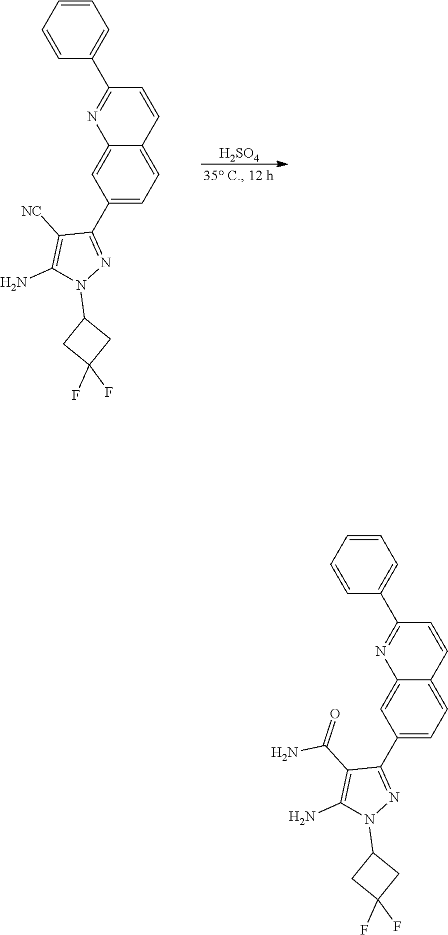

5-Amino-1-(3,3-difluorocyclobutyl)-3-(2-phenylquinolin-7-yl)-1H-pyrazole-4-carbonitrile (80 mg, 0.20 mmol, 1.0 eq) was added to conc. H₂SO₄ (5 mL) and then the mixture was stirred 35° C. for 12 h, then slowly poured into ice and adjusted to pH 7 with saturated NaHCO₃ (aq), and extracted with ethyl acetate (30 mL×3). The combined organic layers were washed with brine (30 mL), dried over anhydrous sodium sulfate and concentrated in vacuum. The resulting residue was purified by Prep-HPLC to afford 5-amino-1-(3,3-difluorocyclobutyl)-3-(2-phenylquinolin-7-yl)-1H-pyrazole-4-carboxamide (45.2 mg, 53.5%) as a white solid. LRMS (M+H⁺) m/z calculated 420.2, found 420.0. ¹H NMR (DMSO-d₆, 400 MHz) δ 8.50 (d, 1H), 8.29-8.32 (m, 2H), 8.22 (s, 1H), 8.18 (d, 1H), 8.06 (d, 1H), 7.78 (dd, 1H), 7.49-7.59 (m, 3H), 6.44 (s, 2H), 4.87-4.92 (m, 1H), 3.06-3.25 (m, 4H).

### Example 31: Preparation of 2-(2-phenylquinolin-7-yl)-4,5,6,7-tetrahydropyrazolo[1,5-a]pyrimidine-3,6-dicarboxamide

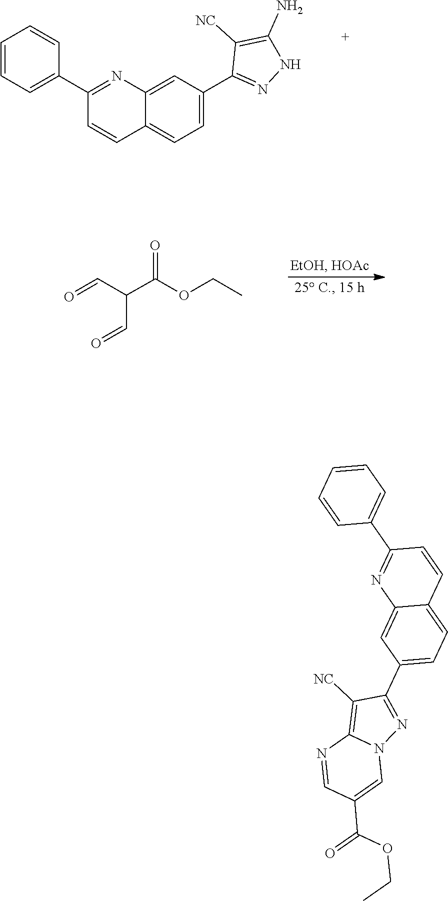

To a solution of 5-amino-3-(2-phenylquinolin-7-yl)-1H-pyrazole-4-carbonitrile (120 mg, 0.39 mmol, 1.0 eq) in EtOH (10 mL) was added ethyl2-formyl-3-oxopropanoate (61.1 mg, 0.42 mmol, 1.1 eq) and HOAc (5 drops). After stirring at 25° C. for 16 h, the mixture was filtered, washed with H₂O (10 mL) and dried in vacuum to afford ethyl 3-cyano-2-(2-phenylquinolin-7-yl) pyrazolo[1,5-a]pyrimidine-6-carboxylate (130 mg, 81.2%) as a yellow solid. LRMS (M+H⁺) m/z calculated 420.1, found 420.0.

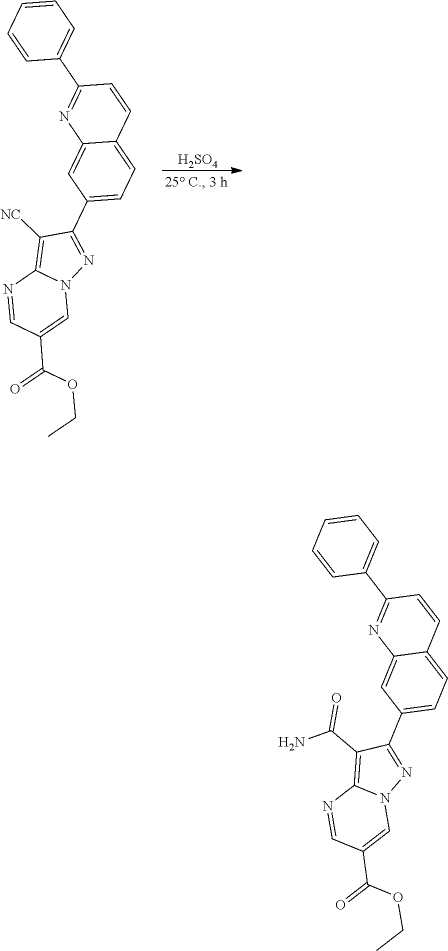

Ethyl 3-cyano-2-(2-phenylquinolin-7-yl) pyrazolo[1,5-a]pyrimidine-6-carboxylate (130 mg, 0.31 mmol, 1.0 eq) was added to conc. H₂SO₄ (5 mL) and then the mixture was stirred 25° C. for 3 h, then slowly poured into ice and adjusted to pH 7 with saturated NaHCO₃ solution, and extracted with ethyl acetate (30 mL×3). The combined organic layers were washed with brine (30 mL), dried over anhydrous sodium sulfate and concentrated in vacuum to afford ethyl 3-carbamoyl-2-(2-phenylquinolin-7-yl) pyrazolo[1,5-a]pyrimidine-6-carboxylate (90 mg, 66.6%) as a yellow solid. LRMS (M+H⁺) m/z calculated 438.1, found 438.0.

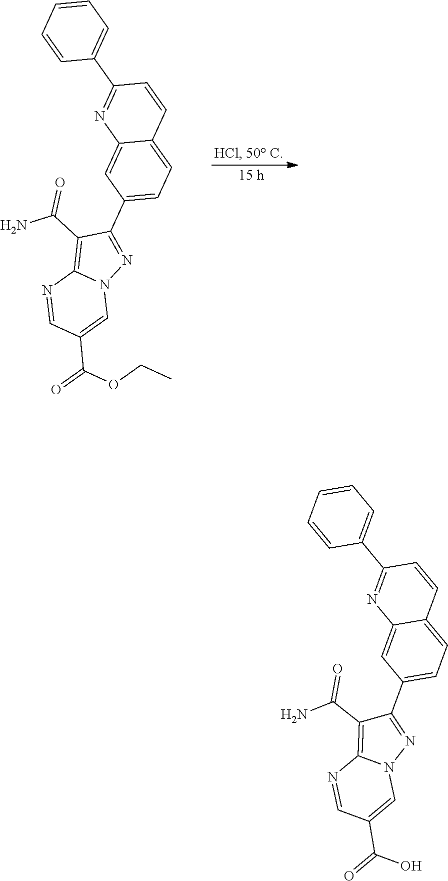 [SMILES: CCOC(=O)c1cnc2c(C(N)=O)c(-c3ccc4ccc(-c5ccccc5)nc4c3)nn2c1 ; NC(=O)c1c(-c2ccc3ccc(-c4ccccc4)nc3c2)nn2cc(C(=O)O)cnc12]

Ethyl 3-carbamoyl-2-(2-phenylquinolin-7-yl) pyrazolo[1,5-a]pyrimidine-6-carboxylate (75 mg, 0.17 mmol, 1.0 eq) was added to conc. HCl (15 mL), and the mixture was stirred 50° C. for 15 h. The reaction mixture was concentrated in vacuum to afford 3-carbamoyl-2-(2-phenylquinolin-7-yl) pyrazolo[1,5-a]pyrimidine-6-carboxylic acid (40 mg, 57.1%) as a yellow solid. LRMS (M+H⁺) m/z calculated 410.1, found 410.0.

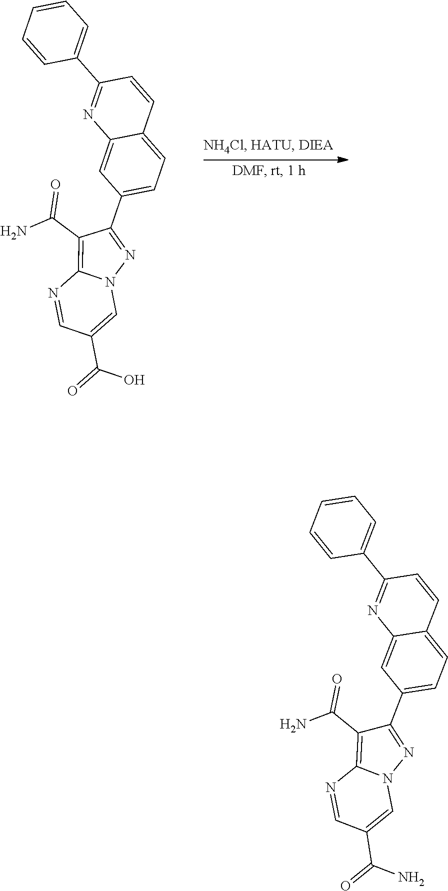 [SMILES: NC(=O)c1c(-c2ccc3ccc(-c4ccccc4)nc3c2)nn2cc(C(=O)O)cnc12 ; NC(=O)c1cnc2c(C(N)=O)c(-c3ccc4ccc(-c5ccccc5)nc4c3)nn2c1]

To a solution of 3-carbamoyl-2-(2-phenylquinolin-7-yl) pyrazolo[1,5-a]pyrimidine-6-carboxylic acid (40 mg, 0.10 mmol, 1.0 eq), NH₄Cl (52.8 mg, 1.0 mmol, 10.0 eq) and HATU (55.7 mg, 0.15 mmol, 1.5 eq) in DMF (10 mL) was added DIEA (63.1 mg, 0.50 mmol, 5.0 eq), and the mixture was stirred at rt for 1 h. The mixture was diluted with water (50 mL), extracted with DCM (50 mL×2) and washed with water (50 mL). The combined organic layers were washed with brine (50 mL), dried over anhydrous sodium sulfate, and concentrated in vacuum. The resulting residue was purified by column chromatography on silica gel (PE/EA=1/1, v/v) to afford 2-(2-phenylquinolin-7-yl) pyrazolo[1,5-a]pyrimidine-3,6-dicarboxamide as a yellow solid (15 mg, 37.5%). LRMS (M+H⁺) m/z calculated 409.1, found 409.0.

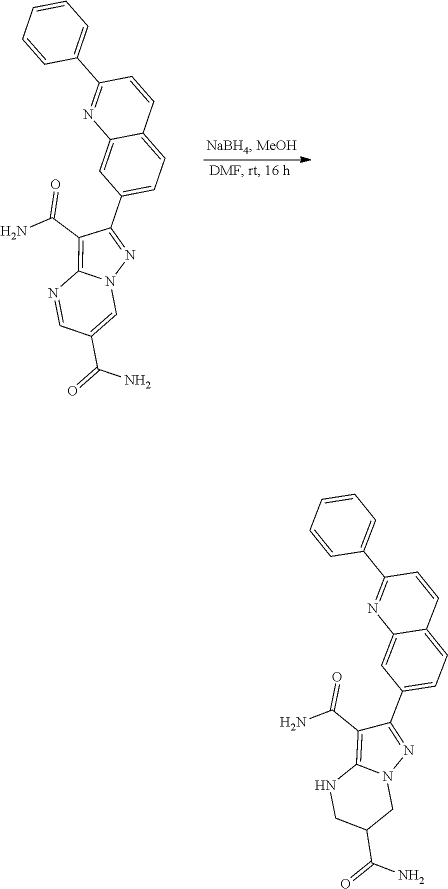 [SMILES: NC(=O)c1cnc2c(C(N)=O)c(-c3ccc4ccc(-c5ccccc5)nc4c3)nn2c1 ; NC(=O)c1c(-c2ccc3ccc(-c4ccccc4)nc3c2)nn2c1NCC(C(N)=O)C2]

To a solution of 2-(2-phenylquinolin-7-yl) pyrazolo[1,5-a]pyrimidine-3,6-dicarboxamide (15 mg, 0.037 mmol, 1.0 eq) in DCM (5 mL) and MeOH (5 mL) was added NaBH₄ (14.0 mg, 0.37 mmol, 10.0 eq). After stirring at rt for 16 h, the mixture was partitioned between DCM/MeOH (50 mL/3 mL) and brine (30 mL). The organic layer was dried over Na₂SO₄ and concentrated in vacuum. The resulting residue was purified by Prep-HPLC to afford 2-(2-phenylquinolin-7-yl)-4,5,6,7-tetrahydropyrazolo[1,5-a]pyrimidine-3,6-dicarboxamide (5.1 mg, 34.0%) as a white solid. LRMS (M+H⁺) m/z calculated 413.2, found 413.1. ¹H NMR (DMSO-d₆, 400 MHz) δ 8.48 (d, 1H), 8.28-8.31 (m, 2H), 8.20 (s, 1H), 8.17 (d, 1H), 8.03 (d, 1H), 7.77 (dd, 1H), 7.51-7.59 (m, 3H), 4.20-4.25 (m, 1H), 4.06-4.12 (m, 1H), 3.46-3.57 (m, 2H), 2.96-2.99 (m, 1H).

### Example 32: Preparation of 5-amino-1-((1s,3s)-3-hydroxy-3-methylcyclobutyl)-3-(4-methoxy-2-phenylquinolin-7-yl)-1H-pyrazole-4-carboxamide

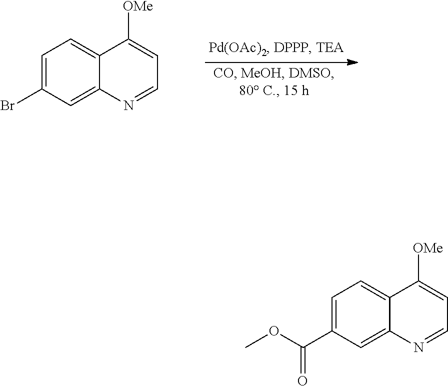 [SMILES: COc1ccnc2cc(Br)ccc12 ; COC(=O)c1ccc2c(OC)ccnc2c1]

To a solution of 7-bromo-4-methoxyquinoline (34 g, 143.4 mmol, 1.0 eq), DPPP (11.8 g, 28.7 mmol, 0.2 eq) and Pd(OAc)₂ (3.2 g, 14.3 mmol, 0.1 eq) in DMSO/MeOH (1000 mL/500 mL) was added TEA (59.5 mL, 430.4 mmol. 3.0 eq). The mixture was stirred at 80° C. for 15 h under CO (1 atm), then concentrated under vacuum. The resulting residue was added into water (300 mL), and the mixture was extracted with EtOAc (550 mL×3). The organic layer was washed with brine (300 mL), dried over anhydrous sodium sulfate, filtered and concentrated under vacuum. The resulting residue was purified by column chromatography on silica gel (PE/EA=5/1, v/v) to afford methyl 4-methoxyquinoline-7-carboxylate (21.2 g, 67.8%) as a yellow oil. LRMS (M+H⁺) m/z calculated 218.1, found 218.1.

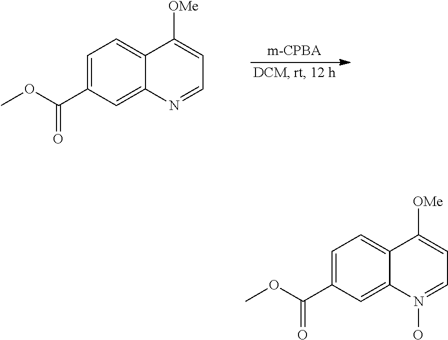

To a solution of methyl 4-methoxyquinoline-7-carboxylate (21.2 g, 97.6 mmol, 1.0 eq) in DCM (300 mL) was added m-CPBA (42.1 g, 244.2 mmol, 2.5 eq) at rt. The mixture was stirred at rt for 12 h, then poured into ice, adjusted to pH 13 with saturated Na₂CO₃ aqueous solution, and extracted with DCM (500 mL×3). The combined organic layers were dried over sodium sulfate, filtered and concentrated under vacuum to afford 4-methoxy-7-(methoxycarbonyl) quinoline 1-oxide (15.2 g, 66.8%) as a yellow oil. LRMS (M+H⁺) m/z calculated 234.1, found 234.2.

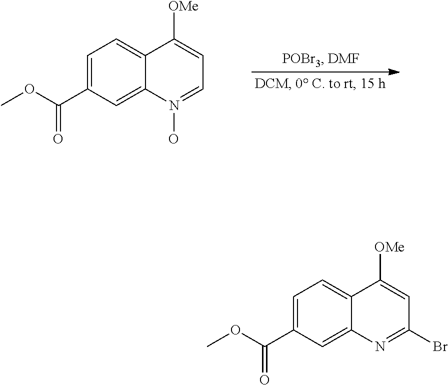

A mixture of 4-methoxy-7-(methoxycarbonyl) quinoline 1-oxide (15.2 g, 65.2 mmol, 1.0 eq), POBr₃ (28.1 g, 97.8 mmol, 1.5 eq) and DMF (2.4 g, 32.6 mmol, 0.5 eq) in DCM (500 mL) was stirred at rt for 15 h. Then the mixture was cooled to rt and poured into ice, adjusted to pH12˜13 with Na₂CO₃ aqueous solution. The aqueous layer was extracted with EtOAc (300 mL×2). The combined organic layers were washed with brine (200 mL), dried over anhydrous sodium sulfate, and concentrated under vacuum. The resulting residue was purified by column chromatography on silica gel (PE/EA=2/1, v/v) to afford methyl 2-bromo-4-methoxyquinoline-7-carboxylate (8.6 g, 44.8%) as a yellow solid. LRMS (M+H⁺) m/z calculated 296.0, found 297.1.

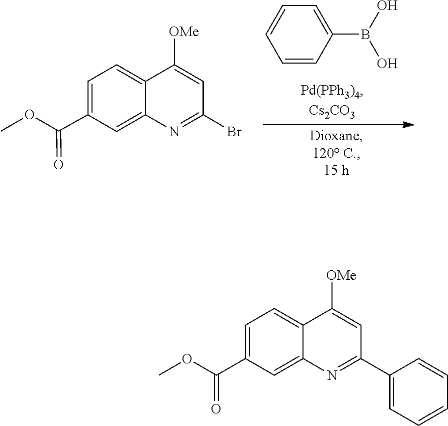 [SMILES: COC(=O)c1ccc2c(OC)cc(Br)nc2c1 ; OB(O)c1ccccc1 ; COC(=O)c1ccc2c(OC)cc(-c3ccccc3)nc2c1]

To a solution of methyl methyl 2-bromo-4-methoxyquinoline-7-carboxylate (8.6 g, 29.0 mmol, 1.0 eq) in dioxane (300 mL) were added phenylboronic acid (7.1 g, 58.1 mmol, 2.0 eq), Pd(PPh₃)₄ (3.4 g, 2.9 mmol, 0.1 eq) and and Cs₂CO₃ (18.9 g, 58.0 mmol, 2.0 eq). The mixture was stirred at 120° C. for 15 h, then concentrated under vacuum, diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 4-methoxy-2-phenylquinoline-7-carboxylate (11 g, ca 100%) as a white solid. LRMS (M+H⁺) m/z calculated 294.1, found 294.1.

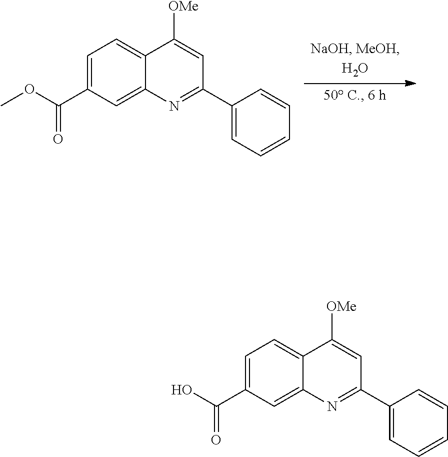 [SMILES: COC(=O)c1ccc2c(OC)cc(-c3ccccc3)nc2c1 ; COc1cc(-c2ccccc2)nc2cc(C(=O)O)ccc12]

To a solution of methyl 4-methoxy-2-phenylquinoline-7-carboxylate (11 g, 37.5 mmol, 1.0 eq) in MeOH (300 mL) and H₂O (50 mL) was added NaOH (2.3 g, 56.3 mmol, 1.5 eq). The mixture was stirred at 50° C. for 5 h, then concentrated under vacuum and diluted with water (100 mL). 37% HCl was added to adjust to pH 2. The resulting mixture was stirred for 5 min, filtered and dried to afford 4-methoxy-2-phenylquinoline-7-carboxylic acid (10 g, 95%) as a white solid, LRMS (M+H⁺) m/z calculated 280.1, found 280.1.

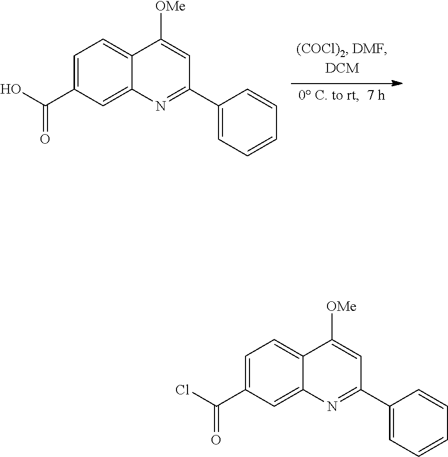 [SMILES: COc1cc(-c2ccccc2)nc2cc(C(=O)O)ccc12 ; COc1cc(-c2ccccc2)nc2cc(C(=O)Cl)ccc12]

To a solution of 4-methoxy-2-phenylquinoline-7-carboxylic acid (10 g, 35.8 mmol, 1.0 eq) in DCM (100 mL) were added (COCl)₂ (9.1 mL, 107.5 mmol, 3.0 eq) and DMF (0.1 mL) at −78° C. The mixture was stirred at rt for 1 h, then concentrated under vacuum to afford 4-methoxy-2-phenylquinoline-7-carbonyl chloride as a yellow solid (12.5 g, ca 100.0%), LRMS (M+H⁺) m/z calculated 294.1, found 294.1 in MeOH.

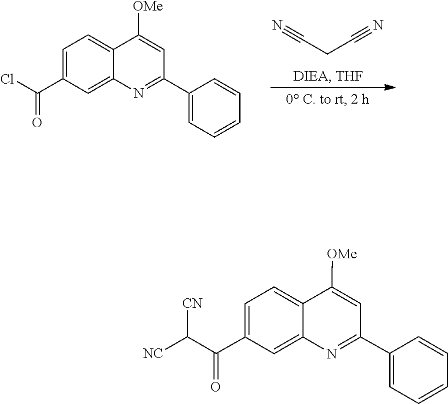 [SMILES: COc1cc(-c2ccccc2)nc2cc(C(=O)Cl)ccc12 ; N#CCC#N ; COc1cc(-c2ccccc2)nc2cc(C(=O)C(C#N)C#N)ccc12]

To a solution of 4-methoxy-2-phenylquinoline-7-carbonyl chloride (12.5 g, 41.9 mmol, 1.0 eq) in THF (200 mL) were added malononitrile (2.8 g, 41.9 mmol, 1.0 eq) and DIEA (21.9 mL, 125.8 mmol, 3.0 eq) at ice bath. The mixture was stirred at rt for 3 h, then concentrated under vacuum, diluted with water (200 mL). The resulting mixture was extracted with EtOAc (300 mL×2). The combined organic layers were washed with brine (300 mL), dried over anhydrous sodium sulfate, filtered and concentrated under vacuum. The resulting residue was purified by column chromatography on silica gel (DCM/MeOH=50/1, v/v) to afford 2-(4-methoxy-2-phenylquinoline-7-carbonyl)malononitrile as a yellow oil (11.6 g, 84%), LRMS (M+H⁺) m/z calculated 328.1, found 328.1.

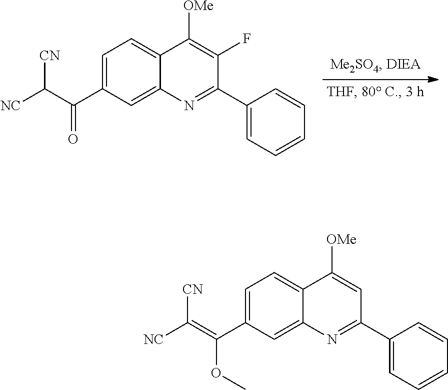 [SMILES: COc1c(F)c(-c2ccccc2)nc2cc(C(=O)C(C#N)C#N)ccc12 ; COC(=C(C#N)C#N)c1ccc2c(OC)cc(-c3ccccc3)nc2c1]

To a solution of 2-(4-methoxy-2-phenylquinoline-7-carbonyl)malononitrile (11.6 g, 35.3 mmol, 1.0 eq) in THF (100 mL) were added Me₂SO₄ (8.9 g, 70.7 mmol, 2.0 eq) and DIEA (18.5 mL, 106.1 mmol, 3.0 eq) at rt. The mixture was stirred at 80° C. for 3 h, then concentrated under vacuum, diluted with water (200 mL), and extracted with EtOAc (500 mL×2). The combined organic layers were washed with brine (500 mL), dried over anhydrous sodium sulfate, filtered and concentrated under vacuum. The resulting residue was purified by column chromatography on silica gel (PE/EA=1/1, v/v) to afford 2-(methoxy(4-methoxy-2-phenylquinolin-7-yl)methylene)malononitrile as a yellow oil (4.8 g, 34%), LRMS (M+H⁺) m/z calculated 342.1, found 342.1.

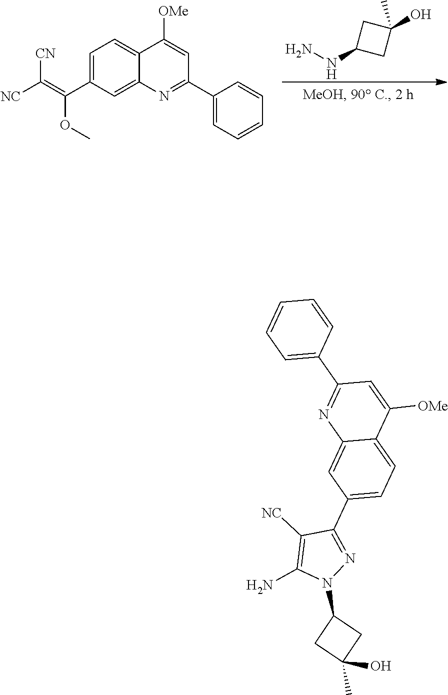

To a solution of 2-(methoxy(4-methoxy-2-phenylquinolin-7-yl)methylene)malononitrile (800 mg, 2.3 mmol, 1.0 eq) and (1s,3s)-3-hydrazineyl-1-methylcyclobutan-1-ol (408.2 mg, 3.5 mmol, 1.5 eq) in MeOH (50 mL) were added TEA (2.6 mL, 18.7 mmol, 8.0 eq) at rt. The reaction mixture was stirred at 90° C. for 2 h then the mixture was concentrated under vacuum. The resulting residue was purified by column chromatography on silica gel (PE/EA=1/1, v/v) to afford 5-amino-1-((1s,3s)-3-hydroxy-3-methylcyclobutyl)-3-(4-methoxy-2-phenylquinolin-7-yl)-1H-pyrazole-4-carbonitrile (800 mg, 80.0%) as a yellow solid. LRMS (M+H⁺) m/z calculated 426.2, found 426.2.

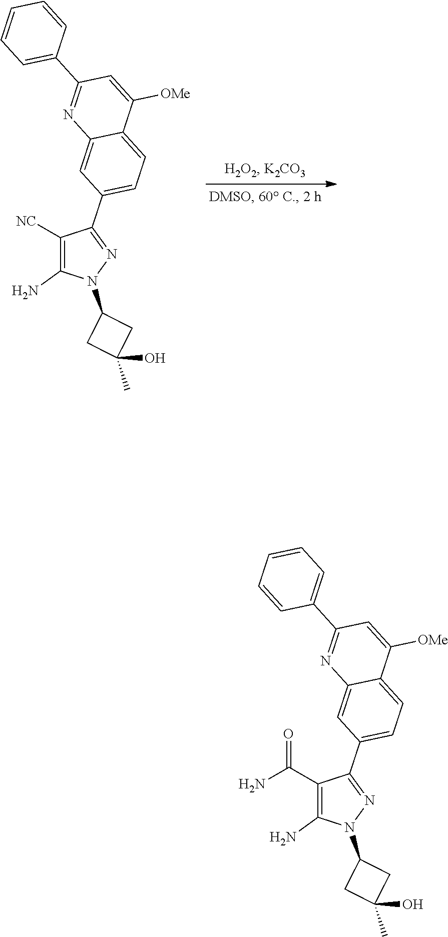 [SMILES: COc1cc(-c2ccccc2)nc2cc(-c3nn([C@H]4C[C@@](C)(O)C4)c(N)c3C#N)ccc12 ; COc1cc(-c2ccccc2)nc2cc(-c3nn([C@H]4C[C@@](C)(O)C4)c(N)c3C(N)=O)ccc12]

To a stirred solution of 5-amino-1-((1s,3s)-3-hydroxy-3-methylcyclobutyl)-3-(4-methoxy-2-phenylquinolin-7-yl)-1H-pyrazole-4-carbonitrile (800 mg, 1.9 mmol, 1.0 eq) and K₂CO₃ (779.3 mg, 5.6 mmol, 3.0 eq) in DMSO (50 mL) was added H₂O₂ (30%, 3.0 mL, 37.6 mmol, 20.0 eq) at rt. After addition was complete, the reaction mixture was stirred at 60° C. for 2 h. The mixture was diluted with water (50 mL) and extracted with EtOAc (50 mL×2). The combined organic layers were washed with brine (100 mL), dried over anhydrous sodium sulfate, filtered, concentrated under vacuum, and purified by Prep-HPLC to afford 5-amino-1-((1s,3s)-3-hydroxy-3-methylcyclobutyl)-3-(4-methoxy-2-phenylquinolin-7-yl)-1H-pyrazole-4-carboxamide (464.2 mg, 56%) as a white solid. LRMS (M+H⁺) m/z calculated 444.2, found 444.2. ¹H NMR (DMSO-d₆, 400 MHz) δ 8.31 (d, 2H), 8.17 (d, 1H), 8.14 (s, 1H), 7.15 (dd, 1H), 7.48-7.57 (m, 4H), 6.30 (s, 2H), 5.20 (s, 1H), 4.44-4.49 (m, 1H), 4.19 (s, 3H), 2.59-2.64 (m, 2H), 2.36-2.41 (m, 2H), 1.34 (s, 3H).

### Example 33: Preparation of 5-amino-3-(4-ethoxy-8-fluoro-2-phenylquinolin-7-yl)-1-((1s,3s)-3-hydroxy-3-methylcyclobutyl)-1H-pyrazole-4-carboxamide

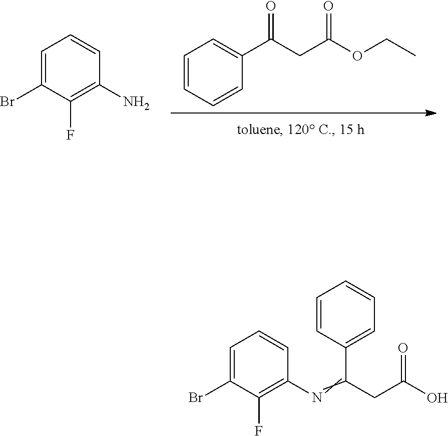 [SMILES: Nc1cccc(Br)c1F ; CCOC(=O)CC(=O)c1ccccc1 ; O=C(O)CC(=Nc1cccc(Br)c1F)c1ccccc1]

To a solution of 3-bromo-2-fluoroaniline (25.0 g, 132.2 mmol, 1.0 eq) in Tol (300 mL) was added ethyl 3-oxo-3-phenylpropanoate (25.4 g, 132.2 mmol, 1.0 eq) at rt. The mixture was stirred at 120° C. for 15 h. The mixture was cooled to rt and diluted with PE (500 mL). The precipitate was filtered, and dried under vacuum to afford 3-((3-bromo-2-fluorophenyl)imino)-3-phenylpropanoic acid (7 g, 15.9%) as a yellow solid. LRMS (M+H⁺) m/z calculated 336.0, found 336.1.

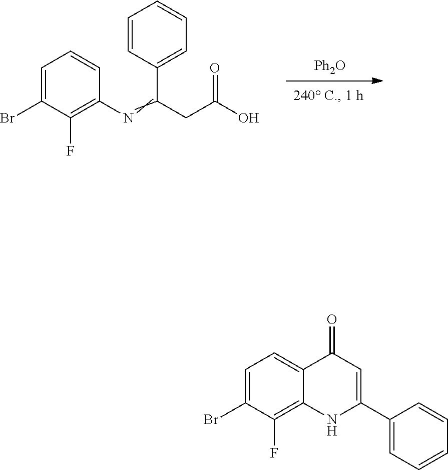 [SMILES: O=C(O)CC(=Nc1cccc(Br)c1F)c1ccccc1 ; "Ph2O" ; O=c1cc(-c2ccccc2)[nH]c2c(F)c(Br)ccc12]

A mixture of 3-((3-bromo-2-fluorophenyl)imino)-3-phenylpropanoic acid (15 g, 44.6 mmol, 1.0 eq) and Ph₂O (150 mL) was stirred at 240° C. for 1 h. The mixture was cooled to rt and diluted with PE (200 mL). The precipitate was filtered, dried to afford 7-bromo-8-fluoro-2-phenylquinolin-4 (1H)-one (3 g, 21.1%) as a brown solid. LRMS (M+H⁺) m/z calculated 318.0, found 318.1.

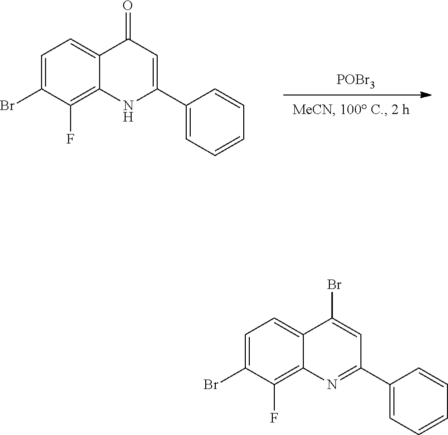 [SMILES: O=c1cc(-c2ccccc2)[nH]c2c(F)c(Br)ccc12 ; "POBr3" ; Fc1c(Br)ccc2c(Br)cc(-c3ccccc3)nc12]

To a solution of 7-bromo-8-fluoro-2-phenylquinolin-4 (1H)-one (8 g, 25.2 mmol, 1.0 eq) in MeCN (120 mL) was added POBr₃ (14.4 g, 50 mmol, 2.0 eq). The mixture was stirred at 100° C. for 2 h. Then the mixture was cooled to rt, poured into ice, adjusted to pH 13 with saturated Na₂CO₃ aqueous solution, and extracted with DCM (500 mL×3). The combined organic layers were dried over anhydrous sodium sulfate, filtered and concentrated under vacuum. The resulting residue was purified by column chromatography on silica gel (PE/EA=5/1, v/v) to afford 4,7-dibromo-8-fluoro-2-phenylquinoline (6 g, 62.3%) as a yellow solid. LRMS (M+H⁺) m/z calculated 381.9, found 381.9.

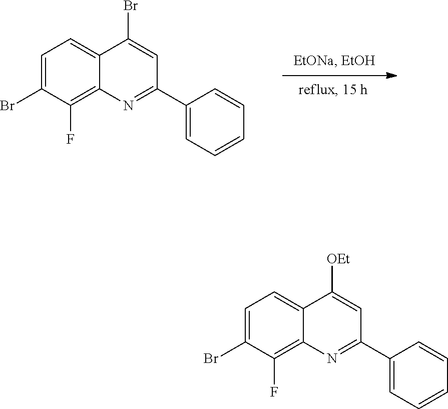 [SMILES: Fc1c(Br)ccc2c(Br)cc(-c3ccccc3)nc12 ; CCOc1cc(-c2ccccc2)nc2c(F)c(Br)ccc12]

A mixture of 4,7-dibromo-8-fluoro-2-(2-fluorophenyl) quinoline (970 mg, 2.4 mmol, 1.0 eq), EtONa (216.0 mg, 3.2 mmol, 1.3 eq) and EtOH (30 mL) was stirred at reflux for 15 h. The mixture was concentrated under vacuum. The resulting residue was purified by column chromatography on silica gel (PE/EA-2/1, v/v) to afford 7-bromo-4-ethoxy-8-fluoro-2-phenylquinoline (420 mg, 51%) as a yellow solid. LRMS (M+H⁺) m/z calculated 346.0, found 346.0.

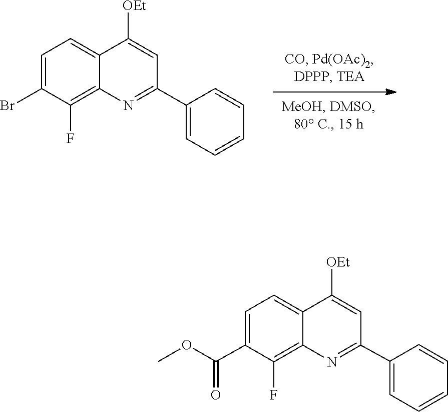 [SMILES: CCOc1cc(-c2ccccc2)nc2c(F)c(Br)ccc12 ; CCOc1cc(-c2ccccc2)nc2c(F)c(C(=O)OC)ccc12]

To a solution of 7-bromo-4-ethoxy-8-fluoro-2-phenylquinoline (420 mg, 1.2 mmol, 1.0 eq), DPPP (200.0 mg, 0.49 mmol, 0.4 eq) and Pd(OAc)₂ (54.3 mg, 0.24 mmol, 0.2 eq) in DMSO/MeOH (100 mL/100 mL) was added TEA (0.9 mL, 6.1 mmol. 5.0 eq). The mixture was stirred at 80° C. for 15 h under CO (1 atm), then the reaction mixture was diluted with water (500 mL) and extracted with EtOAc (200 mL×3). The combined organic layers were washed with brine (300 mL), dried with anhydrous sodium sulfate, filtered, and concentrated under vacuum. The resulting residue was purified by column chromatography on silica gel (PE/EA=1/1, v/v) to afford methyl 4-ethoxy-8-fluoro-2-phenylquinoline-7-carboxylate (420 mg, ca 100%) as a yellow solid. LRMS (M+H⁺) m/z calculated 326.1, found 326.1.

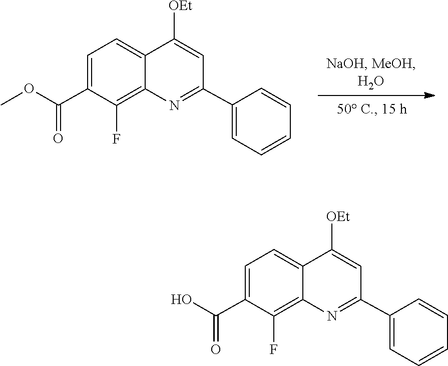 [SMILES: CCOc1cc(-c2ccccc2)nc2c(F)c(C(=O)OC)ccc12 ; CCOc1cc(-c2ccccc2)nc2c(F)c(C(=O)O)ccc12]

To a solution of methyl 4-ethoxy-8-fluoro-2-phenylquinoline-7-carboxylate (420 mg, 1.3 mmol, 1.0 eq) in MeOH (30 mL) and H₂O (3 mL) was added NaOH (77.5 mg, 1.9 mmol, 1.5 eq). The mixture was stirred at 80° C. for 15 h, then concentrated under vacuum and diluted with water (10 mL), adjusted to pH=2 with 37% HCl aq. The resulting mixture was stirred for 5 min, filtered and dried under vacuum to afford 4-ethoxy-8-fluoro-2-phenylquinoline-7-carboxylic acid (349 mg, 87%) as a white solid, LRMS (M+H⁺) m/z calculated 312.1, found 312.1.

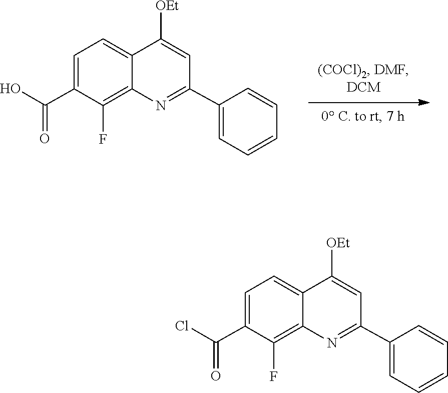 [SMILES: CCOc1cc(-c2ccccc2)nc2c(F)c(C(=O)O)ccc12 ; CCOc1cc(-c2ccccc2)nc2c(F)c(C(=O)Cl)ccc12]

To a solution of 4-ethoxy-8-fluoro-2-phenylquinoline-7-carboxylic acid (349 mg, 1.1 mmol, 1.0 eq) in DCM (30 mL) were added (COCl)₂ (0.5 mL, 5.6 mmol, 5.0 eq) and DMF (2 drop) at −78° C. The mixture was stirred at rt for 7 h, then concentrated under vacuum to afford 4-ethoxy-8-fluoro-2-phenylquinoline-7-carbonyl chloride as a yellow solid (550 mg, ca 100.0%) which was used to the next step directly. LRMS (M+H⁺) m/z calculated 326.1, found 326.1.

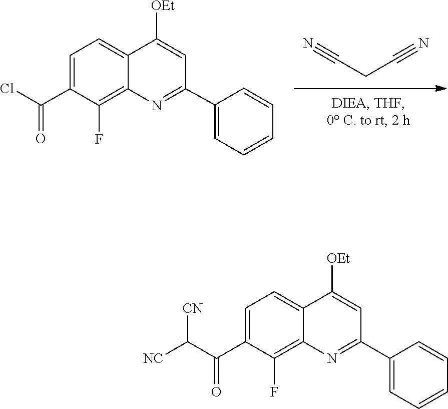 [SMILES: CCOc1cc(-c2ccccc2)nc2c(F)c(C(=O)Cl)ccc12 ; N#CCC#N ; CCOc1cc(-c2ccccc2)nc2c(F)c(C(=O)C(C#N)C#N)ccc12]

To a solution of 4-ethoxy-8-fluoro-2-phenylquinoline-7-carbonyl chloride (550 mg, 1.7 mmol, 1.0 eq) in THF (70 mL) were added malononitrile (110 mg, 1.7 mmol, 1.0 eq) and DIEA (0.9 mL, 5 mmol, 3.0 eq). The mixture was stirred at rt for 2 h, then concentrated, diluted with water (100 mL). The resulting mixture was extracted with EtOAc (150 mL×2). The combined organic layers were washed with brine (150 mL), dried over anhydrous sodium sulfate, filtered and concentrated under vacuum. The resulting residue was purified by column chromatography on silica gel (DCM/MeOH=50/1, v/v) to afford 2-(4-ethoxy-8-fluoro-2-phenylquinoline-7-carbonyl)malononitrile as a yellow oil (320 mg, 79%), LRMS (M+H⁺) m/z calculated 360.1, found 360.0.

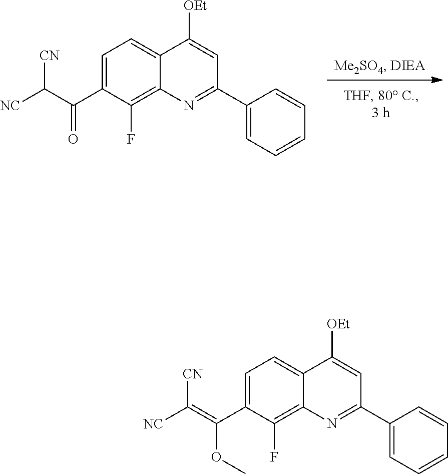 [SMILES: CCOc1cc(-c2ccccc2)nc2c(F)c(C(=O)C(C#N)C#N)ccc12 ; CCOc1cc(-c2ccccc2)nc2c(F)c(C(OC)=C(C#N)C#N)ccc12]

To a solution of 2-(4-ethoxy-8-fluoro-2-phenylquinoline-7-carbonyl)malononitrile (320 mg, 0.90 mmol, 1.0 eq) in THF (50 mL) were added Me₂SO₄ (224.5 mg, 1.8 mmol, 2.0 eq) and DIEA (574.9 mg, 4.5 mmol, 5.0 eq). The mixture was stirred at 80° C. for 3 h, then concentrated under vacuum and diluted with water (100 mL), extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=1/1, v/v) to afford 2-((4-ethoxy-8-fluoro-2-phenylquinolin-7-yl)(methoxy)methylene)malononitrile as a yellow oil (110 mg, 33%), LRMS (M+H⁺) m/z calculated 374.1, found 374.1.

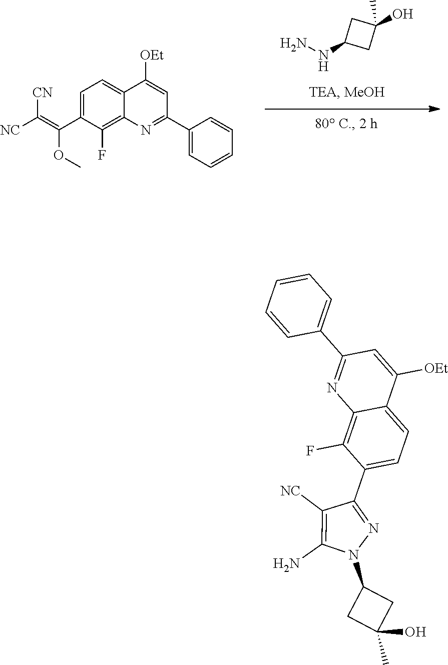

To a solution of 2-((4-ethoxy-8-fluoro-2-phenylquinolin-7-yl)(methoxy)methylene)malononitrile (110 mg, 0.29 mmol, 1.0 eq) and (1s,3s)-3-hydrazineyl-1-methylcyclobutan-1-ol (41.1 mg, 0.35 mmol, 1.2 eq) in MeOH (50 mL) was added TEA (0.33 mL, 2.4 mmol, 8.0 eq) at rt. The reaction mixture was stirred at 80° C. for 2 h, then concentrated under vacuum. The resulting residue was purified by column chromatography on silica gel (PE/EA=2/1, v/v) to afford 5-amino-3-(4-ethoxy-8-fluoro-2-phenylquinolin-7-yl)-1-((1s,3s)-3-hydroxy-3-methylcyclobutyl)-1H-pyrazole-4-carbonitrile (40 mg, 30%) as a yellow solid, LRMS (M+H⁺) m/z calculated 458.2, found 458.2.

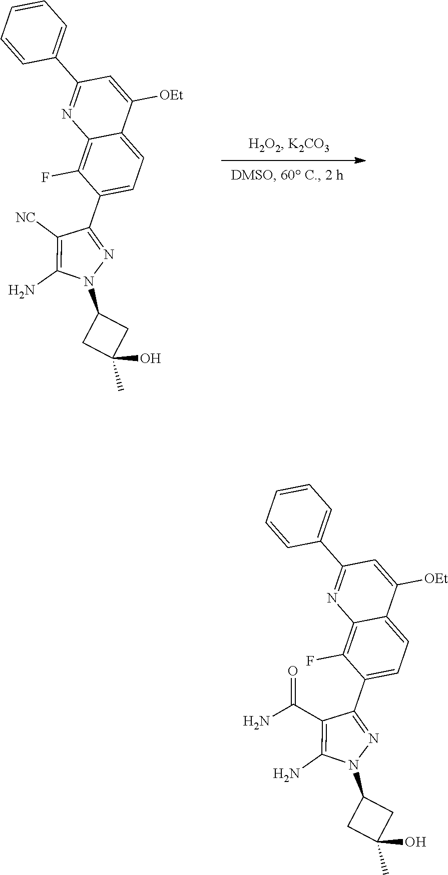 [SMILES: CCOc1cc(-c2ccccc2)nc2c(F)c(-c3nn([C@H]4C[C@@](C)(O)C4)c(N)c3C#N)ccc12 ; CCOc1cc(-c2ccccc2)nc2c(F)c(-c3nn([C@H]4C[C@@](C)(O)C4)c(N)c3C(N)=O)ccc12]

To a stirred solution of 5-amino-3-(4-ethoxy-8-fluoro-2-phenylquinolin-7-yl)-1-((1s,3s)-3-hydroxy-3-methylcyclobutyl)-1H-pyrazole-4-carbonitrile (30 mg, 0.065 mmol, 1.0 eq) in DMSO (20 mL) were added K₂CO₃ (45.3 mg, 0.33 mmol, 5.0 eq) and H₂O₂ (30%, 0.1 mL, 1.3 mmol, 20.0 eq). After addition was complete, the mixture was stirred at 60° C. for 2 h, then diluted with water (100 mL), and extracted with EtOAc (150 mL×2). The combined organic layers were washed with brine (150 mL), dried over anhydrous sodium sulfate, and concentrated under vacuum. The residue was purified by Prep-HPLC to afford 5-amino-3-(4-ethoxy-8-fluoro-2-phenylquinolin-7-yl)-1-((1s,3s)-3-hydroxy-3-methylcyclobutyl)-1H-pyrazole-4-carboxamide as a white solid (340.2 mg, 43%), LRMS (M+H⁺) m/z calculated 476.2, found 476.2. ¹H NMR (DMSO-d₆, 400 MHz) δ 8.32 (d, 2H), 7.99 (d, 1H), 7.67 (s, 1H), 7.52-7.60 (m, 4H), 6.34 (s, 2H), 5.21 (s, 1H), 4.49-4.53 (m, 3H), 2.54-2.60 (m, 2H), 2.35-2.41 (m, 2H), 1.54 (t, 3H), 1.34 (s, 3H).

### Example 34: Preparation of 5-amino-3-(8-fluoro-4-methoxy-2-phenylquinolin-7-yl)-1-((1s,3s)-3-hydroxy-3-methylcyclobutyl)-1H-pyrazole-4-carboxamide

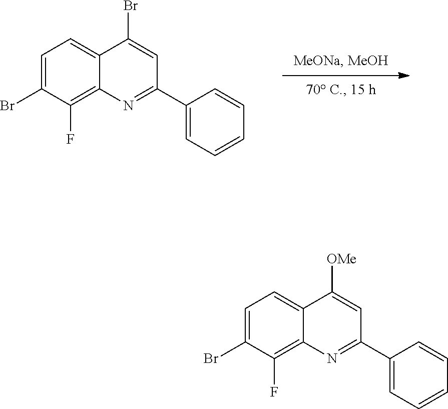 [SMILES: Fc1c(Br)ccc2c(Br)cc(-c3ccccc3)nc12 ; COc1cc(-c2ccccc2)nc2c(F)c(Br)ccc12]

To a solution of 4,7-dibromo-8-fluoro-2-phenylquinoline (6 g, 15.5 mmol, 1.0 eq) in MeOH (100 mL) was added MeONa (1.7 g, 30.1 mmol, 2.0 eq). The mixture was stirred at 70° C. for 15 h. The mixture was concentrated under vacuum, diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford 7-bromo-8-fluoro-4-methoxy-2-phenylquinoline (4.5 g, 88.2%) as a white solid. LRMS (M+H⁺) m/z calculated 332.0, found 332.0.

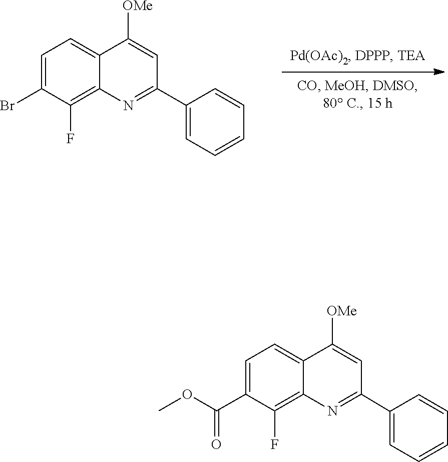 [SMILES: COc1cc(-c2ccccc2)nc2c(F)c(Br)ccc12 ; COC(=O)c1ccc2c(OC)cc(-c3ccccc3)nc2c1F]

To a solution of 7-bromo-8-fluoro-4-methoxy-2-phenylquinoline (2 g, 6.0 mmol, 1.0 eq), DPPP (1 g, 2.4 mmol, 0.4 eq) and Pd(OAc)₂ (270 mg, 1.2 mmol, 0.2 eq) in DMSO/MeOH (50 mL/50 mL) was added TEA (9.6 mL, 30 mmol. 5.0 eq). The mixture was stirred at 80° C. for 15 h under CO (1 atm), then the reaction mixture was diluted with water (100 mL) and extracted with EtOAc (100 mL×3). The combined organic layers were washed with brine (100 mL), dried with anhydrous sodium sulfate, filtered, and concentrated under vacuum. The resulting residue was purified by column chromatography on silica gel (PE/EA=5/1, v/v) to afford methyl 8-fluoro-4-methoxy-2-phenylquinoline-7-carboxylate (1.3 g, 72.2%) as a white solid. LRMS (M+H⁺) m/z calculated 312.1, found 312.2.

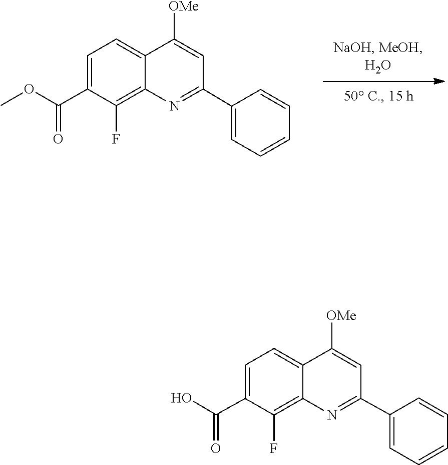 [SMILES: COC(=O)c1ccc2c(OC)cc(-c3ccccc3)nc2c1F ; COc1cc(-c2ccccc2)nc2c(F)c(C(=O)O)ccc12]

To a solution of methyl 8-fluoro-4-methoxy-2-phenylquinoline-7-carboxylate (1.3 g, 4.1 mmol, 1.0 eq) in MeOH (30 mL) and H₂O (10 mL) was added NaOH (501 mg, 12.3 mmol, 3.0 eq). The mixture was stirred at 50° C. for 15 h, then concentrated under vacuum and diluted with water (60 mL). The mixture was adjusted to pH 2 with 37% of HCl aqeuous solution. The resulting mixture was stirred for 5 min, filtered and dried to afford 8-fluoro-4-methoxy-2-phenylquinoline-7-carboxylic acid (1.1 g, 90.2%) as a white solid, LRMS (M+H⁺) m/z calculated 298.1, found 298.2.

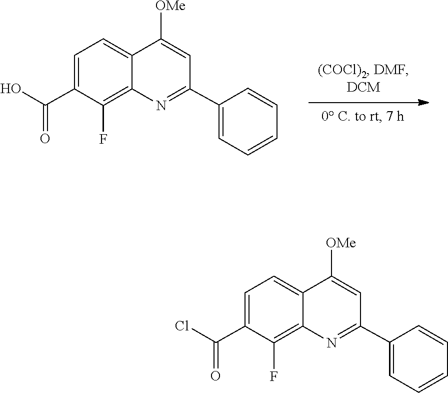 [SMILES: COc1cc(-c2ccccc2)nc2c(F)c(C(=O)O)ccc12 ; COc1cc(-c2ccccc2)nc2c(F)c(C(=O)Cl)ccc12]

To a solution of 8-fluoro-4-methoxy-2-phenylquinoline-7-carboxylic acid (1.1 g, 3.7 mmol, 1.0 eq) in DCM (30 mL) were added (COCl)₂ (0.7 mL, 18.5 mmol, 5.0 eq) and DMF (5 drops) at 0° C. The mixture was stirred at rt for 7 h, then concentrated under vacuum to afford 8-fluoro-4-methoxy-2-phenylquinoline-7-carbonyl chloride (1.3 g, ca 100%) as a yellow solid which was used for the next step directly. LRMS (M+H⁺) m/z calculated 312.1, found 312.2 in MeOH.

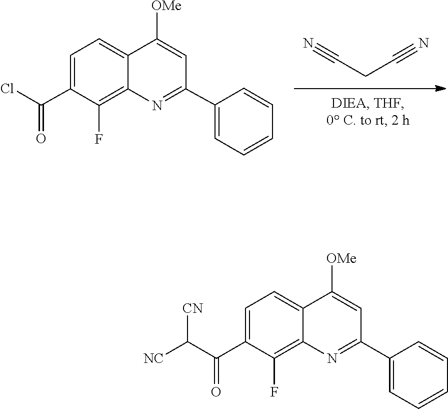 [SMILES: COc1cc(-c2ccccc2)nc2c(F)c(C(=O)Cl)ccc12 ; N#CCC#N ; COc1cc(-c2ccccc2)nc2c(F)c(C(=O)C(C#N)C#N)ccc12]

To a solution of 8-fluoro-4-methoxy-2-phenylquinoline-7-carbonyl chloride (1.3 g, 4.1 mmol, 1.0 eq) in THF (30 mL) were added malononitrile (817 mg, 12.3 mmol, 3.0 eq) and DIEA (3.5 mL, 20.5 mmol, 5.0 eq). The mixture was stirred at rt for 3 h, then concentrated, and diluted with water (20 mL). The resulting mixture was extracted with EtOAc (50 mL×2). The combined organic layers were washed with brine (50 mL), dried over anhydrous sodium sulfate, filtered and concentrated under vacuum. The resulting residue was purified by column chromatography on silica gel (DCM/MeOH-20/1, v/v) to afford 2-(8-fluoro-4-methoxy-2-phenylquinoline-7-carbonyl)malononitrile as a yellow oil (1.2 g, 82.9%), LRMS (M+H⁺) m/z calculated 346.1, found 346.2.

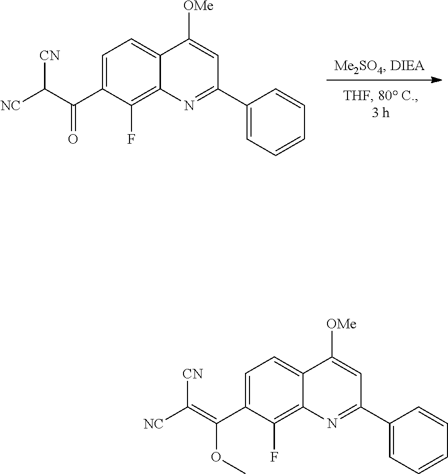 [SMILES: COc1cc(-c2ccccc2)nc2c(F)c(C(=O)C(C#N)C#N)ccc12 ; COC(=C(C#N)C#N)c1ccc2c(OC)cc(-c3ccccc3)nc2c1F]

To a solution of 2-(8-fluoro-4-methoxy-2-phenylquinoline-7-carbonyl)malononitrile (1.2 g, 3.4 mmol, 1.0 eq) in THF (30 mL) were added Me₂SO₄ (0.9 mL, 17 mmol, 5.0 eq) and DIEA (3.1 mL, 34 mmol, 10 eq). The mixture was stirred at 80° C. for 3 h. The mixture was concentrated under vacuum, diluted with water (20 mL), and extracted with EtOAc (50 mL×2). The combined organic layer was washed with brine (50 mL), dried over anhydrous sodium sulfate, filtered and concentrated under vacuum. The resulting residue was purified by column chromatography on silica gel (PE/EA=2/1, v/v) to afford 2-((8-fluoro-4-methoxy-2-phenylquinolin-7-yl)(methoxy)methylene)malononitrile as a yellow oil (800 mg, 66.6%), LRMS (M+H⁺) m/z calculated 360.1, found 360.2.

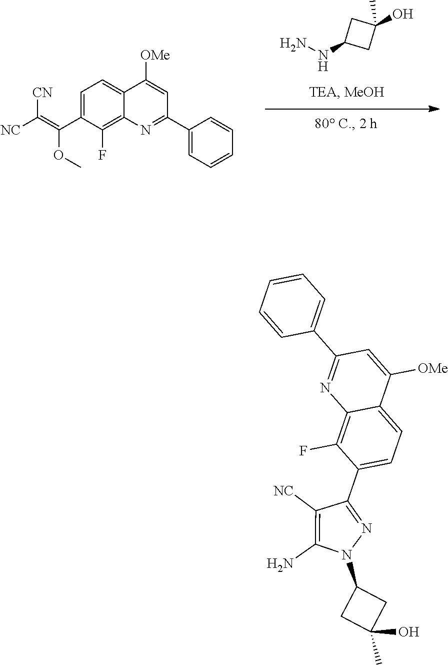

To a solution of 2-((8-fluoro-4-methoxy-2-phenylquinolin-7-yl)(methoxy)methylene)malononitrile (800 mg, 2.2 mmol, 1.0 eq) and (1s,3s)-3-hydrazineyl-1-methylcyclobutan-1-ol (384 mg, 3.3 mmol, 1.5 eq) in MeOH (20 mL) were added TEA (3.4 mL, 17.2 mmol, 8.0 eq) at rt. The reaction mixture was stirred at 90° C. for 2 h then the mixture was concentrated under vacuum. The resulting residue was purified by column chromatography on silica gel (PE/EA=2/1, v/v) to afford 5-amino-3-(8-fluoro-4-methoxy-2-phenylquinolin-7-yl)-1-((1s,3s)-3-hydroxy-3-methylcyclobutyl)-1H-pyrazole-4-carbonitrile (500 mg, 50.9%) as a white solid. LRMS (M+H⁺) m/z calculated 444.2, found 444.3.

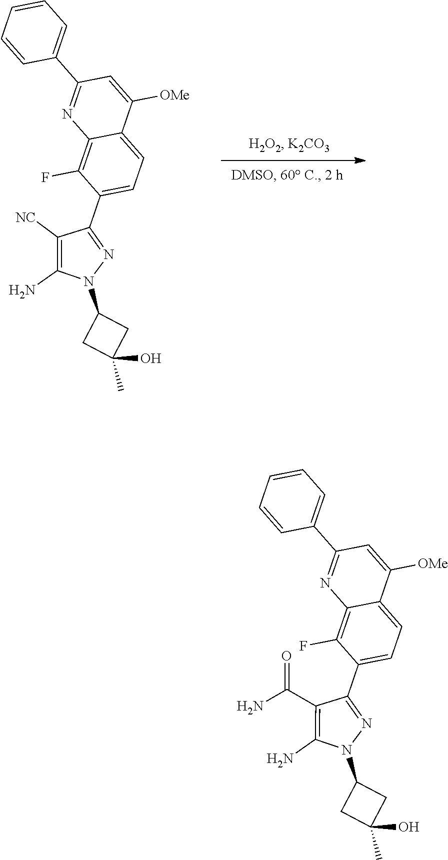 [SMILES: COc1cc(-c2ccccc2)nc2c(F)c(-c3nn([C@H]4C[C@@](C)(O)C4)c(N)c3C#N)ccc12 ; COc1cc(-c2ccccc2)nc2c(F)c(-c3nn([C@H]4C[C@@](C)(O)C4)c(N)c3C(N)=O)ccc12]

To a stirred solution of 5-amino-3-(8-fluoro-4-methoxy-2-phenylquinolin-7-yl)-1-((1s,3s)-3-hydroxy-3-methylcyclobutyl)-1H-pyrazole-4-carbonitrile (500 mg, 1.12 mmol, 1.0 eq) and K₂CO₃ (467 mg, 3.36 mmol, 3.0 eq) in DMSO (5 mL) at rt was added H₂O₂ (30%, 1.3 mL, 11.2 mmol, 10.0 eq). The mixture was stirred at 60° C. for 2 h, then diluted with water (100 mL), extracted with EtOAc (150 mL×2). The combined organic layers were washed with brine (150 mL), dried over anhydrous sodium sulfate, filtered and concentrated under vacuum. The residue was purified by Prep-HPLC to afford 5-amino-3-(8-fluoro-4-methoxy-2-phenylquinolin-7-yl)-1-((1s,3s)-3-hydroxy-3-methylcyclobutyl)-1H-pyrazole-4-carboxamide (374 mg, 72.4%) as a white solid. LRMS (M+H⁺) m/z calculated 462.2, found 462.2, ¹H NMR (DMSO-d₆, 400 MHz) δ 8.33 (d, 2H), 7.98 (d, 1H), 7.68 (s, 1H), 7.52-7.59 (m, 4H), 6.32 (s, 2H), 5.19 (s, 1H), 4.45-4.50 (m, 1H), 4.21 (s, 3H), 2.55-2.60 (m, 2H), 2.36-2.40 (m, 2H), 1.33 (s, 3H).

### Example 35: Preparation of 5-amino-3-(8-fluoro-2-(2-fluorophenyl)-4-methoxyquinolin-7-yl)-1-((1s, 3s)-3-hydroxy-3-methylcyclobutyl)-1H-pyrazole-4-carboxamide

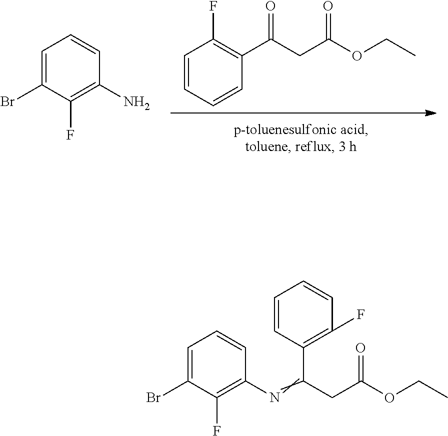 [SMILES: Nc1cccc(Br)c1F ; CCOC(=O)CC(=O)c1ccccc1F ; CCOC(=O)CC(=Nc1cccc(Br)c1F)c1ccccc1F]

To a solution of 3-bromo-2-fluoroaniline (50 g, 264.6 mmol, 1.0 eq) in toluene (800 mL) were added ethyl 3-(2-fluorophenyl)-3-oxopropanoate (55.6 g, 264.6 mmol, 1.0 eq) and p-toluenesulfonic acid (4.6 g, 26.5 mmol, 0.1 eq). The reaction mixture was stirred at reflux for 3 h, then concentrated under vacuum. The resulting residue was purified by column chromatography on silica gel (PE/EA=10/1, v/v) to afford ethyl 3-((3-bromo-2-fluorophenyl)imino)-3-(2-fluorophenyl) propanoate (8.4 g, 8.4%) as a yellow oil. LRMS (M+H⁺) m/z calculated 381.0, found 381.1.

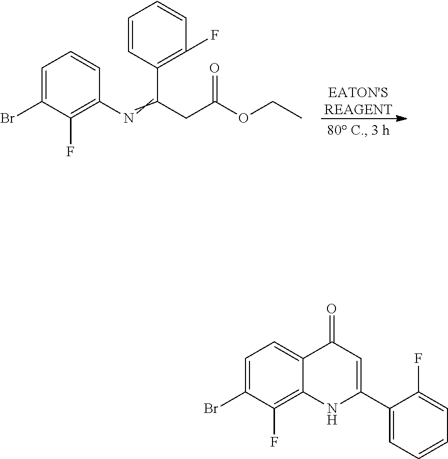 [SMILES: CCOC(=O)CC(=Nc1cccc(Br)c1F)c1ccccc1F ; "EATON'SREAGENT" ; O=c1cc(-c2ccccc2F)[nH]c2c(F)c(Br)ccc12]

A mixture of ethyl 3-((3-bromo-2-fluorophenyl)imino)-3-(2-fluorophenyl) propanoate (8.4 g, 22.1 mmol, 8.4%) and EATON'S REAGENT was stirred at 80° C. for 3 h. Then the mixture was cooled to rt, poured into ice and was adjusted to pH 12˜13 with Na₂CO₃ aq. The aqueous layer was extracted with EtOAc (200 mL×2). The combined organic layers were washed with brine (100 mL) and dried over anhydrous sodium sulfate, filtered, and concentrated under vacuum. The resulting residue was purified by column chromatography on silica gel (PE/EA=5/1, v/v) to afford 7-bromo-8-fluoro-2-(2-fluorophenyl) quinolin-4 (1H)-one (9.4 g, ca. 100%) as a yellow solid. LRMS (M+H⁺) m/z calculated 336.0, found 336.1.

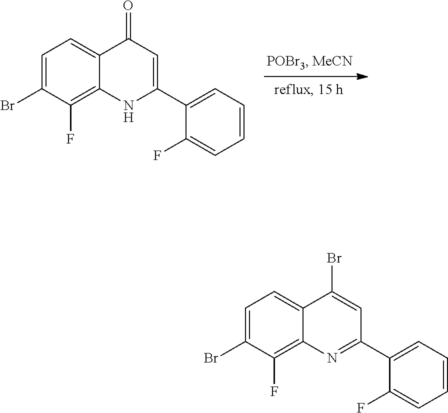 [SMILES: O=c1cc(-c2ccccc2F)[nH]c2c(F)c(Br)ccc12 ; Fc1ccccc1-c1cc(Br)c2ccc(Br)c(F)c2n1]

A mixture of 7-bromo-8-fluoro-2-(2-fluorophenyl) quinolin-4 (1H)-one (9.4 g, 27.9 mmol, 1.0 eq), POBr₃ (24.1 g, 83.9 mmol, 3.0 eq) and MeCN (200 mL) was stirred under reflux for 15 h. The mixture was cooled to rt and poured into ice, adjusted to pH 12˜13 with Na₂CO₃ aqueous solution. The aqueous layer was extracted with EtOAc (200 mL×2). The combined organic layers were washed with brine (100 mL) and dried over anhydrous sodium sulfate, filtered, concentrated under vacuum. The resulting residue was purified by column chromatography on silica gel (PE/EA=2/1, v/v) to afford 4,7-dibromo-8-fluoro-2-(2-fluorophenyl) quinoline (14.7 g, ca. 100%) as a yellow solid. LRMS (M+H⁺) m/z calculated 399.9, found 401.0.

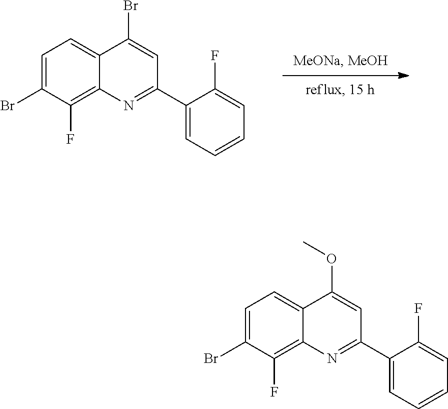 [SMILES: Fc1ccccc1-c1cc(Br)c2ccc(Br)c(F)c2n1 ; COc1cc(-c2ccccc2F)nc2c(F)c(Br)ccc12]

A mixture of 4,7-dibromo-8-fluoro-2-(2-fluorophenyl) quinoline (14.7 g, 36.8 mmol, 1.0 eq), MeONa (6.0 g, 110.5 mmol, 3.0 eq) and MeOH (200 mL) was stirred at reflux for 15 h. Then the mixture was concentrated under vacuum. The resulting residue was purified by column chromatography on silica gel (PE/EA=2/1, v/v) to afford 7-bromo-8-fluoro-2-(2-fluorophenyl)-4-methoxyquinoline (10.8 g, 83.7%) as a yellow solid. LRMS (M+H⁺) m/z calculated 350.0, found 350.0.

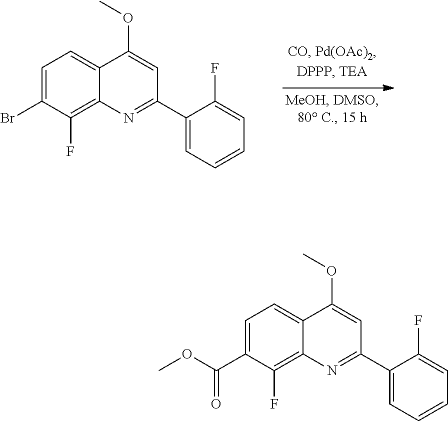 [SMILES: COc1cc(-c2ccccc2F)nc2c(F)c(Br)ccc12 ; COC(=O)c1ccc2c(OC)cc(-c3ccccc3F)nc2c1F]

To a solution of 7-bromo-8-fluoro-2-(2-fluorophenyl)-4-methoxyquinoline (10.8 g, 30.9 mmol, 1.0 eq), DPPP (5.1 g, 12.3 mmol, 0.4 eq) and Pd(OAc)₂ (1.4 g, 6.2 mmol, 0.2 eq) in DMSO/MeOH (200 mL/300 mL) was added TEA (21.3 mL, 154.3 mmol. 5.0 eq). The mixture was stirred at 80° C. for 15 h under CO (1 atm), then the reaction mixture was diluted with water (2000 mL) and extracted with EtOAc (500 mL×3). The combined organic layers were washed with brine (300 mL), dried with anhydrous sodium sulfate, filtered, and concentrated under vacuum. The resulting residue was purified by column chromatography on silica gel (PE/EA=1/1, v/v) to afford methyl 8-fluoro-2-(2-fluorophenyl)-4-methoxyquinoline-7-carboxylate (10.5 g, ca 100%) as a yellow solid. LRMS (M+H⁺) m/z calculated 330.1, found 330.1.

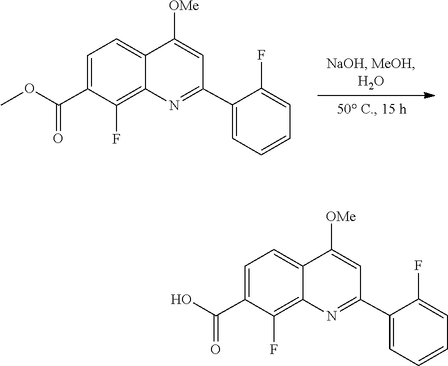 [SMILES: COC(=O)c1ccc2c(OC)cc(-c3ccccc3F)nc2c1F ; COc1cc(-c2ccccc2F)nc2c(F)c(C(=O)O)ccc12]

To a solution of methyl 8-fluoro-2-(2-fluorophenyl)-4-methoxyquinoline-7-carboxylate (10.5 g, 31.9 mmol, 1.0 eq) in MeOH (100 mL) and H₂O (30 mL) was added NaOH (1.9 g, 47.8 mmol, 1.5 eq). The mixture was stirred at 80° C. for 15 h, concentrated under vacuum, diluted with water (60 mL), and adjusted to pH 2 with 37% HCl aqueous solution. The resulting mixture was stirred for 5 min, filtered and concentrated under vacuum to afford 8-fluoro-2-(2-fluorophenyl)-4-methoxyquinoline-7-carboxylic acid (9.9 g, 98%) as a white solid, LRMS (M+H⁺) m/z calculated 316.1, found 316.0.

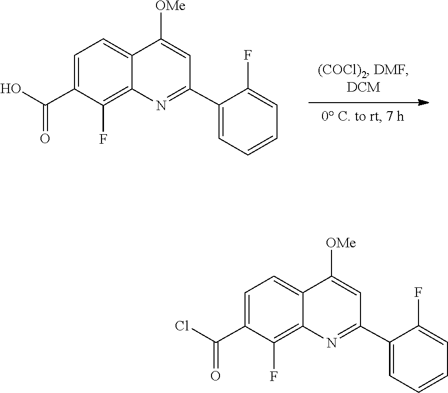 [SMILES: COc1cc(-c2ccccc2F)nc2c(F)c(C(=O)O)ccc12 ; COc1cc(-c2ccccc2F)nc2c(F)c(C(=O)Cl)ccc12]

To a solution of 8-fluoro-2-(2-fluorophenyl)-4-methoxyquinoline-7-carboxylic acid (9.9 g, 31.4 mmol, 1.0 eq) in DCM (100 mL) were added (COCl)₂ (13.3 mL, 157.1 mmol, 5.0 eq) and DMF (0.25 mL, 3.1 mmol, 0.1 eq) at −78° C. The mixture was stirred at rt for 7 h, then concentrated under vacuum to afford 8-fluoro-2-(2-fluorophenyl)-4-methoxyquinoline-7-carbonyl chloride as a yellow solid (12.5 g, ca 100.0%) which was used to the next step directly. LRMS (M+H⁺) m/z calculated 330.1, found 330.1 in MeOH.

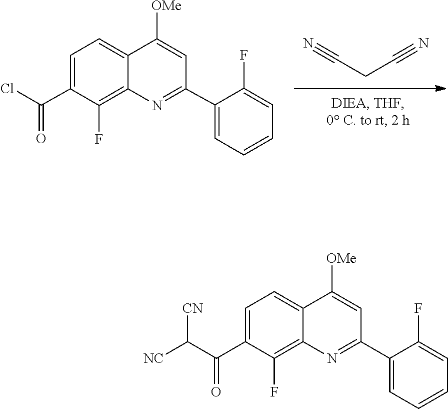 [SMILES: COc1cc(-c2ccccc2F)nc2c(F)c(C(=O)Cl)ccc12 ; N#CCC#N ; COc1cc(-c2ccccc2F)nc2c(F)c(C(=O)C(C#N)C#N)ccc12]

To a solution of 8-fluoro-2-(2-fluorophenyl)-4-methoxyquinoline-7-carbonyl chloride (12.5 g, 37.5 mmol, 1.0 eq) in THF (100 mL) were added malononitrile (2.5 g, 37.5 mmol, 1.0 eq) and DIEA (19.6 mL, 112.6 mmol, 3.0 eq). The mixture was stirred at rt for 2 h, then concentrated, diluted with water (100 mL). The resulting mixture was extracted with EtOAc (150 mL×2). The combined organic layers were washed with brine (150 mL), dried over anhydrous sodium sulfate, filtered and concentrated under vacuum. The resulting residue was purified by column chromatography on silica gel (DCM/MeOH=50/1, v/v) to afford 2-(8-fluoro-2-(2-fluorophenyl)-4-methoxyquinoline-7-carbonyl)malononitrile as a yellow oil (11.7 g, 86%), LRMS (M+H⁺) m/z calculated 364.1, found 364.0.

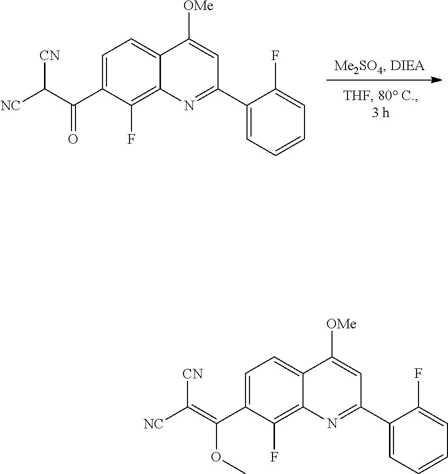 [SMILES: COc1cc(-c2ccccc2F)nc2c(F)c(C(=O)C(C#N)C#N)ccc12 ; COC(=C(C#N)C#N)c1ccc2c(OC)cc(-c3ccccc3F)nc2c1F]

To a solution of 2-(8-fluoro-2-(2-fluorophenyl)-4-methoxyquinoline-7-carbonyl)malononitrile (11.7 g, 32.2 mmol, 1.0 eq) in THF (200 mL) were added Me₂SO₄ (6.3 mL, 64.4 mmol, 2.0 eq) and DIEA (28.1 mL, 161.2 mmol, 5.0 eq). The mixture was stirred at 80° C. for 3 h, concentrated under vacuum, diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=1/1, v/v) to afford 2-((8-fluoro-2-(2-fluorophenyl)-4-methoxyquinolin-7-yl)(methoxy)methylene)malononitrile as a yellow oil (7.1 g, 58.5%), LRMS (M+H⁺) m/z calculated 378.1, found 378.1.

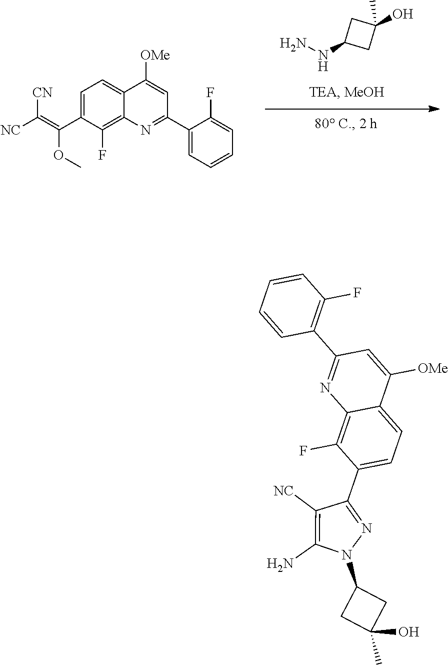

To a solution of 2-((8-fluoro-2-(2-fluorophenyl)-4-methoxyquinolin-7-yl)(methoxy)methylene)malononitrile (1.1 g, 2.9 mmol, 1.0 eq) and (1s,3s)-3-hydrazineyl-1-methylcyclobutan-1-ol (406 mg, 3.5 mmol, 1.2 eq) in MeOH (50 mL) was added TEA (3.2 mL, 23.3 mmol, 8.0 eq) at rt. The reaction mixture was stirred at 80° C. for 2 h, then concentrated under vacuum. The resulting residue was purified by column chromatography on silica gel (PE/EA=2/1, v/v) to afford 5-amino-3-(8-fluoro-2-(2-fluorophenyl)-4-methoxyquinolin-7-yl)-1-((1s,3s)-3-hydroxy-3-methylcyclobutyl)-1H-pyrazole-4-carbonitrile (760 mg, 55.1%) as a yellow solid, LRMS (M+H⁺) m/z calculated 462.2, found 462.4.

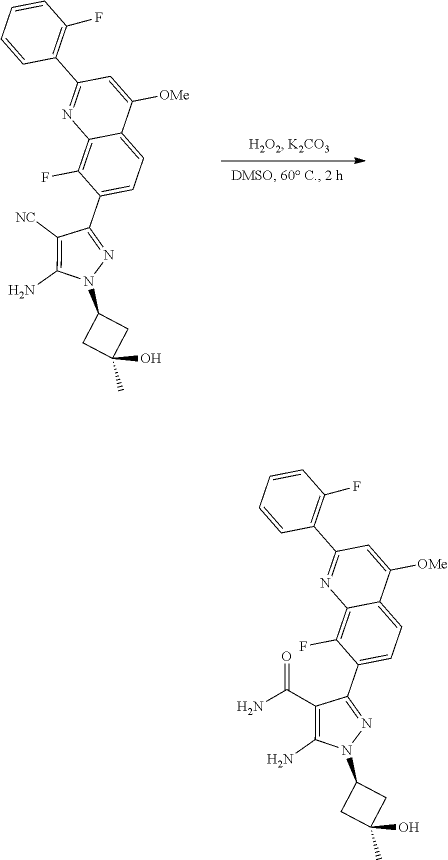 [SMILES: COc1cc(-c2ccccc2F)nc2c(F)c(-c3nn([C@H]4C[C@@](C)(O)C4)c(N)c3C#N)ccc12 ; COc1cc(-c2ccccc2F)nc2c(F)c(-c3nn([C@H]4C[C@@](C)(O)C4)c(N)c3C(N)=O)ccc12]

To a stirred solution of 5-amino-3-(8-fluoro-2-(2-fluorophenyl)-4-methoxyquinolin-7-yl)-1-((1s,3s)-3-hydroxy-3-methylcyclobutyl)-1H-pyrazole-4-carbonitrile (760 mg, 1.6 mmol, 1.0 eq) in DMSO (50 mL) were added K₂CO₃ (1.1 g, 8.2 mmol, 5.0 eq) and H₂O₂ (30%, 3.7 mL, 33.0 mmol, 20.0 eq). After addition was complete, the mixture was stirred at 60° C. for 3 h, then diluted with water (100 mL), and extracted with EtOAc (150 mL×2). The combined organic layers were washed with brine (150 mL), dried over anhydrous sodium sulfate, filtered and concentrated under vacuum. The resulting residue was purified by Prep-HPLC to afford 5-amino-3-(8-fluoro-2-(2-fluorophenyl)-4-methoxyquinolin-7-yl)-1-((1s,3s)-3-hydroxy-3-methylcyclobutyl)-1H-pyrazole-4-carboxamide as a white solid (340.2 mg, 43%). LRMS (M+H⁺) m/z calculated 480.2, found 480.1. ¹H NMR (DMSO-d₆, 400 MHz) δ 7.99-8.05 (m, 2H), 7.55-7.62 (m, 2H), 7.47 (d, 1H), 7.38-7.43 (m, 2H), 6.32 (s, 2H), 5.19 (s, 1H), 4.44-4.49 (m, 1H), 4.14 (s, 3H), 2.54-2.60 (m, 2H), 2.35-2.41 (m, 2H), 1.33 (s, 3H).

### Example 36: Preparation of 5-amino-3-(4-ethoxy-2-phenylquinolin-7-yl)-1-((1s,3s)-3-hydroxy-3-methylcyclobutyl)-1H-pyrazole-4-carboxamide

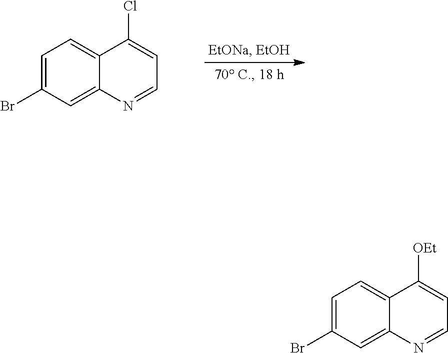 [SMILES: Clc1ccnc2cc(Br)ccc12 ; CCOc1ccnc2cc(Br)ccc12]

To a solution of 7-bromo-4-chloroquinoline (45 g, 185.6 mmol, 1.0 eq) in MeOH (500 mL) was added NaOEt (37.8 g, 556.7 mmol, 3.0 eq). The mixture was stirred at 70° C. for 15 h. The mixture was concentrated under vacuum, diluted with water (100 mL), and extracted with EtOAc (150 mL×2). The combined organic layers were washed with brine (150 mL), dried over anhydrous sodium sulfate, filtered and concentrated to afford 7-bromo-4-ethoxyquinoline (40 g, 85.1%) as a white solid. LRMS (M+H⁺) m/z calculated 252.0, found 252.0.

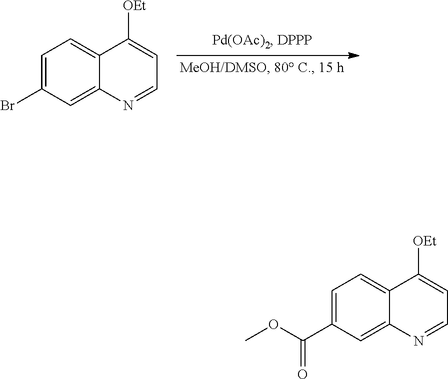 [SMILES: CCOc1ccnc2cc(Br)ccc12 ; CCOc1ccnc2cc(C(=O)OC)ccc12]

To a solution of 7-bromo-4-ethoxyquinoline (40 g, 159.3 mmol, 1.0 eq), DPPP (26.3 g, 63.7 mmol, 0.4 eq) and Pd(OAc)₂ (7.2 g, 31.8 mmol, 0.2 eq) in DMSO/MeOH (500 mL/500 mL) was added TEA (110.3 mL, 796.8 mmol. 5.0 eq). The mixture was stirred at 80° C. for 15 h under CO (1 atm), then the reaction mixture was diluted with water (300 mL), and extracted with EtOAc (300 mL×3). The combined organic layers were washed with brine (300 mL), dried with anhydrous sodium sulfate, filtered, and concentrated under vacuum. The resulting residue was purified by column chromatography on silica gel (PE/EA=5/1, v/v) to afford methyl 4-ethoxyquinoline-7-carboxylate (25 g, 67.5%) as a white solid. LRMS (M+H⁺) m/z calculated 232.1, found 232.2.

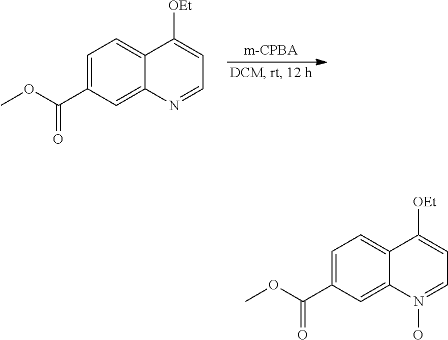

To a stirred solution of methyl 4-ethoxyquinoline-7-carboxylate (25 g, 99.6 mmol, 1.0 eq) in DCM (500 mL) was added m-CPBA (51.5 g, 298.8 mmol, 3.0 eq) at rt. The mixture was stirred at rt for 12 h, then poured into ice, adjusted to pH 13 with addition of saturated Na₂CO₃ aqueous solution, and extracted with DCM (500 mL×3). The combined organic layers were dried over sodium sulfate, filtered and concentrated under vacuum to afford 4-ethoxy-7-(methoxycarbonyl) quinoline 1-oxide (36 g, >100%) as a yellow oil. LRMS (M+H⁺) m/z calculated 248.1, found 248.1.

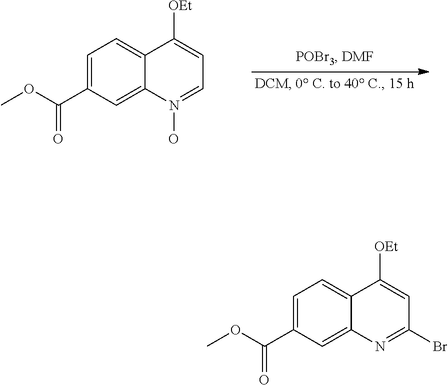

To a solution of 4-ethoxy-7-(methoxycarbonyl) quinoline 1-oxide (36 g, 145.7 mmol, 1.0 eq) in DCM (1000 mL) were added POBr₃ (54.4 g, 189.5 mmol, 1.3 eq) and DMF (5.7 mL, 72.9 mmol, 0.5 eq) at 0° C. The mixture was stirred at 40° C. for 15 h, then poured into ice, adjusted to pH 13 with saturated Na₂CO₃ aqueous solution, extracted with DCM (500 mL×3). The combined organic layers were dried over anhydrous sodium sulfate, filtered and concentrated under vacuum. The resulting residue was purified by column chromatography on silica gel (PE/EA=5/1, v/v) to afford methyl 2-bromoquinoline-7-carboxylate (16 g, 47.9%, 2 steps) as a yellow solid, LRMS (M+H⁺) m/z calculated 310.0, found 310.0.

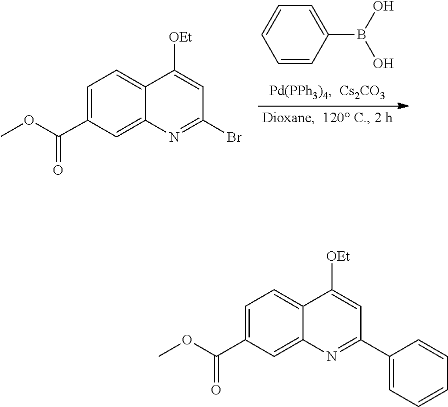 [SMILES: CCOc1cc(Br)nc2cc(C(=O)OC)ccc12 ; OB(O)c1ccccc1 ; CCOc1cc(-c2ccccc2)nc2cc(C(=O)OC)ccc12]

To a solution of methyl 2-bromo-4-ethoxyquinoline-7-carboxylate (16 g, 51.6 mmol, 1.0 eq) in dioxane (300 mL) were added phenylboronic acid (12.6 g, 103.2 mmol, 2.00 eq), Pd(PPh₃)₄ (6.0 g, 5.1 mmol, 0.1 eq) and Cs₂CO₃ (33.6 g, 103.2 mmol, 2.0 eq). The mixture was stirred at 120° C. for 2 h, then concentrated under vacuum and diluted with water (200 mL), extracted with EtOAc (250 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 4-ethoxy-2-phenylquinoline-7-carboxylate (14 g, 88.6%) as a white solid. LRMS (M+H⁺) m/z calculated 308.1, found 308.1

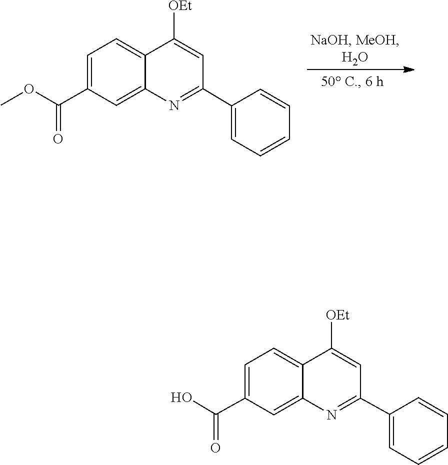 [SMILES: CCOc1cc(-c2ccccc2)nc2cc(C(=O)OC)ccc12 ; CCOc1cc(-c2ccccc2)nc2cc(C(=O)O)ccc12]

To a solution of methyl 4-ethoxy-2-phenylquinoline-7-carboxylate (14 g, 45.6 mmol, 1.0 eq) in MeOH (300 mL) and H₂O (50 mL) was added NaOH (2.7 g, 68.4 mmol, 1.5 eq). The mixture was stirred at 50° C. for 6 h, then concentrated under vacuum and diluted with water (100 mL), 37% HCl was added to adjust to pH 2. The resulting mixture was stirred for 5 min, filtered and dried to afford 4-ethoxy-2-phenylquinoline-7-carboxylic acid (12 g, ca 100%) as a white solid, LRMS (M+H⁺) m/z calculated 294.1, found 294.1.

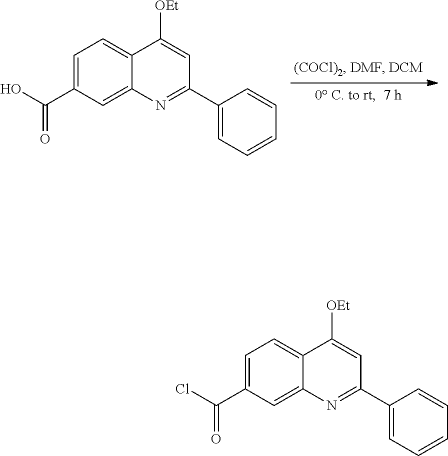 [SMILES: CCOc1cc(-c2ccccc2)nc2cc(C(=O)O)ccc12 ; CCOc1cc(-c2ccccc2)nc2cc(C(=O)Cl)ccc12]

To a solution of 4-ethoxy-2-phenylquinoline-7-carboxylic acid (12 g, 40.9 mmol, 1.0 eq) in DCM (200 mL) were added (COCl)₂ (10.4 mL, 122.9 mmol, 3.0 eq) and DMF (0.1 mL) at −78° C. The mixture was stirred at rt for 1 h, then concentrated under vacuum to afford 4-ethoxy-2-phenylquinoline-7-carbonyl chloride as a yellow solid (15 g, ca 100.0%), LRMS (M+H⁺) m/z calculated 308.1, found 308.1 in MeOH.

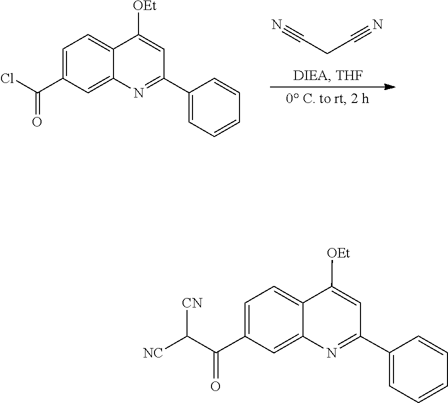 [SMILES: CCOc1cc(-c2ccccc2)nc2cc(C(=O)Cl)ccc12 ; N#CCC#N ; CCOc1cc(-c2ccccc2)nc2cc(C(=O)C(C#N)C#N)ccc12]

To a solution of 4-ethoxy-2-phenylquinoline-7-carbonyl chloride (15 g, 48.2 mmol, 1.0 eq) in THF (400 mL) were added malononitrile (3.2 g, 48.2 mmol, 1.0 eq) and DIEA (19.9 mL, 144.7 mmol, 3.0 eq) at ice bath. The mixture was stirred at rt for 3 h, then concentrated under vacuum, diluted with water (200 mL). The resulting mixture was extracted with EtOAc (300 mL×2). The combined organic layers were washed with brine (300 mL), dried over anhydrous sodium sulfate, filtered and concentrated under vacuum. The resulting residue was purified by column chromatography on silica gel (DCM/MeOH=50/1, v/v) to afford 2-(4-ethoxy-2-phenylquinoline-7-carbonyl)malononitrile as a yellow oil (9 g, 62%), LRMS (M+H⁺) m/z calculated 342.1, found 342.1.

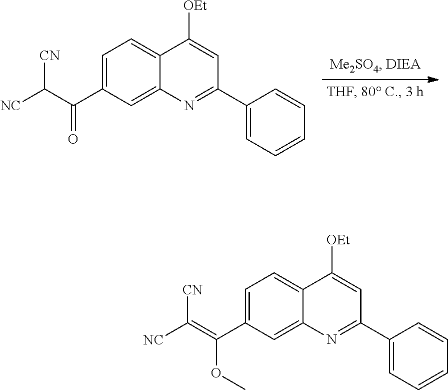 [SMILES: CCOc1cc(-c2ccccc2)nc2cc(C(=O)C(C#N)C#N)ccc12 ; CCOc1cc(-c2ccccc2)nc2cc(C(OC)=C(C#N)C#N)ccc12]

To a solution of 2-(4-ethoxy-2-phenylquinoline-7-carbonyl)malononitrile (3 g, 8.8 mmol, 1.0 eq) in THF (100 mL) were added Me₂SO₄ (1.7 mL, 17.6 mmol, 2.0 eq) and DIEA (4.6 mL, 26.4 mmol, 3.0 eq) at rt. The mixture was stirred at 80° C. for 3 h, then concentrated under vacuum, diluted with water (200 mL), and extracted with EtOAc (500 mL×2). The combined organic layers were washed with brine (500 mL), dried over anhydrous sodium sulfate, filtered and concentrated under vacuum. The resulting residue was purified by column chromatography on silica gel (PE/EA=1/1, v/v) to afford 2-((4-ethoxy-2-phenylquinolin-7-yl)(methoxy)methylene)malononitrile as a yellow oil (1 g, 32%), LRMS (M+H⁺) m/z calculated 356.1, found 356.1.

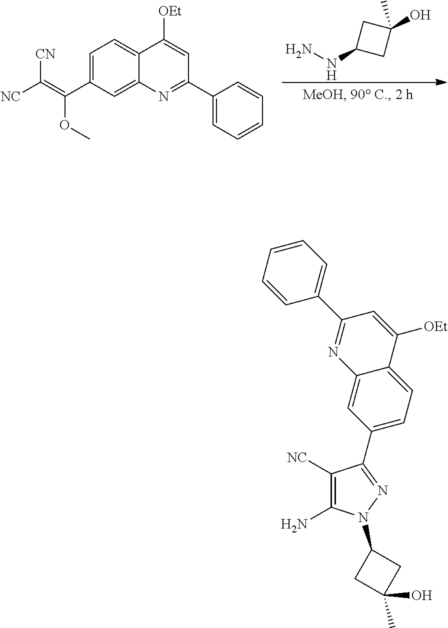

To a solution of 2-((4-ethoxy-2-phenylquinolin-7-yl)(methoxy)methylene)malononitrile (150 mg, 0.42 mmol, 1.0 eq) and (1s,3s)-3-hydrazineyl-1-methylcyclobutan-1-ol (73.5 mg, 0.63 mmol, 1.5 eq) in MeOH (50 mL) were added TEA (0.5 mL, 3.4 mmol, 8.0 eq) at rt. The reaction mixture was stirred at 90° C. for 2 h then the mixture was concentrated under vacuum. The resulting residue was purified by column chromatography on silica gel (PE/EA=1/1, v/v) to afford 5-amino-3-(4-ethoxy-2-phenylquinolin-7-yl)-1-((1s,3s)-3-hydroxy-3-methylcyclobutyl)-1H-pyrazole-4-carbonitrile (120 mg, 64%) as a yellow solid. LRMS (M+H⁺) m/z calculated 440.2, found 440.2.

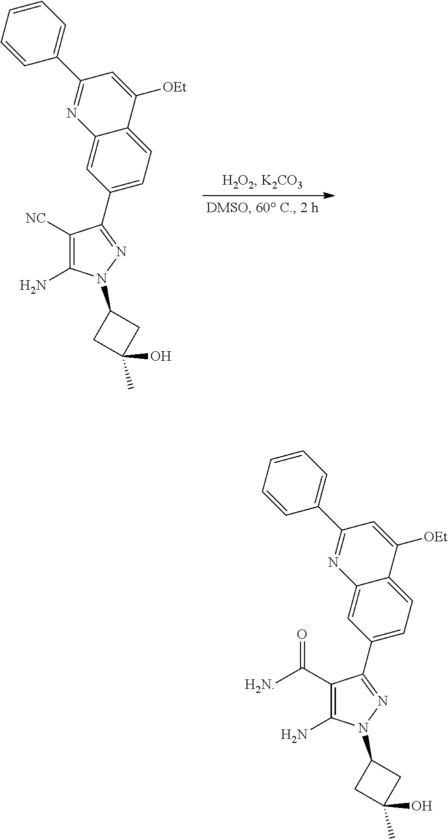 [SMILES: CCOc1cc(-c2ccccc2)nc2cc(-c3nn([C@H]4C[C@@](C)(O)C4)c(N)c3C#N)ccc12 ; CCOc1cc(-c2ccccc2)nc2cc(-c3nn([C@H]4C[C@@](C)(O)C4)c(N)c3C(N)=O)ccc12]

To a stirred solution of 5-amino-3-(4-ethoxy-2-phenylquinolin-7-yl)-1-((1s,3s)-3-hydroxy-3-methylcyclobutyl)-1H-pyrazole-4-carbonitrile (120 mg, 0.27 mmol, 1.0 eq) and K₂CO₃ (113.3 mg, 0.82 mmol, 3.0 eq) in DMSO (20 mL) was added H₂O₂ (30%, 0.6 mL, 5.5 mmol, 20.0 eq) at rt. After addition was complete, the reaction mixture was stirred at 60° C. for 2 h. Water (50 mL) was added and the mixture was extracted with EtOAc (50 mL×2). The organic extract was washed with brine (100 mL), dried with anhydrous sodium sulfate, filtered, concentrated under vacuum and purified by Prep-HPLC to afford 5-amino-3-(4-ethoxy-2-phenylquinolin-7-yl)-1-((1s,3s)-3-hydroxy-3-methylcyclobutyl)-1H-pyrazole-4-carboxamide (14.1 mg, 11%) as a white solid. LRMS (M+H⁺) m/z calculated 458.2, found 458.2. ¹H NMR (DMSO-d₆, 400 MHz) δ 8.29 (d, 2H), 8.18 (d, 1H), 8.12 (d, 1H), 7.69 (dd, 1H), 7.50-7.58 (m, 4H), 6.31 (s, 2H), 5.20 (s, 1H), 4.43-4.52 (m, 3H), 2.59-2.64 (m, 2H), 2.36-2.41 (m, 2H), 1.53 (t, 3H), 1.34 (s, 3H).

### Example 37: Preparation of 5-amino-3-(2-(2-fluorophenyl) quinolin-7-yl)-1-((1s,3s)-3-hydroxy-3-methylcyclobutyl)-1H-pyrazole-4-carboxamide

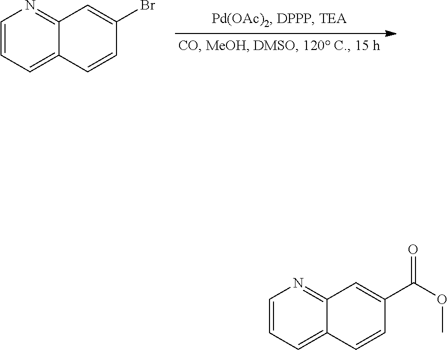

To a solution of 7-bromoquinoline (20 g, 96.6 mmol, 1.0 eq), DPPP (8.0 g, 19.3 mmol, 0.2 eq) and Pd(OAc)₂ (2.1 g, 9.7 mmol, 0.1 eq) in DMSO/MeOH (300 mL/300 mL) was added TEA (40 mL, 289.8 mmol. 3.0 eq). The mixture was stirred at 120° C. for 15 h under CO (5 atm), then concentrated under vacuum. The resulting residue was purified by column chromatography on silica gel (PE/EA=5/1, v/v) to afford methyl quinoline-7-carboxylate (16.4 g, 91.1%) as a yellow solid. LRMS (M+H⁺) m/z calculated 188.1, found 188.0.

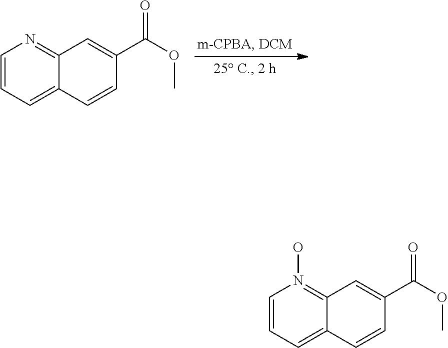

To a stirred solution of methyl quinoline-7-carboxylate (16.4 g, 87.7 mmol, 1.0 eq) in DCM (300 mL) was added m-CPBA (22.7 g, 131.6 mmol, 1.5 eq) at rt. The mixture was stirred at rt for 2 h, then poured into ice, adjusted to pH 13 with addition of saturated Na₂CO₃ aqueous solution, and extracted with DCM (500 mL×3). The combined organic layers were dried over sodium sulfate, filtered and concentrated under vacuum to afford 7-(methoxycarbonyl) quinoline 1-oxide (17.5 g, 98.3%) as a yellow oil. LRMS (M+H⁺) m/z calculated 204.1, found 204.1.

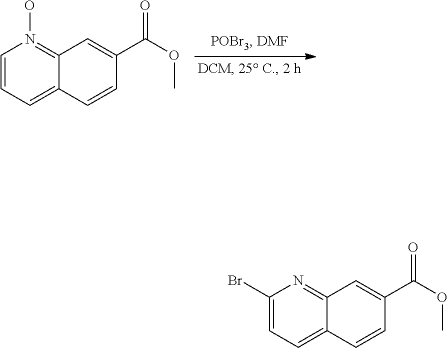

To a solution of 7-(methoxycarbonyl) quinoline 1-oxide (17.5 g, 86.2 mmol, 1.0 eq) in DCM (500 mL) were added POBr₃ (32.1 g, 112.1 mmol, 1.3 eq) and DMF (3.3 mL, 43.1 mmol, 0.5 eq) at −78° C. The mixture was stirred at rt for 2 h, then poured into ice, adjusted to pH 13 with addition of saturated Na₂CO₃ aqueous solution, and extracted with DCM (500 mL×3). The combined organic layers were dried over anhydrous sodium sulfate, filtered and concentrated under vacuum. The resulting residue was purified by column chromatography on silica gel (PE/EA=5/1, v/v) to afford methyl 2-bromoquinoline-7-carboxylate (17.2 g, 75.4%) as a yellow solid, LRMS (M+H⁺) m/z calculated 266.0, found 266.0. ¹H NMR (DMSO-d₆, 400 MHz) δ 8.50 (s, 1H), 8.45 (d, 1H), 8.12-8.21 (m, 2H), 8.86 (d, 1H), 3.95 (s, 3H).

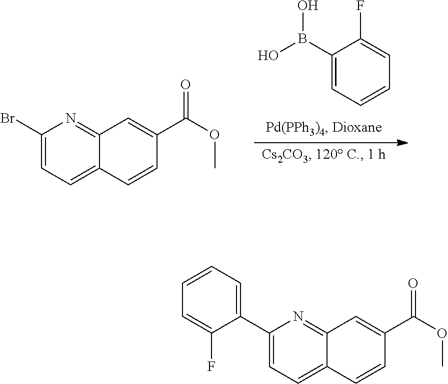 [SMILES: COC(=O)c1ccc2ccc(Br)nc2c1 ; OB(O)c1ccccc1F ; COC(=O)c1ccc2ccc(-c3ccccc3F)nc2c1]

To a solution of methyl 2-bromoquinoline-7-carboxylate (12 g, 45.2 mmol, 1.0 eq) in dioxane (300 mL) were added (2-fluorophenyl) boronic acid (12.7 g, 90.5 mmol, 2.0 eq), Pd(PPh₃)₄ (2.6 g, 2.2 mmol, 0.05 eq) and Cs₂CO₃ (29.5 g, 90.4 mmol, 2.0 eq) . . . . The mixture was stirred at 120° C. for 1 h, then concentrated under vacuum and diluted with water (100 mL), extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 2-(2-fluorophenyl) quinoline-7-carboxylate (12.8 g, ca 100%) as a white solid. LRMS (M+H⁺) m/z calculated 282.1, found 282.1.

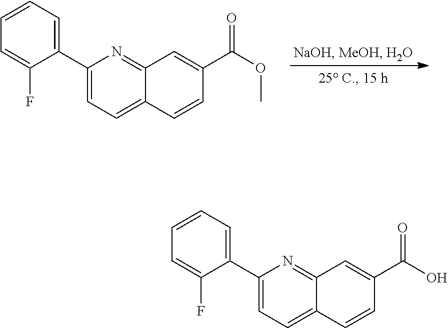 [SMILES: COC(=O)c1ccc2ccc(-c3ccccc3F)nc2c1 ; O=C(O)c1ccc2ccc(-c3ccccc3F)nc2c1]

To a solution of methyl 2-(2-fluorophenyl) quinoline-7-carboxylate (12.8 g, 45.6 mmol, 1.0 eq) in MeOH (200 mL) and H₂O (30 mL) was added NaOH (2.7 g, 68.3 mmol, 1.5 eq). The mixture was stirred at rt for 15 h, then concentrated under vacuum and diluted with water (60 mL), 37% HCl was added to adjust to pH=2. The resulting mixture was stirred for 5 min, filtered and concentrated under vacuum to afford 2-(2-fluorophenyl) quinoline-7-carboxylic acid (10.7 g, 88.5%) as a white solid, LRMS (M+H⁺) m/z calculated 268.1, found 268.1.

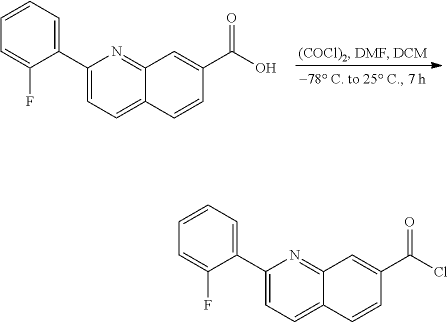 [SMILES: O=C(O)c1ccc2ccc(-c3ccccc3F)nc2c1 ; O=C(Cl)c1ccc2ccc(-c3ccccc3F)nc2c1]

To a solution of 2-(2-fluorophenyl) quinoline-7-carboxylic acid (10.7 g, 40.1 mmol, 1.0 eq) in DCM (200 mL) were added (COCl)₂ (3.4 mL, 42.1 mmol, 5.0 eq) and DMF (5 drops) at −78° C. The mixture was stirred at rt for 7 h, then concentrated under vacuum to afford 2-(2-fluorophenyl) quinoline-7-carbonyl chloride as a yellow solid (12.6 g, ca 100.0%). LRMS (M+H⁺) m/z calculated 282.1, found 282.1 in MeOH.

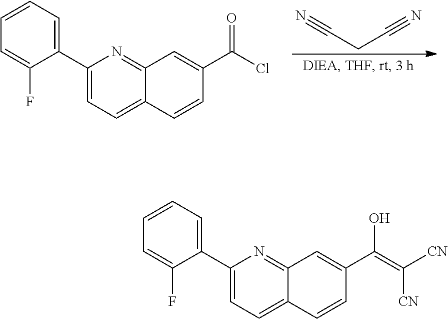 [SMILES: O=C(Cl)c1ccc2ccc(-c3ccccc3F)nc2c1 ; N#CCC#N ; N#CC(C#N)=C(O)c1ccc2ccc(-c3ccccc3F)nc2c1]

To a solution of 2-(2-fluorophenyl) quinoline-7-carbonyl chloride (12.7 g, 44.6 mmol, 1.0 eq) in THF (200 mL) were added malononitrile (2.9 g, 44.5 mmol, 1.0 eq) and DIEA (23.3 mL, 133.7 mmol, 3.0 eq) at ice bath. The mixture was stirred at rt for 3 h, then concentrated, and diluted with water (120 mL). The resulting mixture was extracted with EtOAc (350 mL×2). The combined organic layers were washed with brine (150 mL), dried over anhydrous sodium sulfate, filtered and concentrated under vacuum. The resulting residue was purified by column chromatography on silica gel (DCM/MeOH=50/1, v/v) to afford 2-((2-(2-fluorophenyl) quinolin-7-yl)(hydroxy)methylene)malononitrile as a yellow oil (14.7 g, ca 100%), LRMS (M+H⁺) m/z calculated 316.1, found 316.1.

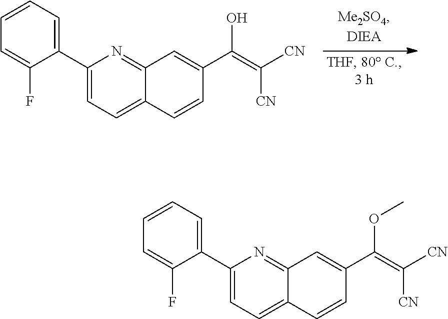 [SMILES: N#CC(C#N)=C(O)c1ccc2ccc(-c3ccccc3F)nc2c1 ; COC(=C(C#N)C#N)c1ccc2ccc(-c3ccccc3F)nc2c1]

To a solution of 2-((2-(2-fluorophenyl) quinolin-7-yl)(hydroxy)methylene)malononitrile (14.7 g, 46.7 mmol, 1.0 eq) in THF (300 mL) were added Me₂SO₄ (9.0 mL, 93.3 mmol, 2.0 eq) and DIEA (40.6 mL, 233.3 mmol, 5.0 eq) at rt. The mixture was stirred at 80° C. for 3 h, then concentrated under vacuum and diluted with water (120 mL), extracted with EtOAc (250 mL×2). The combined organic layers were washed with brine (150 mL), dried over anhydrous sodium sulfate, filtered and concentrated under vacuum. The resulting residue was purified by column chromatography on silica gel (PE/EA=1/1, v/v) to afford 2-((2-(2-fluorophenyl) quinolin-7-yl)(methoxy)methylene)malononitrile as a yellow oil (5 g, 33%). LRMS (M+H⁺) m/z calculated 330.1, found 330.1.

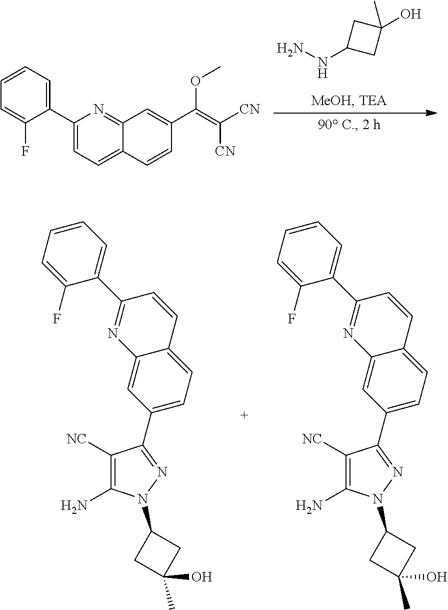

To a solution of 2-((2-(2-fluorophenyl) quinolin-7-yl)(methoxy)methylene)malononitrile (100 mg, 0.30 mmol, 1.0 eq) and 3-hydrazineyl-1-methylcyclobutan-1-ol (52.9 mg, 0.46 mmol, 1.5 eq) in MeOH (50 mL) were added TEA (0.4 mL, 2.4 mmol, 8.0 eq) at rt. The reaction mixture was stirred at 90° C. for 2 h then the mixture was concentrated under vacuum and purified by column chromatography on silica gel (PE/EA=1/1, v/v) to afford 5-amino-3-(2-(2-fluorophenyl) quinolin-7-yl)-1-((1s,3s)-3-hydroxy-3-methylcyclobutyl)-1H-pyrazole-4-carbonitrile (40 mg, 32%) and 5-amino-3-(2-(2-fluorophenyl) quinolin-7-yl)-1-((1r,3r)-3-hydroxy-3-methylcyclobutyl)-1H-pyrazole-4-carbonitrile (45 mg, 36%,) as a yellow solid. LRMS (M+H⁺) m/z calculated 414.2, found 414.3.

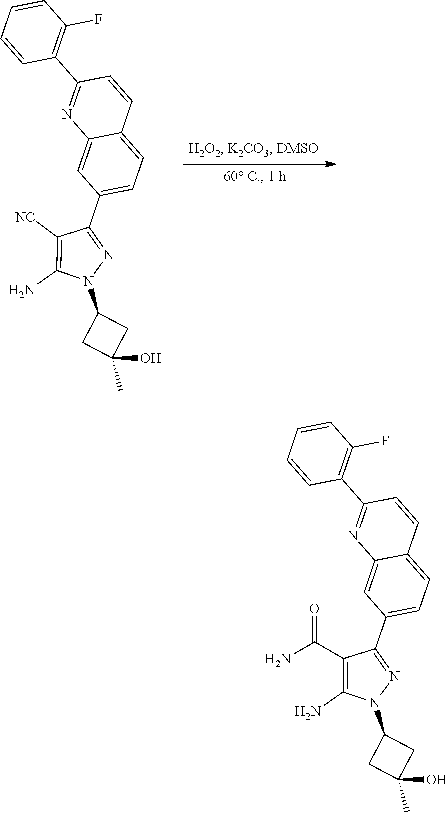 [SMILES: C[C@]1(O)C[C@@H](n2nc(-c3ccc4ccc(-c5ccccc5F)nc4c3)c(C#N)c2N)C1 ; C[C@]1(O)C[C@@H](n2nc(-c3ccc4ccc(-c5ccccc5F)nc4c3)c(C(N)=O)c2N)C1]

To a stirred solution of 5-amino-3-(2-(2-fluorophenyl) quinolin-7-yl)-1-((1s,3s)-3-hydroxy-3-methylcyclobutyl)-1H-pyrazole-4-carbonitrile (40 mg, 0.096 mmol, 1.0 eq) and K₂CO₃ (40.1 mg, 0.29 mmol, 3.0 eq) in DMSO (20 mL) at rt was added H₂O₂ (30%, 0.2 mL, 1.9 mmol, 20.0 eq). After addition was complete, the reaction mixture was stirred at 60° C. for 1 h. The mixture was diluted with water (80 mL) was added and the mixture was extracted with EtOAc (200 mL). The organic extract was washed with brine (100 mL), dried with anhydrous sodium sulfate, and purified by Prep-HPLC to afford 5-amino-3-(2-(2-fluorophenyl) quinolin-7-yl)-1-((1s,3s)-3-hydroxy-3-methylcyclobutyl)-1H-pyrazole-4-carboxamide (11.8 mg, 33%) as a white solid. LRMS (M+H⁺) m/z calculated 432.2, found 432.2. ¹H NMR (DMSO-d₆, 400 MHz) δ 8.50 (d, 1H), 8.22 (s, 1H), 8.05-8.10 (m, 2H), 7.94 (d, 1H), 7.82 (d, 1H), 7.55-7.59 (m, 1H), 7.37-7.43 (m, 2H), 6.29 (s, 2H), 5.19 (s, 1H), 4.42-4.51 (t, 1H), 2.59-2.65 (m, 2H), 2.36-2.42 (m, 2H), 1.35 (s, 3H).

### Example 38: Preparation of 5-amino-3-(2-(2-bromophenyl) quinolin-7-yl)-1-((1s,3s)-3-hydroxy-3-methylcyclobutyl)-1H-pyrazole-4-carboxamide

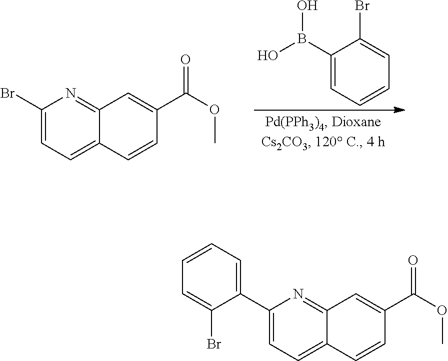 [SMILES: COC(=O)c1ccc2ccc(Br)nc2c1 ; OB(O)c1ccccc1Br ; COC(=O)c1ccc2ccc(-c3ccccc3Br)nc2c1]

To a solution of methyl 2-bromoquinoline-7-carboxylate (3.1 g, 11.7 mmol, 1.0 eq) in dioxane (300 mL) were added (2-bromophenyl) boronic acid (4.7 g, 23.4 mmol, 2.0 eq), Pd(PPh₃)₄ (68.0 mg, 0.059 mmol, 0.05 eq) and Cs₂CO₃ (7.6 g, 23.4 mmol, 2.0 eq). The mixture was stirred at 120° C. for 4 h, then concentrated under vacuum. The resulting residue was diluted with water (100 mL), extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 2-(2-bromophenyl) quinoline-7-carboxylate (2.8 g, 70%) as a white solid. LRMS (M+H⁺) m/z calculated 342.0, found 342.1.

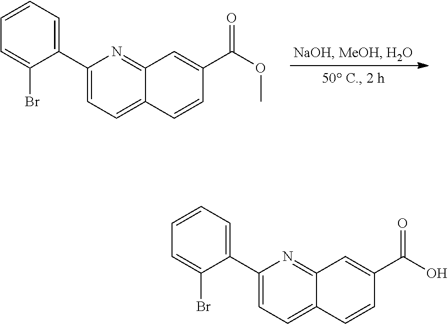 [SMILES: COC(=O)c1ccc2ccc(-c3ccccc3Br)nc2c1 ; O=C(O)c1ccc2ccc(-c3ccccc3Br)nc2c1]

To a solution of methyl 2-(2-bromophenyl) quinoline-7-carboxylate (2.8 g, 8.2 mmol, 1.0 eq) in MeOH (200 mL) and H₂O (30 mL) was added NaOH (492 mg, 12.3 mmol, 1.5 eq). The mixture was stirred at 50° C. for 2 h, then concentrated under vacuum and diluted with water (20 mL), 37% HCl was added to adjust to pH=2. The resulting mixture was stirred for 5 min, filtered and concentrated under vacuum to afford 2-(2-bromophenyl) quinoline-7-carboxylic acid (2.6 g, 97%) as a white solid, LRMS (M+H⁺) m/z calculated 328.0, found 328.0.

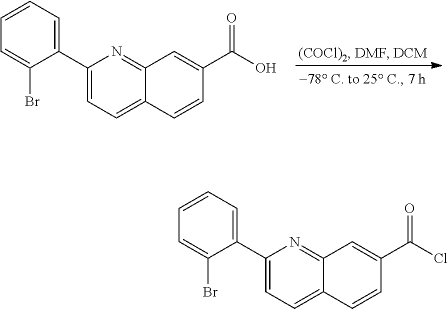 [SMILES: O=C(O)c1ccc2ccc(-c3ccccc3Br)nc2c1 ; O=C(Cl)c1ccc2ccc(-c3ccccc3Br)nc2c1]

To a solution of 2-(2-bromophenyl) quinoline-7-carboxylic acid (2.6 g, 7.9 mmol, 1.0 eq) in DCM (50 mL) were added (COCl)₂ (499 mg, 39.6 mmol, 5.0 eq) and DMF (5 drops) at −78° C. The mixture was stirred at rt for 7 h, then concentrated under vacuum to afford crude 2-(2-bromophenyl) quinoline-7-carbonyl chloride as a yellow solid (4 g, ca 100%), LRMS (M+H⁺) m/z calculated 342.0, found 342.1 in MeOH.

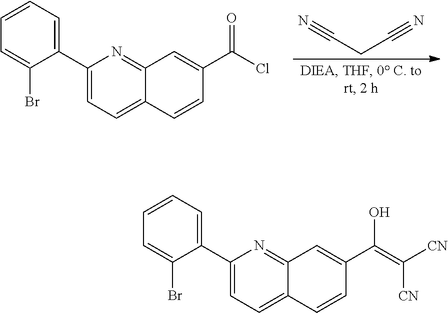 [SMILES: O=C(Cl)c1ccc2ccc(-c3ccccc3Br)nc2c1 ; N#CCC#N ; N#CC(C#N)=C(O)c1ccc2ccc(-c3ccccc3Br)nc2c1]

To a solution of 2-(2-bromophenyl) quinoline-7-carbonyl chloride (4 g crude, 7.92 mmol, 1.0 eq) in THF (50 mL) were added malononitrile (523 mg, 7.92 mmol, 1.0 eq) and DIEA (3.06 g, 23.7 mmol, 3.0 eq) at ice bath. The mixture was stirred at rt for 2 h, then concentrated. The residue was diluted with water (100 mL). The resulting mixture was extracted with EtOAc (150 mL×2). The combined organic layers were washed with brine (100 mL), dried over anhydrous sodium sulfate, filtered and concentrated under vacuum. The resulting residue was purified by column chromatography on silica gel (DCM/MeOH=50/1, v/v) to afford 2-((2-(2-bromophenyl) quinolin-7-yl)(hydroxy)methylene)malononitrile as a yellow oil (3.4 g, 70%), LRMS (M+H⁺) m/z calculated 376.0, found 376.1.

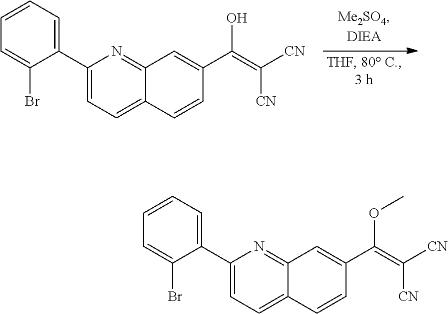 [SMILES: N#CC(C#N)=C(O)c1ccc2ccc(-c3ccccc3Br)nc2c1 ; COC(=C(C#N)C#N)c1ccc2ccc(-c3ccccc3Br)nc2c1]

To a solution of 2-((2-(2-bromophenyl) quinolin-7-yl)(hydroxy)methylene)malononitrile (3.4 g crude, 7.92 mmol, 1.0 eq) in THF (100 mL) were added Me₂SO₄ (2.0 g, 15.8 mmol, 2.0 eq) and DIEA (5.11 g, 39.6 mmol, 5.0 eq) at rt. The mixture was stirred at 80° C. for 3 h, then concentrated under vacuum. The residue was diluted with water (100 mL), extracted with EtOAc (150 mL×2). The combined organic layers were washed with brine (100 mL), dried over anhydrous sodium sulfate, filtered and concentrated under vacuum. The resulting residue was purified by column chromatography on silica gel (PE/EA=1/1, v/v) to afford 2-((2-(2-bromophenyl) quinolin-7-yl)(methoxy)methylene)malononitrile as a yellow oil (130 mg, 4% in 3 steps), LRMS (M+H⁺) m/z calculated 390.0, found 390.1.

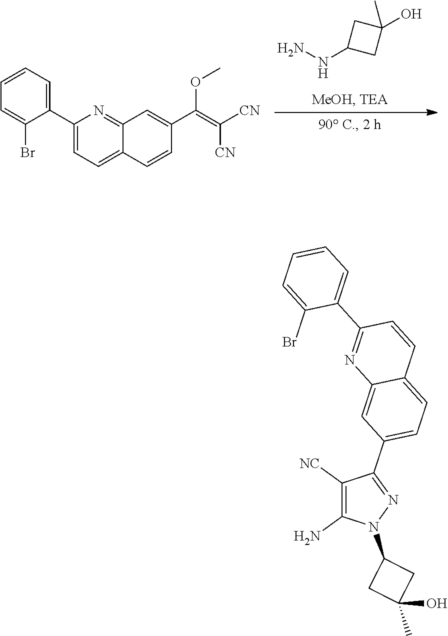

To a solution of 2-((2-(2-bromophenyl) quinolin-7-yl)(methoxy)methylene)malononitrile (130 mg, 0.33 mmol, 1.0 eq) and 3-hydrazineyl-1-methylcyclobutan-1-ol (58 mg, 0.50 mmol, 1.5 eq) in MeOH (50 mL) were added TEA (267 mg, 2.64 mmol, 8.0 eq) at rt. The reaction mixture was stirred at 90° C. for 2 h then the mixture was concentrated under vacuum and purified by column chromatography on silica gel (PE/EA=1/1, v/v) to afford 5-amino-3-(2-(2-bromophenyl) quinolin-7-yl)-1-((1s,3s)-3-hydroxy-3-methylcyclobutyl)-1H-pyrazole-4-carbonitrile (90 mg, 57%) as a yellow solid. LRMS (M+H⁺) m/z calculated 474.1, found 474.2.

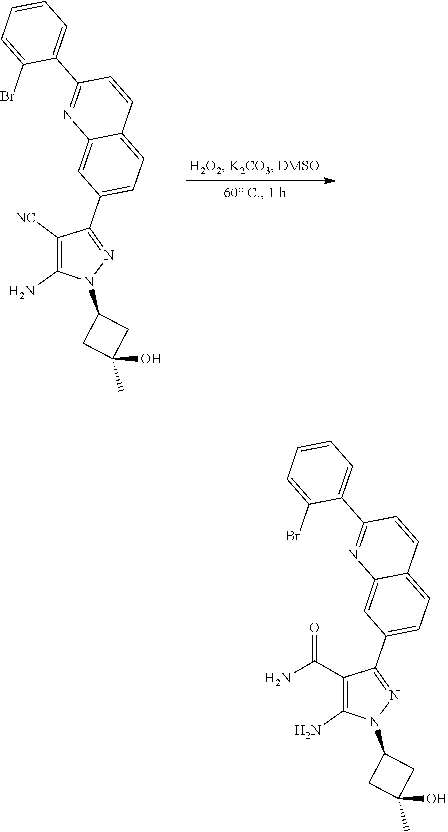 [SMILES: C[C@]1(O)C[C@@H](n2nc(-c3ccc4ccc(-c5ccccc5Br)nc4c3)c(C#N)c2N)C1 ; C[C@]1(O)C[C@@H](n2nc(-c3ccc4ccc(-c5ccccc5Br)nc4c3)c(C(N)=O)c2N)C1]

To a stirred solution of 5-amino-3-(2-(2-bromophenyl) quinolin-7-yl)-1-((1s,3s)-3-hydroxy-3-methylcyclobutyl)-1H-pyrazole-4-carbonitrile (90 mg, 0.19 mmol, 1.0 eq) and K₂CO₃ (79 mg, 0.57 mmol, 3.0 eq) in DMSO (10 mL) was added H₂O₂ (30% in water, 430 mg, 3.8 mmol, 20.0 eq). After addition was complete, the reaction mixture was stirred at 60° C. for 1 h at rt, then diluted with water (50 mL) and extracted with EtOAc (50 mL×2). The combined organic layers were washed with brine (50 mL), dried with anhydrous sodium sulfate and purified by Prep-HPLC to afford 5-amino-3-(2-(2-bromophenyl) quinolin-7-yl)-1-((1s,3s)-3-hydroxy-3-methylcyclobutyl)-1H-pyrazole-4-carboxamide (51 mg, 0.10 mmol, 55%) as a white solid. LRMS (M+H⁺) m/z calculated 492.1, found 492.0. ¹H NMR (DMSO-d₆, 400 MHz) δ 8.49 (d, 1H), 8.19 (s, 1H), 8.09 (d, 1H), 7.74-7.85 (m, 3H), 7.64 (d, 1H), 7.56 (td, 1H), 7.44 (td, 1H), 6.29 (s, 2H), 5.19 (s, 1H), 4.42-4.51 (m, 1H), 2.59-2.65 (m, 2H), 2.36-2.42 (m, 2H), 1.34 (s, 3H).

### Example 39: Preparation of 5-amino-3-(2-(4-fluorophenyl) quinolin-7-yl)-1-((1s,3s)-3-hydroxy-3-methylcyclobutyl)-1H-pyrazole-4-carboxamide

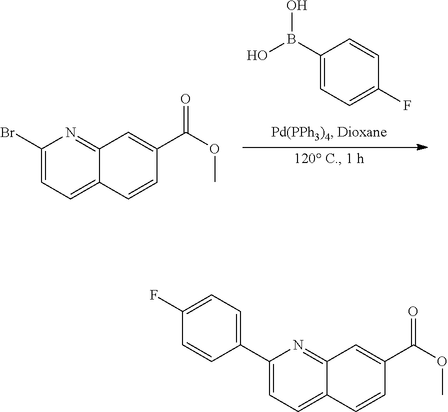 [SMILES: COC(=O)c1ccc2ccc(Br)nc2c1 ; OB(O)c1ccc(F)cc1 ; COC(=O)c1ccc2ccc(-c3ccc(F)cc3)nc2c1]

To a solution of methyl 2-bromoquinoline-7-carboxylate (2 g, 7.5 mmol, 1.0 eq) in dioxane (100 mL) were added (4-fluorophenyl) boronic acid (2.1 g, 15.1 mmol, 2.0 eq), Pd(PPh₃)₄ (436.2 mg, 0.38 mmol, 0.05 eq) and Cs₂CO₃ (11.1 g, 15.0 mmol, 2.0 eq). The mixture was stirred at 120° C. for 1 h, then concentrated under vacuum and diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 2-(4-fluorophenyl) quinoline-7-carboxylate (1.5 g, 71.4%) as a white solid. LRMS (M+H⁺) m/z calculated 282.1, found 282.1.

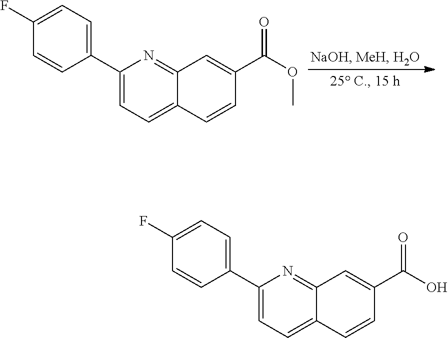 [SMILES: COC(=O)c1ccc2ccc(-c3ccc(F)cc3)nc2c1 ; O=C(O)c1ccc2ccc(-c3ccc(F)cc3)nc2c1]

To a solution of methyl 2-(4-fluorophenyl) quinoline-7-carboxylate (1.5 g, 5.3 mmol, 1.0 eq) in MeOH (100 mL) and H₂O (10 mL) was added NaOH (320.3 mg, 8.0 mmol, 1.5 eq). The mixture was stirred at rt for 15 h, then concentrated under vacuum and diluted with water (60 mL), 37% HCl was added to adjust to pH 2. The resulting mixture was stirred for 5 min, filtered and concentrated under vacuum to afford 2-(4-fluorophenyl) quinoline-7-carboxylic acid (1.4 g, ca 100%) as a white solid, LRMS (M+H⁺) m/z calculated 268.1, found 268.1.

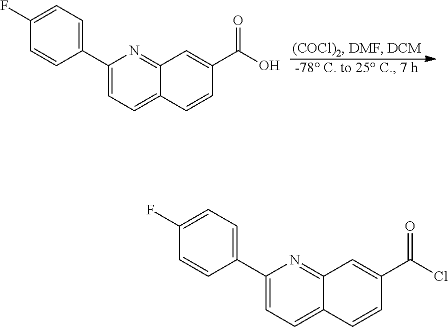 [SMILES: O=C(O)c1ccc2ccc(-c3ccc(F)cc3)nc2c1 ; O=C(Cl)c1ccc2ccc(-c3ccc(F)cc3)nc2c1]

To a solution of 2-(4-fluorophenyl) quinoline-7-carboxylic acid (1.4 g, 5.2 mmol, 1.0 eq) in DCM (100 mL) were added (COCl)₂ (2.2 mL, 26.2 mmol, 5.0 eq) and DMF (5 drops) at −78° C. The mixture was stirred at rt for 7 h, then concentrated under vacuum to afford 2-(4-fluorophenyl) quinoline-7-carbonyl chloride as a yellow solid (2.3 g, ca 100.0%), LRMS (M+H⁺) m/z calculated 282.1, found 282.1 in MeOH.

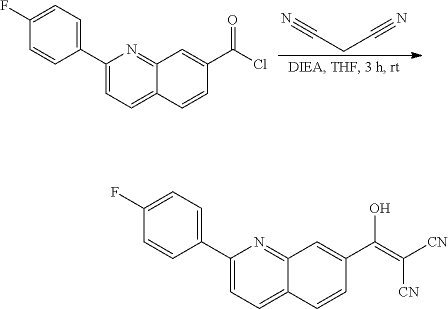 [SMILES: O=C(Cl)c1ccc2ccc(-c3ccc(F)cc3)nc2c1 ; N#CCC#N ; N#CC(C#N)=C(O)c1ccc2ccc(-c3ccc(F)cc3)nc2c1]

To a solution of 2-(4-fluorophenyl) quinoline-7-carbonyl chloride (1.3 g, 4.6 mmol, 1.0 eq) in THF (100 mL) were added malononitrile (301.1 mg, 4.6 mmol, 1.0 eq) and DIEA (2.3 mL, 13.7 mmol, 3.0 eq) at icebath. The mixture was stirred at rt for 3 h, then concentrated, and diluted with water (120 mL). The resulting mixture was extracted with EtOAc (350 mL×2). The combined organic layers were washed with brine (150 mL), dried over anhydrous sodium sulfate, filtered and concentrated under vacuum. The resulting residue was purified by column chromatography on silica gel (DCM/MeOH=50/1, v/v) to afford 2-((2-(4-fluorophenyl) quinolin-7-yl)(hydroxy)methylene)malononitrile as a yellow oil (1.3 g, 86%), LRMS (M+H⁺) m/z calculated 316.1, found 316.1.

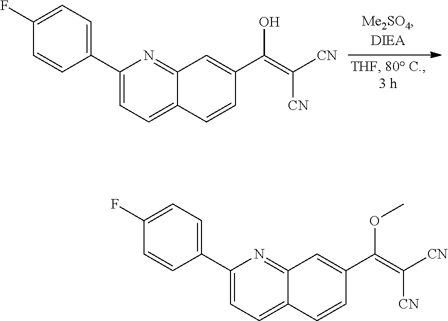 [SMILES: N#CC(C#N)=C(O)c1ccc2ccc(-c3ccc(F)cc3)nc2c1 ; COC(=C(C#N)C#N)c1ccc2ccc(-c3ccc(F)cc3)nc2c1]

To a solution of 2-((2-(4-fluorophenyl) quinolin-7-yl)(hydroxy)methylene)malononitrile (1.3 g, 4.1 mmol, 1.0 eq) in THF (70 mL) were added Me₂SO₄ (0.8 mL, 8.3 mmol, 2.0 eq) and DIEA (2.7 mL, 20.6 mmol, 5.0 eq) at rt. The mixture was stirred at 80° C. for 3 h, then concentrated under vacuum and diluted with water (120 mL), and extracted with EtOAc (250 mL×2). The combined organic layers were washed with brine (150 mL), dried over anhydrous sodium sulfate, filtered and concentrated under vacuum. The resulting residue was purified by column chromatography on silica gel (PE/EA=1/1, v/v) to afford 2-((2-(4-fluorophenyl) quinolin-7-yl)(methoxy)methylene)malononitrile as a yellow oil (410 mg, 31.4%), LRMS (M+H⁺) m/z calculated 330.1, found 330.1.

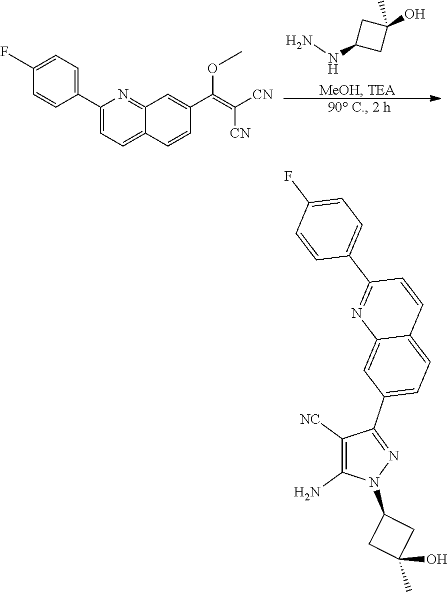

To a solution of 2-((2-(4-fluorophenyl) quinolin-7-yl)(methoxy)methylene)malononitrile (230 mg, 0.70 mmol, 1.0 eq) and (1s,3s)-3-hydrazineyl-1-methylcyclobutan-1-ol (121.6 mg, 1.0 mmol, 1.5 eq) in MeOH (50 mL) were added TEA (0.8 mL, 5.6 mmol, 8.0 eq) at rt. The reaction mixture was stirred at 90° C. for 2 h then the mixture was concentrated under vacuum. The resulting residue was purified by column chromatography on silica gel (PE/EA=1/1, v/v) to afford 5-amino-3-(2-(4-fluorophenyl) quinolin-7-yl)-1-((1s,3s)-3-hydroxy-3-methylcyclobutyl)-1H-pyrazole-4-carbonitrile (140 mg, 48%) as a yellow solid. LRMS (M+H⁺) m/z calculated 414.2, found 414.3.

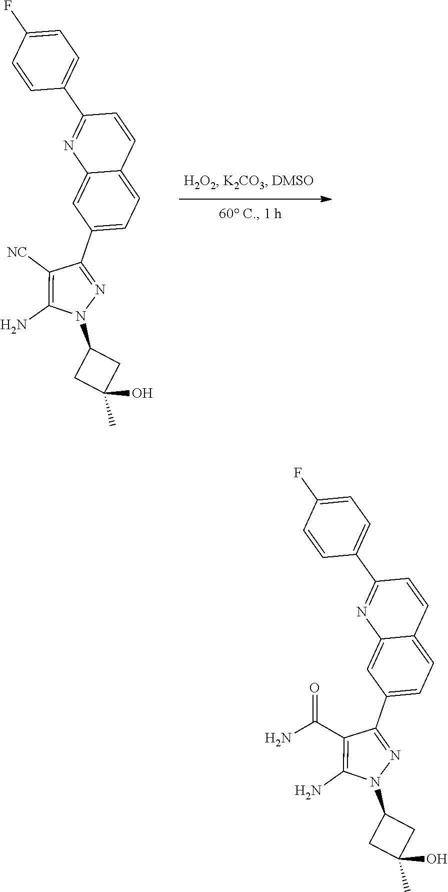 [SMILES: C[C@]1(O)C[C@@H](n2nc(-c3ccc4ccc(-c5ccc(F)cc5)nc4c3)c(C#N)c2N)C1 ; C[C@]1(O)C[C@@H](n2nc(-c3ccc4ccc(-c5ccc(F)cc5)nc4c3)c(C(N)=O)c2N)C1]

To a stirred solution of 5-amino-3-(2-(4-fluorophenyl) quinolin-7-yl)-1-((1s,3s)-3-hydroxy-3-methylcyclobutyl)-1H-pyrazole-4-carbonitrile (140 mg, 0.34 mmol, 1.0 eq) and K₂CO₃ (140.1 mg, 1.0 mmol, 3.0 eq) in DMSO (20 mL) at rt was added H₂O₂ (30%, 0.8 mL, 6.8 mmol, 20.0 eq). After addition was complete, the reaction mixture was stirred at 60° C. for 1 h. The reaction was diluted with water (80 mL) and extracted with EtOAc (200 mL). The combined organic layers were washed with brine (100 mL), dried with anhydrous sodium sulfate, and purified by Prep-HPLC to afford 5-amino-3-(2-(4-fluorophenyl) quinolin-7-yl)-1-((1s,3s)-3-hydroxy-3-methylcyclobutyl)-1H-pyrazole-4-carboxamide (34.4 mg, 23%) as a white solid. LRMS (M+H⁺) m/z calculated 432.2, found 432.2. ¹H NMR (DMSO-d₆, 400 MHz) δ 8.49 (d, 1H), 8.35-8.39 (m, 2H), 8.16-8.20 (m, 2H), 8.05 (d, 1H), 7.77 (dd, 1H), 7.36-7.43 (m, 2H), 6.30 (s, 2H), 5.19 (s, 1H), 4.42-4.51 (m, 1H), 2.59-2.65 (m, 2H), 2.36-2.42 (m, 2H), 1.34 (s, 3H).

### Example 40: Preparation of 5-amino-3-(2-(2-fluorophenyl) quinolin-7-yl)-1-((1r,3r)-3-hydroxy-3-methylcyclobutyl)-1H-pyrazole-4-carboxamide

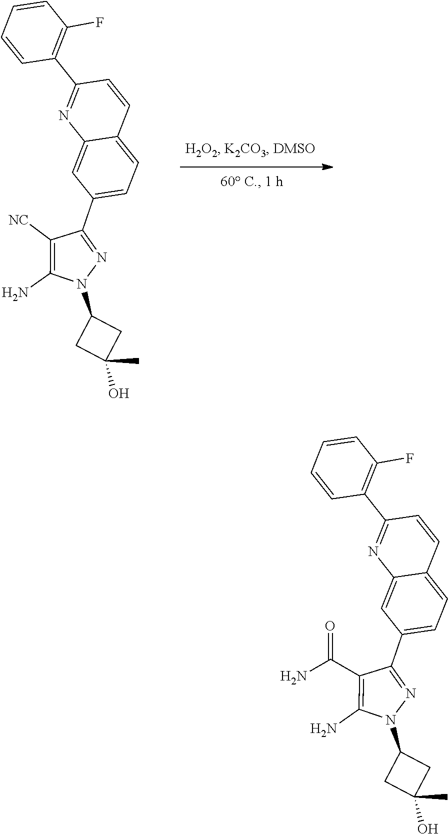 [SMILES: C[C@]1(O)C[C@H](n2nc(-c3ccc4ccc(-c5ccccc5F)nc4c3)c(C#N)c2N)C1 ; C[C@]1(O)C[C@H](n2nc(-c3ccc4ccc(-c5ccccc5F)nc4c3)c(C(N)=O)c2N)C1]

To a stirred solution of 5-amino-3-(2-(2-fluorophenyl) quinolin-7-yl)-1-((1r,3r)-3-hydroxy-3-methylcyclobutyl)-1H-pyrazole-4-carbonitrile (45 mg, 0.11 mmol, 1.0 eq) and K₂CO₃ (40.1 mg, 0.29 mmol, 3.0 eq) in DMSO (10 mL) at rt was added H₂O₂ (30%, 0.2 mL, 2.2 mmol, 20.0 eq). After addition was complete, the reaction mixture was stirred at 60° C. for 1 h. Water (30 mL) was added, and the mixture was extracted with EtOAc (50 mL). The organic extract was washed with brine (30 mL), dried with anhydrous sodium sulfate, and purified by Prep-HPLC to afford 5-amino-3-(2-(2-fluorophenyl) quinolin-7-yl)-1-((1r,3r)-3-hydroxy-3-methylcyclobutyl)-1H-pyrazole-4-carboxamide (15 mg, 0.034 mmol, 32%) as a white solid. LRMS (M+H⁺) m/z calculated 432.2, found 432.2. ¹H NMR (DMSO-d₆, 400 MHz) δ 8.50 (d, 1H), 8.22 (s, 1H), 8.05-8.10 (m, 2H), 7.94 (dd, 1H), 7.82 (d, 1H), 7.55-7.59 (m, 1H), 7.37-7.43 (m, 2H), 6.27 (s, 2H), 4.98 (s, 1H), 4.92-4.96 (m, 1H), 2.39-2.55 (m, 4H), 1.35 (s, 3H).

### Example 41: Preparation of 5-amino-3-(2-(3-fluorophenyl) quinolin-7-yl)-1-((1s,3s)-3-hydroxy-3-methylcyclobutyl)-1H-pyrazole-4-carboxamide

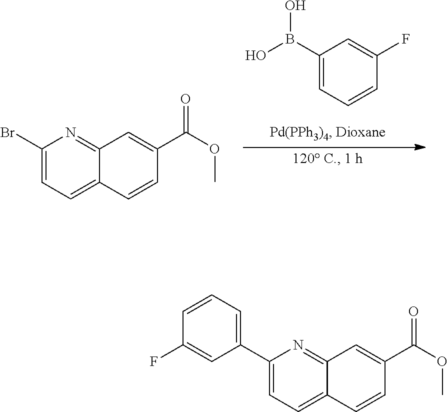 [SMILES: COC(=O)c1ccc2ccc(Br)nc2c1 ; OB(O)c1cccc(F)c1 ; COC(=O)c1ccc2ccc(-c3cccc(F)c3)nc2c1]

To a solution of methyl 2-bromoquinoline-7-carboxylate (6 g, 22.6 mmol, 1.0 eq) in dioxane (100 mL) were added (3-fluorophenyl) boronic acid (6.3 g, 45.3 mmol, 2.0 eq), Pd(PPh₃)₄ (1.3 g, 1.1 mmol, 0.05 eq) and Cs₂CO₃ (14.7 g, 45.2 mmol, 2.0 eq). The mixture was stirred at 120° C. for 1 h, then concentrated under vacuum and diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl methyl 2-(3-fluorophenyl) quinoline-7-carboxylate (2.2 g, 35%) as a white solid. LRMS (M+H⁺) m/z calculated 282.1, found 282.1

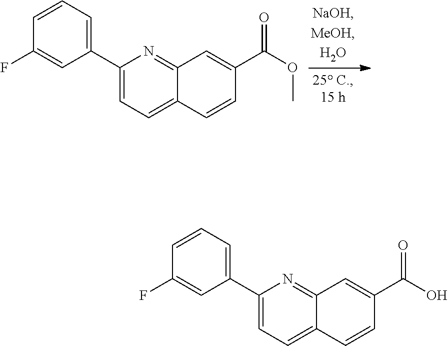 [SMILES: COC(=O)c1ccc2ccc(-c3cccc(F)c3)nc2c1 ; O=C(O)c1ccc2ccc(-c3cccc(F)c3)nc2c1]

To a solution of methyl methyl 2-(3-fluorophenyl) quinoline-7-carboxylate (2.2 g, 7.8 mmol, 1.0 eq) in MeOH (100 mL) and H₂O (10 mL) was added NaOH (469.8 mg, 11.7 mmol, 1.5 eq). The mixture was stirred at rt for 15 h, then concentrated under vacuum and diluted with water (60 mL), 37% HCl was added to adjust to pH 2. The resulting mixture was stirred for 5 min, filtered and concentrated under vacuum to afford 2-(3-fluorophenyl) quinoline-7-carboxylic acid (1.4 g, 62%) as a white solid, LRMS (M+H⁺) m/z calculated 268.1, found 268.1.

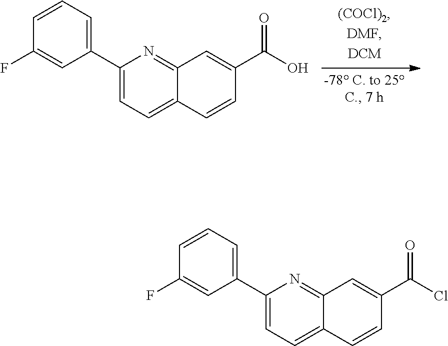 [SMILES: O=C(O)c1ccc2ccc(-c3cccc(F)c3)nc2c1 ; O=C(Cl)c1ccc2ccc(-c3cccc(F)c3)nc2c1]

To a solution of 2-(3-fluorophenyl) quinoline-7-carboxylic acid (1.4 g, 5.2 mmol, 1.0 eq) in DCM (100 mL) were added (COCl)₂ (2.2 mL, 26.2 mmol, 5.0 eq) and DMF (5 drops) at −78° C. The mixture was stirred at rt for 7 h, then concentrated under vacuum to afford 2-(3-fluorophenyl) quinoline-7-carbonyl chloride as a yellow solid (2.0 g, ca 100.0%), LRMS (M+H⁺) m/z calculated 282.1, found 282.1 in MeOH.

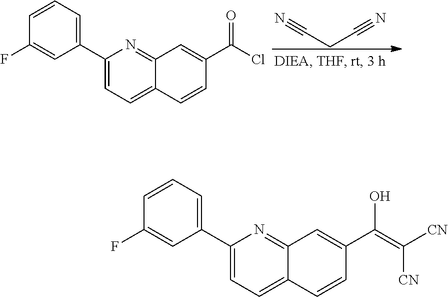 [SMILES: O=C(Cl)c1ccc2ccc(-c3cccc(F)c3)nc2c1 ; N#CCC#N ; N#CC(C#N)=C(O)c1ccc2ccc(-c3cccc(F)c3)nc2c1]

To a solution of 2-(3-fluorophenyl) quinoline-7-carbonyl chloride (2 g, 7.0 mmol, 1.0 eq) in THF (100 mL) were added malononitrile (463.2 mg, 7.0 mmol, 1.0 eq) and DIEA (3.7 mL, 21.1 mmol, 3.0 eq) at icebath. The mixture was stirred at rt for 3 h, then concentrated, diluted with water (120 mL). The resulting mixture was extracted with EtOAc (350 mL×2). The combined organic layers were washed with brine (150 mL), dried over anhydrous sodium sulfate, filtered and concentrated under vacuum. The resulting residue was purified by column chromatography on silica gel (DCM/MeOH=50/1, v/v) to afford 2-((2-(3-fluorophenyl) quinolin-7-yl)(hydroxy)methylene)malononitrile as a yellow oil (1.0 g, 66%), LRMS (M+H⁺) m/z calculated 316.1, found 316.1.

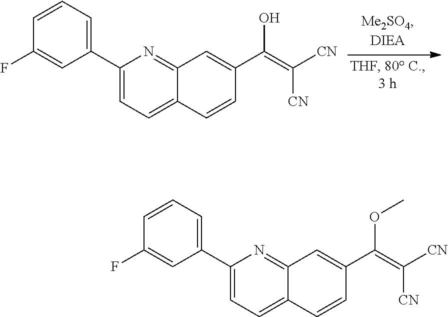 [SMILES: N#CC(C#N)=C(O)c1ccc2ccc(-c3cccc(F)c3)nc2c1 ; COC(=C(C#N)C#N)c1ccc2ccc(-c3cccc(F)c3)nc2c1]

To a solution of 2-((2-(3-fluorophenyl) quinolin-7-yl)(hydroxy)methylene)malononitrile (1.0 g, 3.2 mmol, 1.0 eq) in THF (70 mL) were added Me₂SO₄ (0.6 mL, 6.3 mmol, 2.0 eq) and DIEA (2.7 mL, 15.9 mmol, 5.0 eq) at rt. The mixture was stirred at 80° C. for 3 h, then concentrated under vacuum and diluted with water (120 mL), extracted with EtOAc (250 mL×2). The combined organic layers were washed with brine (150 mL), dried over anhydrous sodium sulfate, filtered and concentrated under vacuum. The resulting residue was purified by column chromatography on silica gel (PE/EA=1/1, v/v) to afford 2-((2-(3-fluorophenyl) quinolin-7-yl)(methoxy)methylene)malononitrile as a yellow oil (100 mg, 10%), LRMS (M+H⁺) m/z calculated 330.1, found 330.1.

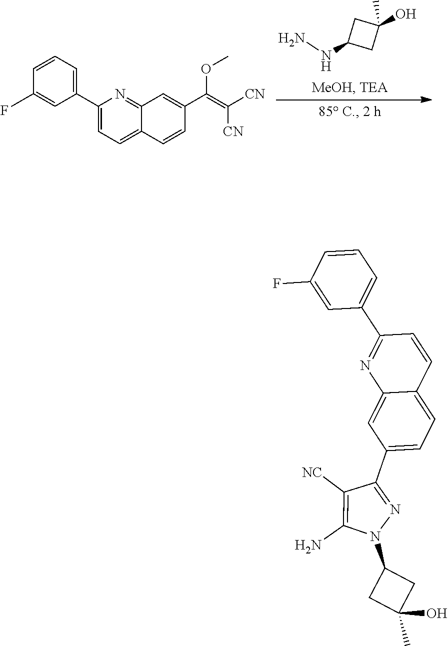

To a solution of 2-((2-(3-fluorophenyl) quinolin-7-yl)(methoxy)methylene)malononitrile (100 mg, 0.30 mmol, 1.0 eq) and (1s,3s)-3-hydrazineyl-1-methylcyclobutan-1-ol (52.9 mg, 0.46 mmol, 1.5 eq) in MeOH (50 mL) were added TEA (0.4 mL, 2.4 mmol, 8.0 eq) at rt. The reaction mixture was stirred at 85° C. for 2 h then the mixture was concentrated under vacuum and purified by column chromatography on silica gel (PE/EA=1/1, v/v) to afford 5-amino-3-(2-(3-fluorophenyl) quinolin-7-yl)-1-((1s,3s)-3-hydroxy-3-methylcyclobutyl)-1H-pyrazole-4-carbonitrile (50 mg, 40%) as a yellow solid. LRMS (M+H⁺) m/z calculated 414.2, found 414.3.

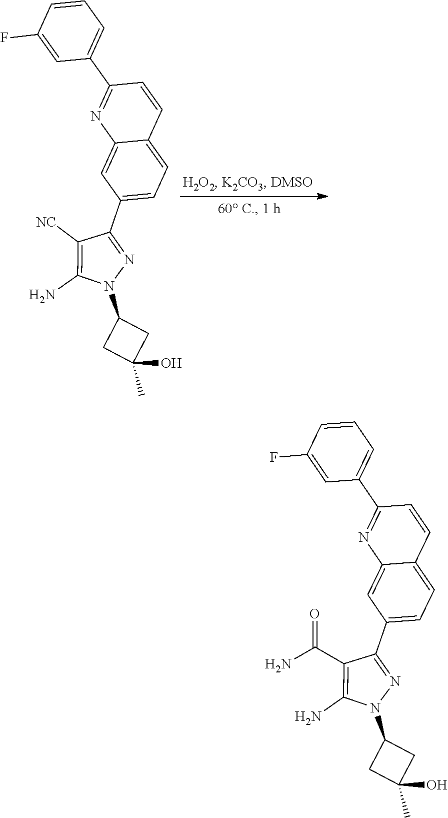 [SMILES: C[C@]1(O)C[C@@H](n2nc(-c3ccc4ccc(-c5cccc(F)c5)nc4c3)c(C#N)c2N)C1 ; C[C@]1(O)C[C@@H](n2nc(-c3ccc4ccc(-c5cccc(F)c5)nc4c3)c(C(N)=O)c2N)C1]

To a stirred solution of 5-amino-3-(2-(3-fluorophenyl) quinolin-7-yl)-1-((1s,3s)-3-hydroxy-3-methylcyclobutyl)-1H-pyrazole-4-carbonitrile (50 mg, 0.12 mmol, 1.0 eq) and K₂CO₃ (50.1 mg, 0.36 mmol, 3.0 eq) in DMSO (20 mL) was added H₂O₂ (30%, 0.3 mL, 2.4 mmol, 20.0 eq) at rt. After addition was complete, the reaction mixture was stirred at 60° C. for 1 h. The mixture was diluted with water (80 mL) and extracted with EtOAc (200 mL). The combined organic layers were washed with brine (100 mL), dried with anhydrous sodium sulfate and purified by Prep-HPLC to afford 5-amino-3-(2-(3-fluorophenyl) quinolin-7-yl)-1-((1s,3s)-3-hydroxy-3-methylcyclobutyl)-1H-pyrazole-4-carboxamide (21.3 mg, 44%) as a white solid. LRMS (M+H⁺) m/z calculated 432.2, found 432.1. ¹H NMR (DMSO-d₆, 400 MHz) δ 8.52 (d, 1H), 8.21-8.24 (m, 2H), 8.16 (d, 1H), 8.11 (d, 1H), 8.07 (d, 1H), 7.79 (dd, 1H), 7.60-7.64 (m, 1H), 7.35-7.39 (m, 1H), 6.30 (s, 2H), 5.19 (s, 1H), 4.42-4.51 (m, 1H), 2.59-2.65 (m, 2H), 2.36-2.42 (m, 2H), 1.34 (s, 3H).

### Example 42: Preparation of 5-amino-3-(8-fluoro-4-hydroxy-2-phenylquinolin-7-yl)-1-((1s,3s)-3-hydroxy-3-methylcyclobutyl)-1H-pyrazole-4-carboxamide

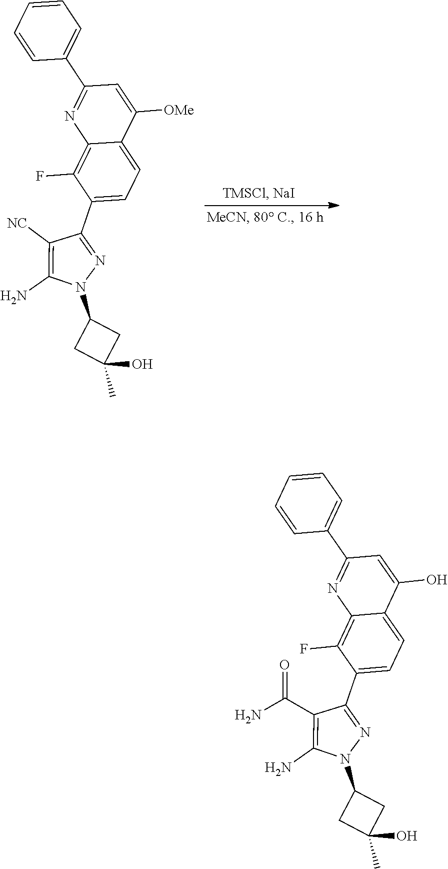 [SMILES: COc1cc(-c2ccccc2)nc2c(F)c(-c3nn([C@H]4C[C@@](C)(O)C4)c(N)c3C#N)ccc12 ; C[C@]1(O)C[C@@H](n2nc(-c3ccc4c(O)cc(-c5ccccc5)nc4c3F)c(C(N)=O)c2N)C1]

To a stirred solution of 5-amino-3-(8-fluoro-4-methoxy-2-phenylquinolin-7-yl)-1-((1s,3s)-3-hydroxy-3-methylcyclobutyl)-1H-pyrazole-4-carboxamide (60 mg, 0.13 mmol, 1.0 eq) in ACN (3 mL) were added TMSI (42 mg, 0.39 mmol, 3.0 eq) and NaI (20 mg, 0.39 mmol, 3.0 eq). The reaction mixture was stirred at 80° C. for 16 h under N₂. The mixture was diluted with water (10 mL) and extracted with EtOAc (20 mL×3). The combined organic layers were washed with brine (100 mL), dried over anhydrous sodium sulfate, filtered and concentrated under vacuum. The residue was purified by Prep-HPLC to afford 5-amino-3-(8-fluoro-4-hydroxy-2-phenylquinolin-7-yl)-1-((1s,3s)-3-hydroxy-3-methylcyclobutyl)-1H-pyrazole-4-carboxamide (30 mg, 51%) as a white solid. LRMS (M+H⁺) m/z calculated 448.2, found 448.2. ¹H NMR (MeOD, 400 MHz) δ 8.18 (d, 1H), 7.85 (t, 2H), 7.48-7.58 (m, 4H), 6.69 (brs, 1H), 4.43-4.48 (m, 1H), 2.73-2.79 (m, 2H), 2.57-2.62 (m, 2H), 1.46 (s, 3H).

### Example 43: Preparation of 5-amino-3-(4-ethoxy-2-phenylquinolin-7-yl)-1-(3-oxocyclobutyl)-1H-pyrazole-4-carboxamide

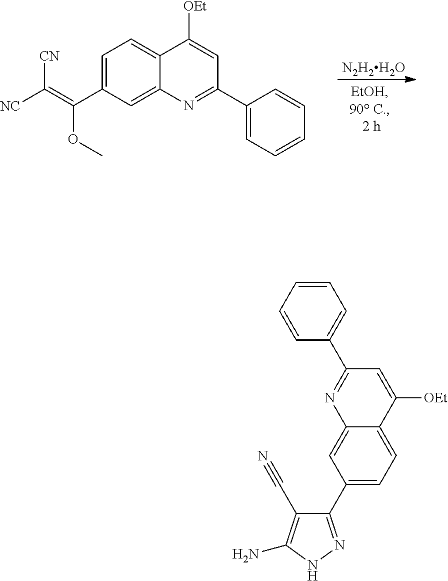

To a solution of 2-((4-ethoxy-2-phenylquinolin-7-yl)(methoxy)methylene)malononitrile (600 mg, 1.7 mmol, 1.0 eq) in EtOH (50 mL) was added NH₂NH₂·H₂O (810 mg, 16.9 mmol, 10.0 eq) at rt. The reaction mixture was stirred at 90° C. for 2 h, then concentrated under vacuum and diluted with water (20 mL). The resulting mixture was stirred for 5 min, and filtered. The solide was dried to afford 5-amino-3-(4-ethoxy-2-phenylquinolin-7-yl)-1H-pyrazole-4-carbonitrile (520 mg, 86%) as a yellow solid, LRMS (M+H⁺) m/z calculated 356.1, found 356.2.

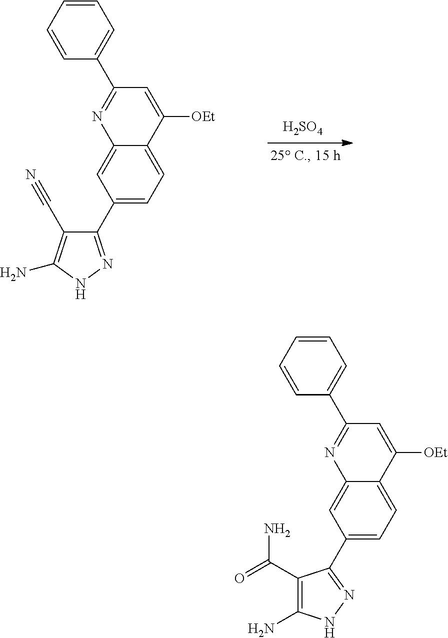 [SMILES: CCOc1cc(-c2ccccc2)nc2cc(-c3n[nH]c(N)c3C#N)ccc12 ; "H2SO4" ; CCOc1cc(-c2ccccc2)nc2cc(-c3n[nH]c(N)c3C(N)=O)ccc12]

To a stirred solution of 5-amino-3-(4-ethoxy-2-phenylquinolin-7-yl)-1H-pyrazole-4-carbonitrile (420 mg, 1.18 mmol, 1.0 eq) was dissolved in conc. H₂SO₄ (5 mL) at 0° C. under N₂. The reaction mixture was stirred at rt for 15 h. The reaction mixture was added carefully to water (100 mL) at 0° C. Na₂CO₃ was added to adjust pH to 12˜13, then the mixture was extracted with DCM (80 mL×2). The combined organic layers were washed with brine (50 mL), dried with anhydrous sodium sulfate and filtered. The filtrate was concentrated to afford 5-amino-3-(4-ethoxy-2-phenylquinolin-7-yl)-1H-pyrazole-4-carboxamide (370 mg, 84%) as a white solid. LRMS (M+H⁺) m/z calculated 374.2, found 374.2.

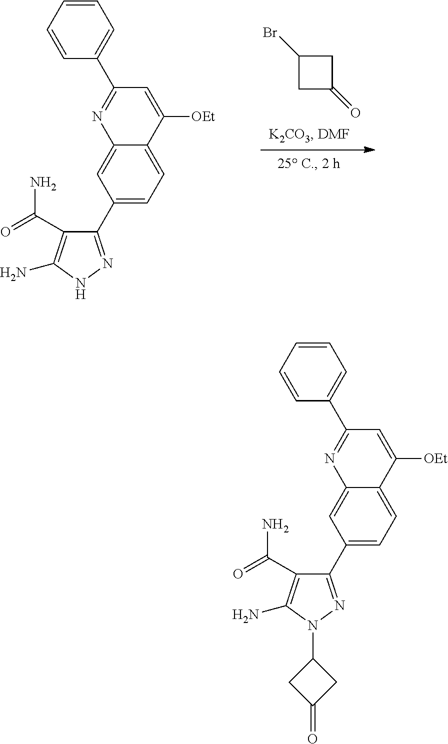

To a solution of 5-amino-3-(4-ethoxy-2-phenylquinolin-7-yl)-1H-pyrazole-4-carboxamide (420 mg, 1.1 mmol, 1.0 eq) in DMF (10 mL) were added 3-bromocyclobutan-1-one (200.0 mg, 1.4 mmol, 1.2 eq) and K₂CO₃ (93.2 mg, 0.68 mmol, 0.6 eq). After addition was complete, the mixture was stirred at rt for 2 h. The reaction mixture was diluted with water (50 mL) and extracted with EtOAc (50 mL×3). The combined organic layers were washed with brine (30 mL×2), dried with anhydrous sodium sulfate, filtered, and concentrated under vacuum. The resulting residue was purified by Prep-HPLC to afford 5-amino-3-(4-ethoxy-2-phenylquinolin-7-yl)-1-(3-oxocyclobutyl)-1H-pyrazole-4-carboxamide (170 mg, 34%). LRMS (M+H⁺) m/z calculated 442.2, found 442.6. ¹H NMR (DMSO-d₆, 400 MHz) δ 8.28-8.31 (m, 2H), 8.18 (d, 1H), 8.14 (d, 1H), 7.71 (dd, 1H), 7.50-7.57 (m, 4H), 6.44 (s, 2H), 5.12-5.16 (m, 1H), 4.48 (q, 2H), 3.59-3.62 (m, 4H), 1.53 (t, 3H).

### Example 44: Preparation of 5-amino-3-(4-(difluoromethoxy)-8-fluoro-2-phenylquinolin-7-yl)-1-((1s,3s)-3-hydroxy-3-methylcyclobutyl)-1H-pyrazole-4-carboxamide

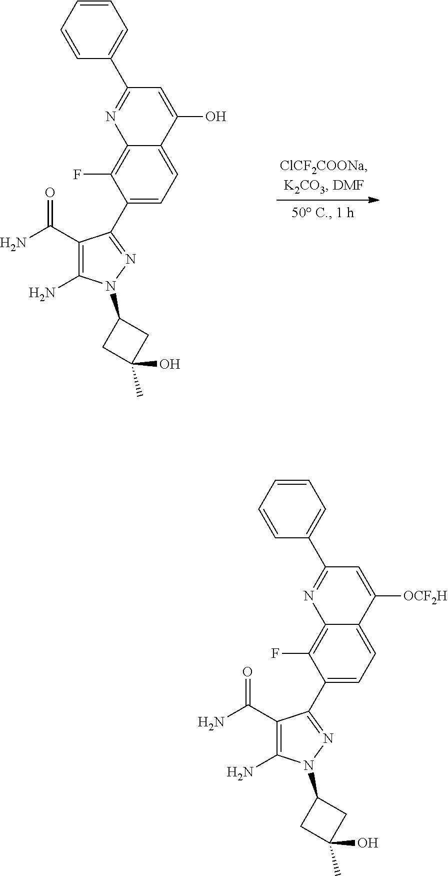

To a solution of 5-amino-3-(8-fluoro-4-hydroxy-2-phenylquinolin-7-yl)-1-((1s,3s)-3-hydroxy-3-methylcyclobutyl)-1H-pyrazole-4-carboxamide (15.0 mg, 0.033 mmol, 1.0 eq) in DMF (10 mL) were added K₂CO₃ (9.3 mg, 0.067 mmol, 2.0 eq) and sodium 2-chloro-2,2-difluoroacetate (7.7 mg, 0.05 mmol, 1.5 eq). After addition was complete, the mixture was stirred at 50° C. for 1 h. The reaction mixture was diluted with water (50 mL) and extracted with EtOAc (50 mL×3). The combined organic layers were washed with brine (30 mL×2), and concentrated under vacuum. The resulting residue was purified by Prep-HPLC to afford 5-amino-3-(4-(difluoromethoxy)-8-fluoro-2-phenylquinolin-7-yl)-1-((1s,3s)-3-hydroxy-3-methylcyclobutyl)-1H-pyrazole-4-carboxamide (2.5 mg, 15.6%). LRMS (M+H⁺) m/z calculated 498.2, found 498.1. ¹H NMR (DMSO-d₆, 400 MHz) δ 8.32 (d, 2H), 7.95-7.98 (m, 2H), 7.56-7.74 (m, 4H), 6.31 (s, 2H), 5.19 (s, 1H), 4.42-4.49 (m, 1H), 2.55-2.61 (m, 2H), 2.36-2.39 (m, 2H), 1.33 (s, 3H).

### Example 45: Preparation of 5-amino-3-(8-fluoro-4-methoxy-2-phenylquinolin-7-yl)-1-((1r,3r)-3-hydroxy-3-methylcyclobutyl)-1H-pyrazole-4-carboxamide

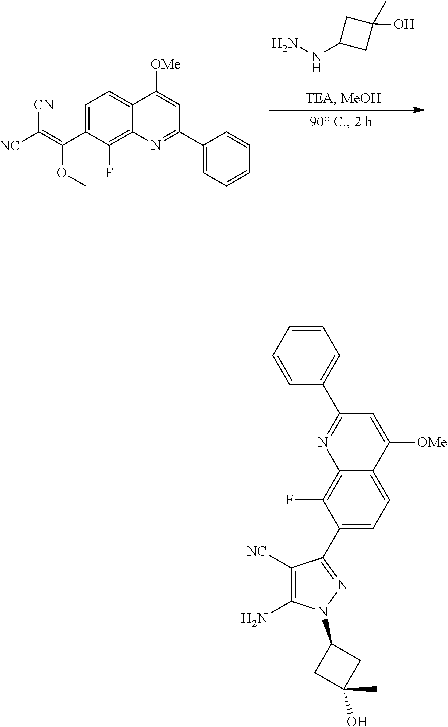

To a solution of 2-((8-fluoro-4-methoxy-2-phenylquinolin-7-yl)(methoxy)methylene)malononitrile (500 mg, 1.39 mmol, 1.0 eq) and 3-hydrazineyl-1-methylcyclobutan-1-ol (242 mg, 2.08 mmol, 1.5 eq) in MeOH (15 mL) were added TEA (1.12 g, 11.1 mmol, 8.0 eq) at rt. The reaction mixture was stirred at 90° C. for 2 h, and concentrated under vacuum. The resulting residue was purified by column chromatography on silica gel (PE/EA=2/1, v/v) to afford 5-amino-3-(8-fluoro-4-methoxy-2-phenylquinolin-7-yl)-1-((1r,3r)-3-hydroxy-3-methylcyclobutyl)-1H-pyrazole-4-carbonitrile (100 mg, 16%) as a white solid. LRMS (M+H⁺) m/z calculated 444.2, found 444.3.

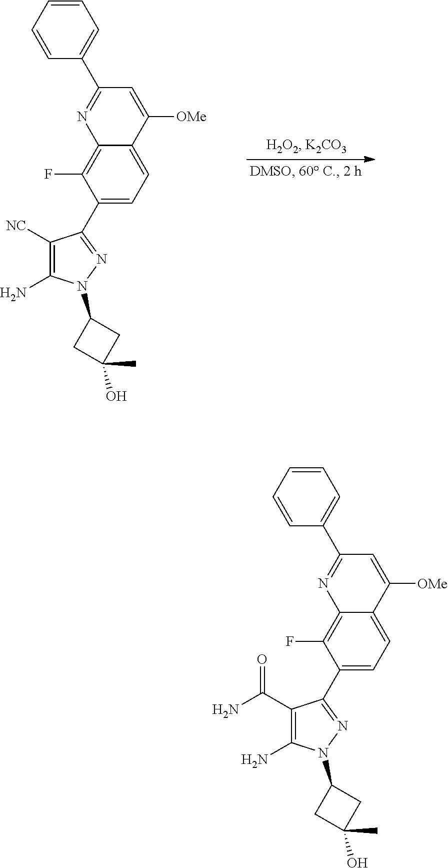

To a stirred solution of 5-amino-3-(8-fluoro-4-methoxy-2-phenylquinolin-7-yl)-1-((1r,3r)-3-hydroxy-3-methylcyclobutyl)-1H-pyrazole-4-carbonitrile (80 mg, 0.18 mmol, 1.0 eq) and K₂CO₃ (75 mg, 0.54 mmol, 3.0 eq) in DMSO (3 mL) was added H₂O₂ (30%, 203 mg, 1.8 mmol, 10.0 eq) at rt. After addition was complete, the mixture was stirred at 60° C. for 2 h, then diluted with water (15 mL), and extracted with EtOAc (30 mL×2). The combined organic layers were washed with brine (10 mL), dried over anhydrous sodium sulfate, filtered and concentrated under vacuum. The resulting residue was purified by Prep-HPLC to afford 5-amino-3-(8-fluoro-4-methoxy-2-phenylquinolin-7-yl)-1-((1r,3r)-3-hydroxy-3-methylcyclobutyl)-1H-pyrazole-4-carboxamide (24 mg, 29%) as a white solid. LRMS (M+H⁺) m/z calculated 462.2, found 462.2, ¹H NMR (DMSO-d₆, 400 MHz) δ 8.32-8.35 (m, 2H), 7.98 (d, 1H), 7.68 (s, 1H), 7.52-7.59 (m, 4H), 6.29 (s, 2H), 4.97 (s, 1H), 4.94-4.96 (m, 1H), 4.21 (s, 3H), 2.40-2.50 (m, 4H), 1.33 (s, 3H).

### Example 46: Preparation of 5-amino-3-(2-(2-fluorophenyl)-4-methoxyquinolin-7-yl)-1-((1s,3s)-3-hydroxy-3-methylcyclobutyl)-1H-pyrazole-4-carboxamide

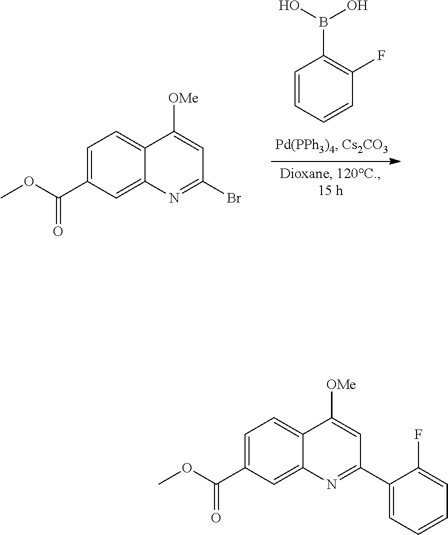 [SMILES: COC(=O)c1ccc2c(OC)cc(Br)nc2c1 ; OB(O)c1ccccc1F ; COC(=O)c1ccc2c(OC)cc(-c3ccccc3F)nc2c1]

To a solution of methyl 2-bromo-4-methoxyquinoline-7-carboxylate (5.0 g, 16.9 mmol, 1.0 eq) in dioxane (100 mL) were added (2-fluorophenyl) boronic acid (4.7 g, 33.9 mmol, 2.0 eq), Pd(PPh₃)₄ (979 mg, 0.8 mmol, 0.05 eq) and Cs₂CO₃ (11.1 g, 33.9 mmol, 2.0 eq). The mixture was stirred at 120° C. for 15 h, then concentrated under vacuum and diluted with water (100 mL), extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 2-(2-fluorophenyl)-4-methoxyquinoline-7-carboxylate (4.5 g, 86%) as a white solid. LRMS (M+H⁺) m/z calculated 312.1. found 312.1.

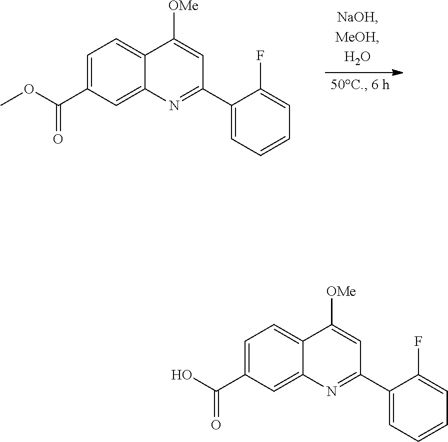 [SMILES: COC(=O)c1ccc2c(OC)cc(-c3ccccc3F)nc2c1 ; COc1cc(-c2ccccc2F)nc2cc(C(=O)O)ccc12]

To a solution of methyl 2-(2-fluorophenyl)-4-methoxyquinoline-7-carboxylate (4.5 g, 14.5 mmol, 1.0 eq) in MeOH (100 mL) and H₂O (10 mL) was added NaOH (868.2 mg, 21.7 mmol, 1.5 eq). The mixture was stirred at 50° C. for 6 h, then concentrated under vacuum and diluted with water (100 mL), 37% HCl was added to adjust to pH 2. The resulting mixture was stirred for 5 min, filtered and dried to afford 2-(2-fluorophenyl)-4-methoxyquinoline-7-carboxylic acid (4 g, 93%) as a white solid, LRMS (M+H⁺) m/z calculated 298.1, found 298.1.

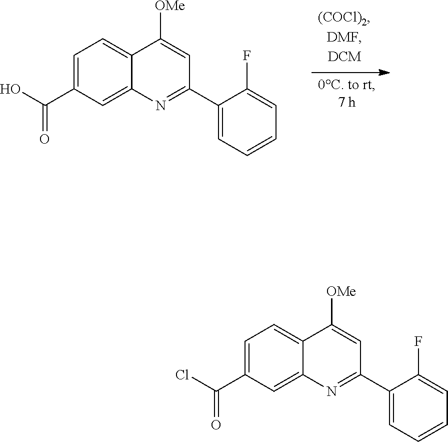 [SMILES: COc1cc(-c2ccccc2F)nc2cc(C(=O)O)ccc12 ; COc1cc(-c2ccccc2F)nc2cc(C(=O)Cl)ccc12]

To a solution of 2-(2-fluorophenyl)-4-methoxyquinoline-7-carboxylic acid (4 g, 13.5 mmol, 1.0 eq) in DCM (100 mL) were added (COCl)₂ (3.4 mL, 40.4 mmol, 3.0 eq) and DMF (0.1 mL) at −78° C. The mixture was stirred at rt for 1 h, then concentrated under vacuum to afford 2-(2-fluorophenyl)-4-methoxyquinoline-7-carbonyl chloride as a yellow solid (5 g, ca 100.0%), LRMS (M+H⁺) m/z calculated 312.1, found 312.1 in MeOH.

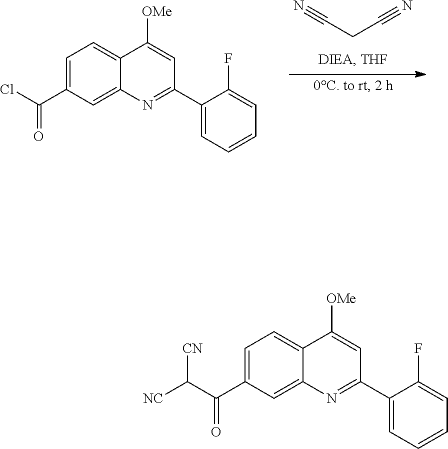 [SMILES: COc1cc(-c2ccccc2F)nc2cc(C(=O)Cl)ccc12 ; N#CCC#N ; COc1cc(-c2ccccc2F)nc2cc(C(=O)C(C#N)C#N)ccc12]

To a solution of 2-(2-fluorophenyl)-4-methoxyquinoline-7-carbonyl chloride (5.0 g, 15.9 mmol, 1.0 eq) in THF (200 mL) were added malononitrile (1.0 g, 15.8 mmol, 1.0 eq) and DIEA (8.3 mL, 47.6 mmol, 3.0 eq) at ice bath. The mixture was stirred at rt for 3 h, then concentrated under vacuum, diluted with water (200 mL). The resulting mixture was extracted with EtOAc (300 mL×2). The combined organic layers were washed with brine (300 mL), dried over anhydrous sodium sulfate, filtered and concentrated under vacuum. The resulting residue was purified by column chromatography on silica gel (DCM/MeOH=50/1, v/v) to afford 2-((2-(2-fluorophenyl)-4-methoxyquinolin-7-yl)(hydroxy)methylene)malononitrile as a yellow oil (10.0 g, 100%), LRMS (M+H⁺) m/z calculated 346.1, found 346.1.

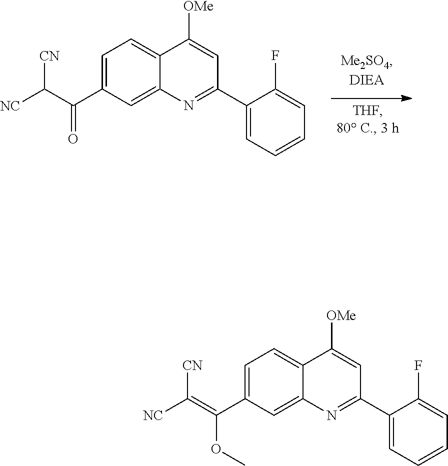 [SMILES: COc1cc(-c2ccccc2F)nc2cc(C(=O)C(C#N)C#N)ccc12 ; COC(=C(C#N)C#N)c1ccc2c(OC)cc(-c3ccccc3F)nc2c1]

To a solution of 2-((2-(2-fluorophenyl)-4-methoxyquinolin-7-yl)(hydroxy)methylene)malononitrile (3.1 g, 9.0 mmol, 1.0 eq) in THF (100 mL) were added Me₂SO₄ (1.7 mL, 18.0 mmol, 2.0 eq) and DIEA (4.7 mL, 26.9 mmol, 3.0 eq) at rt. The mixture was stirred at 80° C. for 3 h, then concentrated under vacuum and diluted with water (200 mL), extracted with EtOAc (500 mL×2). The combined organic layers were washed with brine (500 mL), dried over anhydrous sodium sulfate, filtered and concentrated under vacuum. The resulting residue was purified by column chromatography on silica gel (PE/EA=1/1, v/v) to afford 2-((2-(2-fluorophenyl)-4-methoxyquinolin-7-yl)(methoxy)methylene)malononitrile as a yellow oil (1.2 g, 37%), LRMS (M+H⁺) m/z calculated 360.1, found 360.1.

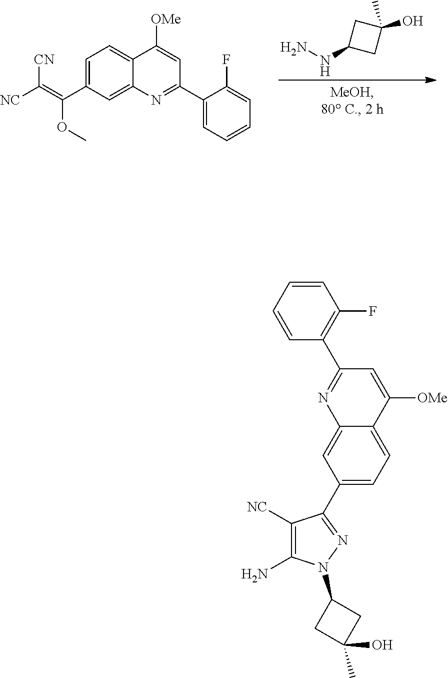

To a solution of 2-((2-(2-fluorophenyl)-4-methoxyquinolin-7-yl)(methoxy)methylene)malononitrile (1.2 g, 3.3 mmol, 1.0 eq) and (1s,3s)-3-hydrazineyl-1-methylcyclobutan-1-ol (581.6 mg, 5.0 mmol, 1.5 eq) in MeOH (80 mL) were added TEA (3.7 mL, 26.7 mmol, 8.0 eq) at rt. The reaction mixture was stirred at 80° C. for 2 h and concentrated under vacuum. The resulting residue was purified by column chromatography on silica gel (PE/EA=1/1, v/v) to afford 5-amino-3-(2-(2-fluorophenyl)-4-methoxyquinolin-7-yl)-1-((1s,3s)-3-hydroxy-3-methylcyclobutyl)-1H-pyrazole-4-carbonitrile (1.2 g, 75%) as a yellow solid. LRMS (M+H⁺) m/z calculated 444.2, found 444.2.

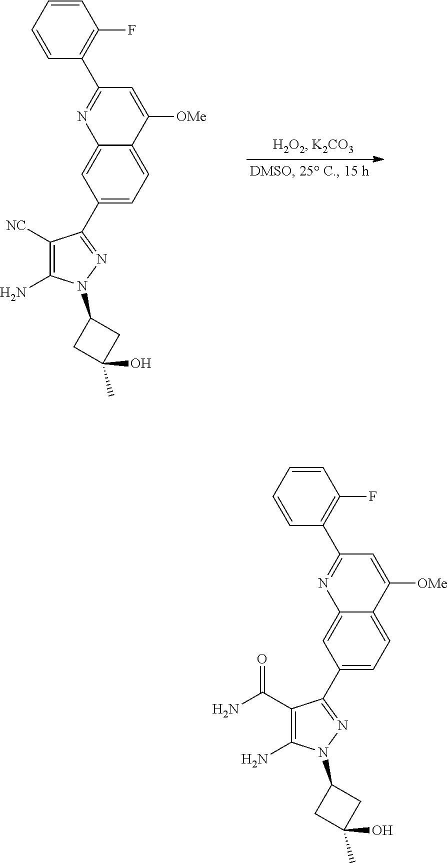

To a stirred solution of 5-amino-3-(2-(2-fluorophenyl)-4-methoxyquinolin-7-yl)-1-((1s,3s)-3-hydroxy-3-methylcyclobutyl)-1H-pyrazole-4-carbonitrile (1.2 g, 2.7 mmol, 1.0 eq) and K₂CO₃ (1.1 g, 8.1 mmol, 3.0 eq) in DMSO (50 mL) at rt was added H₂O₂ (30%, 6.1 mL, 54.2 mmol, 20.0 eq). After addition was complete, the reaction mixture was stirred at rt for 15 h. Water (150 mL) was added and the mixture was extracted with EtOAc (150 mL×2). The organic extract was washed with brine (100 mL), dried with anhydrous sodium sulfate, filtered and concentrated. The resulting residue was purified by Prep-HPLC to afford 5-amino-3-(2-(2-fluorophenyl)-4-methoxyquinolin-7-yl)-1-((1s,3s)-3-hydroxy-3-methylcyclobutyl)-1H-pyrazole-4-carboxamide (720 mg, 60%) as a white solid. LRMS (M+H⁺) m/z calculated 462.2, found 462.2. ¹H NMR (DMSO-d₆, 400 MHz) δ 8.20 (d, 1H), 8.14 (d, 1H), 7.99-8.04 (m, 1H), 7.77 (dd, 1H), 7.53-7.59 (m, 1H), 7.37-7.42 (m, 3H), 6.29 (s, 2H), 5.20 (s, 1H), 4.44-4.49 (m, 1H), 4.12 (s, 3H), 2.59-2.64 (m, 2H), 2.36-2.41 (m, 2H), 1.34 (s, 3H).

### Example 47: Preparation of 5-amino-3-(2-(2-fluorophenyl)-4-methoxyquinolin-7-yl)-1-((1r,3r)-3-hydroxy-3-methylcyclobutyl)-1H-pyrazole-4-carboxamide

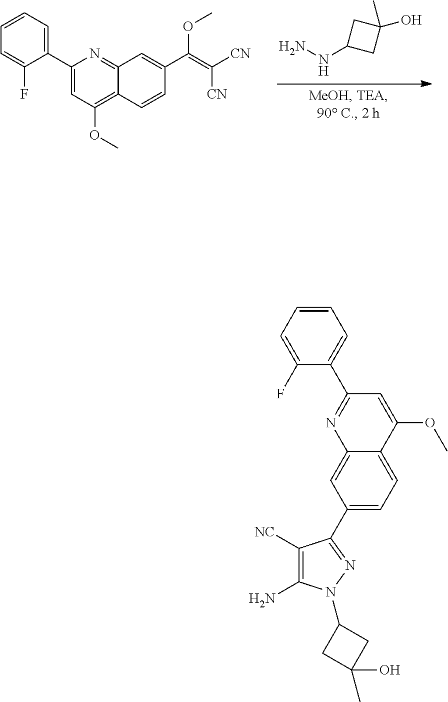

To a solution of 2-((2-(2-fluorophenyl)-4-methoxyquinolin-7-yl)(methoxy)methylene)malononitrile (100 mg, 0.28 mmol, 1.0 eq) and 3-hydrazineyl-1-methylcyclobutan-1-ol (48.5 mg, 0.42 mmol, 1.5 eq) in MeOH (15 mL) was added TEA (225.1 mg, 2.2 mmol, 8.0 eq) at rt. The reaction mixture was stirred at 90° C. for 2 h, and concentrated under vacuum. The resulting residue was purified by column chromatography on silica gel (PE/EA=2/1, v/v) to afford 5-amino-3-(2-(2-fluorophenyl)-4-methoxyquinolin-7-yl)-1-(3-hydroxy-3-methylcyclobutyl)-1H-pyrazole-4-carbonitrile (100 mg, 81%) as a white solid. LRMS (M+H⁺) m/z calculated 444.2, found 444.3.

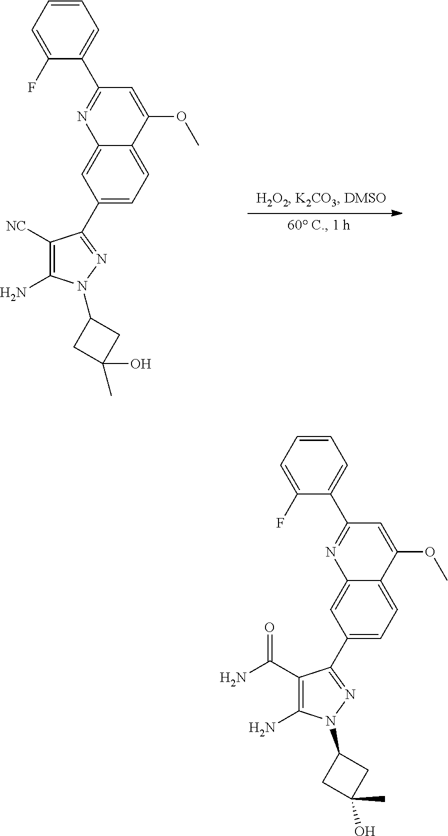 [SMILES: COc1cc(-c2ccccc2F)nc2cc(-c3nn(C4CC(C)(O)C4)c(N)c3C#N)ccc12 ; COc1cc(-c2ccccc2F)nc2cc(-c3nn([C@H]4C[C@](C)(O)C4)c(N)c3C(N)=O)ccc12]

To a stirred solution of 5-amino-3-(2-(2-fluorophenyl)-4-methoxyquinolin-7-yl)-1-(3-hydroxy-3-methylcyclobutyl)-1H-pyrazole-4-carbonitrile (100 mg, 0.22 mmol, 1.0 eq) and K₂CO₃ (30 mg, 0.66 mmol, 3.0 eq) in DMSO (3 mL) was added H₂O₂ (30%, 248 mg, 2.2 mmol, 10.0 eq) at rt. The mixture was stirred at 60° C. for 1 h, then diluted with water (15 mL), and extracted with EtOAc (30 mL×2). The combined organic layers were washed with brine (10 mL), dried over anhydrous sodium sulfate, filtered and concentrated under vacuum. The resulting residue was purified by Prep-HPLC to afford 5-amino-3-(2-(2-fluorophenyl)-4-methoxyquinolin-7-yl)-1-((1r,3r)-3-hydroxy-3-methylcyclobutyl)-1H-pyrazole-4-carboxamide (20 mg, 0.043 mmol, yield 20%) as a white solid. LRMS (M+H⁺) m/z calculated 462.2, found 462.3, ¹H NMR (DMSO-d₆, 400 MHz) δ 8.19 (d, 1H), 8.13 (d, 1H), 8.01 (td, 1H), 7.76 (dd, 1H), 7.53-7.59 (m, 1H), 7.37-7.41 (m, 3H), 6.26 (s, 2H), 4.97 (s, 1H), 4.91-4.96 (m, 1H), 4.12 (s, 3H), 2.38-2.54 (m, 4H), 1.35 (s, 3H).

### Example 48 and 49: Preparation of 5-amino-3-(2-(3-fluorophenyl)-4-methoxyquinolin-7-yl)-1-((1s,3s)-3-hydroxy-3-methylcyclobutyl)-1H-pyrazole-4-carboxamide and 5-amino-3-(2-(3-fluorophenyl)-4-methoxyquinolin-7-yl)-1-((1r,3r)-3-hydroxy-3-methylcyclobutyl)-1H-pyrazole-4-carboxamide

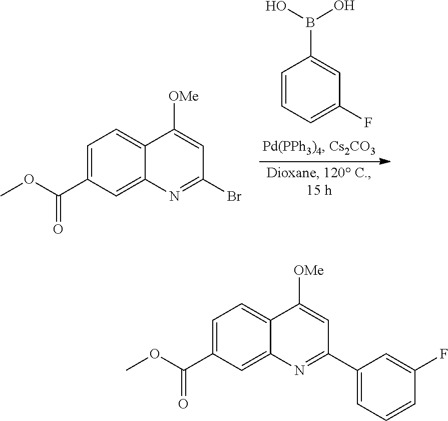 [SMILES: COC(=O)c1ccc2c(OC)cc(Br)nc2c1 ; OB(O)c1cccc(F)c1 ; COC(=O)c1ccc2c(OC)cc(-c3cccc(F)c3)nc2c1]

To a solution of methyl 2-bromo-4-methoxyquinoline-7-carboxylate (4.0 g, 13.6 mmol, 1.0 eq) in dioxane (100 mL) were added (3-fluorophenyl) boronic acid (3.8 g, 27.1 mmol, 2.0 eq), Pd(PPh₃)₄ (783.7 mg, 0.7 mmol, 0.05 eq) and Cs₂CO₃ (8.8 g, 27.1 mmol, 2.0 eq). The mixture was stirred at 120° C. for 15 h, then concentrated under vacuum, diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 2-(3-fluorophenyl)-4-methoxyquinoline-7-carboxylate (1.9 g, 45%) as a yellow solid. LRMS (M+H⁺) m/z calculated 312.1, found 312.1.

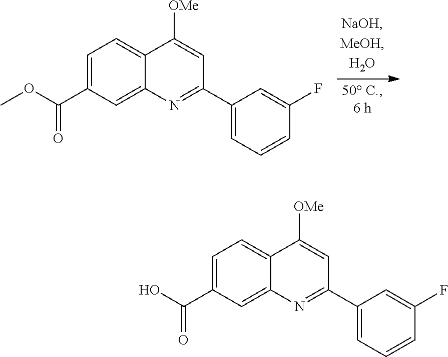 [SMILES: COC(=O)c1ccc2c(OC)cc(-c3cccc(F)c3)nc2c1 ; COc1cc(-c2cccc(F)c2)nc2cc(C(=O)O)ccc12]

To a solution of methyl 2-(3-fluorophenyl)-4-methoxyquinoline-7-carboxylate (1.9 g, 6.1 mmol, 1.0 eq) in MeOH (100 mL) and H₂O (10 mL) was added NaOH (366.6 mg, 9.1 mmol, 1.5 eq). The mixture was stirred at 50° C. for 6 h, then concentrated under vacuum and diluted with water (20 mL), 37% HCl was added to adjust to pH 2. The resulting mixture was stirred for 5 min, filtered and dried to afford 2-(3-fluorophenyl)-4-methoxyquinoline-7-carboxylic acid (1.4 g, 78%) as a white solid, LRMS (M+H⁺) m/z calculated 298.1, found 298.1.

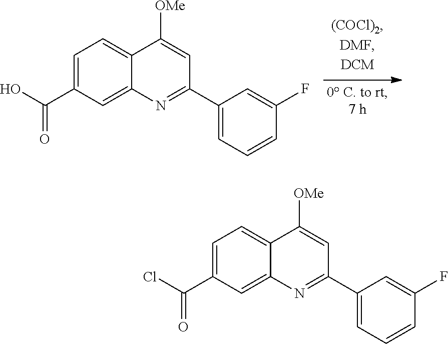 [SMILES: COc1cc(-c2cccc(F)c2)nc2cc(C(=O)O)ccc12 ; COc1cc(-c2cccc(F)c2)nc2cc(C(=O)Cl)ccc12]

To a solution of 2-(3-fluorophenyl)-4-methoxyquinoline-7-carboxylic acid (800 mg, 2.7 mmol, 1.0 eq) in DCM (50 mL) were added (COCl)₂ (0.7 mL, 8.1 mmol, 3.0 eq) and DMF (0.1 mL) at −78° C. The mixture was stirred at rt for 1 h, and concentrated under vacuum to afford 2-(3-fluorophenyl)-4-methoxyquinoline-7-carbonyl chloride as a yellow solid (848 mg, ca 100.0%), LRMS (M+H⁺) m/z calculated 312.1, found 312.1 in MeOH.

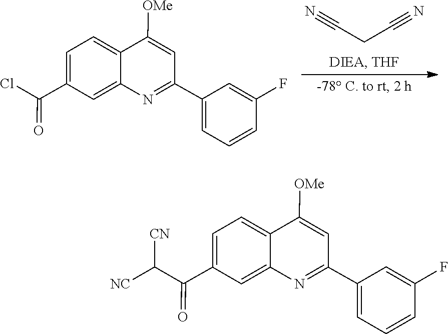 [SMILES: COc1cc(-c2cccc(F)c2)nc2cc(C(=O)Cl)ccc12 ; N#CCC#N ; COc1cc(-c2cccc(F)c2)nc2cc(C(=O)C(C#N)C#N)ccc12]

To a solution of 2-(3-fluorophenyl)-4-methoxyquinoline-7-carbonyl chloride (848 mg, 2.7 mmol, 1.0 eq) in THF (50 mL) were added malononitrile (177.7 mg, 2.7 mmol, 1.0 eq) and DIEA (1.4 mL, 8.1 mmol, 3.0 eq) at ice bath. The mixture was stirred at rt for 3 h, then concentrated under vacuum, and diluted with water (100 mL). The resulting mixture was extracted with EtOAc (100 mL×2). The combined organic layers were washed with brine (100 mL), dried over anhydrous sodium sulfate, filtered and concentrated under vacuum. The resulting residue was purified by column chromatography on silica gel (DCM/MeOH=50/1, v/v) to afford 2-((2-(3-fluorophenyl)-4-methoxyquinolin-7-yl)(hydroxy)methylene)malononitrile as a yellow oil (600 mg, 64.6%), LRMS (M+H⁺) m/z calculated 346.1, found 346.1.

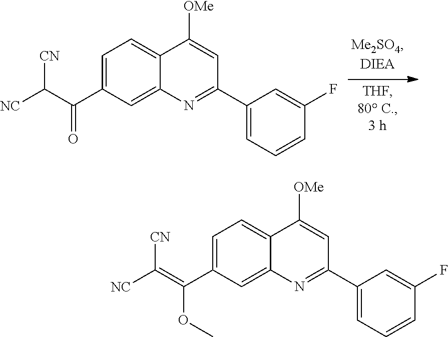

To a solution of 2-((2-(3-fluorophenyl)-4-methoxyquinolin-7-yl)(hydroxy)methylene)malononitrile (600 mg, 1.7 mmol, 1.0 eq) in THF (50 mL) were added Me₂SO₄ (0.4 mL, 3.5 mmol, 2.0 eq) and DIEA (0.9 mL, 5.2 mmol, 3.0 eq) at rt. The mixture was stirred at 80° C. for 3 h, then concentrated under vacuum and diluted with water (100 mL), extracted with EtOAc (100 mL×2). The combined organic layers were washed with brine (100 mL), dried over anhydrous sodium sulfate, filtered and concentrated under vacuum. The resulting residue was purified by column chromatography on silica gel (PE/EA=1/1, v/v) to afford 2-((2-(3-fluorophenyl)-4-methoxyquinolin-7-yl)(methoxy)methylene)malononitrile as a yellow oil (200 mg, 32%), LRMS (M+H⁺) m/z calculated 360.1, found 360.1.

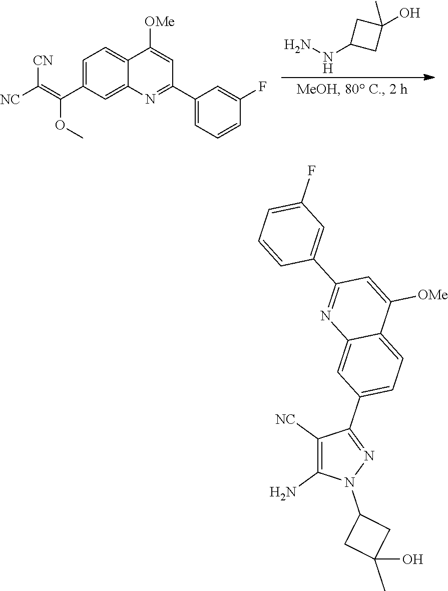

To a solution of 2-((2-(3-fluorophenyl)-4-methoxyquinolin-7-yl)(methoxy)methylene)malononitrile (200 mg, 0.56 mmol, 1.0 eq) and 3-hydrazineyl-1-methylcyclobutan-1-ol (96.9 mg, 0.84 mmol, 1.5 eq) in MeOH (80 mL) were added TEA (0.6 mL, 4.5 mmol, 8.0 eq) at rt. The reaction mixture was stirred at 80° C. for 2 h, then concentrated under vacuum and purified by column chromatography on silica gel (PE/EA=1/1, v/v) to afford 5-amino-3-(2-(3-fluorophenyl)-4-methoxyquinolin-7-yl)-1-(3-hydroxy-3-methylcyclobutyl)-1H-pyrazole-4-carbonitrile (128.0 mg, 50%) as a yellow solid. LRMS (M+H⁺) m/z calculated 444.2, found 444.2.

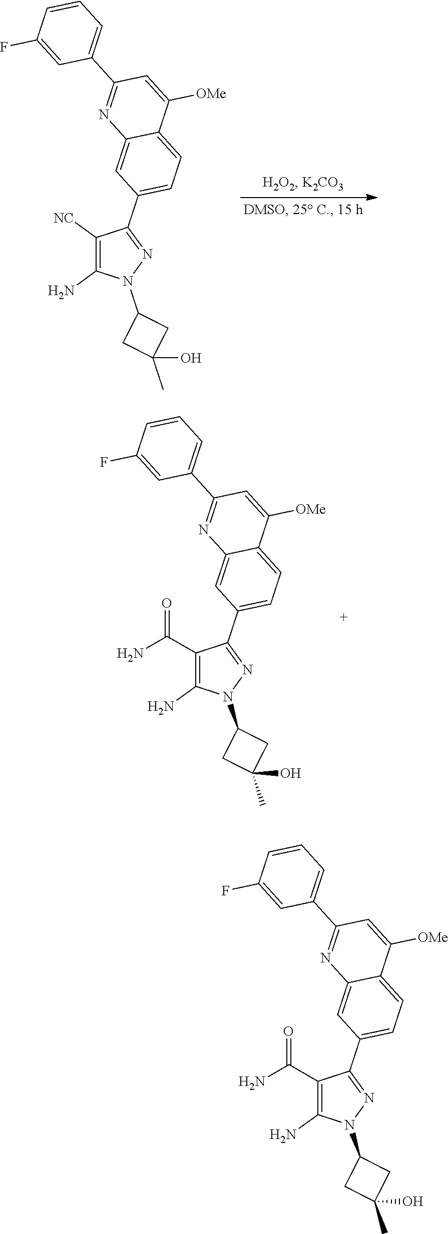

To a stirred solution of 5-amino-3-(2-(3-fluorophenyl)-4-methoxyquinolin-7-yl)-1-((1r,3r)-3-hydroxy-3-methylcyclobutyl)-1H-pyrazole-4-carbonitrile (120 mg, 0.27 mmol, 1.0 eq) and K₂CO₃ (112 mg, 0.81 mmol, 3.0 eq) in DMSO (5 mL) was added H₂O₂ (30%, 612 mg, 5.4 mmol, 20.0 eq) at rt. After addition was complete, the reaction mixture was stirred at rt for 15 h. Water (50 mL) was added and the mixture was extracted with EtOAc (50 mL×2). The organic extract was washed with brine (50 mL), dried with anhydrous sodium sulfate, filtered, and concentrated under vacuum. The resulting residue was purified by Prep-HPLC to afford 5-amino-3-(2-(3-fluorophenyl)-4-methoxyquinolin-7-yl)-1-((1s,3s)-3-hydroxy-3-methylcyclobutyl)-1H-pyrazole-4-carboxamide (25.5 mg, 50%) and 5-amino-3-(2-(3-fluorophenyl)-4-methoxyquinolin-7-yl)-1-((1r,3r)-3-hydroxy-3-methylcyclobutyl)-1H-pyrazole-4-carboxamide (22 mg, 18%) as a white solid.

5-amino-3-(2-(3-fluorophenyl)-4-methoxyquinolin-7-yl)-1-((1s,3s)-3-hydroxy-3-methylcyclobutyl)-1H-pyrazole-4-carboxamide: LRMS (M+H⁺) m/z calculated 462.2, found 462.3. ¹H NMR (DMSO-d₆, 400 MHz) δ 8.13-8.21 (m, 4H), 7.73 (dd, 1H), 7.63 (s, 1H), 7.57-7.62 (m, 1H), 7.34-7.38 (m, 1H), 6.29 (s, 2H), 5.20 (s, 1H), 4.44-4.49 (m, 1H), 4.20 (s, 3H), 2.59-2.64 (m, 2H), 2.36-2.41 (m, 2H), 1.34 (s, 3H).

5-amino-3-(2-(3-fluorophenyl)-4-methoxyquinolin-7-yl)-1-((1r,3r)-3-hydroxy-3-methylcyclobutyl)-1H-pyrazole-4-carboxamide: LRMS (M+H⁺) m/z calculated 462.2, found 462.3. ¹H NMR (DMSO-d₆, 400 MHz) δ 8.13-8.21 (m, 4H), 7.73 (dd, 1H), 7.63 (s, 1H), 7.57-7.62 (m, 1H), 7.34-7.38 (m, 1H), 6.27 (s, 2H), 4.92-4.98 (m, 2H), 4.20 (s, 3H), 2.38-2.54 (m, 4H), 1.35 (s, 3H).

### Example 50: Preparation of 5-amino-3-(8-fluoro-2-(2-fluorophenyl)-4-methoxyquinolin-7-yl)-1-((1r,3r)-3-hydroxy-3-methylcyclobutyl)-1H-pyrazole-4-carboxamide

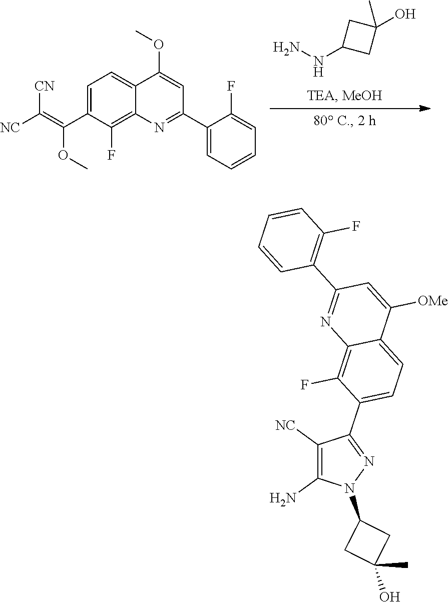

To a solution of 2-((8-fluoro-2-(2-fluorophenyl)-4-methoxyquinolin-7-yl)(methoxy)methylene)malononitrile (1.1 g, 2.9 mmol, 1.0 eq) and 3-hydrazineyl-1-methylcyclobutan-1-ol (406 mg, 3.5 mmol, 1.2 eq) in MeOH (50 mL) was added TEA (3.2 mL, 23.3 mmol, 8.0 eq) at rt. The reaction mixture was stirred at 80° C. for 2 h, and concentrated under vacuum. The resulting residue was purified by column chromatography on silica gel (PE/EA=2/1, v/v) to afford 5-amino-3-(8-fluoro-2-(2-fluorophenyl)-4-methoxyquinolin-7-yl)-1-((1s,3s)-3-hydroxy-3-methylcyclobutyl)-1H-pyrazole-4-carbonitrile (240 mg, 18%) as a yellow solid, LRMS (M+H⁺) m/z calculated 462.2, found 462.4.

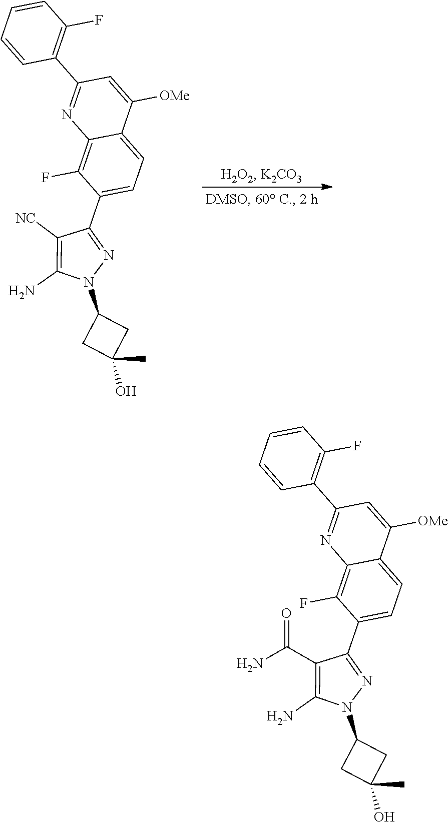 [SMILES: COc1cc(-c2ccccc2F)nc2c(F)c(-c3nn([C@H]4C[C@](C)(O)C4)c(N)c3C#N)ccc12 ; COc1cc(-c2ccccc2F)nc2c(F)c(-c3nn([C@H]4C[C@](C)(O)C4)c(N)c3C(N)=O)ccc12]

To a stirred solution of 5-amino-3-(8-fluoro-2-(2-fluorophenyl)-4-methoxyquinolin-7-yl)-1-((1r,3r)-3-hydroxy-3-methylcyclobutyl)-1H-pyrazole-4-carbonitrile (220 mg, 0.48 mmol, 1.0 eq) in DMSO (6 mL) were added K₂CO₃ (331 mg, 2.4 mmol, 5.0 eq) and H₂O₂ (30%, 1.1 g, 9.6 mmol, 20.0 eq). After addition was complete, the mixture was stirred at 60° C. for 2 h, then diluted with water (20 mL), and extracted with EtOAc (50 mL×2). The combined organic layers were washed with brine (30 mL), dried over anhydrous sodium sulfate, filtered and concentrated under vacuum. The resulting residue was purified by Prep-HPLC to afford 5-amino-3-(8-fluoro-2-(2-fluorophenyl)-4-methoxyquinolin-7-yl)-1-((1r,3r)-3-hydroxy-3-methylcyclobutyl)-1H-pyrazole-4-carboxamide as a white solid (135 mg, 59%), LRMS (M+H⁺) m/z calculated 480.2, found 480.3. ¹H NMR (DMSO-d₆, 400 MHz) δ 7.99-8.05 (m, 2H), 7.55-7.62 (m, 2H), 7.47 (d, 1H), 7.38-7.43 (m, 2H), 6.29 (s, 2H), 4.98 (s, 1H), 4.92-4.97 (m, 1H), 4.14 (s, 3H), 2.38-2.51 (m, 4H), 1.33 (s, 3H).

### Example 51 and 52: Preparation of 5-amino-3-(6-fluoro-4-methoxy-2-phenylquinolin-7-yl)-1-((1s,3s)-3-hydroxy-3-methylcyclobutyl)-1H-pyrazole-4-carboxamide and 5-amino-3-(6-fluoro-4-methoxy-2-phenylquinolin-7-yl)-1-((1r,3r)-3-hydroxy-3-methylcyclobutyl)-1H-pyrazole-4-carboxamide

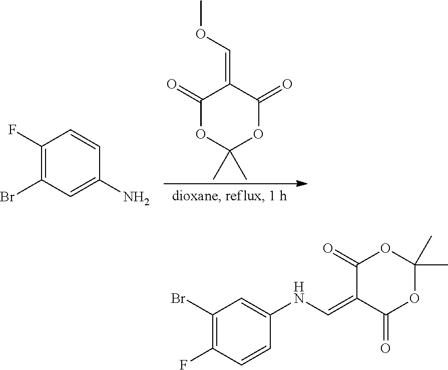 [SMILES: Nc1ccc(F)c(Br)c1 ; COC=C1C(=O)OC(C)(C)OC1=O ; CC1(C)OC(=O)C(=CNc2ccc(F)c(Br)c2)C(=O)O1]

A mixture of 3-bromo-4-fluoroaniline (13.0 g, 68.4 mmol, 1.0 eq) and 5-(methoxymethylene)-2,2-dimethyl-1,3-dioxane-4,6-dione (12.7 g, 132.2 mmol, 1.0 eq) in dioxane (200 mL). The mixture was stirred at 120° C. for 1 h, then cooled to rt, and diluted with PE (500 mL). The precipitate was filtered to afford 5-(((3-bromo-4-fluorophenyl)amino)methylene)-2,2-dimethyl-1,3-dioxane-4,6-dione (21 g, 88%) as a yellow solid.

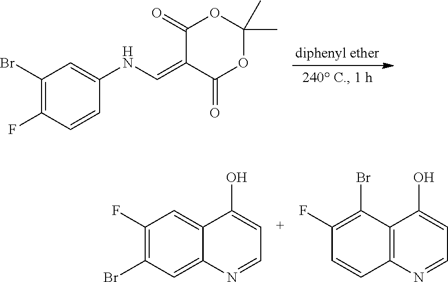

A mixture of 5-(((3-bromo-4-fluorophenyl)amino)methylene)-2,2-dimethyl-1,3-dioxane-4,6-dione (21 g, 61.2 mmol, 1.0 eq) and Ph₂O (250 mL) was stirred at 240° C. for 1 h. The mixture was cooled to rt and diluted with PE (200 mL), and precipitate was filtered to afford a mixture of 7-bromo-6-fluoroquinolin-4-ol and 5-bromo-6-fluoroquinolin-4-ol (11.6 g, 79%) as a brown solid. LRMS (M+H⁺) m/z calculated 242.0, found 242.1.

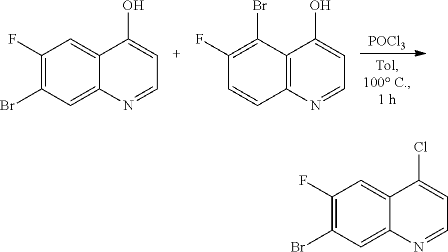

To a solution of 7-bromo-6-fluoroquinolin-4-ol and 5-bromo-6-fluoroquinolin-4-ol (11.6 g, 48.1 mmol, 1.0 eq) in Tol (120 mL) were added POCl₃ (9.2 mL, 96.2 mmol, 2 eq). The mixture was stirred at 100° C. for 1 h, then poured into ice, adjusted to pH 13 with saturated Na₂CO₃ aqueous solution, and extracted with DCM (500 mL×3). The combined organic layers were dried over anhydrous sodium sulfate, filtered and concentrated under vacuum. The resulting residue was purified by column chromatography on silica gel (PE/EA=5/1, v/v) to afford 7-bromo-4-chloro-6-fluoroquinoline (5.5 g, 50.0%) as a yellow solid, LRMS (M+H⁺) m/z calculated 259.9, found 260.0. ¹H NMR (DMSO-d₆, 400 MHz) δ 8.86 (d, 1H), 8.52 (d, 1H), 8.03 (d, 1H), 7.85 (d, 1H).

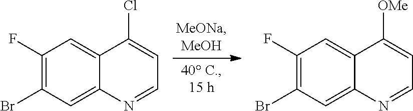 [SMILES: Fc1cc2c(Cl)ccnc2cc1Br ; COc1ccnc2cc(Br)c(F)cc12]

To a solution of 7-bromo-4-chloro-6-fluoroquinoline (1.9 g, 7.3 mmol, 1.0 eq) in MeOH (80 mL) was added MeONa (789.2 mg, 14.6 mmol, 2.0 eq). The mixture was stirred at 40° C. for 15 h, then concentrated under vacuum, diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford 7-bromo-6-fluoro-4-methoxyquinoline (850 mg, 50%) as a white solid. LRMS (M+H⁺) m/z calculated 256.0, found 256.0.

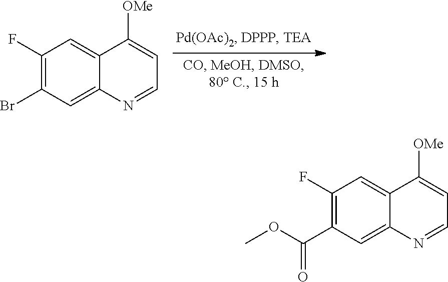 [SMILES: COc1ccnc2cc(Br)c(F)cc12 ; COC(=O)c1cc2nccc(OC)c2cc1F]

To a solution of 7-bromo-6-fluoro-4-methoxyquinoline (850 mg, 3.3 mmol, 1.0 eq), DPPP (547.2 mg, 1.3 mmol, 0.4 eq) and Pd(OAc)₂ (148.8 mg, 0.66 mmol, 0.2 eq) in DMSO/MeOH (50 mL/50 mL) was added TEA (2.3 mL, 16.6 mmol. 5.0 eq). The mixture was stirred at 80° C. for 15 h under CO (1 atm), then the reaction mixture was diluted with water (100 mL) and extracted with EtOAc (100 mL×3). The combined organic layers were washed with brine (100 mL), dried with anhydrous sodium sulfate, filtered, and concentrated under vacuum. The resulting residue was purified by column chromatography on silica gel (PE/EA=5/1, v/v) to afford methyl 6-fluoro-4-methoxyquinoline-7-carboxylate (468 mg, 61%) as a white solid. LRMS (M+H⁺) m/z calculated 236.1, found 236.2.

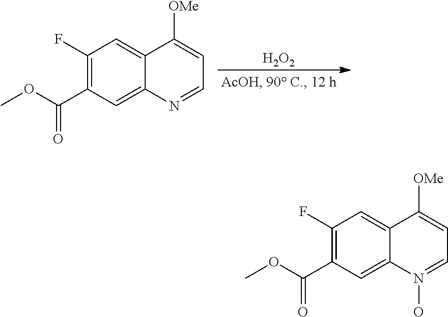

A mixture of methyl 6-fluoro-4-methoxyquinoline-7-carboxylate (468 mg, 2.0 mmol, 1.0 eq) and hydrogen peroxide (0.9 mL of 30% solution, 0.34 mol, 4.0 eq) AcOH (20 mL) was stirred at 90° C. for 15 h, then concentrated in vacuo to afford 6-fluoro-4-methoxy-7-(methoxycarbonyl) quinoline 1-oxide (400 mg, 80%) as a yellow solid. LRMS (M+H⁺) m/z calculated 252.1, found 252.2.

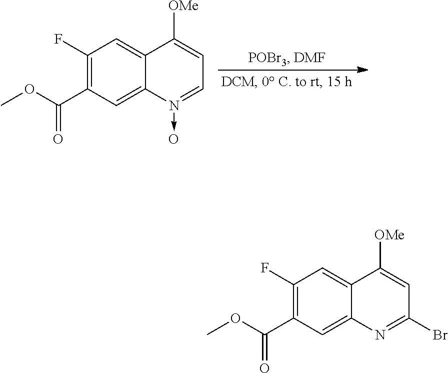

To a solution of 6-fluoro-4-methoxy-7-(methoxycarbonyl) quinoline 1-oxide (400 mg, 1.6 mmol, 1.0 eq) in DCM (80 mL) were added POBr₃ (594.5 mg, 2.1 mmol, 1.3 eq) and DMF (2 drops) at 0° C. The mixture was stirred at rt for 15 h, then poured into ice, adjusted to pH 13 with saturated Na₂CO₃ aqueous solution, and extracted with DCM (100 mL×3). The combined organic layers were dried over anhydrous sodium sulfate, filtered and concentrated under vacuum. The resulting residue was purified by column chromatography on silica gel (PE/EA=5/1, v/v) to afford methyl 2-bromo-6-fluoro-4-methoxyquinoline-7-carboxylate (124.7 mg, 24%) as a yellow solid, LRMS (M+H⁺) m/z calculated 314.0, found 314.0.

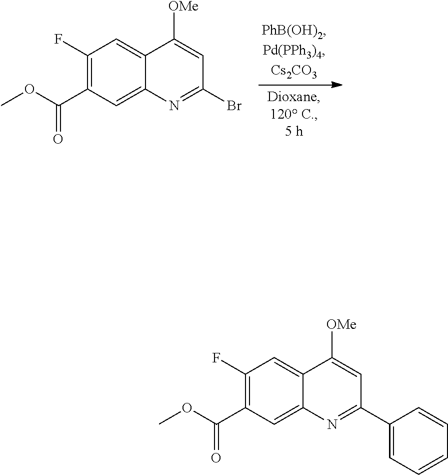 [SMILES: COC(=O)c1cc2nc(Br)cc(OC)c2cc1F ; COC(=O)c1cc2nc(-c3ccccc3)cc(OC)c2cc1F]

To a solution of methyl 2-bromo-6-fluoro-4-methoxyquinoline-7-carboxylate (125 mg, 0.40 mmol, 1.0 eq) in dioxane (30 mL) were added phenylboronic acid (97.4 mg, 0.80 mmol, 2.0 eq), Pd(PPh₃)₄ (46.2 mg, 0.04 mmol, 0.1 eq) and Cs₂CO₃ (260.4 mg, 0.80 mmol, 2.0 eq). The mixture was stirred at 120° C. for 5 h, then concentrated under vacuum and diluted with water (50 mL), extracted with EtOAc (50 mL×2). The combined organic layers were washed with brine (50 mL), dried over anhydrous sodium sulfate, filtered and concentrated under vacuum. The resulting residue was purified by column chromatography on silica gel (PE/EA=5/1, v/v) to afford methyl 6-fluoro-4-methoxy-2-phenylquinoline-7-carboxylate (100 mg, 80%) as a yellow solid. LRMS (M+H⁺) m/z calculated 312.1, found 312.1.

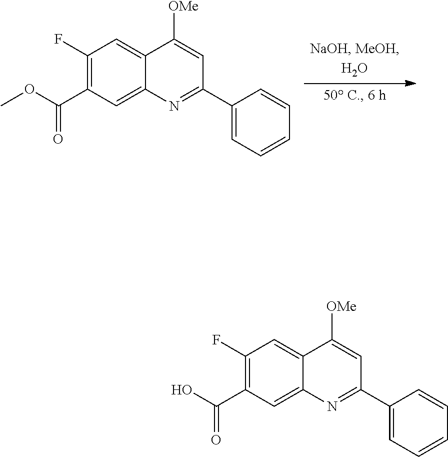 [SMILES: COC(=O)c1cc2nc(-c3ccccc3)cc(OC)c2cc1F ; COc1cc(-c2ccccc2)nc2cc(C(=O)O)c(F)cc12]

To a solution of methyl 6-fluoro-4-methoxy-2-phenylquinoline-7-carboxylate (100 mg, 0.32 mmol, 1.0 eq) in MeOH (50 mL) and H₂O (10 mL) was added NaOH (19.3 mg, 0.48 mmol, 1.5 eq). The mixture was stirred at 50° C. for 6 h, then concentrated under vacuum and diluted with water (20 mL), 37% HCl was added to adjust to pH=2. The resulting mixture was stirred for 5 min, filtered and dried to afford 6-fluoro-4-methoxy-2-phenylquinoline-7-carboxylic acid (100 mg, 100%) as a yellow solid, LRMS (M+H⁺) m/z calculated 298.1, found 298.1.

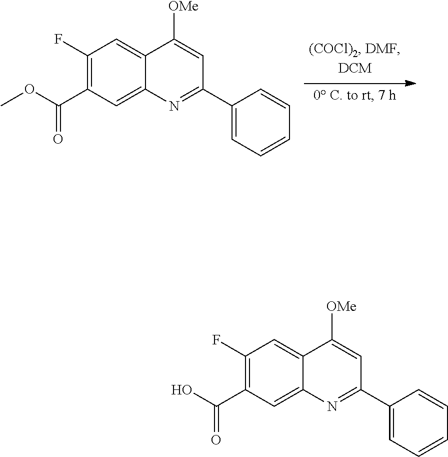 [SMILES: COC(=O)c1cc2nc(-c3ccccc3)cc(OC)c2cc1F ; COc1cc(-c2ccccc2)nc2cc(C(=O)O)c(F)cc12]

To a solution of 6-fluoro-4-methoxy-2-phenylquinoline-7-carboxylic acid (100 mg, 0.33 mmol, 1.0 eq) in DCM (50 mL) were added (COCl)₂ (0.1 mL, 1.0 mmol, 3.0 eq) and DMF (2 drops) at −78° C. The mixture was stirred at rt for 1 h, then concentrated under vacuum to afford 6-fluoro-4-methoxy-2-phenylquinoline-7-carbonyl chloride as a yellow solid (120 mg, ca 100.0%), LRMS (M+H⁺) m/z calculated 312.1, found 312.1 in MeOH.

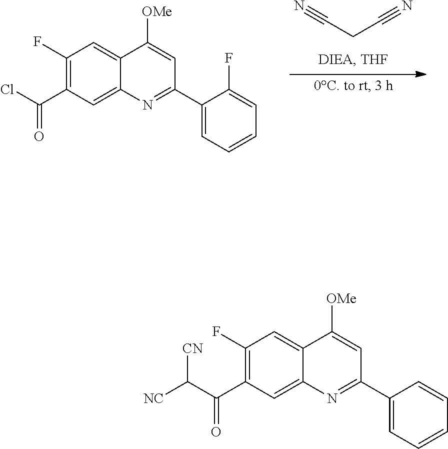 [SMILES: COc1cc(-c2ccccc2F)nc2cc(C(=O)Cl)c(F)cc12 ; N#CCC#N ; COc1cc(-c2ccccc2)nc2cc(C(=O)C(C#N)C#N)c(F)cc12]

To a solution of 6-fluoro-4-methoxy-2-phenylquinoline-7-carbonyl chloride (120 mg, 0.38 mmol, 1.0 eq) in THF (20 mL) were added malononitrile (25.1 mg, 0.38 mmol, 1.0 eq) and DIEA (0.2 mL, 1.1 mmol, 3.0 eq) at ice bath. The mixture was stirred at rt for 3 h, then concentrated under vacuum, and diluted with water (100 mL). The resulting mixture was extracted with EtOAc (100 mL×2). The combined organic layers were washed with brine (100 mL), dried over anhydrous sodium sulfate, filtered and concentrated under vacuum. The resulting residue was purified by column chromatography on silica gel (DCM/MeOH=50/1, v/v) to afford 2-(6-fluoro-4-methoxy-2-phenylquinoline-7-carbonyl)malononitrile as a yellow oil (91 mg, 70%), LRMS (M+H⁺) m/z calculated 346.1, found 346.1.

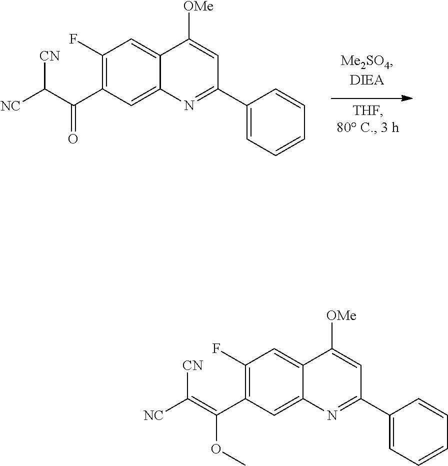 [SMILES: COc1cc(-c2ccccc2)nc2cc(C(=O)C(C#N)C#N)c(F)cc12 ; COC(=C(C#N)C#N)c1cc2nc(-c3ccccc3)cc(OC)c2cc1F]

To a solution of 2-(6-fluoro-4-methoxy-2-phenylquinoline-7-carbonyl)malononitrile (91 mg, 0.26 mmol, 1.0 eq) in THF (30 mL) were added Me₂SO₄ (66.5 mg, 0.53 mmol, 2.0 eq) and DIEA (0.2 mL, 0.8 mmol, 3.0 eq) at rt. The mixture was stirred at 80° C. for 3 h, then concentrated under vacuum and diluted with water (50 mL), extracted with EtOAc (50 mL×2). The combined organic layers were washed with brine (100 mL), dried over anhydrous sodium sulfate, filtered and concentrated under vacuum. The resulting residue was purified by column chromatography on silica gel (PE/EA=1/1, v/v) to afford 2-((6-fluoro-4-methoxy-2-phenylquinolin-7-yl)(methoxy)methylene)malononitrile as a yellow oil (50 mg, 53%), LRMS (M+H⁺) m/z calculated 360.1, found 360.1.

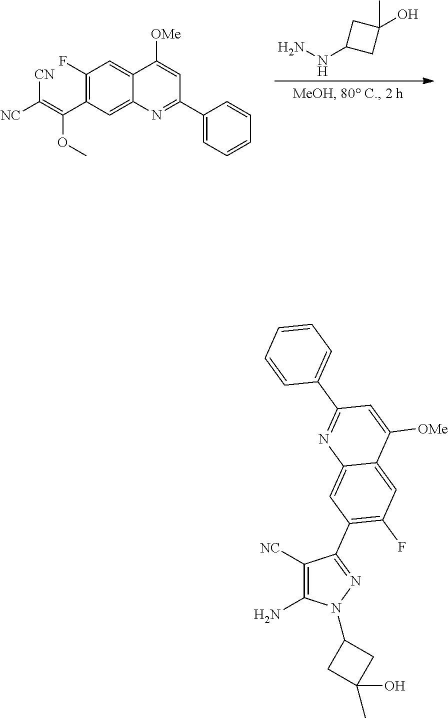

To a solution of 2-((6-fluoro-4-methoxy-2-phenylquinolin-7-yl)(methoxy)methylene)malononitrile (50 mg, 0.14 mmol, 1.0 eq) and 3-hydrazineyl-1-methylcyclobutan-1-ol (24.2 mg, 0.21 mmol, 1.5 eq) in MeOH (30 mL) were added TEA (0.2 mL, 1.1 mmol, 8.0 eq) at rt. The reaction mixture was stirred at 80° C. for 2 h, then the mixture was concentrated under vacuum and purified by column chromatography on silica gel (PE/EA=1/1, v/v) to afford 5-amino-3-(6-fluoro-4-methoxy-2-phenylquinolin-7-yl)-1-(3-hydroxy-3-methylcyclobutyl)-1H-pyrazole-4-carbonitrile (20.0 mg, 32.7%) as a yellow solid. LRMS (M+H⁺) m/z calculated 444.2, found 444.2.

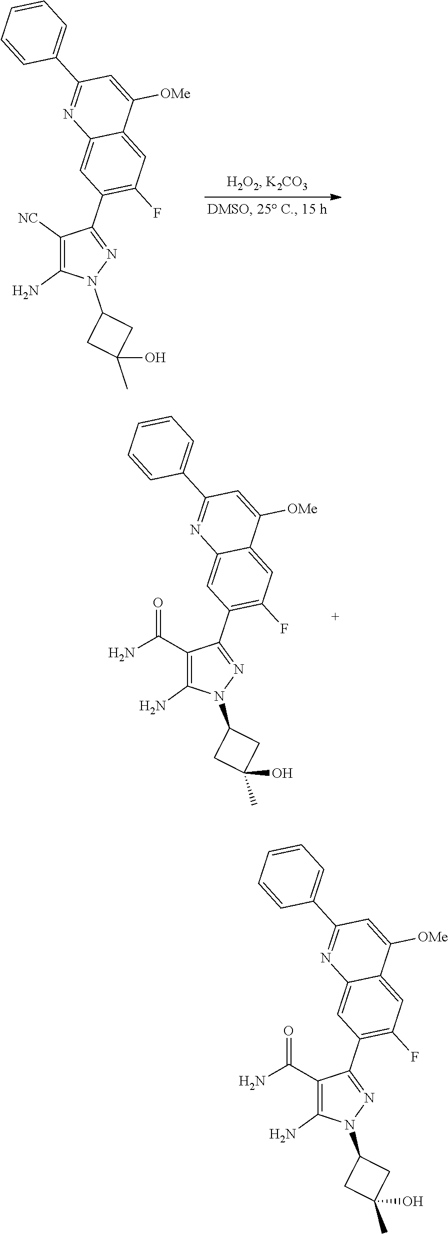

To a stirred solution of 5-amino-3-(6-fluoro-4-methoxy-2-phenylquinolin-7-yl)-1-(3-hydroxy-3-methylcyclobutyl)-1H-pyrazole-4-carbonitrile (20 mg, 0.045 mmol, 1.0 eq) and K₂CO₃ (18.6 mg, 0.14 mmol, 3.0 eq) in DMSO (20 mL) at rt was added H₂O₂ (30%, 0.1 mL, 0.9 mmol, 20.0 eq). After addition was complete, the reaction mixture was stirred at rt for 15 h. The mixture was diluted with water (50 mL) and extracted with EtOAc (50 mL×2). The combined organic layers were washed with brine (50 mL), dried with anhydrous sodium sulfate, and concentrated under vacuum. The resulting residue was purified by Prep-HPLC to afford to afford 5-amino-3-(6-fluoro-4-methoxy-2-phenylquinolin-7-yl)-1-((1s,3s)-3-hydroxy-3-methylcyclobutyl)-1H-pyrazole-4-carboxamide (2.2 mg, 11%) and 5-amino-3-(6-fluoro-4-methoxy-2-phenylquinolin-7-yl)-1-((1r,3r)-3-hydroxy-3-methylcyclobutyl)-1H-pyrazole-4-carboxamide (4.2 mg, 12%) as a white solid.

5-amino-3-(6-fluoro-4-methoxy-2-phenylquinolin-7-yl)-1-((1s,3s)-3-hydroxy-3-methylcyclobutyl)-1H-pyrazole-4-carboxamide: LRMS (M+H⁺) m/z calculated 462.2, found 462.1. ¹H NMR (DMSO-d₆, 400 MHz) δ 8.31 (d, 2H), 8.07 (d, 1H), 7.82 (d, 1H), 7.63 (s, 1H), 7.51-7.59 (m, 3H), 6.28 (s, 2H), 4.44-4.49 (m, 1H), 4.20 (s, 3H), 2.59-2.64 (m, 2H), 2.36-2.41 (m, 2H), 1.34 (s, 3H). ¹⁹F NMR (DMSO-d₆, 377 MHz) δ −115.5 (s, 1F).

5-amino-3-(6-fluoro-4-methoxy-2-phenylquinolin-7-yl)-1-((1r,3r)-3-hydroxy-3-methylcyclobutyl)-1H-pyrazole-4-carboxamide: LRMS (M+H⁺) m/z calculated 462.2, found 462.1. ¹H NMR (DMSO-d₆, 400 MHz) δ 8.31 (d, 2H), 8.07 (d, 1H), 7.82 (d, 1H), 7.63 (s, 1H), 7.51-7.59 (m, 3H), 6.25 (s, 2H), 4.92-4.97 (m, 1H), 4.20 (s, 3H), 2.38-2.51 (m, 4H), 1.33 (s, 3H). ¹⁹F NMR (DMSO-d₆, 377 MHz) δ −115.5 (s, 1F).

### Example 53: Preparation of 5-amino-3-(4-(difluoromethoxy)-2-phenylquinolin-7-yl)-1-((1s,3s)-3-hydroxy-3-methylcyclobutyl)-1H-pyrazole-4-carboxamide

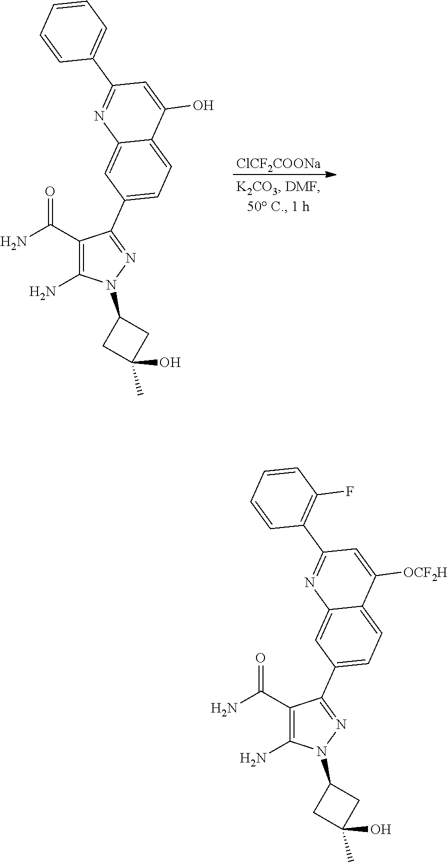

To a solution of 5-amino-3-(4-hydroxy-2-phenylquinolin-7-yl)-1-((1s,3s)-3-hydroxy-3-methylcyclobutyl)-1H-pyrazole-4-carboxamide (40.0 mg, 0.09 mmol, 1.0 eq) in DMF (10 mL) were added K₂CO₃ (25.6 mg, 0.18 mmol, 2.0 eq) and sodium 2-chloro-2,2-difluoroacetate (21.6 mg, 0.14 mmol, 1.5 eq). The mixture was stirred at 50° C. for 1 h. The reaction mixture was diluted with water (50 mL) and extracted with EtOAc (50 mL×3). The combined organic layers were washed with brine (30 mL×2) and concentrated under vacuum. The resulting residue was purified by Prep-HPLC to afford 5-amino-3-(4-(difluoromethoxy)-2-phenylquinolin-7-yl)-1-((1s,3s)-3-hydroxy-3-methylcyclobutyl)-1H-pyrazole-4-carboxamide (1.8 mg, 5%). LRMS (M+H⁺) m/z calculated 480.2, found 480.1. ¹H NMR (400 MHz, DMSO) δ 8.29.8.32 (m, 2H), 8.24 (d, 1H), 8.16 (d, 1H), 7.84-7.90 (m, 2H), 7.54-7.93 (m, 3H), 6.28 (s, 2H), 5.21 (s, 1H), 4.44-4.49 (m, 1H), 2.57-2.68 (m, 2H), 2.32-2.41 (m, 2H), 1.34 (s, 3H).

### Example 54: Preparation of 5-amino-1-((1s,3s)-3-hydroxy-3-methylcyclobutyl)-3-(2-phenylquinazolin-7-yl)-1H-pyrazole-4-carboxamide

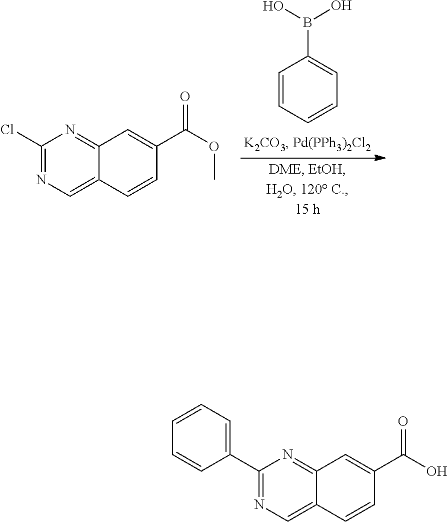 [SMILES: COC(=O)c1ccc2cnc(Cl)nc2c1 ; OB(O)c1ccccc1 ; O=C(O)c1ccc2cnc(-c3ccccc3)nc2c1]

To a solution of methyl 2-chloroquinazoline-7-carboxylate (1.0 g, 4.5 mmol, 1.0 eq) in DME/EtOH/H₂O (10 mL/10 mL/10 mL) were added phenylboronic acid (823.5 mg, 6.8 mmol, 1.5 eq), Pd(PPh₃)₂Cl₂ (315.9 mg, 0.45 mmol, 0.1 eq) and K₂CO₃ (1.8 g, 13.5 mmol, 3.0 eq). The mixture was stirred at 120° C. for 15 h. The mixture was diluted with water (50 mL) and extracted by EtOAc (50 mL×3). The combined organic layers were washed with brine (100 mL), dried over anhydrous sodium sulfate, filtered and concentrated under vacuum. The resulting residue was purified by column chromatography on silica gel (DCM/MeOH=10/1, v/v) to afford 2-phenylquinazoline-7-carboxylic acid (500 mg, 44.2%) as a yellow solid. LRMS (M+H⁺) m/z calculated 251.1, found 251.1.

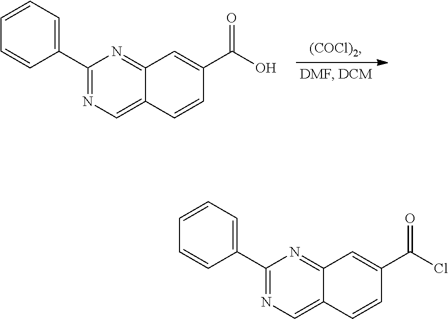 [SMILES: O=C(O)c1ccc2cnc(-c3ccccc3)nc2c1 ; O=C(Cl)c1ccc2cnc(-c3ccccc3)nc2c1]

To a solution of 2-phenylquinazoline-7-carboxylic acid (500 mg, 2.0 mmol, 1.0 eq) in DCM (10 mL) was added (COCl)₂ (0.9 mL, 10.0 mmol, 5.0 eq) and DMF (2 drops) at −78° C. The mixture was stirred at rt for 2 h, then concentrated under vacuum to afford 2-phenylquinazoline-7-carbonyl chloride as a yellow solid (500 mg, ca 100.0%). LRMS (M+H⁺) m/z calculated 265.1, found 265.1 in MeOH.

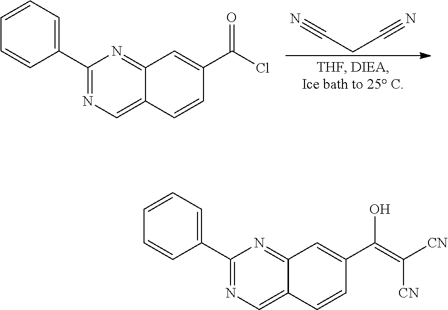 [SMILES: O=C(Cl)c1ccc2cnc(-c3ccccc3)nc2c1 ; N#CCC#N ; N#CC(C#N)=C(O)c1ccc2cnc(-c3ccccc3)nc2c1]

To a solution of 2-phenylquinazoline-7-carbonyl chloride (500 mg, 1.9 mmol, 1.0 eq) in THF (10 mL) were added malononitrile (250.8 mg, 3.8 mmol, 2.0 eq) and DIEA (1.0 mL, 5.7 mmol, 3.0 eq) at ice bath. The mixture was stirred at rt for 2 h, then concentrated and diluted with water (20 mL). The resulting mixture was extracted with EtOAc (30 mL×3). The combined organic layers were washed with brine (50 mL), dried over anhydrous sodium sulfate, filtered and concentrated under vacuum. The resulting residue was purified by column chromatography on silica gel (DCM/MeOH=10/1, v/v) to afford 2-(hydroxy (2-phenylquinazolin-7-yl)methylene)malononitrile as a yellow oil (400 mg, 71.9%). LRMS (M+H⁺) m/z calculated 299.1, found 299.1.

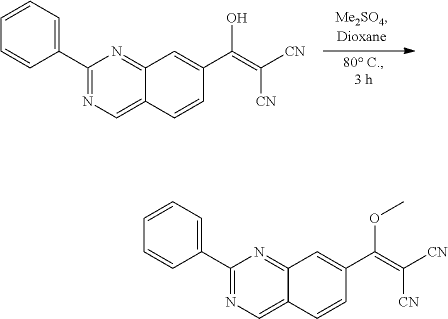 [SMILES: N#CC(C#N)=C(O)c1ccc2cnc(-c3ccccc3)nc2c1 ; COC(=C(C#N)C#N)c1ccc2cnc(-c3ccccc3)nc2c1]

To a solution of 2-(hydroxy (2-phenylquinazolin-7-yl)methylene)malononitrile (400 mg, 1.3 mmol, 1.0 eq) in THF (10 mL) were added Me₂SO₄ (0.3 mL, 2.6 mmol, 2.0 eq) and DIEA (0.5 mL, 2.6 mmol, 2.0 eq) at rt. The mixture was stirred at 80° C. for 3 h, then concentrated under vacuum, diluted with water (20 mL) and extracted with EtOAc (30 mL×2). The combined organic layers were washed with brine (50 mL), dried over anhydrous sodium sulfate, filtered and concentrated under vacuum. The resulting residue was purified by column chromatography on silica gel (PE/EA=1/1, v/v) to afford 2-(methoxy(2-phenylquinazolin-7-yl)methylene)malononitrile as a yellow oil (200 mg, 47.8%). LRMS (M+H⁺) m/z calculated 313.1, found 313.1.

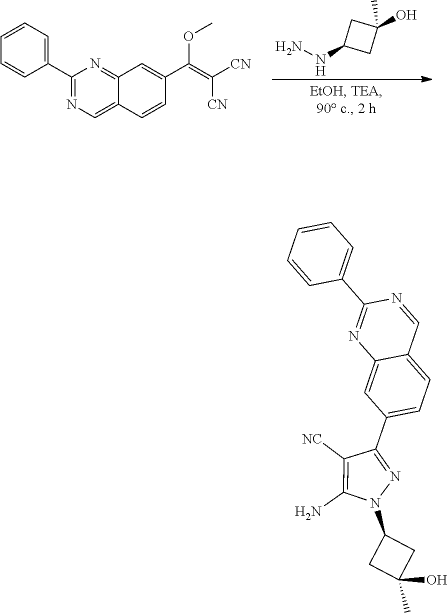

A mixture of 2-(methoxy(2-phenylquinazolin-7-yl)methylene)malononitrile (200 mg, 0.64 mmol, 1.0 eq), (1s,3s)-3-hydrazinyl-1-methylcyclobutan-1-ol (111.2 mg, 0.96 mmol, 1.5 eq) and TEA (0.4 mL, 1.92 mmol, 3.0 eq) in 5.0 mL EtOH was stirred at 80° C. for 2 h. The mixture was concentrated, and the resulting residue was purified by column chromatography on silica gel (DCM/MeOH=10/1, v/v) to afford 5-amino-1-((1s,3s)-3-hydroxy-3-methylcyclobutyl)-3-(2-phenylquinazolin-7-yl)-1H-pyrazole-4-carbonitrile as a yellow oil (150 mg, 59.3%). LRMS (M+H⁺) m/z calculated 397.2, found 397.2.

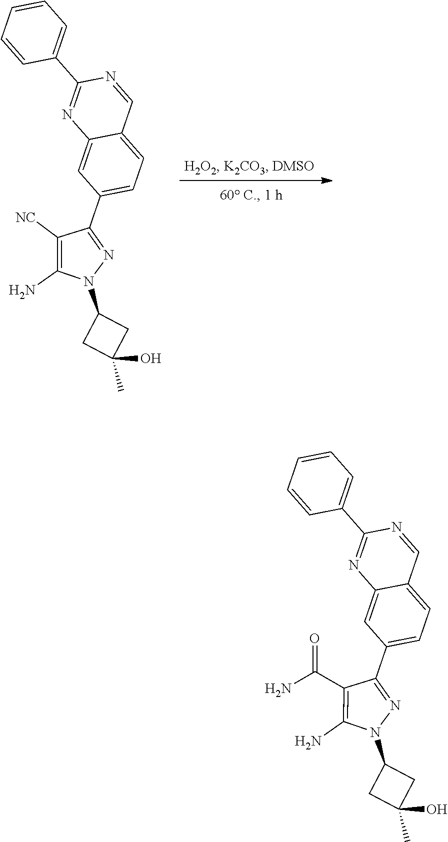 [SMILES: C[C@]1(O)C[C@@H](n2nc(-c3ccc4cnc(-c5ccccc5)nc4c3)c(C#N)c2N)C1 ; C[C@]1(O)C[C@@H](n2nc(-c3ccc4cnc(-c5ccccc5)nc4c3)c(C(N)=O)c2N)C1]

To a solution of 5-amino-1-((1s,3s)-3-hydroxy-3-methylcyclobutyl)-3-(2-phenylquinazolin-7-yl)-1H-pyrazole-4-carbonitrile (50 mg, 0.13 mmol, 1.0 eq) in in DMSO (3.0 mL) were added K₂CO₃ (54 mg, 0.39 mmol, 3.0 eq) and H₂O₂ (1 mL) at rt. After addition was complete, the mixture was stirred at 60° C. for 1 h. Water (20 mL) was added and the mixture was extracted with EtOAc (50 mL×3). The combined organic layers were washed with brine (30 mL), dried with anhydrous sodium sulfate, filtered and concentrated under vacuum. The resulting residue was purified by Prep-HPLC to afford 5-amino-1-((1s,3s)-3-hydroxy-3-methylcyclobutyl)-3-(2-phenylquinazolin-7-yl)-1H-pyrazole-4-carboxamide as a white solid (8.0 mg, 15.4%). LRMS (M+H⁺) m/z calculated 415.2, found 415.2. ¹H NMR (DMSO-d₆, 400 MHz) δ 9.71 (s, 1H), 8.58-8.60 (m, 2H), 8.18-8.20 (d, 2H), 7.92-7.95 (m, 1H), 7.56-7.59 (m, 3H), 6.26 (s, 2H), 5.19 (s, 1H), 4.45-4.49 (m, 1H), 2.59-2.67 (m, 2H), 2.37-2.41 (m, 2H), 1.34 (s, 3H).

### Example 55: Preparation of 5-amino-3-(5-fluoro-4-methoxy-2-phenylquinolin-7-yl)-1-((1s,3s)-3-hydroxy-3-methylcyclobutyl)-1H-pyrazole-4-carboxamide

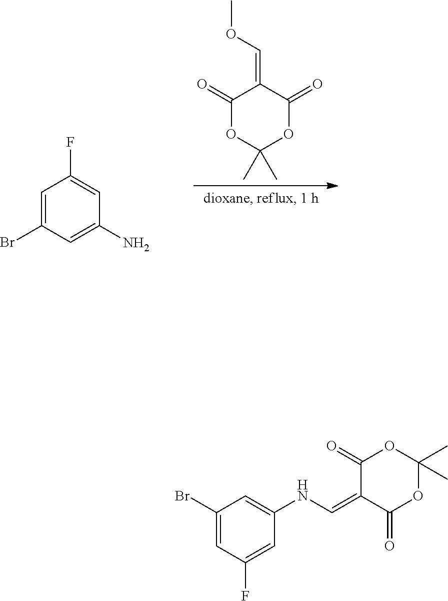 [SMILES: Nc1cc(F)cc(Br)c1 ; COC=C1C(=O)OC(C)(C)OC1=O ; CC1(C)OC(=O)C(=CNc2cc(F)cc(Br)c2)C(=O)O1]

A mixture of 3-bromo-5-fluoroaniline (5.0 g, 26.4 mmol, 1.0 eq) and 5-(methoxymethylene)-2,2-dimethyl-1,3-dioxane-4,6-dione (4.9 g, 26.4 mmol, 1.0 eq) in dioxane (100 mL) was stirred at 120° C. for 1 h. The mixture was cooled to rt and diluted with PE (100 mL). The precipitate was filtered to afford 5-(((3-bromo-5-fluorophenyl)amino)methylene)-2,2-dimethyl-1,3-dioxane-4,6-dione (8.2 g, >100%) as a yellow solid. LRMS (M+H⁺) m/z calculated 344.0, found 286.0, 304.1.

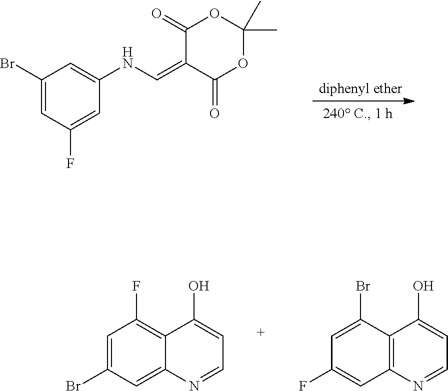 [SMILES: CC1(C)OC(=O)C(=CNc2cc(F)cc(Br)c2)C(=O)O1 ; Oc1ccnc2cc(Br)cc(F)c12 ; Oc1ccnc2cc(F)cc(Br)c12]

A mixture of 5-(((3-bromo-5-fluorophenyl)amino)methylene)-2,2-dimethyl-1,3-dioxane-4,6-dione (8.2 g, 23.9 mmol, 1.0 eq) and Ph₂O (100 mL) was stirred at 240° C. for 1 h. The mixture was cooled to rt and diluted with PE (100 mL). The precipitate was filtered to afford a mixture of 7-bromo-5-fluoroquinolin-4-ol and 5-bromo-7-fluoroquinolin-4-ol (4.5 g, 78%) as a brown solid. LRMS (M+H⁺) m/z calculated 242.0, found 242.1.

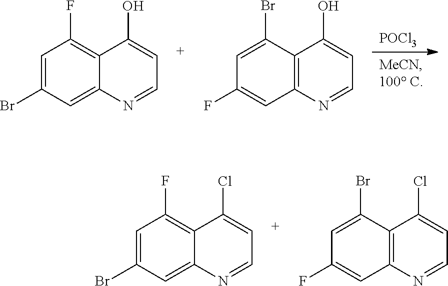

To a solution of 7-bromo-5-fluoroquinolin-4-ol and and 5-bromo-7-fluoroquinolin-4-ol (4.5 g, 18.7 mmol, 1.0 eq) in Tol (120 mL) were added POCl₃ (5.7 g, 37.3 mmol, 2 eq) The mixture was stirred at 100° C. for 1 h, then poured into ice, adjusted to pH 13 with saturated Na₂CO₃ aqueous solution, extracted with DCM (500 mL×3). The combined organic layers were dried over anhydrous sodium sulfate, filtered and concentrated under vacuum. The resulting residue was purified by column chromatography on silica gel (PE/EA=5/1, v/v) to afford a mixture of 7-bromo-4-chloro-5-fluoroquinoline and 5-bromo-4-chloro-7-fluoroquinoline (3.9 g, 79%) as a yellow solid, LRMS (M+H⁺) m/z calculated 259.9, found 260.0

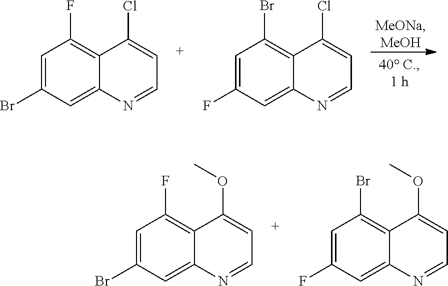

A mixture of 7-bromo-4-chloro-5-fluoroquinoline and 5-bromo-4-chloro-7-fluoroquinoline (3.1 g, 12 mmol, 1.0 eq) and MeONa (970 mg, 18 mmol, 1.5 eq) in MeOH (50 mL) was stirred at 40° C. for 1 h. LCMS showed the reaction was completed. The reaction was quenched by H₂O (50 mL) and extracted by EtOAc (50 mL×3), the combined organic layers were dried over Na₂SO₄, filtered, and concentrated under vacuum. The resulting residue was purified by by column chromatography on silica gel (PE/EtOAc=10/1, v/v) to afford a mixture of 7-bromo-5-fluoro-4-methoxyquinoline and 5-bromo-7-fluoro-4-methoxyquinoline as a yellow oil (2.4 g, 80%). LRMS (M+H⁺) m/z calculated 256.0, found 256.0.

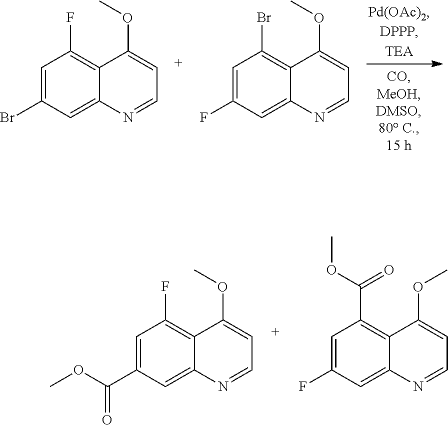 [SMILES: COc1ccnc2cc(Br)cc(F)c12 ; COc1ccnc2cc(F)cc(Br)c12 ; "Pd(OAc)2,DPPP,TEA" ; COC(=O)c1cc(F)c2c(OC)ccnc2c1 ; COC(=O)c1cc(F)cc2nccc(OC)c12]

To a solution of 7-bromo-5-fluoro-4-methoxyquinoline and 5-bromo-7-fluoro-4-methoxyquinoline (2.4 g, 9.4 mmol, 1.0 eq), DPPP (780 mg, 1.8 mmol, 0.2 eq) and Pd(OAc)₂ (210.6 mg, 0.94 mmol, 0.1 eq) in DMSO/MeOH (20 mL/20 mL) was added TEA (3.8 mL, 28.2 mmol. 3.0 eq). The mixture was stirred at 80° C. for 15 h under CO (5 atm), and concentrated under vacuum. The resulting residue was purified by column chromatography on silica gel (PE/EA=10/1, v/v) to afford a mixture of methyl 5-fluoro-4-methoxyquinoline-7-carboxylate and methyl 7-fluoro-4-methoxyquinoline-5-carboxylate (1.8 g, 72%) as a yellow oil. LRMS (M+H⁺) m/z calculated 236.1, found 236.1.

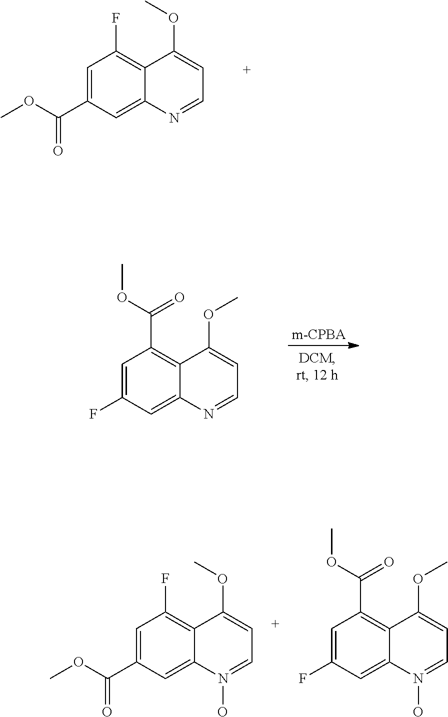

To a stirred solution of methyl 5-fluoro-4-methoxyquinoline-7-carboxylate and methyl 7-fluoro-4-methoxyquinoline-5-carboxylate (1.8 g, 7.7 mmol, 1.0 eq) in DCM (20 mL) was added m-CPBA (1.9 g, 11.5 mmol, 1.5 eq) at rt. The mixture was stirred at rt for 12 h, then poured into ice, adjusted to pH 13 with addition of saturated Na₂CO₃ aqueous solution, and extracted with DCM (30 mL×3). The combined organic layers were dried over sodium sulfate and concentrated under vacuum to afford a mixture of 5-fluoro-4-methoxy-7-(methoxycarbonyl) quinoline 1-oxide and 7-fluoro-4-methoxy-5-(methoxycarbonyl) quinoline 1-oxide (1.5 g, 78.9%) as a yellow oil. LRMS (M+H⁺) m/z calculated 252.1, found 252.1.

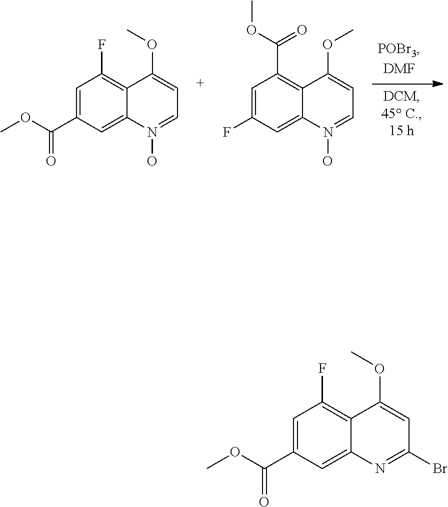

To a solution of 5-fluoro-4-methoxy-7-(methoxycarbonyl) quinoline 1-oxide and 7-fluoro-4-methoxy-5-(methoxycarbonyl) quinoline 1-oxide (1.5 g, 5.9 mmol, 1.0 eq) in DCM (20 mL) were added POBr₃ (2.2 g, 7.8 mmol, 1.3 eq) and DMF (2.3 mL, 2.9 mmol, 0.5 eq) at −78° C. The mixture was stirred at 45° C. for 15 h, then poured into ice, adjusted to pH 13 with addition of saturated Na₂CO₃ aqueous solution, and extracted with DCM (30 mL×3). The combined organic layers were dried over anhydrous sodium sulfate, filtered and concentrated under vacuum. The resulting residue was purified by column chromatography on silica gel (PE/EA=5/1, v/v) to afford methyl 2-bromo-5-fluoro-4-methoxyquinoline-7-carboxylate (600 mg, 31.6%) as a yellow solid. LRMS (M+H⁺) m/z calculated 314.0, found 314.0. ¹H NMR (DMSO-d₆, 400 MHz) δ 8.16 (s, 1H), 7.66 (dd, 1H), 7.37 (s, 1H), 4.07 (s, 3H), 3.94 (s, 3H).

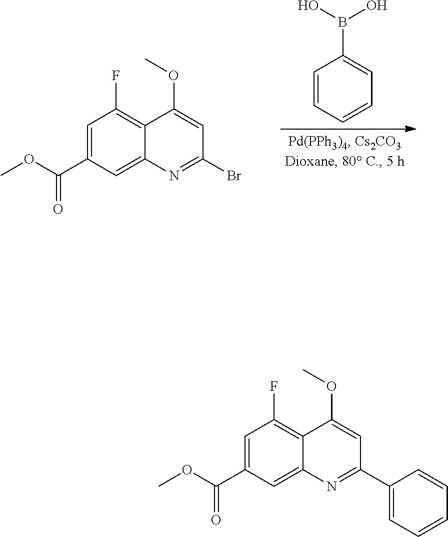 [SMILES: COC(=O)c1cc(F)c2c(OC)cc(Br)nc2c1 ; OB(O)c1ccccc1 ; COC(=O)c1cc(F)c2c(OC)cc(-c3ccccc3)nc2c1]

To a solution of methyl 2-bromo-5-fluoro-4-methoxyquinoline-7-carboxylate (600 mg, 1.9 mmol, 1.0 eq) in dioxane (10 mL) were added phenylboronic acid (467.7 mg, 3.8 mmol, 2.00 eq), Pd(PPh₃)₄ (219 mg, 0.19 mmol, 0.1 eq) and Cs₂CO₃ (1.8 g, 5.7 mmol, 3.0 eq). The mixture was stirred at 80° C. for 5 h, then concentrated under vacuum and diluted with water (100 mL), and extracted with EtOAc (150 mL×2). The combined organic layers were washed with brine (150 mL), dried over anhydrous sodium sulfate, filtered and concentrated under vacuum. The resulting residue was purified by column chromatography on silica gel (PE/EA=5/1, v/v) to afford methyl 5-fluoro-4-methoxy-2-phenylquinoline-7-carboxylate (410 mg, 68.3%) as a white solid. LRMS (M+H⁺) m/z calculated 312.1, found 312.1.

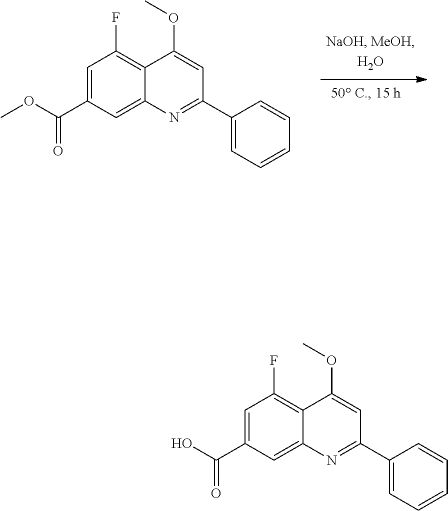 [SMILES: COC(=O)c1cc(F)c2c(OC)cc(-c3ccccc3)nc2c1 ; COc1cc(-c2ccccc2)nc2cc(C(=O)O)cc(F)c12]

To a solution of methyl 5-fluoro-4-methoxy-2-phenylquinoline-7-carboxylate (410 mg, 1.3 mmol, 1.0 eq) in MeOH (6.0 mL) and H₂O (2.0 mL) was added NaOH (80 mg, 2.0 mmol, 1.5 eq). The mixture was stirred at 50° C. for 15 h, then concentrated under vacuum and diluted with water (50 mL). 37% HCl was added to adjust to pH 2. The resulting mixture was stirred for 5 min, filtered and concentrated under vacuum to afford 5-fluoro-4-methoxy-2-phenylquinoline-7-carboxylic acid (350 mg, 89.7%) as a white solid. LRMS (M+H⁺) m/z calculated 298.1, found 298.1.

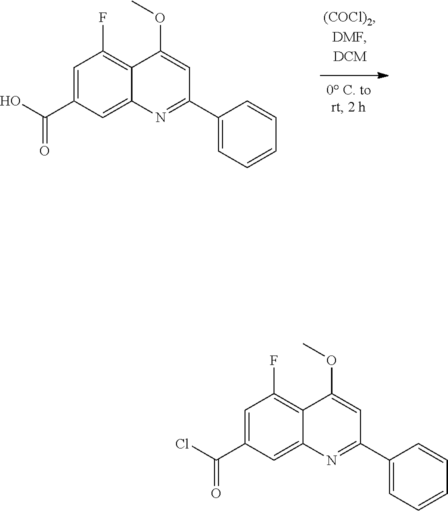 [SMILES: COc1cc(-c2ccccc2)nc2cc(C(=O)O)cc(F)c12 ; COc1cc(-c2ccccc2)nc2cc(C(=O)Cl)cc(F)c12]

To a solution of 5-fluoro-4-methoxy-2-phenylquinoline-7-carboxylic acid (350 mg, 1.2 mmol, 1.0 eq) in DCM (10 mL) was added (COCl)₂ (0.6 mL, 6.0 mmol, 5.0 eq) and DMF (2 drops) at 0° C. The mixture was stirred at rt for 2 h, then concentrated under vacuum to afford 5-fluoro-4-methoxy-2-phenylquinoline-7-carbonyl chloride as a yellow solid (340 mg, 91.9%). LRMS (M+H⁺) m/z calculated 312.1, found 312.1 in MeOH.

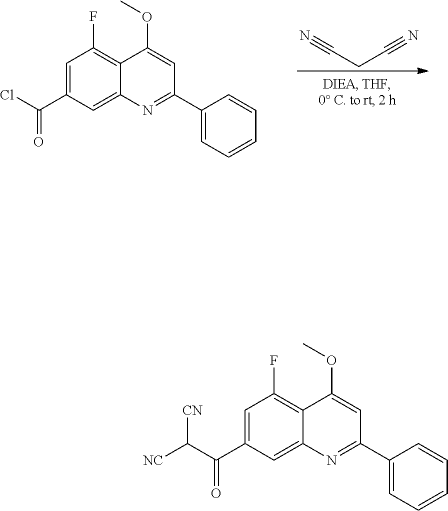 [SMILES: COc1cc(-c2ccccc2)nc2cc(C(=O)Cl)cc(F)c12 ; N#CCC#N ; COc1cc(-c2ccccc2)nc2cc(C(=O)C(C#N)C#N)cc(F)c12]

To a solution of 5-fluoro-4-methoxy-2-phenylquinoline-7-carbonyl chloride (340 mg, 1.1 mmol, 1.0 eq) in THF (10 mL) were added malononitrile (145.2 mg, 2.2 mmol, 2.0 eq) and DIEA (0.6 mL, 3.3 mmol, 3.0 eq) at ice bath. The mixture was stirred at rt for 2 h, then concentrated and diluted with water (20 mL). The resulting mixture was extracted with EtOAc (30 mL×3). The combined organic layers were washed with brine (40 mL), dried over anhydrous sodium sulfate, filtered and concentrated under vacuum. The resulting residue was purified by column chromatography on silica gel (DCM/MeOH=10/1, v/v) to afford 2-(5-fluoro-4-methoxy-2-phenylquinoline-7-carbonyl)malononitrile as a yellow oil (350 mg, 94.1%). LRMS (M+H⁺) m/z calculated 346.1, found 346.1.

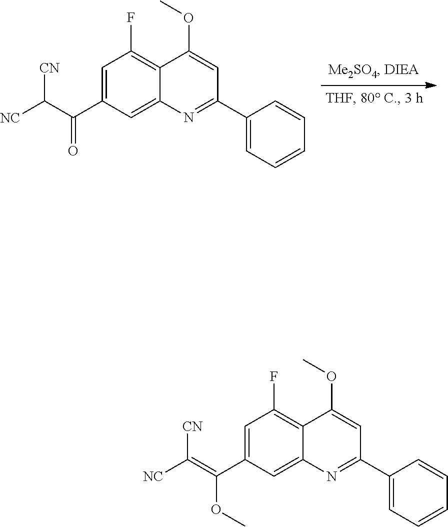 [SMILES: COc1cc(-c2ccccc2)nc2cc(C(=O)C(C#N)C#N)cc(F)c12 ; COC(=C(C#N)C#N)c1cc(F)c2c(OC)cc(-c3ccccc3)nc2c1]

To a solution of 2-(5-fluoro-4-methoxy-2-phenylquinoline-7-carbonyl)malononitrile (350 mg, 1.0 mmol, 1.0 eq) in THF (10 mL) were added Me₂SO₄ (0.2 mL, 2.0 mmol, 2.0 eq) and DIEA (0.5 mL, 2.0 mmol, 2.0 eq) at rt. The mixture was stirred at 80° C. for 3 h, then concentrated under vacuum, diluted with water (20 mL), and extracted with EtOAc (30 mL×2). The combined organic layers were washed with brine (50 mL), dried over anhydrous sodium sulfate, filtered, and concentrated under vacuum. The resulting residue was purified by column chromatography on silica gel (PE/EA=5/1, v/v) to afford 2-((5-fluoro-4-methoxy-2-phenylquinolin-7-yl)(methoxy)methylene)malononitrile as a yellow oil (130 mg, 36.1%). LRMS (M+H⁺) m/z calculated 360.1, found 360.1.

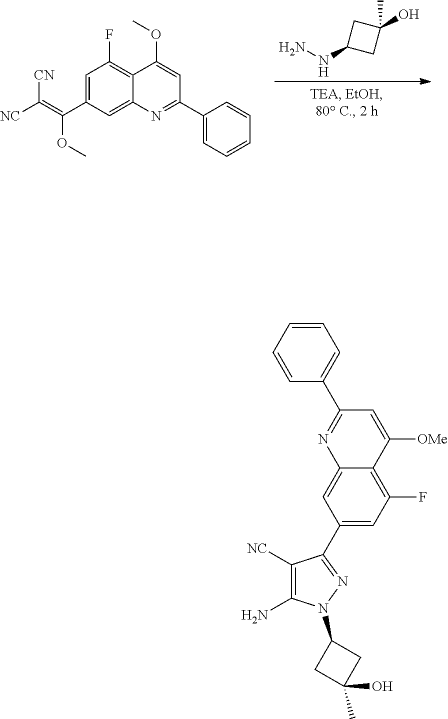

To a solution of 2-((5-fluoro-4-methoxy-2-phenylquinolin-7-yl)(methoxy)methylene)malononitrile (130 mg, 0.36 mmol, 1.0 eq) in EtOH (5.0 mL) were added (1s,3s)-3-hydrazinyl-1-methylcyclobutan-1-ol (63 mg, 0.54 mmol, 1.5 eq) and TEA (1.4 mL, 10.1 mmol, 3.0 eq). The reaction mixture was stirred at 80° C. for 2 h, then concentrated under vacuum, and purified by column chromatography on silica gel (DCM/MeOH=10/1, v/v) to afford 5-amino-3-(5-fluoro-4-methoxy-2-phenylquinolin-7-yl)-1-((1s,3s)-3-hydroxy-3-methylcyclobutyl)-1H-pyrazole-4-carbonitrile as a yellow oil (100 mg, 62.5%). LRMS (M+H⁺) m/z calculated 444.2, found 444.2.

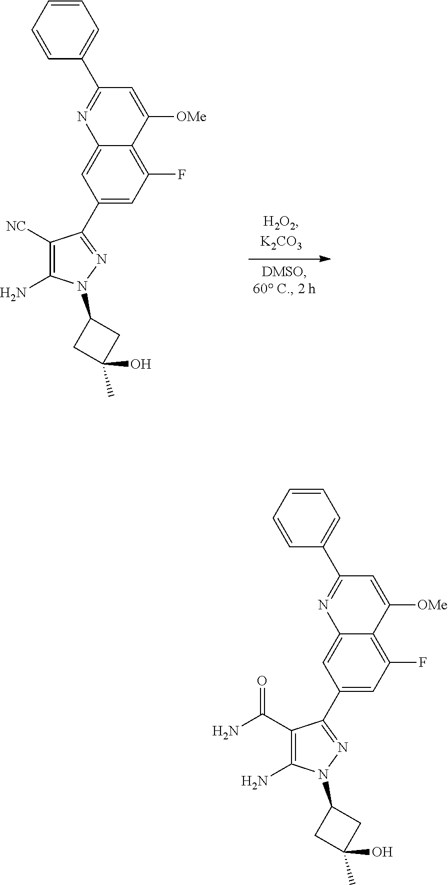 [SMILES: COc1cc(-c2ccccc2)nc2cc(-c3nn([C@H]4C[C@@](C)(O)C4)c(N)c3C#N)cc(F)c12 ; COc1cc(-c2ccccc2)nc2cc(-c3nn([C@H]4C[C@@](C)(O)C4)c(N)c3C(N)=O)cc(F)c12]

To a stirred solution of 5-amino-3-(5-fluoro-4-methoxy-2-phenylquinolin-7-yl)-1-((1s,3s)-3-hydroxy-3-methylcyclobutyl)-1H-pyrazole-4-carbonitrile (100 mg, 0.22 mmol, 1.0 eq) in DMSO (3.0 mL) were added K₂CO₃ (93.4 mg, 0.66 mmol, 3.0 eq) and H₂O₂ (2.0 mL) at rt. After addition was complete, the mixture was stirred at 60° C. for 2 h. Water (20 mL) was added, and the mixture was extracted with EtOAc (50 mL×3). The combined organic layers were washed with brine (30 mL), dried with anhydrous sodium sulfate, filtered and concentrated under vacuum. The resulting residue was purified by Prep-HPLC to afford 5-amino-3-(5-fluoro-4-methoxy-2-phenylquinolin-7-yl)-1-((1s,3s)-3-hydroxy-3-methylcyclobutyl)-1H-pyrazole-4-carboxamide as a white solid (76.0 mg, 73.1%). LRMS (M+H⁺) m/z calculated 462.2, found 462.2. ¹H NMR (DMSO-d₆, 400 MHz) δ 8.30-8.32 (m, 2H), 8.00-8.01 (d, 1H), 7.52-7.59 (m, 4H), 7.42-7.45 (d, 1H), 6.25 (s, 2H), 5.20 (s, 1H), 4.50-4.59 (m, 1H), 4.16 (s, 3H), 2.58-2.63 (m, 2H), 2.35-2.40 (m, 2H), 1.33 (s, 3H).

### Example 56: Preparation of 5-amino-3-(8-fluoro-2-(2-fluorophenyl) quinolin-7-yl)-1-((1s,3s)-3-hydroxy-3-methylcyclobutyl)-1H-pyrazole-4-carboxamide

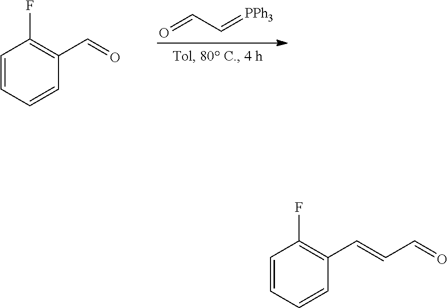

A mixture of 2-fluorobenzaldehyde (5.0 g, 40.3 mmol, 1.0 eq) and 2-(triphenyl-phosphanylidene) acetaldehyde (12.3 g, 40.3 mmol, 1.0 eq) in toluene (50.0 mL) was stirred at 80° C. for 4 h. The mixture was concentrated under vacuum, and the resulting residue was purified by column chromatography on silica gel (DCM/MeOH=10/1, v/v) to afford (E)-3-(2-fluorophenyl) acrylaldehyde as a yellow oil (3.9 g, 65%).

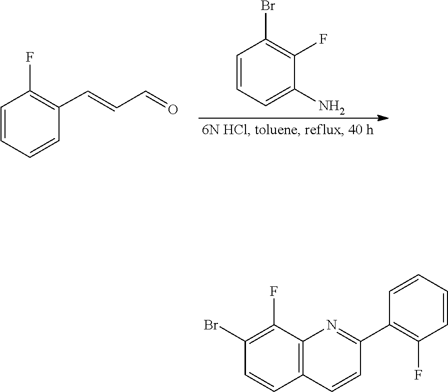 [SMILES: O=C/C=C/c1ccccc1F ; Nc1cccc(Br)c1F ; Fc1ccccc1-c1ccc2ccc(Br)c(F)c2n1]

A mixture of (E)-3-(2-fluorophenyl) acrylaldehyde (3.9 g, 26 mmol, 1.0 eq), 3-bromo-2-fluoroaniline (4.9 g, 26 mmol, 1.0 eq) in toluene (40.0 mL) and 6N HCl (40 mL) was heated to reflux for 40 h. The mixture was concentrated and the residue was diluted by H₂O (50 mL). The mixture was adjusted to pH 13 with sat. NaHCO₃, and extracted with EtOAc (50 mL×3). The combined organic layers were washed with brine (50 mL), dried over anhydrous sodium sulfate, filtered and concentrated under vacuum. The resulting residue was purified by column chromatography on silica gel (PE/EA=5/1, v/v) to afford 7-bromo-8-fluoro-2-(2-fluorophenyl) quinoline as a yellow oil (3.1 g, 37.3%). LRMS (M+H⁺) m/z calculated 320.0, found 320.0.

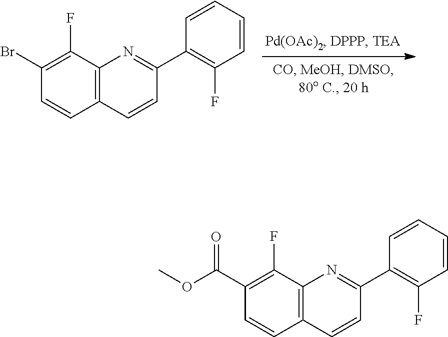 [SMILES: Fc1ccccc1-c1ccc2ccc(Br)c(F)c2n1 ; COC(=O)c1ccc2ccc(-c3ccccc3F)nc2c1F]

To a solution of 7-bromo-8-fluoro-2-(2-fluorophenyl) quinoline (3.1 g, 9.7 mmol, 1.0 eq), DPPF (799 mg, 1.9 mmol, 0.2 eq) and Pd(OAc)₂ (217.6 mg, 0.97 mmol, 0.1 eq) in DMSO/MeOH (30 mL/30 mL) was added TEA (3.9 mL, 29.1 mmol. 3.0 eq). The mixture was stirred at 80° C. for 20 h under CO (5 atm), and concentrated under vacuum. The resulting residue was purified by column chromatography on silica gel (PE/EA-10/1, v/v) to afford methyl 8-fluoro-2-(2-fluorophenyl) quinoline-7-carboxylate (960 mg, 33.1%) as a yellow solid. LRMS (M+H⁺) m/z calculated 300.1, found 300.1

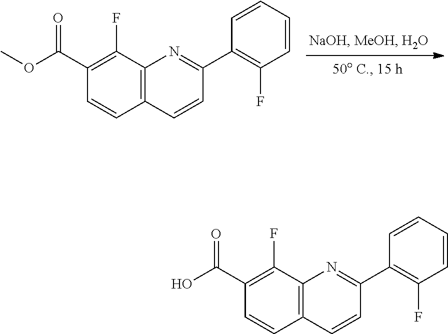 [SMILES: COC(=O)c1ccc2ccc(-c3ccccc3F)nc2c1F ; O=C(O)c1ccc2ccc(-c3ccccc3F)nc2c1F]

To a solution of methyl 8-fluoro-2-(2-fluorophenyl) quinoline-7-carboxylate (960 mg, 3.2 mmol, 1.0 eq) in MeOH (6.0 mL) and H₂O (2.0 mL) was added NaOH (192.6 mg, 4.8 mmol, 1.5 eq). The mixture was stirred at 50° C. for 15 h, then concentrated under vacuum and diluted with water (50 mL). 37% HCl was added to adjust to pH 2. The resulting mixture was stirred for 5 min, filtered and concentrated under vacuum to afford 8-fluoro-2-(2-fluorophenyl) quinoline-7-carboxylic acid (800 mg, 87.4%) as a white solid. LRMS (M+H⁺) m/z calculated 286.1, found 286.1.

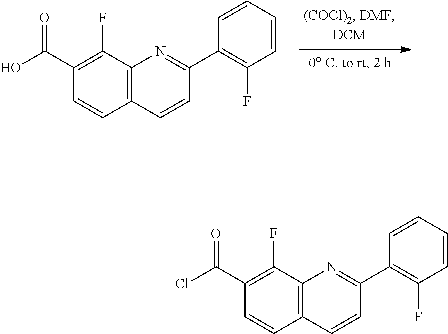 [SMILES: O=C(O)c1ccc2ccc(-c3ccccc3F)nc2c1F ; O=C(Cl)c1ccc2ccc(-c3ccccc3F)nc2c1F]

To a solution of 8-fluoro-2-(2-fluorophenyl) quinoline-7-carboxylic acid (800 mg, 2.8 mmol, 1.0 eq) in DCM (10 mL) was added (COCl)₂ (1.4 mL, 14 mmol, 5.0 eq) and DMF (2 drops) at 0° C. The mixture was stirred at rt for 2 h, and concentrated under vacuum to afford 8-fluoro-2-(2-fluorophenyl) quinoline-7-carbonyl chloride as a yellow solid (800 mg, 94.1%). LRMS (M+H⁺) m/z calculated 300.1, found 300.1 in MeOH.

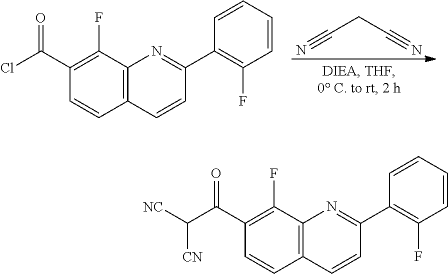

To a solution of 8-fluoro-2-(2-fluorophenyl) quinoline-7-carbonyl chloride (800 mg, 2.6 mmol, 1.0 eq) in THF (10 mL) were added malononitrile (348.5 mg, 5.2 mmol, 2.0 eq) and DIEA (1.4 mL, 7.8 mmol, 3.0 eq) at ice bath. The mixture was stirred at rt for 2 h, then concentrated and diluted with water (20 mL). The resulting mixture was extracted with EtOAc (30 mL×3). The combined organic layers were washed with brine (40 mL), dried over anhydrous sodium sulfate, filtered and concentrated under vacuum. The resulting residue was purified by column chromatography on silica gel (DCM/MeOH=10/1, v/v) to afford 2-(8-fluoro-2-(2-fluorophenyl) quinoline-7-carbonyl)malononitrile as a yellow oil (300 mg, 34.1%). LRMS (M+H⁺) m/z calculated 334.1, found 334.1

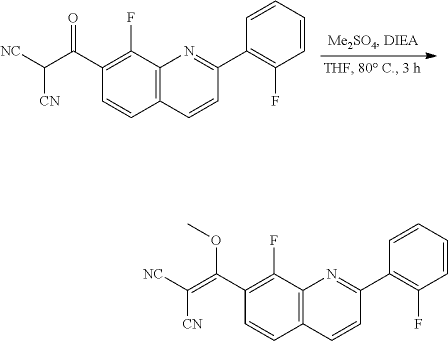 [SMILES: N#CC(C#N)C(=O)c1ccc2ccc(-c3ccccc3F)nc2c1F ; COC(=C(C#N)C#N)c1ccc2ccc(-c3ccccc3F)nc2c1F]

To a solution of 2-(8-fluoro-2-(2-fluorophenyl) quinoline-7-carbonyl)malononitrile (300 mg, 0.9 mmol, 1.0 eq) in THF (10 mL) were added Me₂SO₄ (0.2 mL, 1.8 mmol, 2.0 eq) and DIEA (0.5 mL, 1.8 mmol, 2.0 eq) at rt. The mixture was stirred at 80° C. for 3 h, then concentrated under vacuum, diluted with water (20 mL) and extracted with EtOAc (30 mL×2). The combined organic layers were washed with brine (30 mL), dried over anhydrous sodium sulfate, filtered and concentrated under vacuum. The resulting residue was purified by column chromatography on silica gel (PE/EA=5/1, v/v) to afford 2-((8-fluoro-2-(2-fluorophenyl) quinolin-7-yl)(methoxy)methylene)malononitrile as a yellow oil (90 mg, 28.8%). LRMS (M+H⁺) m/z calculated 348.1, found 348.1.

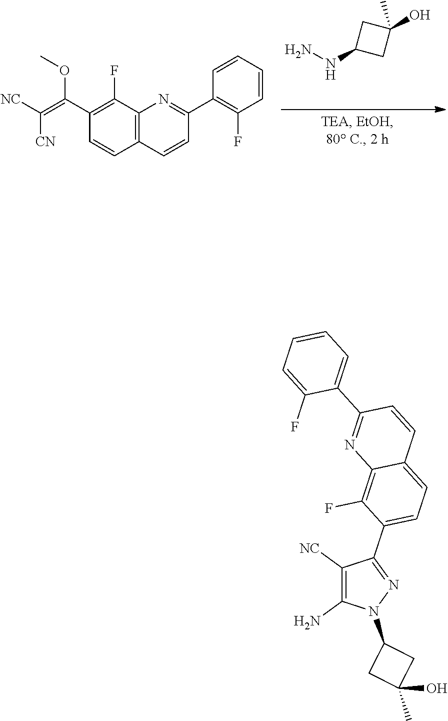

To a solution of 2-((8-fluoro-2-(2-fluorophenyl) quinolin-7-yl)(methoxy)methylene)malononitrile (90 mg, 0.26 mmol, 1.0 eq) in EtOH (5.0 mL) were added (1s,3s)-3-hydrazinyl-1-methylcyclobutan-1-ol (64 mg, 0.39 mmol, 1.5 eq) and TEA (0.1 mL, 0.78 mmol, 3.0 eq). The reaction mixture was stirred at 80° C. for 2 h, then concentrated under vacuum, and purified by column chromatography on silica gel (DCM/MeOH=10/1, v/v) to afford 5-amino-3-(8-fluoro-2-(2-fluorophenyl) quinolin-7-yl)-1-((1s,3s)-3-hydroxy-3-methylcyclobutyl)-1H-pyrazole-4-carbonitrile as a yellow oil (100 mg, 90.1%). LRMS (M+H⁺) m/z calculated 432.2, found 432.2.

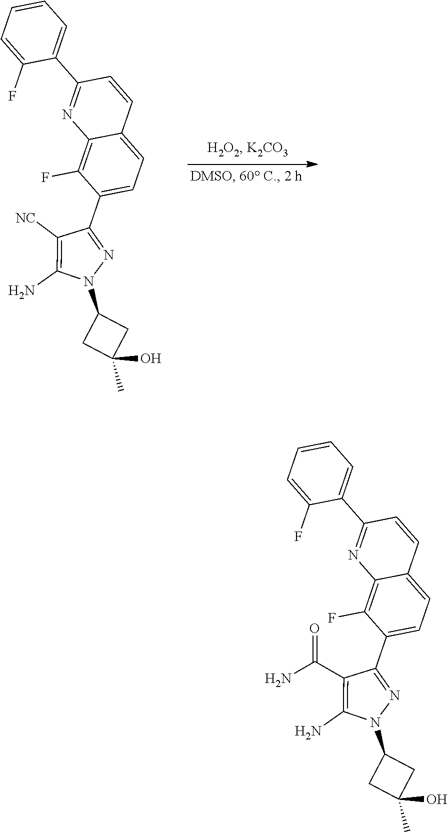 [SMILES: C[C@]1(O)C[C@@H](n2nc(-c3ccc4ccc(-c5ccccc5F)nc4c3F)c(C#N)c2N)C1 ; C[C@]1(O)C[C@@H](n2nc(-c3ccc4ccc(-c5ccccc5F)nc4c3F)c(C(N)=O)c2N)C1]

To a solution of 5-amino-3-(8-fluoro-2-(2-fluorophenyl) quinolin-7-yl)-1-((1s,3s)-3-hydroxy-3-methylcyclobutyl)-1H-pyrazole-4-carbonitrile (100 mg, 0.23 mmol, 1.0 eq) in DMSO (3.0 mL) were added K₂CO₃ (94.2 mg, 0.69 mmol, 3.0 eq) and H₂O₂ (2.0 mL). After addition was complete, the mixture was stirred at 60° C. for 2 h. The reaction was diluted water (50 mL) and extracted with EtOAc (30 mL×3). The combined organic layers were washed with brine (30 mL), dried over anhydrous sodium sulfate, filtered and concentrated under vacuum. The resulting residue was purified by Prep-HPLC to afford 5-amino-3-(8-fluoro-2-(2-fluorophenyl) quinolin-7-yl)-1-((1s,3s)-3-hydroxy-3-methylcyclobutyl)-1H-pyrazole-4-carboxamide as a white solid (13.4 mg, 12.9%). LRMS (M+H⁺) m/z calculated 450.2, found 450.2. ¹H NMR (DMSO-d₆, 400 MHz) δ 8.57-8.59 (d, 1H), 8.02-8.09 (m, 2H), 7.90-7.92 (d, 1H), 7.44-7.67 (m, 2H), 7.39-7.41 (m, 2H), 6.31 (s, 2H), 5.18 (s, 1H), 4.45-4.49 (m, 1H), 2.50-2.60 (m, 2H), 2.36-2.40 (m, 2H), 1.33 (s, 3H).

### Example 57: Preparation of 5-amino-3-(8-fluoro-2-(pyridin-2-yl) quinolin-7-yl)-1-((1s,3s)-3-hydroxy-3-methylcyclobutyl)-1H-pyrazole-4-carboxamide

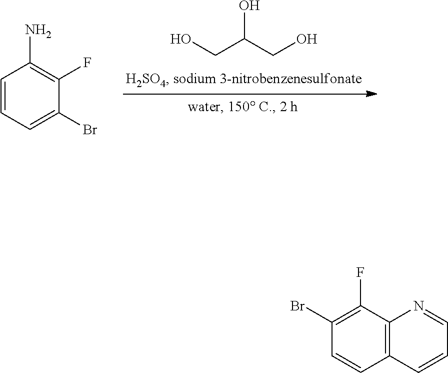 [SMILES: Nc1cccc(Br)c1F ; OCC(O)CO ; Fc1c(Br)ccc2cccnc12]

To a solution of 3-bromo-2-fluoroaniline (50.0 g, 0.26 mol, 1.0 eq), sodium 3-nitrobenzenesulfonate (105.3 g, 0.47 mol, 1.8 eq) and propane-1,2,3-triol (66.9 g, 0.73 mol, 2.8 eq) in H₂O (56.0 mL) was added Con. H₂SO₄ (105.5 mL). The mixture was stirred at 150° C. for 2 h. The mixture was poured to ice water and acidified to pH 14 with 5N aq sodium hydroxide. The mixture was extracted with EtOAc (300 mL×3). The combined organic layers were washed with brine (300 mL), dried over anhydrous sodium sulfate, filtered and concentrated under vacuum. The resulting residue was purified by by column chromatography on silica gel (PE/EtOAc=5/1, v/v) to afford 7-bromo-8-fluoroquinoline (26.0 g, 43.3%) as a yellow solid. LRMS (M+H⁺) m/z calculated 226.0, found 226.0.

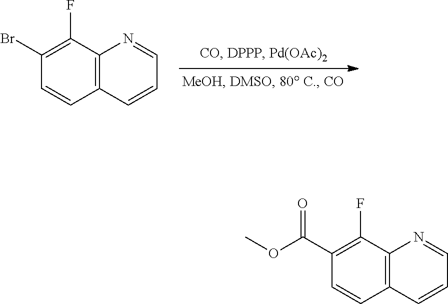

To a solution of 7-bromo-8-fluoroquinoline (26.0 g, 0.12 mol, 1.0 eq), DPPP (9.5 g, 23.1 mmol, 0.2 eq) and Pd(OAc)₂ (2.7 g, 0.012 mol, 0.1 eq) in DMSO/MeOH (260 mL/260 mL) was added TEA (49.0 mL, 0.36 mol. 3.0 eq). The mixture was stirred at 80° C. for 15 h under CO (5 atm), then concentrated under vacuum. The resulting residue was purified by column chromatography on silica gel (PE/EA=10/1, v/v) to afford methyl 8-fluoroquinoline-7-carboxylate (11.2 g, 47.5%) as a yellow oil. LRMS (M+H⁺) m/z calculated 206.1, found 206.1.

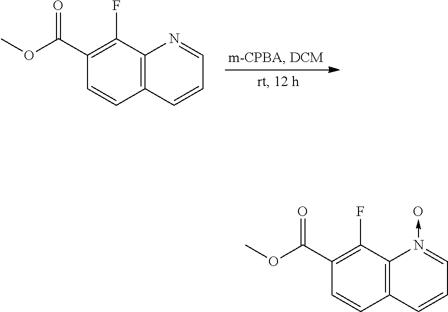 [SMILES: COC(=O)c1ccc2cccnc2c1F ; COC(=O)c1ccc2ccc[n+]([O-])c2c1F]

To a stirred solution of methyl 8-fluoroquinoline-7-carboxylate (6.5 g, 31.7 mmol, 1.0 eq) in DCM (80 mL) was added m-CPBA (8.17 g, 47.6 mmol, 1.5 eq) at rt. The mixture was stirred at rt for 12 h, then poured into ice, adjusted to pH 13 with addition of saturated Na₂CO₃ aqueous solution, and extracted with DCM (100 mL×3). The combined organic layers were dried over sodium sulfate and concentrated under vacuum to afford 8-fluoro-7-(methoxycarbonyl) quinoline 1-oxide (6.3 g, 90%) as a yellow oil. LRMS (M+H⁺) m/z calculated 222.1, found 222.1.

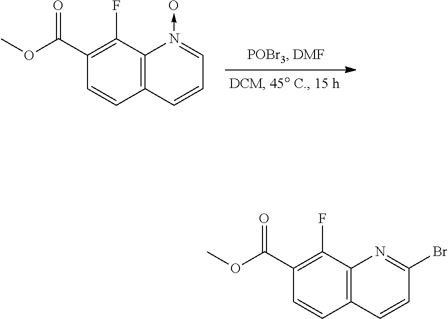 [SMILES: COC(=O)c1ccc2ccc[n+]([O-])c2c1F ; COC(=O)c1ccc2ccc(Br)nc2c1F]

To a solution of 8-fluoro-7-(methoxycarbonyl) quinoline 1-oxide (3.1 g, 14.0 mmol, 1.0 eq) in DCM (30 mL) were added POBr₃ (5.2 g, 18.2 mmol, 1.3 eq) and DMF (0.5 mL, 7.0 mmol, 0.5 eq) at −78° C. The mixture was stirred at 45° C. for 15 h, then poured into ice, adjusted to pH 13 with saturated Na₂CO₃ aqueous solution, and extracted with DCM (50 mL×3). The combined organic layers were dried over anhydrous sodium sulfate, filtered and concentrated under vacuum. The resulting residue was purified by column chromatography on silica gel (PE/EA=5/1, v/v) to afford methyl 2-bromo-8-fluoroquinoline-7-carboxylate (1.3 g, 33.3%) as a yellow solid. LRMS (M+H⁺) m/z calculated 284.0, found 284.0.

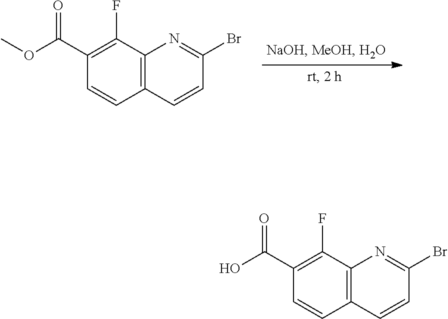 [SMILES: COC(=O)c1ccc2ccc(Br)nc2c1F ; O=C(O)c1ccc2ccc(Br)nc2c1F]

To a solution of methyl 2-bromo-8-fluoroquinoline-7-carboxylate (1.2 g, 4.23 mmol, 1.0 eq) in MeOH (50 mL) and H₂O (8 mL) was added NaOH (254 mg, 6.35 mmol, 1.5 eq). The mixture was stirred at 50° C. for 5 h, then concentrated under vacuum and diluted with water (10 mL), 37% HCl was added to adjust to pH 2. The resulting mixture was stirred for 5 min, filtered and concentrated under vacuum to afford 8-fluoro-2-phenylquinoline-7-carboxylic acid (1.0 g, 3.69 mmol, 87.2%) as a white solid, LRMS (M+H⁺) m/z calculated 269.9, found 269.9.

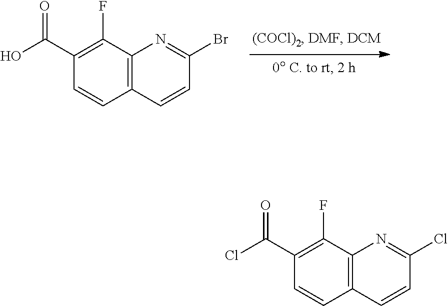 [SMILES: O=C(O)c1ccc2ccc(Br)nc2c1F ; O=C(Cl)c1ccc2ccc(Cl)nc2c1F]

To a solution of 8-fluoro-2-phenylquinoline-7-carboxylic acid (1.0 g, 3.69 mmol, 1.0 eq) in DCM (50 mL) were added (COCl)₂ (1.6 mL, 18.4 mmol, 5.0 eq) and DMF (1 drop) at −78° C. The mixture was stirred at rt for 2 h, then concentrated under vacuum to afford 2-chloro-8-fluoroquinoline-7-carbonyl chloride as a yellow solid (1.3 g, ca 100.0%), LRMS (M+H⁺) m/z calculated 240.0, found 240.0 in MeOH.

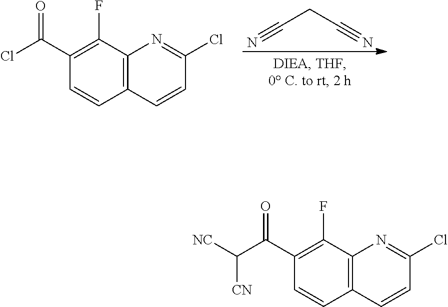

To a solution of 2-chloro-8-fluoroquinoline-7-carbonyl chloride (1.3 g, 5.33 mmol, 1.0 eq) in THF (40 mL) were added malononitrile (352 mg, 5.33 mmol, 1.0 eq) and DIEA (2.06 g, 16.0 mmol, 3.0 eq) at ice bath. The mixture was stirred at rt for 3 h, then concentrated under vacuum, diluted with water (50 mL). The resulting mixture was extracted with EtOAc (50 mL×3). The combined organic layers were washed with brine (50 mL), dried over anhydrous sodium sulfate, filtered and concentrated under vacuum. The resulting residue was purified by column chromatography on silica gel (DCM/MeOH=50/1, v/v) to afford 2-(2-chloro-8-fluoroquinoline-7-carbonyl)malononitrile as a yellow oil (1.1 g, 4.03 mmol, 75.6%), LRMS (M+H⁺) m/z calculated 274.1, found 274.1.

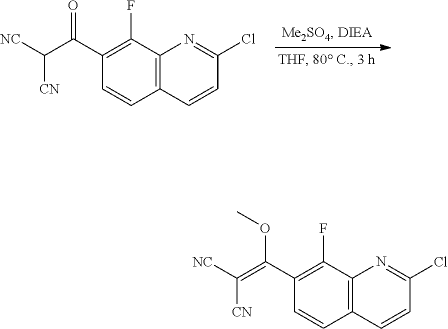 [SMILES: N#CC(C#N)C(=O)c1ccc2ccc(Cl)nc2c1F ; COC(=C(C#N)C#N)c1ccc2ccc(Cl)nc2c1F]

To a solution of 2-(2-chloro-8-fluoroquinoline-7-carbonyl)malononitrile (1.0 g, 3.65 mmol, 1.0 eq) in THF (30 mL) were added Me₂SO₄ (920 mg, 7.30 mmol, 2.0 eq) and DIEA (942 mg, 7.30 mmol, 2.0 eq) at rt. The mixture was stirred at 80° C. for 3 h, then concentrated under vacuum and diluted with water (50 mL), extracted with EtOAc (50 mL×3). The combined organic layers were washed with brine (50 mL), dried over anhydrous sodium sulfate, filtered and concentrated under vacuum. The resulting residue was purified by column chromatography on silica gel (PE/EA=1/1, v/v) to afford 2-(2-chloro-8-fluoroquinoline-7-carbonyl)malononitrile as a yellow oil (400 mg, 1.39 mmol, 38.1%), LRMS (M+H⁺) m/z calculated 288.1, found 288.1.

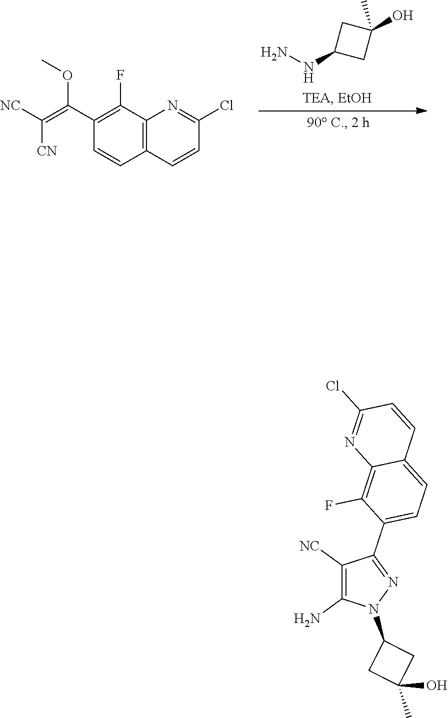

To a solution of 2-(2-chloro-8-fluoroquinoline-7-carbonyl)malononitrile (400 mg, 1.39 mmol, 1.0 eq) and (1s,3s)-3-hydrazinyl-1-methylcyclobutan-1-ol (242 mg, 2.09 mmol, 1.5 eq) in EtOH (30 mL) was added TEA (1.12 g, 11.1 mmol, 8.0 eq) at rt. The reaction mixture was stirred at 90° C. for 2 h, and concentrated under vacuum. The resulting residue was purified by column chromatography on silica gel (PE/EA=1/1, v/v) to afford 5-amino-3-(2-chloro-8-fluoroquinolin-7-yl)-1-((1s,3s)-3-hydroxy-3-methylcyclobutyl)-1H-pyrazole-4-carbonitrile (120 mg, 0.322 mmol, 23.2%) as a yellow solid. LRMS (M+H⁺) m/z calculated 372.1, found 372.1.

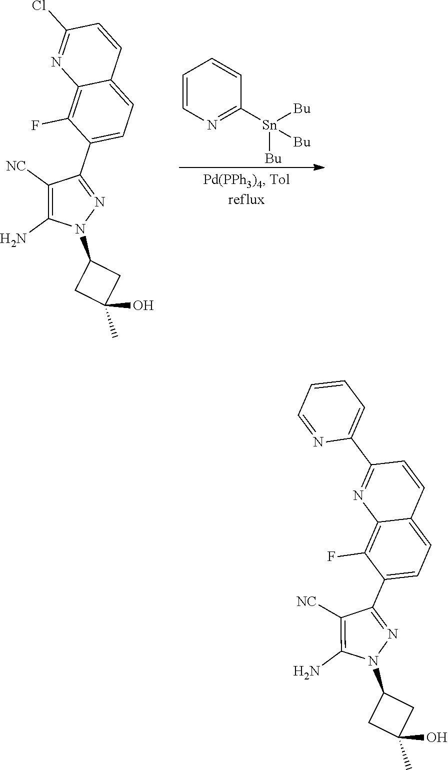

A mixture of 5-amino-3-(2-chloro-8-fluoroquinolin-7-yl)-1-((1s,3s)-3-hydroxy-3-methylcyclobutyl)-1H-pyrazole-4-carbonitrile (50 mg, 0.13 mmol, 1.0 eq), 2-(tributylstannyl)pyridine (99.5 mg, 0.26 mmol, 2.0 eq) and Pd(PPh₃)₄ (15 mg, 0.013 mmol, 0.1 eq) in toluene (6.0 mL) was stirred at reflux for 12 h. The mixture was concentrated, and the resulting residue was purified by column chromatography on silica gel (DCM/MeOH=10/1, v/v) to afford 5-amino-3-(8-fluoro-2-(pyridin-2-yl) quinolin-7-yl)-1-((1s,3s)-3-hydroxy-3-methylcyclobutyl)-1H-pyrazole-4-carbonitrile (30 mg, 53.6%) as a yellow solid. LRMS (M+H⁺) m/z calculated 415.2, found 415.2.

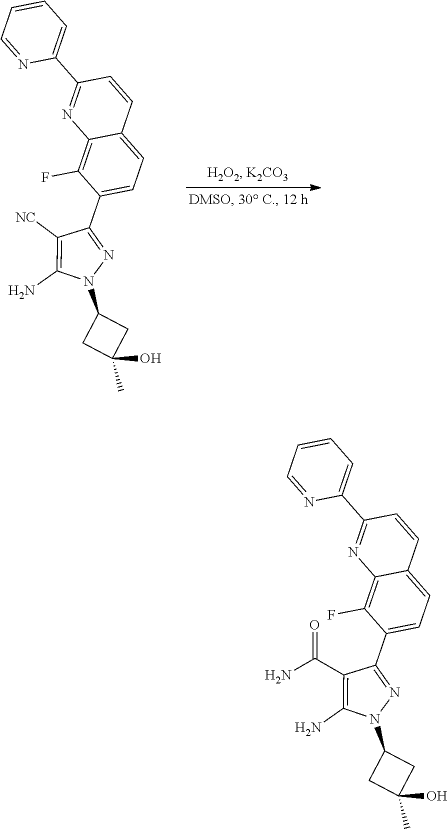 [SMILES: C[C@]1(O)C[C@@H](n2nc(-c3ccc4ccc(-c5ccccn5)nc4c3F)c(C#N)c2N)C1 ; C[C@]1(O)C[C@@H](n2nc(-c3ccc4ccc(-c5ccccn5)nc4c3F)c(C(N)=O)c2N)C1]

To a stirred solution of 5-amino-3-(8-fluoro-2-(pyridin-2-yl) quinolin-7-yl)-1-((1s,3s)-3-hydroxy-3-methylcyclobutyl)-1H-pyrazole-4-carbonitrile (40 mg, 0.108 mmol, 1.0 eq) and K₂CO₃ (44.7 mg, 0.324 mmol, 3.0 eq) in DMSO (10 mL) was added H₂O₂ (30%, 237 mg, 1.5 mmol, 20.0 eq) at rt. After addition was complete, the reaction mixture was stirred at 60° C. for 1 h. Water (20 mL) was added and the mixture was extracted with EtOAc (50 mL). The organic layer was washed with brine (100 mL), dried with anhydrous sodium sulfate, and purified by Prep-HPLC to afford 5-amino-3-(8-fluoro-2-(pyridin-2-yl) quinolin-7-yl)-1-((1s,3s)-3-hydroxy-3-methylcyclobutyl)-1H-pyrazole-4-carboxamide (1.0 mg, 0.002 mmol, 2.1%) as a white solid. LRMS (M+H⁺) m/z calculated 433.2, found 433.2. ¹H NMR (CDCl₃, 400 MHz) δ 8.74-8.77 (m, 3H), 8.34-8.36 (d, 1H), 7.91-7.93 (t, 1H), 7.74-7.76 (d, 1H), 7.60-7.64 (m, 1H), 7.26-7.43 (t, 1H), 5.51 (br, 1H), 5.12 (br, 2H), 4.33-4.36 (m, 1H), 2.74-2.76 (m, 4H), 1.25 (s, 3H).

### Example 58: Preparation of 5-amino-3-(3,8-difluoro-2-(2-fluorophenyl) quinolin-7-yl)-1-((1s,3s)-3-hydroxy-3-methylcyclobutyl)-1H-pyrazole-4-carboxamide

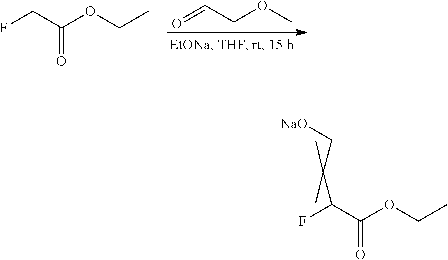

A mixture of ethyl 2-fluoroacetate (50.0 g, 0.47 mol, 1.0 eq), ethyl formate (52.4 g, 0.71 mol, 1.5 eq) and EtONa (63.9 g, 0.94 mol, 2.0 eq) in THF (1000 mL) was stirred at rt for 15 h. The mixture was concentrated, and the resulting residue was used in the next step without further purification.

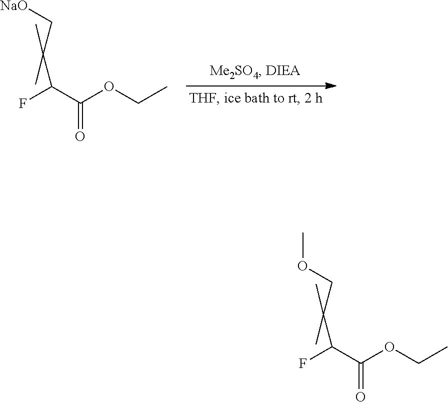

DIEA (181.9 g, 1.4 mol, 3.0 eq) was added to a mixture of sodium 3-ethoxy-2-fluoro-3-oxoprop-1-en-1-olate (73.0 g, 0.47 mol, 1.0 eq) and Me₂SO₄ (119.4 g, 0.94 mol, 2.0 eq) in THF (1000 mL) at ice bath and the mixture was stirred at rt for 2.0 h. The mixture was concentrated, and the resulting residue was purified by column chromatography on silica gel (PE/EtOAc=10/1, v/v) to afford ethyl 2-fluoro-3-methoxyacrylate (36.3 g, 52.2%) as a yellow oil. LRMS (M+H⁺) m/z calculated 149.1, found 149.1.

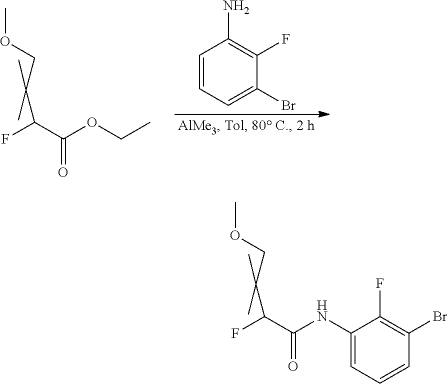 [SMILES: CCOC(=O)C(F)C(C)(C)COC ; Nc1cccc(Br)c1F ; COCC(C)(C)C(F)C(=O)Nc1cccc(Br)c1F]

A mixture of ethyl 2-fluoro-3-methoxyacrylate (11.3 g, 38.8 mmol, 1.0 eq), 3-bromo-2-fluoroaniline (11.0 g, 58.2 mmol, 1.5 eq) and AlMe₃ (77.6 mL, 77.6 mmol, 2.0 eq) in toluene (200 mL) was stirred at 80° C. for 2 h under N₂. The mixture was concentrated, and the resulting residue was purified by column chromatography on silica gel (PE/EtOAc=10/1, v/v) to afford N-(3-bromo-2-fluorophenyl)-2-fluoro-3-methoxyacrylamide (16.0 g, 72.7%) as a yellow solid. LRMS (M+H⁺) m/z calculated 292.0, found 292.0.

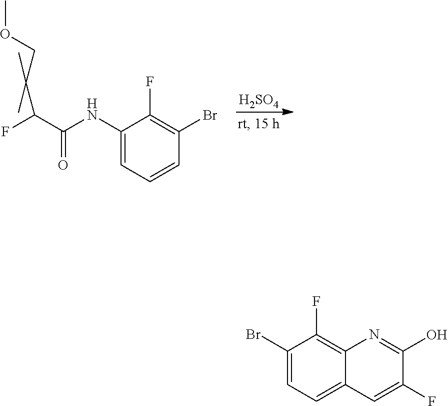 [SMILES: COCC(C)(C)C(F)C(=O)Nc1cccc(Br)c1F ; "H2SO4" ; Oc1nc2c(F)c(Br)ccc2cc1F]

A mixture of N-(3-bromo-2-fluorophenyl)-2-fluoro-3-methoxyacrylamide (16.0 g, 54.9 mmol, 1.0 eq) in con. H₂SO₄ (100 mL) was stirred at rt for 15 h. The mixture was poured to ice water and extracted with EtOAc (100 mL×5). The combined organic layers were washed with brine (100 mL), dried over anhydrous sodium sulfate, filtered and concentrated under vacuum. The resulting residue was purified by column chromatography on silica gel (PE/EA=5/1, v/v) to afford 7-bromo-3,8-difluoroquinolin-2-ol as a yellow solid (11.6 g, 81.7%), LRMS (M+H⁺) m/z calculated 260.0, found 260.0.

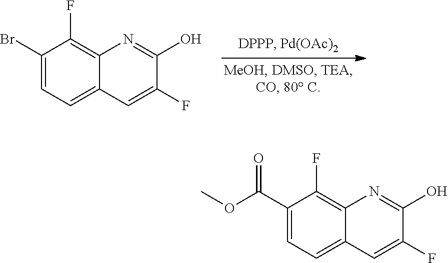 [SMILES: Oc1nc2c(F)c(Br)ccc2cc1F ; COC(=O)c1ccc2cc(F)c(O)nc2c1F]

To a solution of 7-bromo-3,8-difluoroquinolin-2-ol (11.6 g, 44.8 mmol, 1.0 eq), DPPP (3.7 g, 8.96 mmol, 0.2 eq) and Pd(OAc)₂ (1.0 g, 4.5 mmol, 0.1 eq) in DMSO/MeOH (100 mL/100 mL) was added TEA (18.4 mL, 134.4 mmol. 3.0 eq) and the mixture was stirred at 80° C. for 15 h under CO (5 atm). The reaction was quenched by H₂O (500 mL) and extracted with EtOAc (100 mL×5). The combined organic layers were washed with brine (200 mL), dried over anhydrous sodium sulfate, filtered and concentrated under vacuum and then concentrated under vacuum. The resulting residue was purified by column chromatography on silica gel (PE/EA=2/1, v/v) to afford methyl 3,8-difluoro-2-hydroxyquinoline-7-carboxylate (9.0 g, 84.1%) as a yellow solid. LRMS (M+H⁺) m/z calculated 240.0, found 240.0.

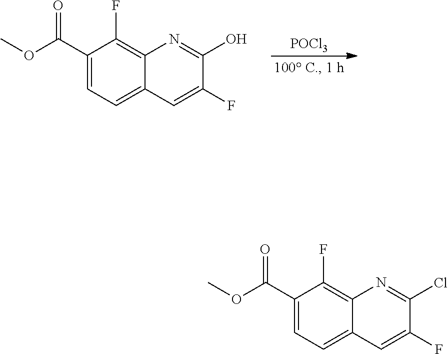 [SMILES: COC(=O)c1ccc2cc(F)c(O)nc2c1F ; "POCl3" ; COC(=O)c1ccc2cc(F)c(Cl)nc2c1F]

A mixture of methyl 3,8-difluoro-2-hydroxyquinoline-7-carboxylate (9.0 g, 37.7 mmol, 1.0 eq) and POCl₃ (80.0 mL) was stirred at 100° C. for 1 h. The mixture was concentrated, and the resulting residue was purified by column chromatography on silica gel (PE/EtOAc=5/1, v/v) to afford methyl 2-chloro-3,8-difluoroquinoline-7-carboxylate (6.3 g, 65.6%) as a yellow solid. LRMS (M+H⁺) m/z calculated 258.0, found 258.0

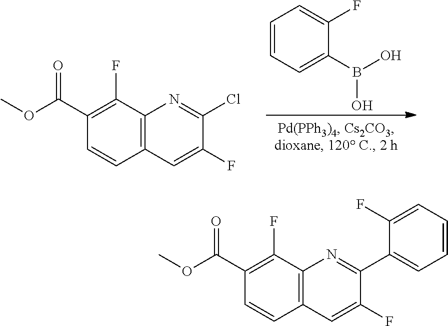 [SMILES: COC(=O)c1ccc2cc(F)c(Cl)nc2c1F ; OB(O)c1ccccc1F ; COC(=O)c1ccc2cc(F)c(-c3ccccc3F)nc2c1F]

To a stirred solution of methyl 2-chloro-3,8-difluoroquinoline-7-carboxylate (3.0 g, 11.7 mmol, 1.0 eq) and (2-fluorophenyl) boronic acid (2.46 g, 17.6 mmol, 1.5 eq) in dioxane (100 mL) and water (10 mL) was added Cs₂CO₃ (11.5 g, 35.1 mmol, 3.0 eq.) and Pd(PPh₃)₄ (1.35 g, 1.17 mmol, 0.1 eq.) at rt. The reaction mixture was stirred at 120° C. for 2 h under N₂, and then filtered and concentrated under vacuum. The resulting residue was purified by column chromatography on silica gel (PE/EA=10/1, v/v) to afford methyl 3,8-difluoro-2-(2-fluorophenyl) quinoline-7-carboxylate (2.7 g, 8.51 mmol, 72.7%) as a white solid. LRMS (M+H⁺) m/z calculated 318.1, found 318.1

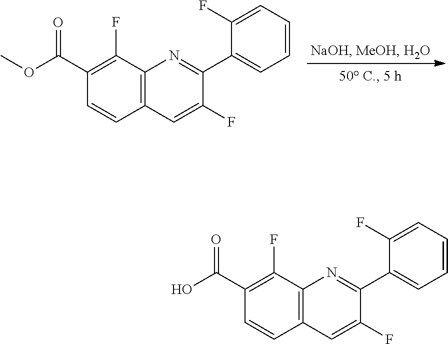 [SMILES: COC(=O)c1ccc2cc(F)c(-c3ccccc3F)nc2c1F ; O=C(O)c1ccc2cc(F)c(-c3ccccc3F)nc2c1F]

To a solution of methyl 3,8-difluoro-2-(2-fluorophenyl) quinoline-7-carboxylate (2.7 g, 8.51 mmol, 1.0 eq) in MeOH (50 mL) and H₂O (8 mL) was added NaOH (512 mg, 12.8 mmol, 1.5 eq). The mixture was stirred at 50° C. for 5 h, then concentrated under vacuum and diluted with water (20 mL). 37% HCl was added to adjust to pH 2. The resulting mixture was stirred for 5 min, filtered and concentrated under vacuum to afford 3,8-difluoro-2-(2-fluorophenyl) quinoline-7-carboxylic acid (2.6 g, 8.5 mmol, ca. 100%) as a white solid, LRMS (M+H⁺) m/z calculated 304.1, found 304.1.

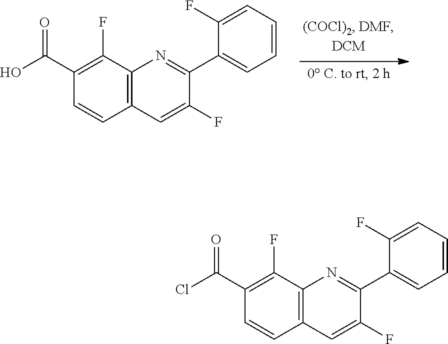 [SMILES: O=C(O)c1ccc2cc(F)c(-c3ccccc3F)nc2c1F ; O=C(Cl)c1ccc2cc(F)c(-c3ccccc3F)nc2c1F]

To a solution of 3,8-difluoro-2-(2-fluorophenyl) quinoline-7-carboxylic acid (1.0 g, 3.30 mmol, 1.0 eq) in DCM (50 mL) were added (COCl)₂ (20.9 g, 165 mmol, 5.0 eq) and DMF (1 drop) at −78° C. The mixture was stirred at rt for 1 h, then concentrated under vacuum to afford 3,8-difluoro-2-(2-fluorophenyl) quinoline-7-carbonyl chloride (1.3 g, ca 100.0%), LRMS (M+H⁺) m/z calculated 318.1, found 318.1 in MeOH.

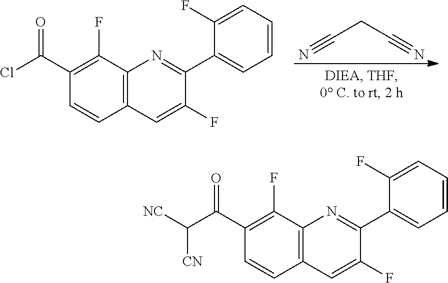

To a solution of 3,8-difluoro-2-(2-fluorophenyl) quinoline-7-carbonyl chloride (390 mg, 1.21 mmol, 1.0 eq) in THF (10 mL) were added malononitrile (80.0 mg, 1.21 mmol, 1.0 eq) and DIEA (468 mg, 3.63 mmol, 3.0 eq) at ice bath. The mixture was stirred at rt for 3 h, then concentrated under vacuum, and diluted with water (20 mL). The resulting mixture was extracted with EtOAc (40 mL×3). The combined organic layers were washed with brine (20 mL), dried over anhydrous sodium sulfate, filtered and concentrated under vacuum. The resulting residue was purified by column chromatography on silica gel (DCM/MeOH=50/1, v/v) to afford 2-(3,8-difluoro-2-(2-fluorophenyl) quinoline-7-carbonyl)malononitrile as a yellow oil (330 mg, 0.939 mmol, 78%), LRMS (M+H⁺) m/z calculated 352.1, found 352.1.

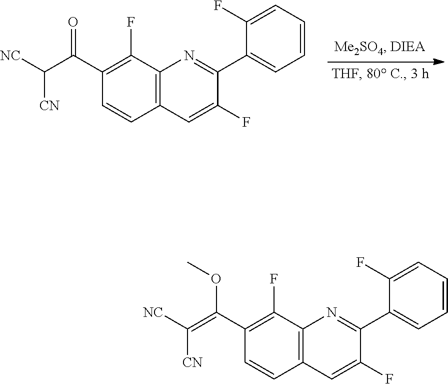 [SMILES: N#CC(C#N)C(=O)c1ccc2cc(F)c(-c3ccccc3F)nc2c1F ; COC(=C(C#N)C#N)c1ccc2cc(F)c(-c3ccccc3F)nc2c1F]

To a solution of 2-(3,8-difluoro-2-(2-fluorophenyl) quinoline-7-carbonyl)malononitrile (330 mg, 0.939 mmol, 1.0 eq) in THF (10 mL) were added Me₂SO₄ (237 mg, 1.88 mmol, 2.0 eq) and DIEA (243 mg, 1.88 mmol, 2.0 eq) at rt. The mixture was stirred at 80° C. for 3 h, then concentrated under vacuum, diluted with water (20 mL), and extracted with EtOAc (40 mL×3). The combined organic layers were washed with brine (20 mL), dried over anhydrous sodium sulfate, filtered and concentrated under vacuum. The resulting residue was purified by column chromatography on silica gel (PE/EA=1/1, v/v) to afford 2-((3,8-difluoro-2-(2-fluorophenyl) quinolin-7-yl)(methoxy)methylene)malononitrile as a yellow oil (170 mg, 0.465 mmol, 49.6%), LRMS (M+H⁺) m/z calculated 366.1, found 366.1.

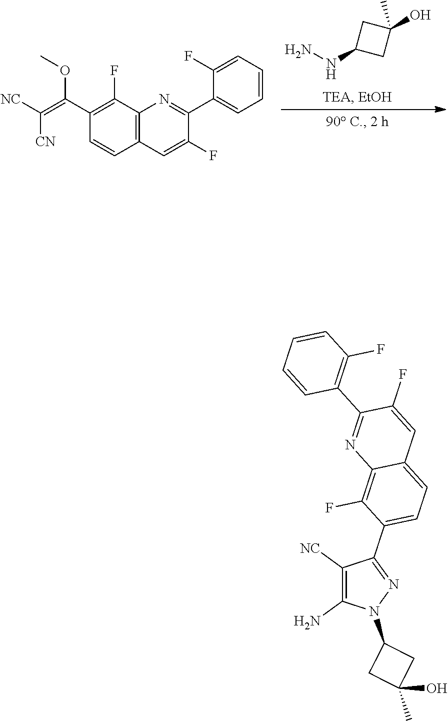

To a solution of 2-((3,8-difluoro-2-(2-fluorophenyl) quinolin-7-yl)(methoxy)methylene)malononitrile (170 mg, 0.465 mmol, 1.0 eq) and (1s,3s)-3-hydrazinyl-1-methylcyclobutan-1-ol (81.1 mg, 0.698 mmol, 1.5 eq) in EtOH (80 mL) were added TEA (376 mg, 3.72 mmol, 8.0 eq) at rt. The reaction mixture was stirred at 90° C. for 2 h, then concentrated under vacuum and purified by column chromatography on silica gel (PE/EA=1/1, v/v) to afford 5-amino-3-(3,8-difluoro-2-(2-fluorophenyl) quinolin-7-yl)-1-((1s,3s)-3-hydroxy-3-methylcyclobutyl)-1H-pyrazole-4-carbonitrile (160 mg, 0.356 mmol, 76.6%) as a yellow solid. LRMS (M+H⁺) m/z calculated 450.1, found 450.2.

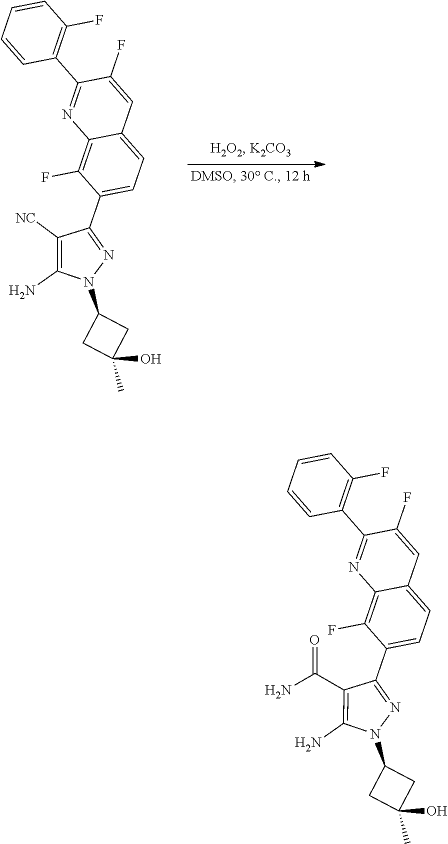 [SMILES: C[C@]1(O)C[C@@H](n2nc(-c3ccc4cc(F)c(-c5ccccc5F)nc4c3F)c(C#N)c2N)C1 ; C[C@]1(O)C[C@@H](n2nc(-c3ccc4cc(F)c(-c5ccccc5F)nc4c3F)c(C(N)=O)c2N)C1]

To a stirred solution of 5-amino-3-(3,8-difluoro-2-(2-fluorophenyl) quinolin-7-yl)-1-((1s,3s)-3-hydroxy-3-methylcyclobutyl)-1H-pyrazole-4-carbonitrile (160 mg, 0.356 mmol, 1.0 eq) and K₂CO₃ (147 mg, 1.07 mmol, 3.0 eq) in DMSO (10 mL) was added H₂O₂ (30%, 1.12 g, 7.12 mmol, 20.0 eq) at rt. After addition was complete, the reaction mixture was stirred at 60° C. for 1 h. Water (20 mL) was added, and the mixture was extracted with EtOAc (50 mL). The organic layer was washed with brine (100 mL), dried with anhydrous sodium sulfate and purified by Prep-HPLC to afford 5-amino-3-(3,8-difluoro-2-(2-fluorophenyl) quinolin-7-yl)-1-((1s,3s)-3-hydroxy-3-methylcyclobutyl)-1H-pyrazole-4-carboxamide (43 mg, 0.092 mmol, 25.8%) as a white solid. LRMS (M+H⁺) m/z calculated 468.2, found 468.2. ¹H NMR (DMSO-d₆, 400 MHz) δ 8.53-8.56 (d, 1H), 7.91-7.93 (d, 1H), 7.70-7.76 (m, 2H), 7.62-7.64 (m, 1H), 7.40-7.46 (m, 2H), 6.30 (s, 2H), 5.17 (br, 1H), 4.44-4.48 (m, 1H), 2.54-2.59 (m, 2H), 2.35-2.40 (m, 2H), 1.33 (s, 3H).

### Example 59: Preparation of 5-amino-3-(3,8-difluoro-2-(2-fluorophenyl) quinolin-7-yl)-1-((1s,3s)-3-hydroxy-3-methylcyclobutyl)-1H-pyrazole-4-carboxamide

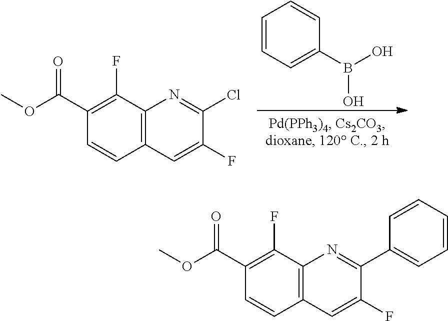 [SMILES: COC(=O)c1ccc2cc(F)c(Cl)nc2c1F ; OB(O)c1ccccc1 ; COC(=O)c1ccc2cc(F)c(-c3ccccc3)nc2c1F]

To a stirred solution of methyl 2-chloro-3,8-difluoroquinoline-7-carboxylate (500 mg, 1.95 mmol, 1.0 eq) and phenylboronic acid (357 mg, 2.93 mmol, 1.5 eq) in dioxane (15 mL) and water (1.5 mL) was added Cs₂CO₃ (1.91 g, 35.1 mmol, 3.0 eq.) and Pd(PPh₃)₄ (225 mg, 0.1951.3 mmol, 0.1 eq.) at rt. The reaction mixture was stirred at 120° C. for 2 h under N₂. The reaction mixture was filtered and concentrated, and the resulting residue was purified by column chromatography on silica gel (PE/EA=10/1, v/v) to afford methyl 3,8-difluoro-2-phenylquinoline-7-carboxylate (280 mg, 0.936 mmol, 48.0%) as a white solid. LRMS (M+H⁺) m/z calculated 300.1, found 300.1.

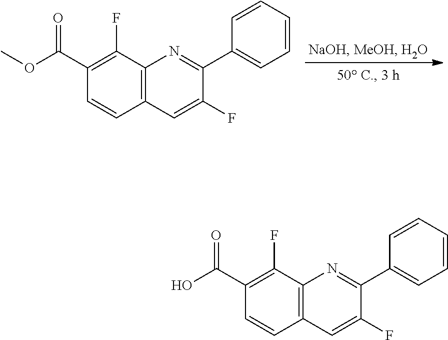 [SMILES: COC(=O)c1ccc2cc(F)c(-c3ccccc3)nc2c1F ; O=C(O)c1ccc2cc(F)c(-c3ccccc3)nc2c1F]

To a solution of methyl 3,8-difluoro-2-phenylquinoline-7-carboxylate (280 mg, 0.936 mmol, 1.0 eq) in MeOH (20 mL) and H₂O (3 mL) was added NaOH (117 mg, 2.93 mmol, 1.5 eq). The mixture was stirred at 50° C. for 3 h, then concentrated under vacuum and diluted with water (10 mL). 37% HCl was added to adjust to pH 2. The resulting mixture was stirred for 5 min, filtered and concentrated under vacuum to afford 3,8-difluoro-2-phenylquinoline-7-carboxylic acid (250 mg, 0.877 mmol, 93.7%) as a white solid, LRMS (M+H⁺) m/z calculated 286.1, found 286.1.

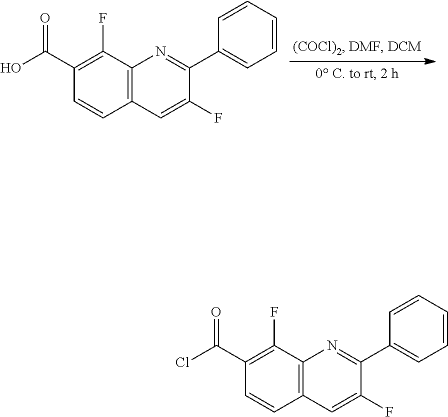 [SMILES: O=C(O)c1ccc2cc(F)c(-c3ccccc3)nc2c1F ; O=C(Cl)c1ccc2cc(F)c(-c3ccccc3)nc2c1F]

To a solution of 3,8-difluoro-2-phenylquinoline-7-carboxylic acid (250 mg, 0.877 mmol, 1.0 eq) in DCM (20 mL) were added (COCl)₂ (552 mg, 4.39 mmol, 5.0 eq) and DMF (1 drop) at 0° C. The mixture was stirred at rt for 1 h, and concentrated under vacuum to afford 3,8-difluoro-2-phenylquinoline-7-carbonyl chloride (270 mg, ca 100.0%), LRMS (M+H⁺) m/z calculated 300.1, found 300.1 in MeOH.

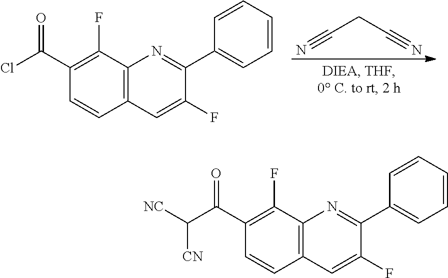

To a solution of 3,8-difluoro-2-phenylquinoline-7-carbonyl chloride (240 mg, 0.792 mmol, 1.0 eq) in THF (10 mL) were added malononitrile (52.7 mg, 0.792 mmol, 1.0 eq) and DIEA (307 mg, 2.38 mmol, 3.0 eq) at ice bath. The mixture was stirred at rt for 3 h, then concentrated under vacuum, and diluted with water (10 mL). The resulting mixture was extracted with EtOAc (20 mL×3). The combined organic layers were washed with brine (10 mL), dried over anhydrous sodium sulfate, filtered and concentrated under vacuum. The resulting residue was purified by column chromatography on silica gel (DCM/MeOH=50/1, v/v) to afford 2-(3,8-difluoro-2-phenylquinoline-7-carbonyl)malononitrile as a yellow oil (180 mg, 0.541 mmol, 68.2%), LRMS (M+H⁺) m/z calculated 334.1, found 334.1.

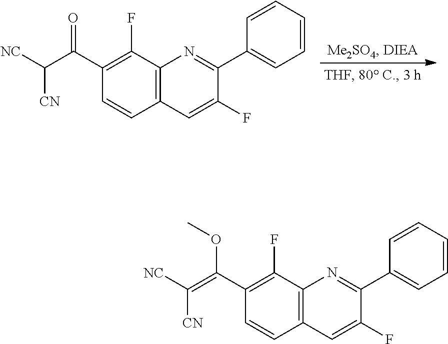 [SMILES: N#CC(C#N)C(=O)c1ccc2cc(F)c(-c3ccccc3)nc2c1F ; COC(=C(C#N)C#N)c1ccc2cc(F)c(-c3ccccc3)nc2c1F]

To a solution of 2-(3,8-difluoro-2-phenylquinoline-7-carbonyl)malononitrile (210 mg, 0.631 mmol, 1.0 eq) in THF (10 mL) were added Me₂SO₄ (159 mg, 1.26 mmol, 2.0 eq) and DIEA (163 mg, 1.26 mmol, 2.0 eq) at rt. The mixture was stirred at 80° C. for 3 h, then concentrated under vacuum, diluted with water (10 mL), and extracted with EtOAc (20 mL×3). The combined organic layers were washed with brine (10 mL), dried over anhydrous sodium sulfate, filtered and concentrated under vacuum. The resulting residue was purified by column chromatography on silica gel (PE/EA=1/1, v/v) to afford 2-((3,8-difluoro-2-phenylquinolin-7-yl)(methoxy)methylene)malononitrile as a yellow oil (70 mg, 0.202 mmol, 32.0%), LRMS (M+H⁺) m/z calculated 348.1, found 348.1.

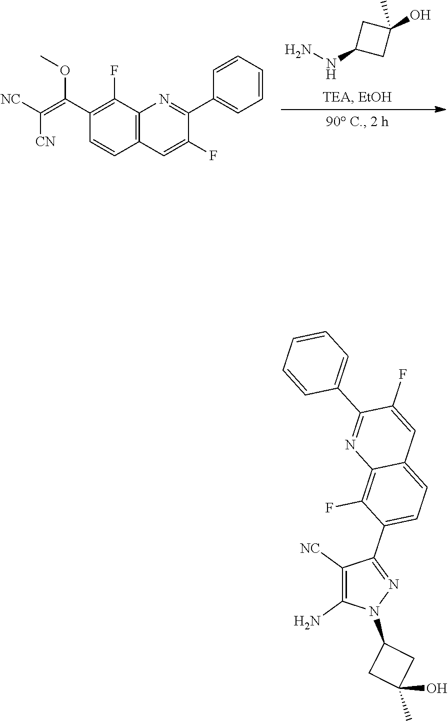

To a solution of 2-((3,8-difluoro-2-phenyl) quinolin-7-yl)(methoxy)methylene)malononitrile (70 mg, 0.202 mmol, 1.0 eq) and (1s,3s)-3-hydrazinyl-1-methylcyclobutan-1-ol (35.1 mg, 0.303 mmol, 1.5 eq) in EtOH (10 mL) was added TEA (163 mg, 1.62 mmol, 8.0 eq) at rt. The reaction mixture was stirred at 90° C. for 2 h, then concentrated under vacuum and purified by column chromatography on silica gel (PE/EA=1/1, v/v) to afford 5-amino-3-(3,8-difluoro-2-phenylquinolin-7-yl)-1-((1s,3s)-3-hydroxy-3-methylcyclobutyl)-1H-pyrazole-4-carbonitrile (70 mg, 0.162 mmol, 80.2%) as a yellow solid. LRMS (M+H⁺) m/z calculated 432.2, found 432.2.

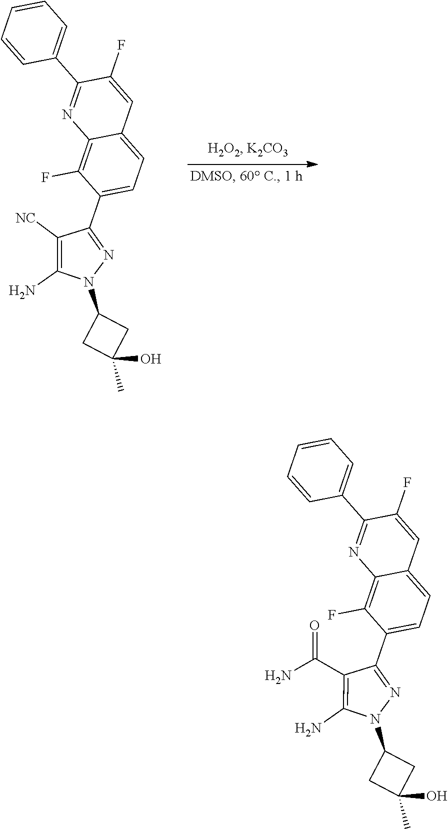 [SMILES: C[C@]1(O)C[C@@H](n2nc(-c3ccc4cc(F)c(-c5ccccc5)nc4c3F)c(C#N)c2N)C1 ; C[C@]1(O)C[C@@H](n2nc(-c3ccc4cc(F)c(-c5ccccc5)nc4c3F)c(C(N)=O)c2N)C1]

To a stirred solution of 5-amino-3-(3,8-difluoro-2-phenylquinolin-7-yl)-1-((1s,3s)-3-hydroxy-3-methylcyclobutyl)-1H-pyrazole-4-carbonitrile (70 mg, 0.162 mmol, 1.0 eq) and K₂CO₃ (67.3 mg, 0.488 mmol, 3.0 eq) in DMSO (10 mL) was added H₂O₂ (30%, 370 mg, 3.26 mmol, 20.0 eq) at rt. After addition was complete, the reaction mixture was stirred at 60° C. for 1 h. Water (10 mL) was added, and the mixture was extracted with EtOAc (30 mL). The organic layer was washed with brine (100 mL), dried with anhydrous sodium sulfate and purified by Prep-HPLC to afford 5-amino-3-(3,8-difluoro-2-phenyl) quinolin-7-yl)-1-((1s,3s)-3-hydroxy-3-methylcyclobutyl)-1H-pyrazole-4-carboxamide (15.4 mg, 0.0342 mmol, 21.2%) as a white solid. LRMS (M+H⁺) m/z calculated 450.2, found 450.2. ¹H NMR (DMSO-d₆, 400 MHz) δ 8.57 (d, 1H), 8.27-8.33 (m, 2H), 7.89 (d, 1H), 7.51-7.63 (m, 4H), 6.32 (s, 2H), 5.18 (s, 1H), 4.45-4.50 (m, 1H), 2.55-2.61 (m, 2H), 2.36-2.41 (m, 2H), 1.34 (s, 3H).

### Example 60: Preparation of 5-amino-3-(3-fluoro-4-methoxy-2-phenylquinolin-7-yl)-1-((1s,3s)-3-hydroxy-3-methylcyclobutyl)-1H-pyrazole-4-carboxamide

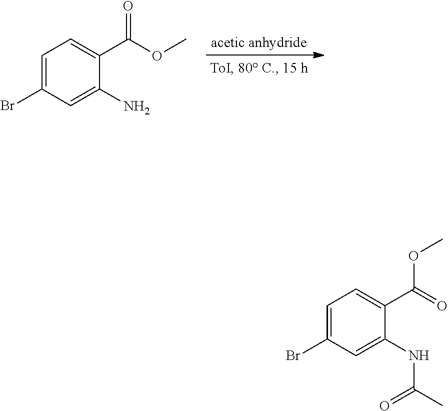

To a suspension of methyl 2-amino-4-bromobenzoate (25 g, 108.7 mmol) in toluene (300 mL) was added acetic anhydride (15.3 mL, 163.1 mmol, 1.5 eq) at 28° C. The mixture was stirred at 80° C. for 15 h, and concentrated under vacuum. The residual solid was triturated with PE/EtOAc (300 mL, 10:1) and dried to afford methyl 2-acetamido-4-bromobenzoate (24.5 g, 83%) as a light yellow solid. LRMS (M+H⁺) m/z calculated 272.0, found 272.0. ¹H NMR (DMSO-d₆, 400 MHz) δ 10.62 (s, 1H), 8.53 (d, 1H), 7.83 (d, 1H), 7.39 (dd, 1H), 3.86 (s, 3H), 2.15 (s, 3H).

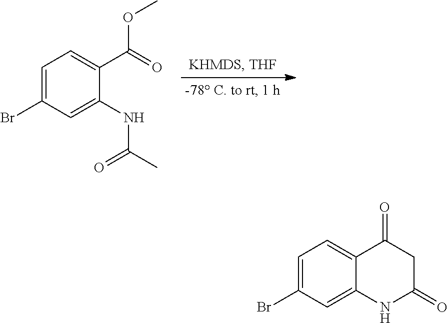

To solutions of KHMDS in THF (1 M, 270.2 mL, 270.2 mmol, 3.0 eq) were added suspensions of methyl 2-acetamido-4-bromobenzoate (24.5 g, 90.1 mmol, 1.0 eq) in THF (500 mL) dropwise at −78° C. under N₂. After stirring at this temperature for 1 h, the mixture was allowed to warm up to 10° C. within 1 h. The mixture was quenched with water (1000 mL) and extracted with EtOAc (500 mL×2). The separated aqueous layer was cooled to 0° C. and the pH was adjusted to 2.5-3.5 with the addition of 5N HCl aqueous solution. The solid was collected by filtration, washed with EtOAc (500 mL) and dried under vacuum to afford 7-bromo-4-hydroxyquinolin-2 (1H)-one (20.3 g, 87%) as an off-white solid. LRMS (M+H⁺) m/z calculated 240.0, found 240.0.

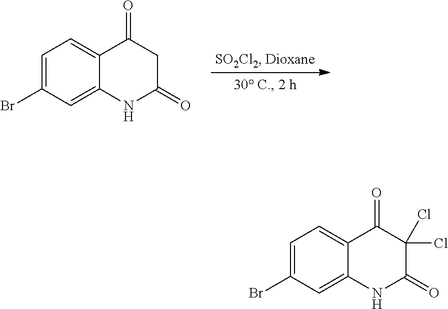 [SMILES: O=C1CC(=O)c2ccc(Br)cc2N1 ; O=C1Nc2cc(Br)ccc2C(=O)C1(Cl)Cl]

To a suspensions of 7-bromo-4-hydroxyquinolin-2 (1H)-one (20.3 g, 84.6 mmol, 1.0 eq) in dioxane (300 mL) was added SO₂Cl₂ (20.6 mL, 253.8 mol, 3.0 eq). The mixture was stirred at 30° C. for 2 h, then poured into ice/water (1 L), and extracted with EtOAc (500 mL×3). The combined organic layers were dried over anhydrous sodium sulfate, filtered and concentrated under vacuum. The resulting residue was purified by column chromatography on silica gel (PE/EA=3/1, v/v) to afford 7-bromo-3,3-dichloroquinoline-2,4(1H,3H)-dione (16.3 g, 62%) as a yellow solid. LRMS (M+H⁺) m/z calculated 307.9, found 307.9. ¹H NMR (DMSO-d₆, 400 MHz) δ 11.57 (s, 1H), 7.73.7.83 (m, 1H), 7.27-7.42 (m, 2H).

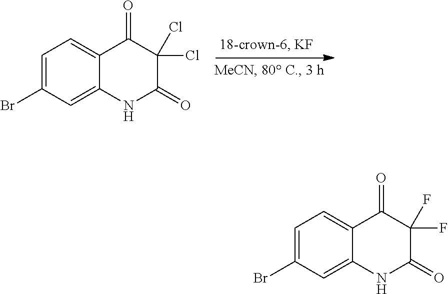 [SMILES: O=C1Nc2cc(Br)ccc2C(=O)C1(Cl)Cl ; O=C1Nc2cc(Br)ccc2C(=O)C1(F)F]

A mixture of 7-bromo-3,3-Dichloroquinoline-2,4(1H,3H)-dione (9.1 g, 29.6 mmol, 1.0 eq), KF (5.2 g, 88.8 mol, 3.0 eq), and 18-crown-6 (781 mg, 3.0 mmol, 0.1 eq) in MeCN (200 mL) was stirred at 80° C. for 3 h. The reaction mixture was filtered, and the filtrate was concentrated under vacuum. The resulting residue was purified by column chromatography on silica gel (PE/EA=1/1, v/v) to afford 7-bromo-3, 3-difluoroquinoline-2, 4 (1H,3H)-dione (5.9 g, 72%) as a yellow solid. LRMS (M+H⁺) m/z calculated 275.9, found 275.9.

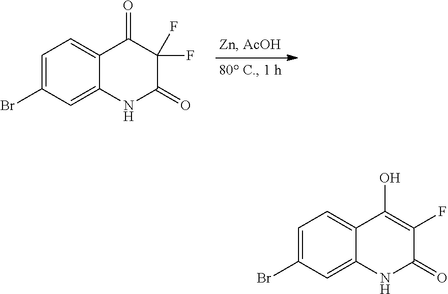 [SMILES: O=C1Nc2cc(Br)ccc2C(=O)C1(F)F ; O=c1[nH]c2cc(Br)ccc2c(O)c1F]

To solutions of 7-bromo-3,3-difluoroquinoline-2,4(1H,3H)-dione (5.9 g, 21.4 mmol, 1.0 eq) in AcOH (100 mL) was added Zn (2.8 g, 42.8 mmol, 2.0 eq) in portions. The mixture was stirred at 80° C. for 1 h. The reaction mixture was concentrated under vacuum. HCl (100 mL, 1 M in water) was added and the mixture was stirred for 20 min. The resulting solid was filtered, washed with EtOAc (20 mL×2) and dried to afford 7-bromo-3-fluoro-4-hydroxyquinolin-2 (1H)-one (4.4 g, 80%) as a gray-white solid. LRMS (M+H⁺) m/z calculated 257.9, found 257.9.

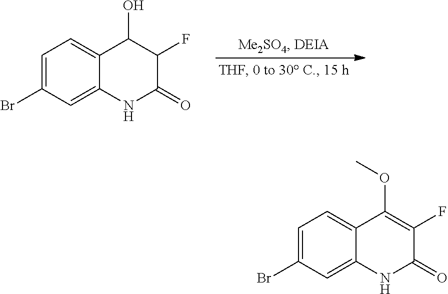

To a suspension of 7-bromo-3-fluoro-4-hydroxyquinolin-2 (1H)-one (3.9 g, 15.1 mmol, 1.0 eq) and DIEA (5.3 mL, 30.2 mmol, 2.0 eq) in THF (100 mL) was added Me₂SO₄ (1.6 mL, 16.6 mmol) dropwise at 30° C. The reaction mixture was stirred at 30° C. for 15 h, then poured into water (200 mL) and extracted with EtOAc (200 mL×3). The combined organic layers were dried over anhydrous sodium sulfate, filtered and concentrated under vacuum. The resulting residue was purified by column chromatography on silica gel (DCM/MeOH=50/1, v/v) to afford 7-bromo-3-fluoro-4-methoxyquinolin-2 (1H)-one (3.2 g, 78%) as a yellow solid. LRMS (M+H⁺) m/z calculated 272.0, found 272.0. ¹H NMR (DMSO-d₆, 400 MHz) δ 12.26 (s, 1H), 7.45-7.54 (m, 2H), 4.23 (d, 3H).

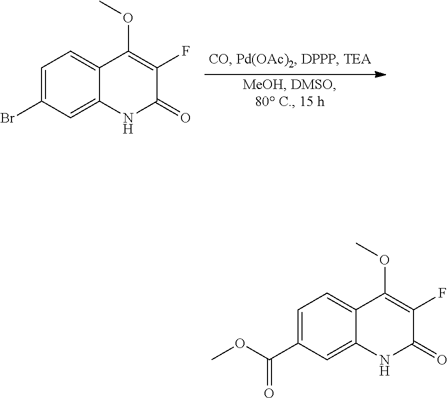 [SMILES: COc1c(F)c(=O)[nH]c2cc(Br)ccc12 ; COC(=O)c1ccc2c(OC)c(F)c(=O)[nH]c2c1]

To a solution of 7-bromo-3-fluoro-4-methoxyquinolin-2 (1H)-one (4 g, 14.7 mmol, 1.0 eq), DPPP (1.2 g, 2.9 mmol, 0.2 eq) and Pd(OAc)₂ (244 mg, 1.5 mmol, 0.1 eq) in DMSO/MeOH (100 mL/100 mL) was added TEA (14.8 mL, 147.1 mmol. 10.0 eq). The mixture was stirred at 80° C. for 15 h under CO (1 atm), then concentrated under vacuum, diluted with water (300 mL), and extracted with EtOAc (550 mL×3). The combined organic layers were washed with brine (300 mL), dried over anhydrous sodium sulfate, filtered, concentrated under vacuum. The resulting residue was purified by column chromatography on silica gel (DCM/MeOH=50/1, v/v) to afford methyl 3-fluoro-4-methoxy-2-oxo-1,2-dihydroquinoline-7-carboxylate (2.7 g, 72%) as a yellow solid. LRMS (M+H⁺) m/z calculated 252.1, found 252.1.

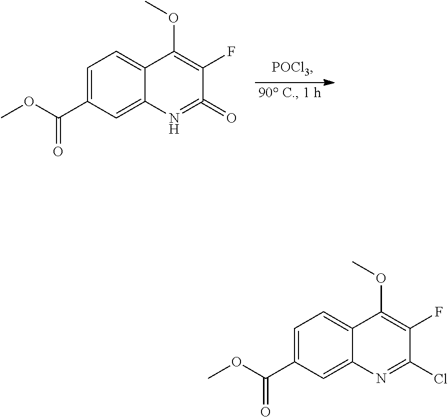

A mixture of 3-fluoro-4-methoxy-2-oxo-1,2-dihydroquinoline-7-carboxylate (2.7 g, 10.8 mmol, 1.0 eq) and POCl₃ (50 mL) was stirred at 90° C. for 1 h. The mixture was concentrated under vacuum and purified by column chromatography on silica gel (PE/EA=5/1, v/v) to afford methyl 2-chloro-3-fluoro-4-methoxyquinoline-7-carboxylate (1.6 g, 52%) as a yellow solid. LRMS (M+H⁺) m/z calculated 270.0, found 270.0.

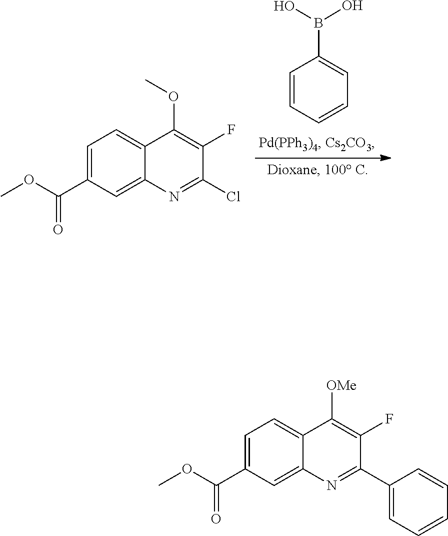 [SMILES: COC(=O)c1ccc2c(OC)c(F)c(Cl)nc2c1 ; OB(O)c1ccccc1 ; COC(=O)c1ccc2c(OC)c(F)c(-c3ccccc3)nc2c1]

To a solution of methyl 2-chloro-3-fluoro-4-methoxyquinoline-7-carboxylate (400 mg, 1.5 mmol, 1.0 eq) in dioxane (50 mL) were added phenylboronic acid (414.5 mg, 3.0 mmol, 2.0 eq), Pd(PPh₃)₄ (171.3 mg, 0.15 mmol, 0.1 eq) and and Cs₂CO₃ (965.9 mg, 3.0 mmol, 2.0 eq). The mixture was stirred at 100° C. for 15 h, and concentrated under vacuum. The resulting residue was purified by column chromatography on silica gel (PE/EA=5/1, v/v) to afford methyl 3-fluoro-4-methoxy-2-phenylquinoline-7-carboxylate (190 mg, 41%) as a white solid. LRMS (M+H⁺) m/z calculated 312.1, found 312.1.

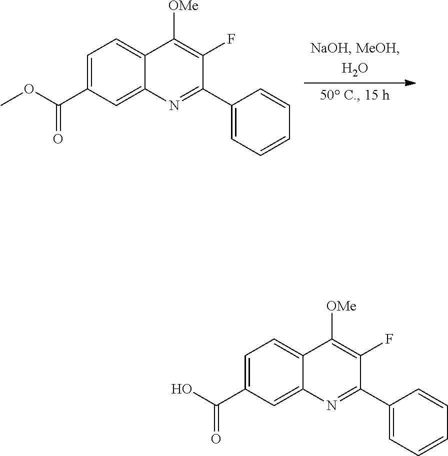 [SMILES: COC(=O)c1ccc2c(OC)c(F)c(-c3ccccc3)nc2c1 ; COc1c(F)c(-c2ccccc2)nc2cc(C(=O)O)ccc12]

To a solution of methyl 3-fluoro-4-methoxy-2-phenylquinoline-7-carboxylate (190 mg, 0.61 mmol, 1.0 eq) in MeOH (3.0 mL) and H₂O (1.0 mL) was added NaOH (36.6 mg, 0.92 mmol, 1.5 eq). The mixture was stirred at 80° C. for 15 h, then concentrated under vacuum and diluted with water (10 mL), and adjusted to pH 2 with 37% HCl aqueous solution. The resulting mixture was stirred for 5 min, filtered and concentrated under vacuum to afford 3-fluoro-4-methoxy-2-phenylquinoline-7-carboxylic acid (140 mg, 86%) as a white solid, LRMS (M+H⁺) m/z calculated 298.1, found 298.1.

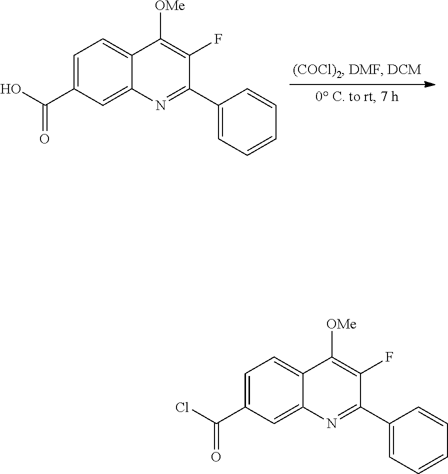 [SMILES: COc1c(F)c(-c2ccccc2)nc2cc(C(=O)O)ccc12 ; COc1c(F)c(-c2ccccc2)nc2cc(C(=O)Cl)ccc12]

To a solution of 3-fluoro-4-methoxy-2-phenylquinoline-7-carboxylic acid (140 mg, 0.47 mmol, 1.0 eq) in DCM (10 mL) were added (COCl)₂ (0.2 mL, 2.35 mmol, 5.0 eq) and DMF (1 drop) at 0° C. The mixture was stirred at rt for 7 h, and concentrated under vacuum to afford 3-fluoro-4-methoxy-2-phenylquinoline-7-carbonyl chloride as a yellow solid (160 mg, ca 100.0%) which was used to the next step directly. LRMS (M+H⁺) m/z calculated 312.1, found 312.1 in MeOH.

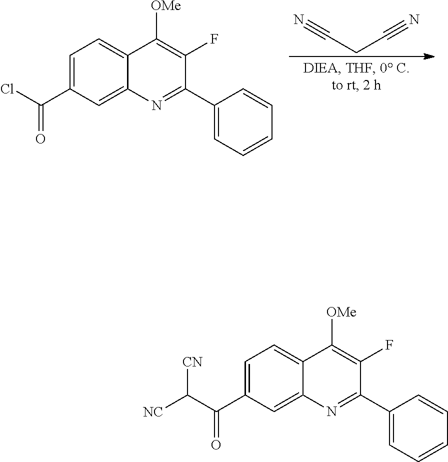 [SMILES: COc1c(F)c(-c2ccccc2)nc2cc(C(=O)Cl)ccc12 ; N#CCC#N ; COc1c(F)c(-c2ccccc2)nc2cc(C(=O)C(C#N)C#N)ccc12]

To a solution of 3-fluoro-4-methoxy-2-phenylquinoline-7-carbonyl chloride (160 mg, 0.51 mmol, 1.0 eq) in THF (10 mL) were added malononitrile (33.7 mg, 0.51 mmol, 1.0 eq) and DIEA (0.27 mL, 1.5 mmol, 3.0 eq). The mixture was stirred at rt for 2 h, then concentrated, diluted with water (20 mL). The resulting mixture was extracted with EtOAc (30 mL×2). The combined organic layers were washed with brine (50 mL), dried over anhydrous sodium sulfate, filtered and concentrated under vacuum. The resulting residue was purified by column chromatography on silica gel (DCM/MeOH=50/1, v/v) to afford 2-(3-fluoro-4-methoxy-2-phenylquinoline-7-carbonyl)malononitrile as a yellow oil (120 mg, 74%), LRMS (M+H⁺) m/z calculated 346.1, found 346.1.

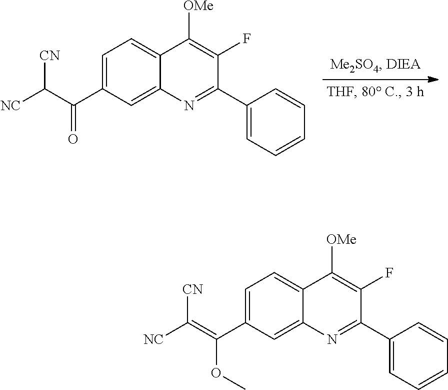 [SMILES: COc1c(F)c(-c2ccccc2)nc2cc(C(=O)C(C#N)C#N)ccc12 ; COC(=C(C#N)C#N)c1ccc2c(OC)c(F)c(-c3ccccc3)nc2c1]

To a solution of 2-(3-fluoro-4-methoxy-2-phenylquinoline-7-carbonyl)malononitrile (120 mg, 0.35 mmol, 1.0 eq) in THF (10 mL) were added Me₂SO₄ (0.07 mL, 0.7 mmol, 2.0 eq) and DIEA (0.31 mL, 1.8 mmol, 5.0 eq). The mixture was stirred at 80° C. for 3 h, then concentrated under vacuum, diluted with water (30 mL), and extracted with EtOAc (30 mL×2). The combined organic layers were washed with brine (40 mL), dried over anhydrous sodium sulfate, filtered and concentrated under vacuum. The resulting residue was purified by column chromatography on silica gel (PE/EA=1/1, v/v) to afford 2-((3-fluoro-4-methoxy-2-phenylquinoline-7-yl)(methoxy)methylene)malononitrile as a yellow oil (40 mg, 32%), LRMS (M+H⁺) m/z calculated 360.1, found 360.1.

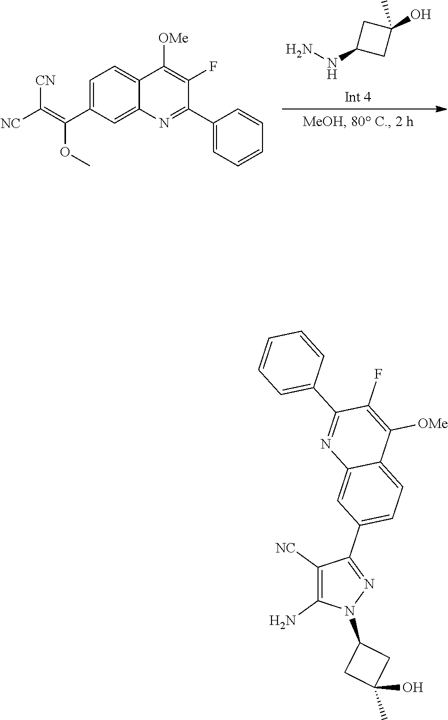

To a solution of 2-((3-fluoro-4-methoxy-2-phenylquinoline-7-yl)(methoxy)methylene) malononitrile (40 mg, 0.11 mmol, 1.0 eq) and (1s,3s)-3-hydrazineyl-1-methylcyclobutan-1-ol (14 mg, 0.12 mmol, 1.1 eq) in MeOH (5 mL) was added TEA (0.2 mL, 1.1 mmol, 10.0 eq) at rt. The reaction mixture was stirred at 80° C. for 2 h, then concentrated under vacuum, diluted with water (10 mL), and extracted with EtOAc (20 mL×2). The combined organic layers were washed with brine (20 mL), dried over anhydrous sodium sulfate, filtered and concentrated under vacuum to afford 5-amino-3-(3-fluoro-4-methoxy-2-phenylquinoline-7-yl)-1-((1s,3s)-3-hydroxy-3-methylcyclobutyl)-1H-pyrazole-4-carbonitrile (70 mg, >100%) as a yellow solid, LRMS (M+H⁺) m/z calculated 444.2, found 444.4.

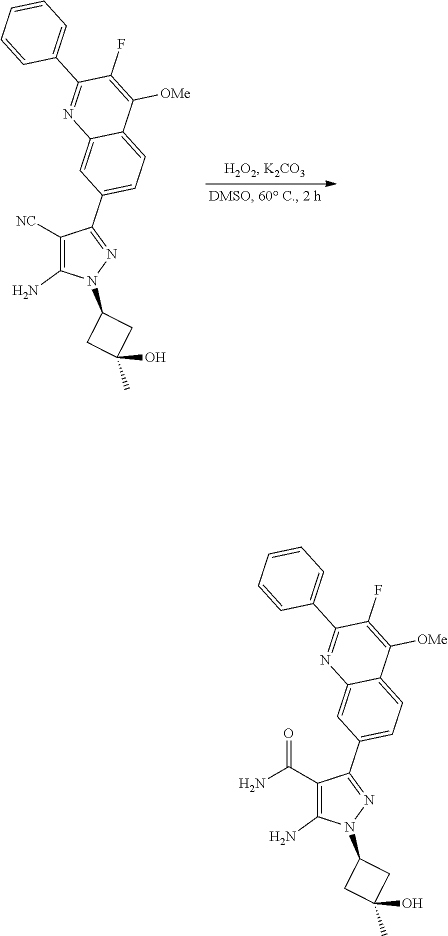 [SMILES: COc1c(F)c(-c2ccccc2)nc2cc(-c3nn([C@H]4C[C@@](C)(O)C4)c(N)c3C#N)ccc12 ; COc1c(F)c(-c2ccccc2)nc2cc(-c3nn([C@H]4C[C@@](C)(O)C4)c(N)c3C(N)=O)ccc12]

To a suspension of 5-amino-3-(3-fluoro-4-methoxy-2-phenylquinoline-7-yl)-1-((1s,3s)-3-hydroxy-3-methylcyclobutyl)-1H-pyrazole-4-carbonitrile (70 mg, 0.16 mmol, 1.0 eq) in DMSO (5 mL) were added K₂CO₃ (110.4 mg, 0.8 mmol, 5.0 eq) and H₂O₂ (30%, 0.36 mL, 3.2 mmol, 20.0 eq) at rt. After addition was complete, the mixture was stirred at 60° C. for 2 h, then diluted with water (20 mL), extracted with EtOAc (20 mL×2). The combined organic layers were washed with brine (40 mL), dried over anhydrous sodium sulfate, filtered and concentrated under vacuum. The resulting residue was purified by column chromatography on silica gel (DCM/MeOH=40/1, v/v) to afford 5-amino-3-(3-fluoro-4-methoxy-2-phenylquinoline-7-yl)-1-((1s,3s)-3-hydroxy-3-methylcyclobutyl)-1H-pyrazole-4-carboxamide as a white solid (10.5 mg, yield 21%), LRMS (M+H⁺) m/z calculated 462.2, found 462.2. ¹H NMR (DMSO-d₆, 400 MHz) δ 8.14-8.18 (m, 2H), 7.96-7.98 (d, 2H), 7.80-7.82 (d, 1H), 7.54-7.60 (m, 3H), 6.27 (s, 2H), 5.19 (s, 1H), 4.43-4.47 (m, 1H), 4.33-4.34 (d, 3H), 2.57-2.62 (m, 2H), 2.35-2.39 (m, 2H), 1.33 (s, 3H). ¹⁹F NMR (DMSO-d₆, 377 MHz) δ −146.6 (s, 1F).

### Example 61: Preparation of 5-amino-3-(3,8-difluoro-2-(2-fluorophenyl)-4-methoxyquinolin-7-yl)-1-((1s,3s)-3-hydroxy-3-methylcyclobutyl)-1H-pyrazole-4-carboxamide

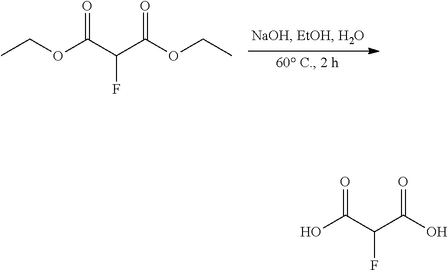

Diethyl 2-fluoromalonate (100.0 g, 561.8 mmol, 1.0 eq) was added dropwise to a stirred solution of NaOH (48.0 g, 1.2 mol, 2.0 eq) in EtOH (1200 mL) and water (300 mL) at 60° C. The mixture was stirred at 60° C. for 2 h, the precipitate was collected. The solid was dissolved in 4 N hydrochloric acid and stirred at rt for 1 h. The mixture was concentrated under vacuum and filtered (washed with MTBE). The filtrate was concentrated under vacuum to afford 2-fluoromalonic acid (66 g, 96%) as a white solid. ¹H NMR (400 MHz, DMSO-d₆) δ 5.48 (d, 1H).

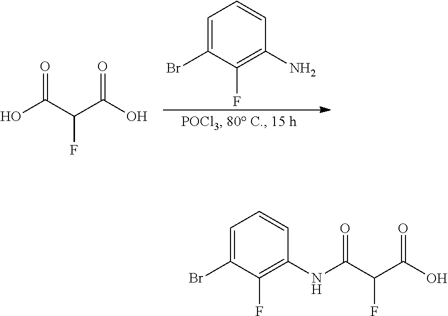 [SMILES: O=C(O)C(F)C(=O)O ; Nc1cccc(Br)c1F ; O=C(O)C(F)C(=O)Nc1cccc(Br)c1F]

A mixture of 2-fluoromalonic acid (66 g, 541 mmol, 1.2 eq) and POCl₃ (1500 mL) was stirred at 80° C. for 30 minutes. Then 3-bromo-2-fluoroaniline (85.2 g, 451 mmol, 1.0 eq) was added and the reaction mixture was stirred at 80° C. for 15 h. After cooling to rt, the mixture was poured into ice. The precipitate was collected by filtration, dissolved in EtOAc (1000 mL) and washed with saturated Na₂CO₃ solution (750 mL×2). The aqueous layer was adjusted to pH 1 by conc. HCl. The precipitate was collected by filtration and and dried to afford 3-((3-bromo-2-fluorophenyl)amino)-2-fluoro-3-oxopropanoic acid (45 g, 28.4%) as a white solid. ¹H NMR (400 MHz, DMSO-d₆) δ 10.70 (s, 1H), 7.92-7.74 (m, 1H), 7.62-7.43 (m, 1H), 7.17 (td, 1H), 5.65 (d, 1H).

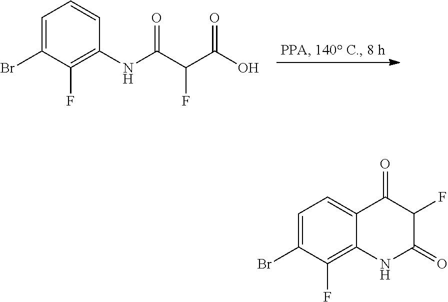 [SMILES: O=C(O)C(F)C(=O)Nc1cccc(Br)c1F ; O=C1Nc2c(ccc(Br)c2F)C(=O)C1F]

A mixture of 3-((3-bromo-2-fluorophenyl)amino)-2-fluoro-3-oxopropanoic acid (45 g, 163.6 mmol) and PPA (500 mL) was stirred at 140° C. for 8 hours. The reaction mixture was poured into ice water with stirring, and extracted with EtOAc (500 mL×2). The combined organic layers were washed with saturated Na₂CO₃ solution (500 mL×2). The aqueous layer was adjusted to pH 1 by conc. HCl, extracted with EtOAc (300 mL×2), dried over anhydrous sodium sulfate, filtered, and concentrated under vacuum to afford 7-bromo-3,8-difluoroquinoline-2,4(1H,3H)-dione (10.1 g, 23.9%) as a white solid. ¹H NMR (400 MHz, DMSO-d₆) δ 12.03 (s, 2H), 7.61 (dd, 1H), 7.47 (dd, 1H).

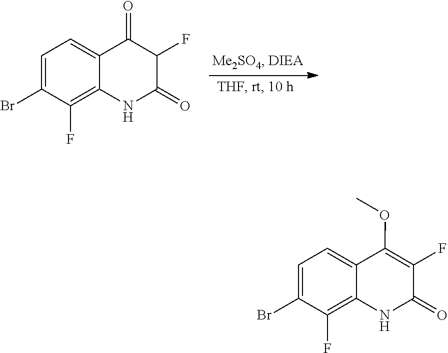 [SMILES: O=C1Nc2c(ccc(Br)c2F)C(=O)C1F ; COc1c(F)c(=O)[nH]c2c(F)c(Br)ccc12]

To a mixture of 7-bromo-3,8-difluoroquinoline-2,4(1H,3H)-dione (4.0 g, 14.5 mmol, 1.0 eq) in THF (50 mL) were added dimethyl sulfate (4.1 g, 29 mmol, 2.0 eq) and DIEA (12.8 g, 72.5 mmol, 5.0 eq). The reaction mixture was stirred at rt for 10 h, and concentrated under vacuum. The resulting residue was purified by column chromatography on silica gel (DCM/MeOH=10/1, v/v) to obtain 7-bromo-3,8-difluoro-4-methoxyquinolin-2 (1H)-one (1.0 g, 23.8%) as a white solid. LRMS (M+H⁺) m/z calculated 291.1, found 291.1

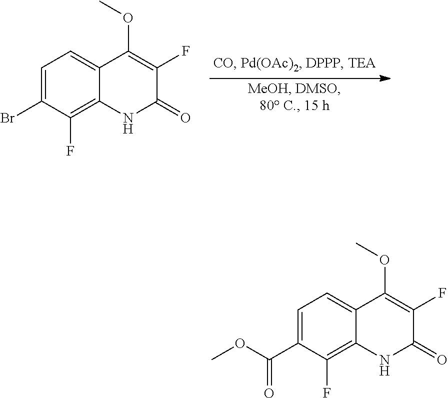 [SMILES: COc1c(F)c(=O)[nH]c2c(F)c(Br)ccc12 ; COC(=O)c1ccc2c(OC)c(F)c(=O)[nH]c2c1F]

To a solution of 7-bromo-3,8-difluoro-4-methoxyquinolin-2 (1H)-one (1.0 g, 3.5 mmol, 1.0 eq) in MeOH (80.0 mL) and DMSO (80.0 mL) were added 1,3-Bis(diphenylphosphino)propane (570 mg, 1.4 mmol, 0.4 eq), Pa(OAc)₂ (233 mg, 1.1 mmol, 0.3 eq) and TEA (1.05 g, 10.5 mmol, 3.0 eq). The mixture was stirred at 80° C. for 15 h under CO. The reaction was quenched with H₂O (500 mL) and extracted with EtOAc (50 mL×3). The combined organic layers were dried over anhydrous sodium sulfate, filtered and concentrated under vacuum. The resulting residue was purified by column chromatography on silica gel (DCM/MeOH=10/1, v/v) to obtain methyl 3,8-difluoro-4-methoxy-2-oxo-1,2-dihydroquinoline-7-carboxylate (500 mg, 51.5%) as a white solid. LRMS (M+H⁺) m/z calculated 270.1, found 270.1.

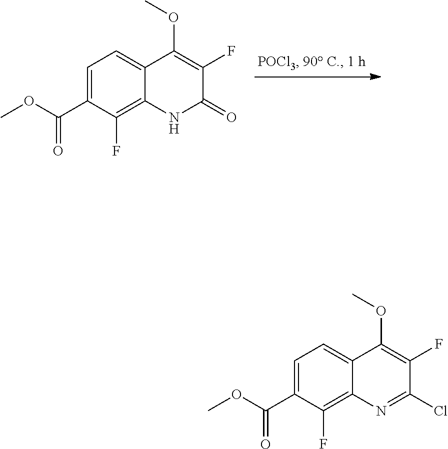 [SMILES: COC(=O)c1ccc2c(OC)c(F)c(=O)[nH]c2c1F ; COC(=O)c1ccc2c(OC)c(F)c(Cl)nc2c1F]

A mixture of methyl 3,8-difluoro-4-methoxy-2-oxo-1,2-dihydroquinoline-7-carboxylate (500 mg, 1.9 mmol, 1.0 eq) and POCl₃ (5.0 mL) was stirred at 90° C. for 1 h. The mixture was concentrated under vacuum, and the resulting residue was purified by column chromatography on silica gel (PE/EtOAc=5/1, v/v) to afford methyl 2-chloro-3,8-difluoro-4-methoxyquinoline-7-carboxylate (400 mg, 75.1%) as a white solid. LRMS (M+H⁺) m/z calculated 288.0, found 288.0.

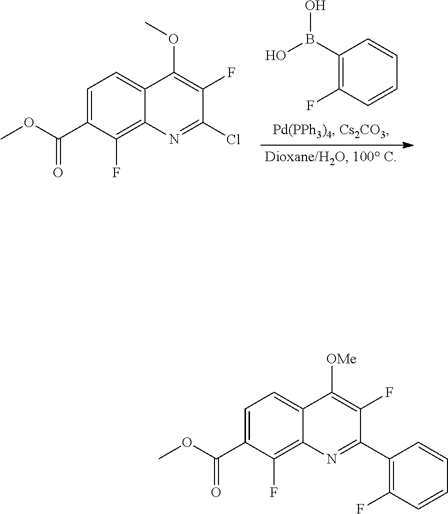 [SMILES: COC(=O)c1ccc2c(OC)c(F)c(Cl)nc2c1F ; OB(O)c1ccccc1F ; COC(=O)c1ccc2c(OC)c(F)c(-c3ccccc3F)nc2c1F]

To a solution of methyl 2-chloro-3,8-difluoro-4-methoxyquinoline-7-carboxylate (200 mg, 0.7 mmol, 1.0 eq) in dioxane (5.0 mL)/H₂O (5.0 mL) were added (2-fluorophenyl) boronic acid (128 mg, 1.0 mmol, 1.4 eq), Pd(PPh₃)₄ (80 mg, 0.07 mmol, 0.1 eq) and cesium carbonate (680 mg, 2.1 mmol, 3.0 eq). The mixture was stirred at 100° C. for 2 h, and concentrated under vacuum. The resulting residue was purified by column chromatography on silica gel (PE/EtOAc=5/1, v/v) to get methyl 3,8-difluoro-2-(2-fluorophenyl)-4-methoxyquinoline-7-carboxylate (190 mg, 72.2%) as a white solid. LRMS (M+H⁺) m/z calculated 348.1, found 348.1.

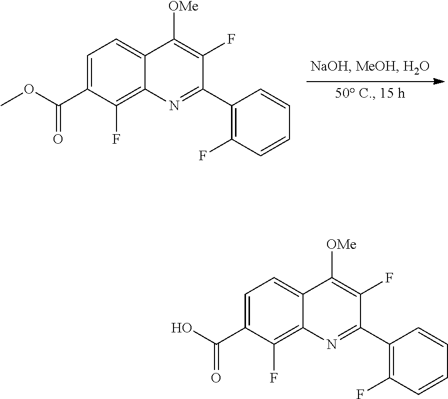 [SMILES: COC(=O)c1ccc2c(OC)c(F)c(-c3ccccc3F)nc2c1F ; COc1c(F)c(-c2ccccc2F)nc2c(F)c(C(=O)O)ccc12]

To a solution of methyl 3,8-difluoro-2-(2-fluorophenyl)-4-methoxyquinoline-7-carboxylate (190 mg, 0.55 mmol, 1.0 eq) in MeOH (10 mL) and H₂O (3 mL) was added NaOH (32.9 mg, 0.82 mmol, 1.5 eq). The mixture was stirred at 80° C. for 15 h, then concentrated under vacuum, diluted with water (20 mL), and adjusted to pH 2 with 37% HCl aqueous solution. The resulting mixture was stirred for 5 min, filtered and concentrated under vacuum to afford 3,8-difluoro-2-(2-fluorophenyl)-4-methoxyquinoline-7-carboxylic acid (160 mg, 87%) as a white solid, LRMS (M+H⁺) m/z calculated 334.1, found 334.0.

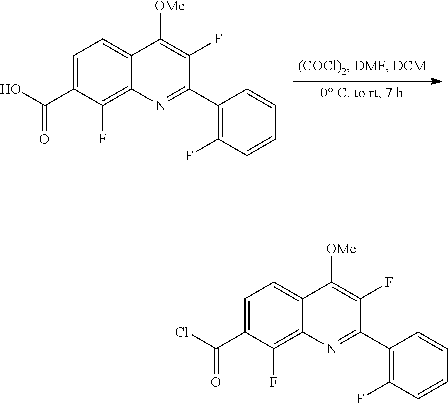 [SMILES: COc1c(F)c(-c2ccccc2F)nc2c(F)c(C(=O)O)ccc12 ; COc1c(F)c(-c2ccccc2F)nc2c(F)c(C(=O)Cl)ccc12]

To a solution of 3,8-difluoro-2-(2-fluorophenyl)-4-methoxyquinoline-7-carboxylic acid (160 mg, 0.48 mmol, 1.0 eq) in DCM (10 mL) were added (COCl)₂ (0.2 mL, 2.4 mmol, 5.0 eq) and DMF (1 drop) at 0° C. The mixture was stirred at rt for 7 h, then concentrated under vacuum to afford 3,8-difluoro-2-(2-fluorophenyl)-4-methoxyquinoline-7-carbonyl chloride as a yellow solid (200 mg, ca 100.0%) which was used in the next step without further purification. LRMS (M+H⁺) m/z calculated 348.1, found 348.1 in MeOH.

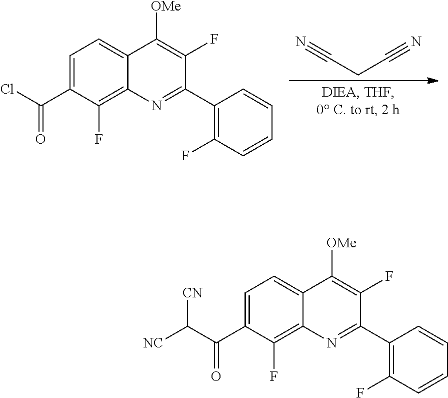 [SMILES: COc1c(F)c(-c2ccccc2F)nc2c(F)c(C(=O)Cl)ccc12 ; N#CCC#N ; COc1c(F)c(-c2ccccc2F)nc2c(F)c(C(=O)C(C#N)C#N)ccc12]

To a solution of 3,8-difluoro-2-(2-fluorophenyl)-4-methoxyquinoline-7-carbonyl chloride (200 mg, 0.52 mmol, 1.0 eq) in THF (10 mL) were added malononitrile (34.6 mg, 0.52 mmol, 1.0 eq) and DIEA (0.28 mL, 1.6 mmol, 3.0 eq). The mixture was stirred at rt for 2 h, then concentrated, diluted with water (20 mL). The resulting mixture was extracted with EtOAc (20 mL×2). The combined organic layers were washed with brine (30 mL), dried over anhydrous sodium sulfate, filtered and concentrated under vacuum. The resulting residue was purified by column chromatography on silica gel (DCM/MeOH=50/1, v/v) to afford 2-(3,8-difluoro-2-(2-fluorophenyl)-4-methoxyquinoline-7-carbonyl)malononitrile as a yellow oil (150 mg, 81%), LRMS (M+H⁺) m/z calculated 382.1, found 382.0.

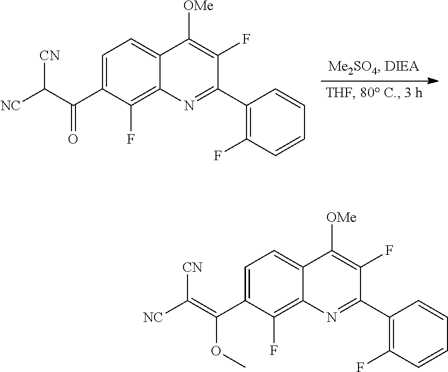 [SMILES: COc1c(F)c(-c2ccccc2F)nc2c(F)c(C(=O)C(C#N)C#N)ccc12 ; COC(=C(C#N)C#N)c1ccc2c(OC)c(F)c(-c3ccccc3F)nc2c1F]

To a solution of 2-(3,8-difluoro-2-(2-fluorophenyl)-4-methoxyquinoline-7-carbonyl) malononitrile (150 mg, 0.39 mmol, 1.0 eq) in THF (10 mL) were added Me₂SO₄ (0.08 mL, 0.78 mmol, 2.0 eq) and DIEA (0.34 mL, 1.95 mmol, 5.0 eq). The mixture was stirred at 80° C. for 3 h, then concentrated under vacuum, diluted with water (30 mL), and extracted with EtOAc (30 mL×2). The combined organic layers were washed with brine (150 mL), dried over anhydrous sodium sulfate, filtered and concentrated under vacuum. The resulting residue was purified by column chromatography on silica gel (PE/EA=1/1, v/v) to afford 2-((3,8-difluoro-2-(2-fluorophenyl)-4-methoxyquinolin-7-yl)(methoxy)methylene)malononitrile as a yellow oil (50 mg, 32%), LRMS (M+H⁺) m/z calculated 396.1, found 396.1.

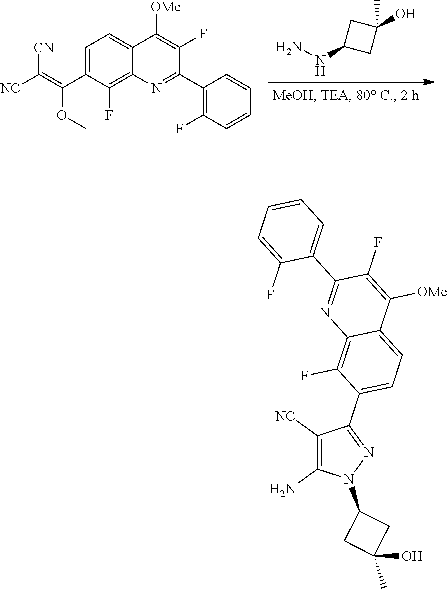

To a solution of 2-((3,8-difluoro-2-(2-fluorophenyl)-4-methoxyquinolin-7-yl)(methoxy)methylene)malononitrile (50 mg, 0.13 mmol, 1.0 eq) and (1s,3s)-3-hydrazineyl-1-methylcyclobutan-1-ol (23.1 mg, 0.14 mmol, 1.1 eq) in MeOH (5 mL) was added TEA (0.2 mL, 1.3 mmol, 10.0 eq) at rt. The reaction mixture was stirred at 80° C. for 2 hours, then concentrated under vacuum, diluted with water (20 mL), and extracted with EtOAc (20 mL×2). The combined organic layers were washed with brine (30 mL), dried over anhydrous sodium sulfate, filtered and concentrated under vacuum to afford 5-amino-3-(3,8-difluoro-2-(2-fluorophenyl)-4-methoxyquinolin-7-yl)-1-((1s,3s)-3-hydroxy-3-methylcyclobutyl)-1H-pyrazole-4-carbonitrile (70 mg, >100%) as a yellow solid, LRMS (M+H⁺) m/z calculated 480.2, found 480.2.

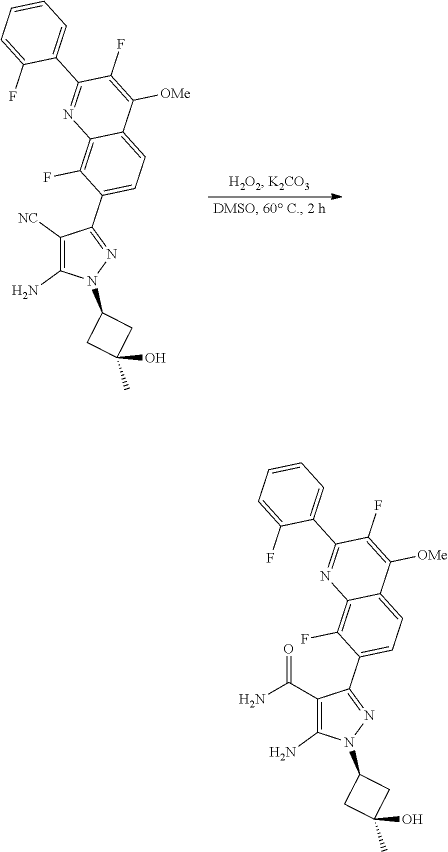 [SMILES: COc1c(F)c(-c2ccccc2F)nc2c(F)c(-c3nn([C@H]4C[C@@](C)(O)C4)c(N)c3C#N)ccc12 ; COc1c(F)c(-c2ccccc2F)nc2c(F)c(-c3nn([C@H]4C[C@@](C)(O)C4)c(N)c3C(N)=O)ccc12]

To a suspension of 5-amino-3-(3,8-difluoro-2-(2-fluorophenyl)-4-methoxyquinolin-7-yl)-1-((1s,3s)-3-hydroxy-3-methylcyclobutyl)-1H-pyrazole-4-carbonitrile (70 mg, 0.15 mmol, 1.0 eq) in DMSO (3 mL) were added K₂CO₃ (103.5 mg, 0.75 mmol, 5.0 eq) and H₂O₂ (30%, 0.3 mL, 3 mmol, 20.0 eq) at rt. After addition was complete, the mixture was stirred at 60° C. for 2 h, then diluted with water (10 mL), and extracted with EtOAc (20 mL×2). The combined organic layers were washed with brine (20 mL), dried over anhydrous sodium sulfate, filtered and concentrated under vacuum. The resulting residue was purified by column chromatography on silica gel (DCM/MeOH=40/1, v/v) to afford 5-amino-3-(3,8-difluoro-2-(2-fluorophenyl)-4-methoxyquinolin-7-yl)-1-((1s,3s)-3-hydroxy-3-methylcyclobutyl)-1H-pyrazole-4-carboxamide as a white solid (15.9 mg, 0.03 mmol, yield 21.8%), LRMS (M+H⁺) m/z calculated 498.2, found 498.2. ¹H NMR (DMSO-d₆, 400 MHz) δ 8.00-8.02 (d, 1H), 7.63-7.73 (m, 3H), 7.40-7.44 (m, 2H), 6.30 (s, 2H), 5.18 (s, 1H), 4.44-4.48 (m, 1H), 4.34-4.35 (d, 3H), 2.51-2.58 (m, 2H), 2.35-2.40 (m, 2H), 1.32 (s, 3H). ¹⁹F NMR (DMSO-d₆, 377 MHz) δ −115.1 (d, 1F), −124.7 (d, 1F), −144.7 (t, 1F).

### Example 62: Preparation of 5-amino-3-(3,8-difluoro-4-methoxy-2-phenylquinolin-7-yl)-1-((1s,3s)-3-hydroxy-3-methylcyclobutyl)-1H-pyrazole-4-carboxamide

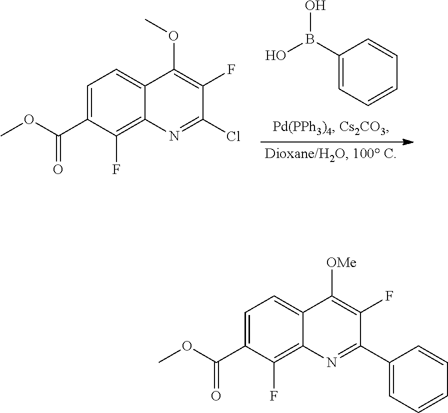 [SMILES: COC(=O)c1ccc2c(OC)c(F)c(Cl)nc2c1F ; OB(O)c1ccccc1 ; COC(=O)c1ccc2c(OC)c(F)c(-c3ccccc3)nc2c1F]

To a solution of methyl 2-chloro-3,8-difluoro-4-methoxyquinoline-7-carboxylate (200 mg, 0.7 mmol, 1.0 eq) in dioxane (5.0 mL)/H₂O (5.0 mL) were added phenylboronic acid (122 mg, 1.0 mmol, 1.4 eq), Pd(PPh₃)₄ (80 mg, 0.07 mmol, 0.1 eq) and cesium carbonate (680 mg, 2.1 mmol, 3.0 eq). The mixture was stirred at 100° C. for 2 h, and concentrated under vacuum. The resulting residue was purified by column chromatography on silica gel (PE/EtOAc=5/1, v/v) to get methyl 3,8-difluoro-4-methoxy-2-phenylquinoline-7-carboxylate (160 mg, 69.9%) as a white solid. LRMS (M+H⁺) m/z calculated 330.1, found 330.1.

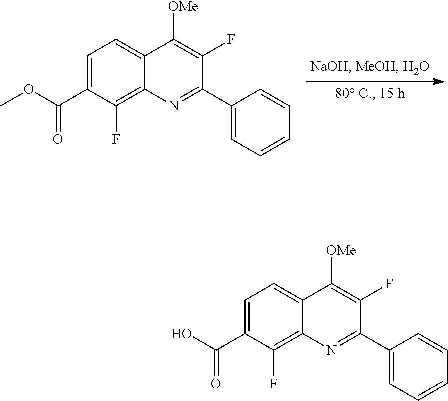 [SMILES: COC(=O)c1ccc2c(OC)c(F)c(-c3ccccc3)nc2c1F ; COc1c(F)c(-c2ccccc2)nc2c(F)c(C(=O)O)ccc12]

To a solution of methyl 3,8-difluoro-4-methoxy-2-phenylquinoline-7-carboxylate (160 mg, 0.49 mmol, 1.0 eq) in MeOH (10 mL) and H₂O (3 mL) was added NaOH (29.1 mg, 0.73 mmol, 1.5 eq). The mixture was stirred at 80° C. for 15 h, then concentrated under vacuum and diluted with water (20 mL), adjusted to pH 2 with 37% HCl aqueous solution. The resulting mixture was stirred for 5 min, filtered and concentrated under vacuum to afford 3,8-difluoro-4-methoxy-2-phenylquinoline-7-carboxylic acid (140 mg, 91.5%) as a white solid, LRMS (M+H⁺) m/z calculated 316.1, found 316.1.

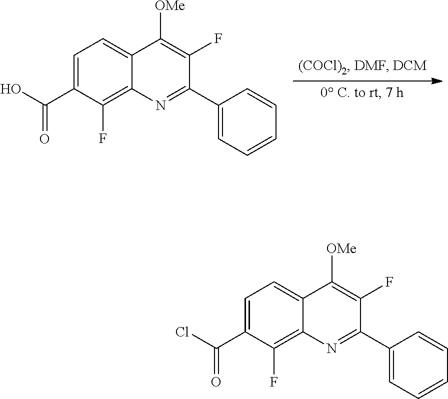 [SMILES: COc1c(F)c(-c2ccccc2)nc2c(F)c(C(=O)O)ccc12 ; COc1c(F)c(-c2ccccc2)nc2c(F)c(C(=O)Cl)ccc12]

To a solution of 3,8-difluoro-4-methoxy-2-phenylquinoline-7-carboxylic acid (140 mg, 0.44 mmol, 1.0 eq) in DCM (10 mL) were added (COCl)₂ (0.2 mL, 2.2 mmol, 5.0 eq) and DMF (1 drop) at 0° C. The mixture was stirred at rt for 7 h, and concentrated under vacuum to afford 3,8-difluoro-4-methoxy-2-phenylquinoline-7-carbonyl chloride as a yellow solid (150 mg, ca 100.0%) which was used to the next step directly. LRMS (M+H⁺) m/z calculated 330.1, found 330.1 in MeOH.

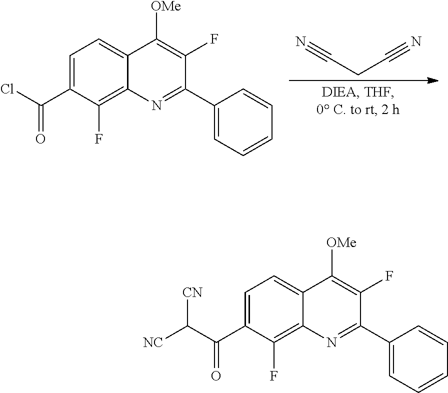 [SMILES: COc1c(F)c(-c2ccccc2)nc2c(F)c(C(=O)Cl)ccc12 ; N#CCC#N ; COc1c(F)c(-c2ccccc2)nc2c(F)c(C(=O)C(C#N)C#N)ccc12]

To a solution of 3,8-difluoro-4-methoxy-2-phenylquinoline-7-carbonyl chloride (150 mg, 0.45 mmol, 1.0 eq) in THF (10 mL) were added malononitrile (29.7 mg, 0.45 mmol, 1.0 eq) and DIEA (0.28 mL, 1.4 mmol, 3.0 eq). The mixture was stirred at rt for 2 h, concentrated, and diluted with water (20 mL). The resulting mixture was extracted with EtOAc (20 mL×2). The combined organic layers were washed with brine (30 mL), dried over anhydrous sodium sulfate, filtered and concentrated under vacuum. The resulting residue was purified by column chromatography on silica gel (DCM/MeOH=50/1, v/v) to afford 2-(3,8-difluoro-4-methoxy-2-phenylquinoline-7-carbonyl)malononitrile as a yellow oil (200 mg, >100%), LRMS (M+H⁺) m/z calculated 364.1, found 364.0.

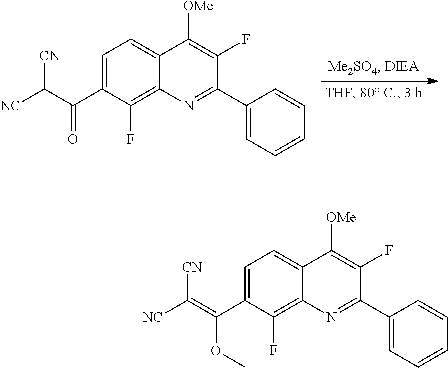 [SMILES: COc1c(F)c(-c2ccccc2)nc2c(F)c(C(=O)C(C#N)C#N)ccc12 ; COC(=C(C#N)C#N)c1ccc2c(OC)c(F)c(-c3ccccc3)nc2c1F]

To a solution of 2-(3,8-difluoro-4-methoxy-2-phenylquinoline-7-carbonyl)malononitrile (200 mg, 0.76 mmol, 1.0 eq) in THF (10 mL) were added Me₂SO₄ (0.15 mL, 1.5 mmol, 2.0 eq) and DIEA (0.66 mL, 3.8 mmol, 5.0 eq). The mixture was stirred at 80° C. for 3 h, then concentrated under vacuum, diluted with water (30 mL), and extracted with EtOAc (30 mL×2). The combined organic layers were washed with brine (40 mL), dried over anhydrous sodium sulfate, filtered and concentrated under vacuum. The resulting residue was purified by column chromatography on silica gel (PE/EA=1/1, v/v) to afford 2-((3,8-difluoro-4-methoxy-2-phenylquinoline-7-yl)(methoxy)methylene)malononitrile as a yellow oil (30 mg, 14.5%), LRMS (M+H⁺) m/z calculated 378.1, found 378.1.

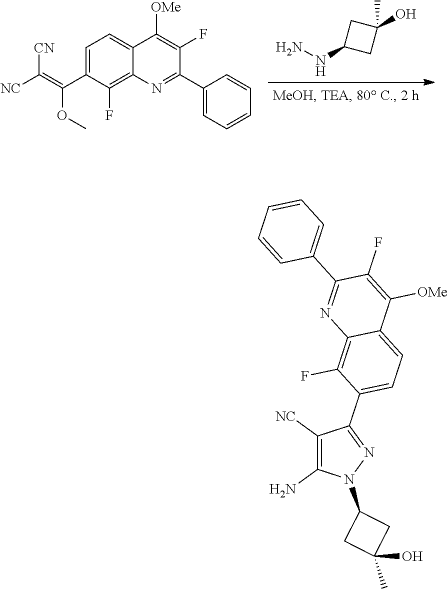

To a solution of 2-((3,8-difluoro-4-methoxy-2-phenylquinoline-7-yl)(methoxy)methylene)malononitrile (30 mg, 0.06 mmol, 1.0 eq) and (1s,3s)-3-hydrazineyl-1-methylcyclobutan-1-ol (11.4 mg, 0.07 mmol, 1.1 eq) in MeOH (5 mL) was added TEA (0.1 mL, 0.6 mmol, 10.0 eq) at rt. The reaction mixture was stirred at 80° C. for 2 hours, then concentrated under vacuum, diluted with water (20 mL), and extracted with EtOAc (20 mL×2). The combined organic layers were washed with brine (30 mL), dried over anhydrous sodium sulfate, filtered and concentrated under vacuum to afford 5-amino-3-(3,8-difluoro-4-methoxy-2-phenylquinoline-7-yl)-1-((1s,3s)-3-hydroxy-3-methylcyclobutyl)-1H-pyrazole-4-carbonitrile (20 mg, 55.6%) as a yellow solid, LRMS (M+H⁺) m/z calculated 462.2, found 462.2.

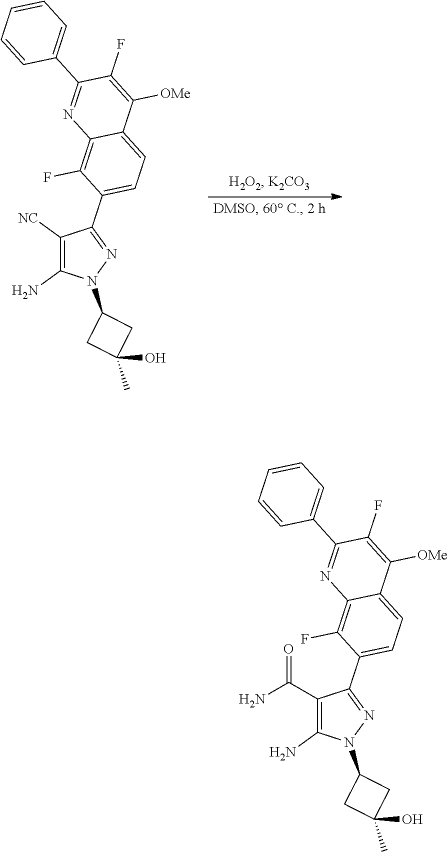 [SMILES: COc1c(F)c(-c2ccccc2)nc2c(F)c(-c3nn([C@H]4C[C@@](C)(O)C4)c(N)c3C#N)ccc12 ; COc1c(F)c(-c2ccccc2)nc2c(F)c(-c3nn([C@H]4C[C@@](C)(O)C4)c(N)c3C(N)=O)ccc12]

To a suspension of 5-amino-3-(3,8-difluoro-4-methoxy-2-phenylquinoline-7-yl)-1-((1s,3s)-3-hydroxy-3-methylcyclobutyl)-1H-pyrazole-4-carbonitrile (20 mg, 0.04 mmol, 1.0 eq) in DMSO (3 mL) were added K₂CO₃ (27.6 mg, 0.2 mmol, 5.0 eq) and H₂O₂ (30%, 0.2 mL, 0.8 mmol, 20.0 eq) at rt. After addition was complete, the mixture was stirred at 60° C. for 2 h, then diluted with water (10 mL), and extracted with EtOAc (20 mL×2). The combined organic layers were washed with brine (20 mL), dried over anhydrous sodium sulfate, filtered and concentrated under vacuum. The resulting residue was purified by column chromatography on silica gel (DCM/MeOH=40/1, v/v) to afford 5-amino-3-(3,8-difluoro-4-methoxy-2-phenylquinolin-7-yl)-1-((1s,3s)-3-hydroxy-3-methylcyclobutyl)-1H-pyrazole-4-carboxamide as a white solid (7.7 mg, 38.5%), LRMS (M+H⁺) m/z calculated 480.2, found 480.2. ¹H NMR (DMSO-d₆, 400 MHz) δ 7.96-7.99 (t, 3H), 7.56-7.64 (m, 4H), 6.30 (s, 2H), 5.18 (s, 1H), 4.44-4.48 (m, 1H), 4.35-4.36 (d, 3H), 2.54-2.59 (m, 2H), 2.35-2.40 (m, 2H), 1.32 (s, 3H). ¹⁹F NMR (DMSO-d₆, 377 MHz) δ −124.8 (d, 1F), −145.4 (d, 1F).

### Example 63: Preparation of 5-amino-3-(3-fluoro-2-(2-fluorophenyl)-4-methoxyquinolin-7-yl)-1-((1s,3s)-3-hydroxy-3-methylcyclobutyl)-1H-pyrazole-4-carboxamide

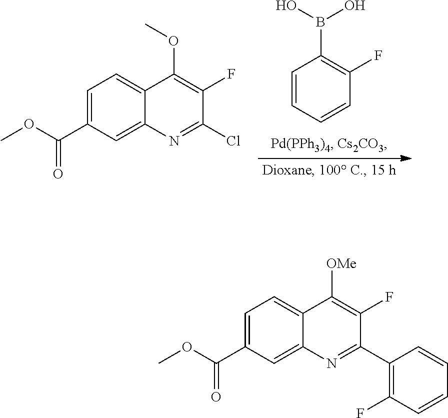 [SMILES: COC(=O)c1ccc2c(OC)c(F)c(Cl)nc2c1 ; OB(O)c1ccccc1F ; COC(=O)c1ccc2c(OC)c(F)c(-c3ccccc3F)nc2c1]

To a solution of methyl 2-chloro-3-fluoro-4-methoxyquinoline-7-carboxylate (400 mg, 1.5 mmol, 1.0 eq) in dioxane (50 mL) were added (2-fluorophenyl) boronic acid (420 mg, 3.0 mmol, 2.0 eq), Pd(PPh₃)₄ (171.3 mg, 0.15 mmol, 0.1 eq) and and Cs₂CO₃ (965.9 mg, 3.0 mmol, 2.0 eq). The mixture was stirred at 100° C. for 15 h. The reaction mixture was concentrated under vacuum. The resulting residue was purified by column chromatography on silica gel (PE/EA=5/1, v/v) to afford methyl 3-fluoro-2-(2-fluorophenyl)-4-methoxyquinoline-7-carboxylate (210 mg, 42.9%) as a white solid. LRMS (M+H⁺) m/z calculated 330.1, found 330.1.

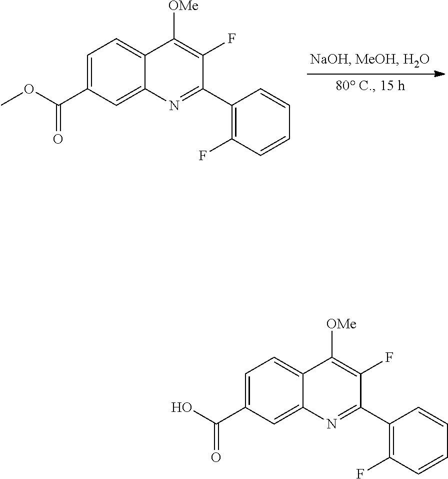 [SMILES: COC(=O)c1ccc2c(OC)c(F)c(-c3ccccc3F)nc2c1 ; COc1c(F)c(-c2ccccc2F)nc2cc(C(=O)O)ccc12]

To a solution of methyl 3-fluoro-2-(2-fluorophenyl)-4-methoxyquinoline-7-carboxylate (210 mg, 0.64 mmol, 1.0 eq) in MeOH (3.0 mL) and H₂O (1.0 mL) was added NaOH (38.2 mg, 0.96 mmol, 1.5 eq). The mixture was stirred at 80° C. for 15 h, then concentrated under vacuum, diluted with water (10 mL), and adjusted to pH=2 with 37% HCl aqueous solution. The resulting mixture was stirred for 5 min, filtered and concentrated under vacuum to afford 3-fluoro-2-(2-fluorophenyl)-4-methoxyquinoline-7-carboxylic acid (170 mg, 84.6%) as a white solid, LRMS (M+H⁺) m/z calculated 316.1, found 316.1.

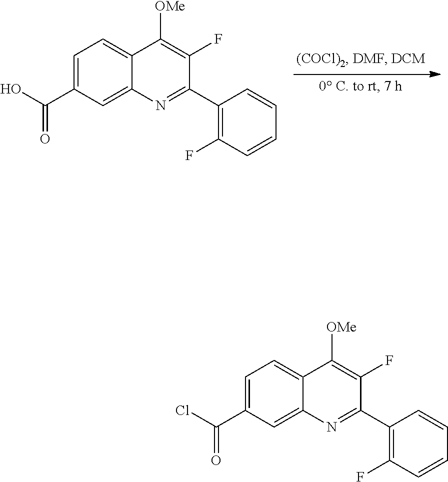 [SMILES: COc1c(F)c(-c2ccccc2F)nc2cc(C(=O)O)ccc12 ; COc1c(F)c(-c2ccccc2F)nc2cc(C(=O)Cl)ccc12]

To a solution of 3-fluoro-2-(2-fluorophenyl)-4-methoxyquinoline-7-carboxylic acid (170 mg, 0.54 mmol, 1.0 eq) in DCM (10 mL) were added (COCl)₂ (0.2 mL, 2.7 mmol, 5.0 eq) and 1 drop DMF at 0° C. The mixture was stirred at rt for 7 h, then concentrated under vacuum to afford 3-fluoro-2-(2-fluorophenyl)-4-methoxyquinoline-7-carbonyl chloride as a yellow solid (170 mg, ca 100.0%) which was used in the next step without further purification. LRMS (M+H⁺) m/z calculated 330.1, found 330.1 in MeOH.

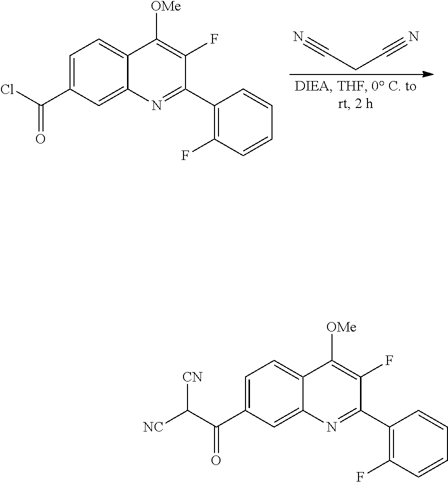 [SMILES: COc1c(F)c(-c2ccccc2F)nc2cc(C(=O)Cl)ccc12 ; N#CCC#N ; COc1c(F)c(-c2ccccc2F)nc2cc(C(=O)C(C#N)C#N)ccc12]

To a solution of 3-fluoro-2-(2-fluorophenyl)-4-methoxyquinoline-7-carbonyl chloride (170 mg, 0.51 mmol, 1.0 eq) in THF (10 mL) were added malononitrile (33.7 mg, 0.51 mmol, 1.0 eq) and DIEA (0.27 mL, 1.5 mmol, 3.0 eq). The mixture was stirred at rt for 2 h, then concentrated, diluted with water (20 mL). The resulting mixture was extracted with EtOAc (30 mL×2). The combined organic layers were washed with brine (50 mL), dried over anhydrous sodium sulfate, filtered and concentrated under vacuum. The resulting residue was purified by column chromatography on silica gel (DCM/MeOH=50/1, v/v) to afford 2-(3-fluoro-2-(2-fluorophenyl)-4-methoxyquinoline-7-carbonyl)malononitrile as a yellow oil (220 mg, ca 100%), LRMS (M+H⁺) m/z calculated 364.1, found 364.1.

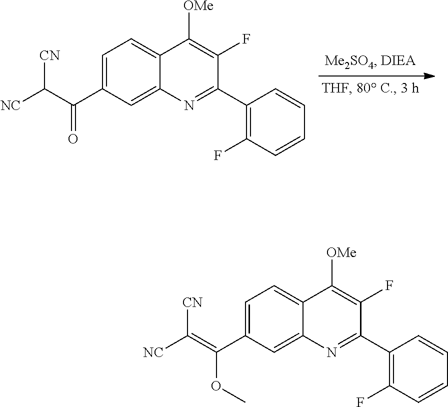 [SMILES: COc1c(F)c(-c2ccccc2F)nc2cc(C(=O)C(C#N)C#N)ccc12 ; COC(=C(C#N)C#N)c1ccc2c(OC)c(F)c(-c3ccccc3F)nc2c1]

To a solution of 2-(3-fluoro-2-(2-fluorophenyl)-4-methoxyquinoline-7-carbonyl)malononitrile (220 mg, 0.61 mmol, 1.0 eq) in THF (10 mL) were added Me₂SO₄ (0.1 mL, 1.2 mmol, 2.0 eq) and DIEA (0.53 mL, 3.1 mmol, 5.0 eq). The mixture was stirred at 80° C. for 3 h, then concentrated under vacuum, diluted with water (30 mL), and extracted with EtOAc (30 mL×2). The combined organic layers were washed with brine (40 mL), dried over anhydrous sodium sulfate, filtered and concentrated under vacuum. The resulting residue was purified by column chromatography on silica gel (PE/EA=1/1, v/v) to afford 2-((3-fluoro-2-(2-fluorophenyl)-4-methoxyquinolin-7-yl)(methoxy)methylene)malononitrile as a yellow oil (70 mg, 30.7%), LRMS (M+H⁺) m/z calculated 378.1, found 378.1.

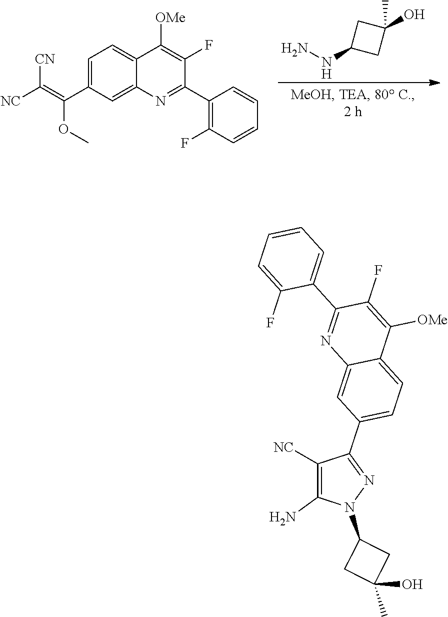

To a solution of 2-((3-fluoro-2-(2-fluorophenyl)-4-methoxyquinolin-7-yl)(methoxy)methylene) malononitrile (70 mg, 0.19 mmol, 1.0 eq) and (1s,3s)-3-hydrazineyl-1-methylcyclobutan-1-ol (34.7 mg, 0.21 mmol, 1.1 eq) in MeOH (5 mL) was added TEA (0.3 mL, 1.9 mmol, 10.0 eq) at rt. The reaction mixture was stirred at 80° C. for 2 h, then concentrated under vacuum, diluted with water (10 mL), and extracted with EtOAc (20 mL×2). The combined organic layers were washed with brine (20 mL), dried over anhydrous sodium sulfate, filtered and concentrated under vacuum to afford 5-amino-3-(3-fluoro-2-(2-fluorophenyl)-4-methoxyquinolin-7-yl)-1-((1s,3s)-3-hydroxy-3-methylcyclobutyl)-1H-pyrazole-4-carbonitrile (100 mg, ca.) as a yellow solid, LRMS (M+H⁺) m/z calculated 462.2, found 462.2.

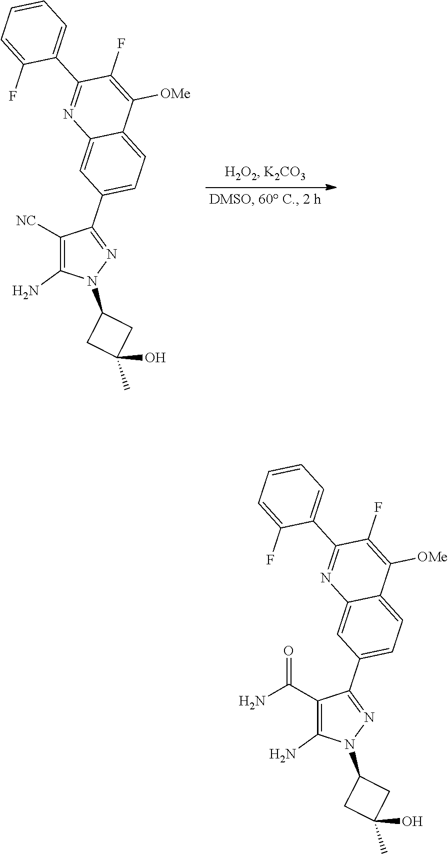 [SMILES: COc1c(F)c(-c2ccccc2F)nc2cc(-c3nn([C@H]4C[C@@](C)(O)C4)c(N)c3C#N)ccc12 ; COc1c(F)c(-c2ccccc2F)nc2cc(-c3nn([C@H]4C[C@@](C)(O)C4)c(N)c3C(N)=O)ccc12]

To a stirred solution of 5-amino-3-(3-fluoro-2-(2-fluorophenyl)-4-methoxyquinolin-7-yl)-1-((1s,3s)-3-hydroxy-3-methylcyclobutyl)-1H-pyrazole-4-carbonitrile (100 mg, 0.22 mmol, 1.0 eq) in DMSO (5 mL) were added K₂CO₃ (151.8 mg, 1.1 mmol, 5.0 eq) and H₂O₂ (30%, 0.42 mL, 4.4 mmol, 20.0 eq) at rt. After addition was complete, the mixture was stirred at 60° C. for 2 h, then diluted with water (20 mL), extracted with EtOAc (20 mL×2). The combined organic layers were washed with brine (40 mL), dried over anhydrous sodium sulfate, filtered and concentrated under vacuum. The resulting residue was purified by column chromatography on silica gel (DCM/MeOH=40/1, v/v) to afford 5-amino-3-(3-fluoro-2-(2-fluorophenyl)-4-methoxyquinolin-7-yl)-1-((1s,3s)-3-hydroxy-3-methylcyclobutyl)-1H-pyrazole-4-carboxamide as a white solid (29.1 mg, 28.2%), LRMS (M+H⁺) m/z calculated 480.2, found 480.2. ¹H NMR (DMSO-d₆, 400 MHz) δ 8.20-8.22 (d, 1H), 8.15-8.16 (d, 1H), 7.83-7.86 (t, 1H), 7.70-7.72 (d, 1H), 7.61-7.63 (d, 1H), 7.38-7.43 (m, 2H), 6.27 (s, 2H), 5.19 (s, 1H), 4.33-4.47 (m, 1H), 4.32-4.33 (d, 3H), 2.57-2.62 (m, 2H), 2.35-2.39 (m, 2H), 1.33 (s, 3H). ¹⁹F NMR (DMSO-d₆, 377 MHz) δ −115.2 (d, 1F), −145.8 (d, 1F).

### Example 64: Preparation of 5-amino-1-((1s,3s)-3-hydroxy-3-methylcyclobutyl)-3-(4-methoxy-2-phenylquinazolin-7-yl)-1H-pyrazole-4-carboxamide

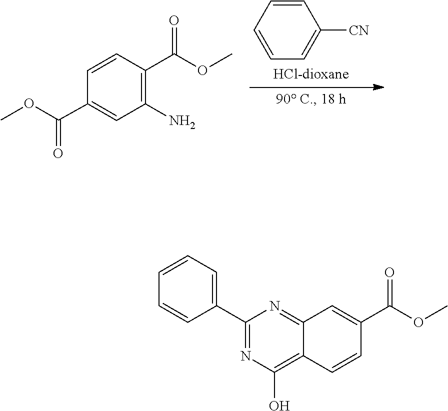

A mixture of dimethyl 2-aminoterephthalate (5 g, 23.9 mmol, 1.0 eq) and benzonitrile (37.1 mg, 0.16 mmol, 0.1 eq) was dissolved in a 4N HCl solution in dioxane (4N, 100 mL). The mixture was stirred at 90° C. for 18 h under N₂, and concentrated under vacuum. The resulting residue was purified by column chromatography on silica gel (PE/EA=5/1, v/v) to afford methyl 4-hydroxy-2-phenylquinazoline-7-carboxylate (6.0 g, 21.4 mmol, 89.5%) as a yellow oil. LRMS (M+H⁺) m/z calculated 281.1, found 281.1.

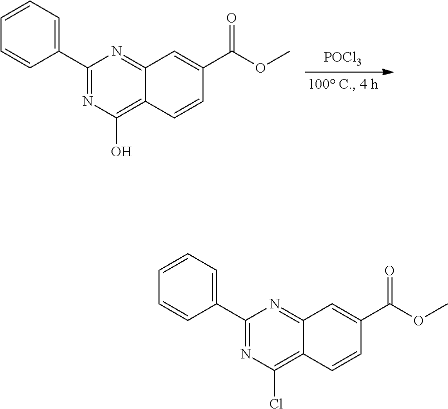

To a solution of methyl 4-hydroxy-2-phenylquinazoline-7-carboxylate (6 g, 21.4 mmol, 1.0 eq), in POCl₃ (50 mL) was stirred at 100° C. for 4 h under N₂. The resulting residue was concentrated under vacuum, and partitioned between EA (100 mL) and sat. aq. NaHCO₃. The organic layer was dried over anhydrous sodium sulfate, filtered and concentrated, the resulting residue was purified by column chromatography on silica gel (PE/EA=5/1, v/v) to afford methyl 4-chloro-2-phenylquinazoline-7-carboxylate (5.0 g, 16.8 mmol, 78.5%) as a yellow oil. LRMS (M+H⁺) m/z calculated 299.1, found 299.1.

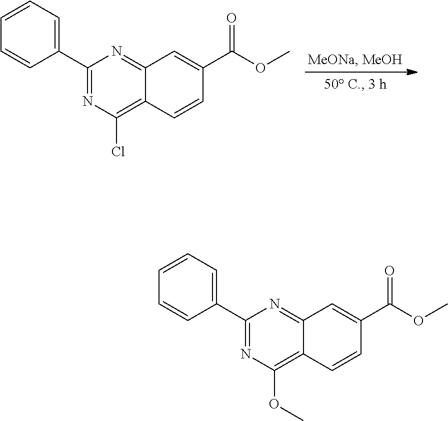

To a solution of methyl 4-chloro-2-phenylquinazoline-7-carboxylate (5.0 g, 16.8 mmol, 1.0 eq), in MeOH (100 mL) was added NaOMe (2.72 g, 50.4 mmol. 3.0 eq). The mixture was stirred at 50° C. for 3 h under N₂. The reaction mixture was concentrated under vacuum. The resulting residue was partitioned between sat. aq. NH₄Cl and EA. The organic layer was dried over anhydrous sodium sulfate, filtered and concentrated. The resulting residue was purified by column chromatography on silica gel (PE/EA=5/1, v/v) to afford methyl 4-methoxy-2-phenylquinazoline-7-carboxylate (4.3 g, 14.6 mmol, 87.0%) as a yellow oil. LRMS (M+H⁺) m/z calculated 295.1, found 295.1.

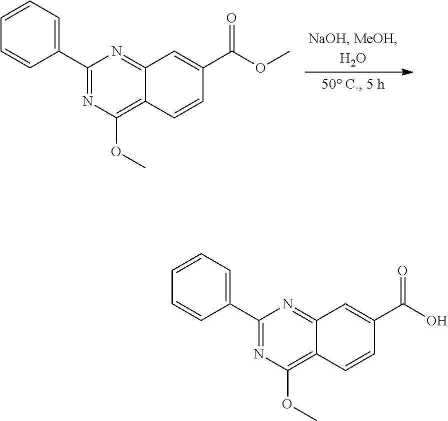

To a solution of methyl 4-methoxy-2-phenylquinazoline-7-carboxylate (4.3 g, 14.6 mmol, 1.0 eq) in MeOH (100 mL) and H₂O (2 mL) was added NaOH (876 mg, 21.9 mmol, 1.5 eq). The mixture was stirred at 50° C. for 5 h, then concentrated under vacuum, and diluted with water (20 mL). 37% HCl was added to adjust to pH 2. The resulting mixture was stirred for 5 min, and filtered. The filter cake was further washed with water (5 mL×2), and dried under vacuum to afford 4-methoxy-2-phenylquinazoline-7-carboxylic acid (4.0 g, 14.3 mmol, 97.8%) as a white solid, LRMS (M+H⁺) m/z calculated 281.1, found 281.1.

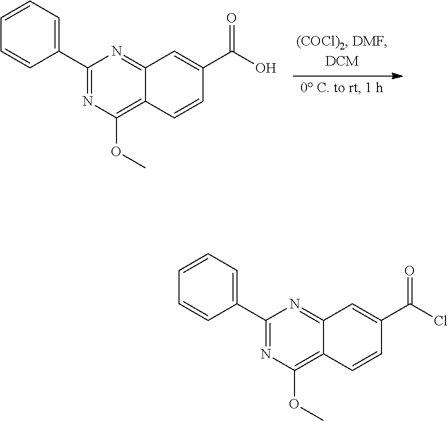 [SMILES: COc1nc(-c2ccccc2)nc2cc(C(=O)O)ccc12 ; COc1nc(-c2ccccc2)nc2cc(C(=O)Cl)ccc12]

To a solution of 4-methoxy-2-phenylquinazoline-7-carboxylic acid (2.0 g, 7.14 mmol, 1.0 eq) in DCM (100 mL) were added (COCl)₂ (9.3 mL, 1.5 mmol, 5.0 eq) and DMF (3 drops) at 0° C. The mixture was stirred at rt for 1 h, and concentrated under vacuum to afford 4-methoxy-2-phenylquinazoline-7-carbonyl chloride as a yellow solid (2.1 g, 7.04 mmol, 98.7%), LRMS (M+H⁺) m/z calculated 295.1, found 295.1 in MeOH.

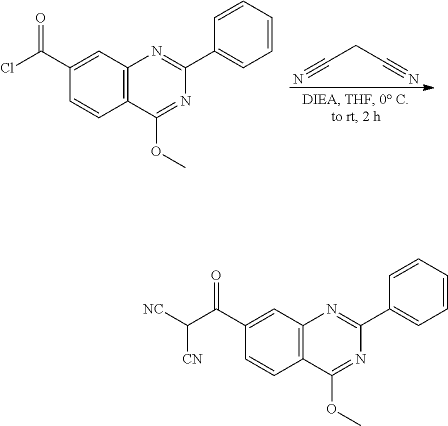

To a solution of 4-methoxy-2-phenylquinazoline-7-carbonyl chloride (2.1 g, 7.04 mmol, 1.0 eq) in THF (100 mL) were added malononitrile (465 mg, 7.04 mmol, 1.0 eq) and DIEA (3.67 mL, 21.1 mmol, 3.0 eq) on ice bath. The mixture was stirred at rt for 3 h, then concentrated under vacuum, and diluted with water (100 mL). The resulting mixture was extracted with EtOAc (100 mL×3). The combined organic layers were washed with brine (50 mL), dried over anhydrous sodium sulfate, filtered and concentrated under vacuum. The resulting residue was purified by column chromatography on silica gel (DCM/MeOH=50/1, v/v) to afford 2-(4-methoxy-2-phenylquinazoline-7-carbonyl)malononitrile as a yellow oil (2.0 g, 6.10 mmol, 86.6%), LRMS (M+H⁺) m/z calculated 329.1, found 329.1.

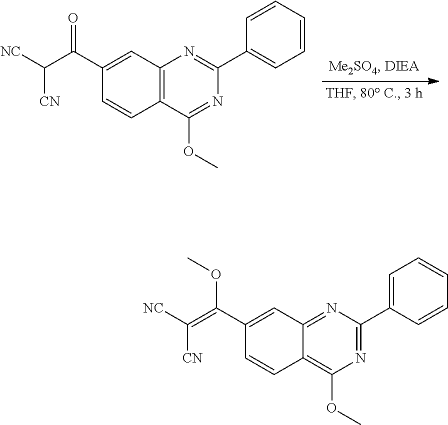 [SMILES: COc1nc(-c2ccccc2)nc2cc(C(=O)C(C#N)C#N)ccc12 ; COC(=C(C#N)C#N)c1ccc2c(OC)nc(-c3ccccc3)nc2c1]

To a solution of 2-(4-methoxy-2-phenylquinazoline-7-carbonyl)malononitrile (500 mg, 1.52 mmol, 1.0 eq) in THF (20 mL) were added Me₂SO₄ (378 mg, 3.0 mmol, 2.0 eq) and DIEA (378 mg, 3.0 mmol, 2.0 eq) at rt. The mixture was stirred at 80° C. for 3 h, then concentrated under vacuum and diluted with water (50 mL), extracted with EtOAc (50 mL×3). The combined organic layers were washed with brine (50 mL), dried over anhydrous sodium sulfate, filtered and concentrated under vacuum. The resulting residue was purified by column chromatography on silica gel (PE/EA=1/1, v/v) to afford 2-(methoxy(4-methoxy-2-phenylquinazolin-7-yl)methylene)malononitrile as a yellow oil (300 mg, 0.877 mmol, 58.4%), LRMS (M+H⁺) m/z calculated 343.1, found 343.1.

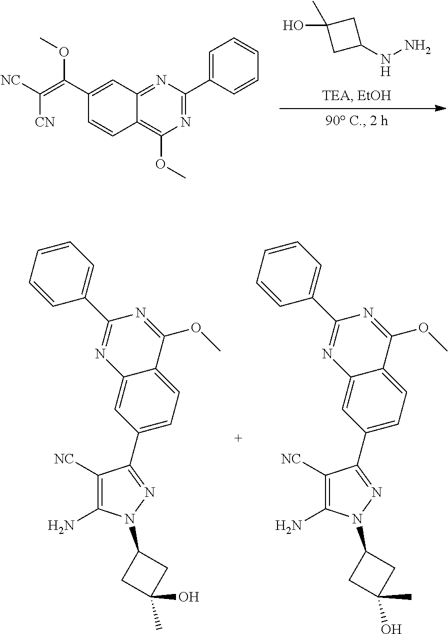

To a solution of 2-(methoxy(4-methoxy-2-phenylquinazolin-7-yl)methylene)malononitrile (300 mg, 0.88 mmol, 1.0 eq) and 3-hydrazinyl-1-methylcyclobutan-1-ol (153 mg, 1.32 mmol, 1.5 eq) in EtOH (20 mL) was added TEA (711, 7.04 mmol, 8.0 eq) at rt. The reaction mixture was stirred at 90° C. for 2 h, and concentrated under vacuum. The resulting residue was purified by column chromatography on silica gel (PE/EA=1/1, v/v) to afford 5-amino-1-((1s,3s)-3-hydroxy-3-methylcyclobutyl)-3-(4-methoxy-2-phenylquinazolin-7-yl)-1H-pyrazole-4-carbonitrile (120 mg, 32.1%) and 5-amino-1-((1r,3r)-3-hydroxy-3-methylcyclobutyl)-3-(4-methoxy-2-phenylquinazolin-7-yl)-1H-pyrazole-4-carbonitrile (180 mg, 80.3%) as a white solid.

5-amino-1-((1s,3s)-3-hydroxy-3-methylcyclobutyl)-3-(4-methoxy-2-phenylquinazolin-7-yl)-1H-pyrazole-4-carbonitrile: ¹H NMR (DMSO-d₆, 400 MHz) δ 8.56-8.58 (t, 2H), 8.40 (s, 1H), 8.22-8.24 (d, 1H), 8.11-8.13 (d, 1H), 7.57-7.58 (d, 3H), 6.87 (s, 2H), 5.28 (s, 1H), 4.46-4.50 (m, 1H), 4.28 (s, 3H), 2.60-2.64 (t, 2H), 2.40-2.43 (t, 2H), 1.34 (s, 3H). LRMS (M+H⁺) m/z calculated 427.2, found 427.2.

5-amino-1-((1r,3r)-3-hydroxy-3-methylcyclobutyl)-3-(4-methoxy-2-phenylquinazolin-7-yl)-1H-pyrazole-4-carbonitrile: ¹H NMR (DMSO-d₆, 400 MHz) δ 8.55-8.58 (m, 2H), 8.39-8.40 (d, 1H), 8.22-8.24 (d, 1H), 8.09-8.11 (m, 1H), 7.56-7.58 (t, 3H), 6.84 (s, 2H), 5.04 (s, 1H), 4.95-4.99 (m, 1H), 4.29 (s, 3H), 2.42-2.55 (m, 4H), 1.38 (s, 3H). LRMS (M+H⁺) m/z calculated 427.2, found 427.2.

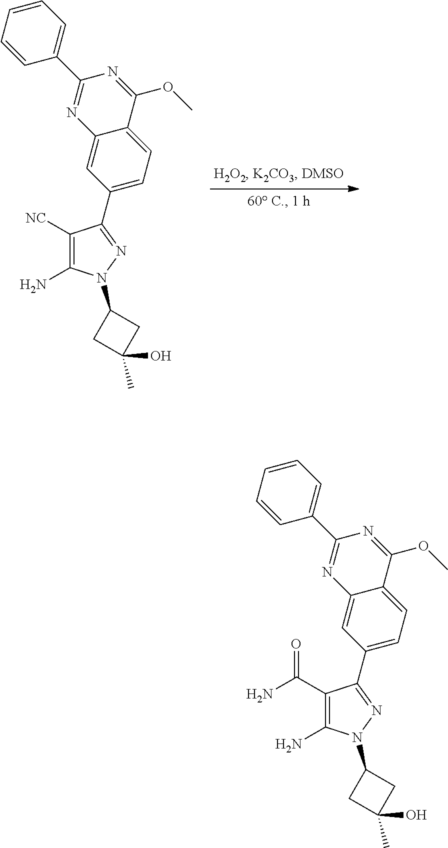 [SMILES: COc1nc(-c2ccccc2)nc2cc(-c3nn([C@H]4C[C@@](C)(O)C4)c(N)c3C#N)ccc12 ; COc1nc(-c2ccccc2)nc2cc(-c3nn([C@H]4C[C@@](C)(O)C4)c(N)c3C(N)=O)ccc12]

To a stirred solution of 5-amino-1-((1s,3s)-3-hydroxy-3-methylcyclobutyl)-3-(4-methoxy-2-phenylquinazolin-7-yl)-1H-pyrazole-4-carbonitrile (100 mg, 0.23 mmol, 1.0 eq) and K₂CO₃ (97.2 mg, 0.70 mmol, 3.0 eq) in DMSO (10 mL) was added H₂O₂ (30%, 520 mg, 4.6 mmol, 20.0 eq) at rt. After addition was complete, the reaction mixture was stirred at 60° C. for 1 h. Water (20 mL) was added, and the mixture was extracted with EtOAc (50 mL). The organic layer was washed with brine (100 mL), dried with anhydrous sodium sulfate, filtered and concentrated. The resulting residue was purified by Prep-HPLC to afford 5-amino-3-(8-fluoro-2-phenylquinolin-7-yl)-1-((1s,3s)-3-hydroxy-3-methylcyclobutyl)-1H-pyrazole-4-carboxamide (61.3 mg, 58.6%) as a white solid. ¹H NMR (DMSO-d₆, 400 MHz) δ 8.56-8.58 (m, 2H), 8.18-8.20 (d, 1H), 9.10-8.11 (d, 1H), 7.82-7.85 (m, 1H), 7.55-7.56 (m, 3H), 6.27 (s, 2H), 5.21 (s, 1H), 4.44-4.48 (m, 1H), 4.29 (s, 3H), 2.58-2.63 (m, 2H), 2.36-2.40 (m, 2H), 1.33 (s, 3H). LRMS (M+H⁺) m/z calculated 445.2, found 445.2.

### II. Biological Evaluation

The ability of the compounds in Table 2 to inhibit IGF-1R was determined.

TABLE 2

| Chemical | Com- | |
|---|---|---|
| Synthesis | pound | |
| Example | No. | Chemical Name |
| 1 | C001 | 5-amino-1-(3-oxocyclobutyl)- |
| | | 3-(2-phenylquinolin-7-yl)-1H- |
| | | pyrazole-4-carboxamide |
| 2 | C002 | 5-amino-3-(2-phenylquinolin- |
| | | 7-yl)-1-(piperidin-4-yl)-1H- |
| | | pyrazole-4-carboxamide |
| 3 | C003 | 4-(5-amino-4-carbamoyl-3-(2- |
| | | phenylquinolin-7-yl)-1H- |
| | | pyrazol-1-yl)piperidine-1-carboxamide |
| 4 | C004 | methyl 4-(5-amino-4-carbamoyl- |
| | | 3-(2-phenylquinolin-7-yl)- |
| | | 1H-pyrazol-1-yl)piperidine-1-carboxylate |
| 5 | C005 | 5-amino-1-((1s,3s)-3-hydroxy- |
| | | 3-methylcyclobutyl)-3-(2- |
| | | phenylquinolin-7-yl)-1H-pyrazole-4-carboxamide |
| 6 | C006 | 5-amino-1-((1r,3r)-3-hydroxy- |
| | | 3-methylcyclobutyl)-3-(2- |
| | | phenylquinolin-7-yl)-1H-pyrazole-4-carboxamide |
| 7 | C007 | 5-amino-1-(1-methylpiperidin- |
| | | 4-yl)-3-(2-phenylquinolin-7- |
| | | yl)-1H-pyrazole-4-carboxamide |
| 8 | C008 | 5-amino-1-(1-(2-hydroxyethyl)piperidin-4-yl)-3-(2- |
| | | phenylquinolin-7-yl)-1H-pyrazole-4-carboxamide |
| 9 | C009 | 5-amino-1-((1s,3s)-3-hydroxycyclobutyl)-3-(2- |
| | | phenylquinolin-7-yl)-1H-pyrazole-4-carboxamide |
| 10 | C010 | 1-(1-acetylpiperidin-4-yl)-5- |
| | | amino-3-(2-phenylquinolin-7- |
| | | yl)-1H-pyrazole-4-carboxamide |
| 11 | C011 | ethyl (1s,3s)-3-(5-amino-4- |
| | | carbamoyl-3-(2-phenylquinolin- |
| | | 7-yl)-1H-pyrazol-1-yl)cyclobutane-1-carboxylate |
| 12 | C012 | 5-amino-1-((1s,3s)-3- |
| | | (morpholinomethyl)cyclobutyl)-3-(2- |
| | | phenylquinolin-7-yl)-1H-pyrazole-4-carboxamide |
| 13 | C013 | 5-amino-1-((1r,3r)-3- |
| | | (morpholinomethyl)cyclobutyl)-3-(2- |
| | | phenylquinolin-7-yl)-1H-pyrazole-4-carboxamide |
| 14 | C014 | 5-amino-1-((1r,3r)-3-(azetidin-1- |
| | | ylmethyl)cyclobutyl)-3-(2- |
| | | phenylquinolin-7-yl)-1H-pyrazole-4-carboxamide |
| 15 | C015 | 5-amino-3-(2-phenylquinolin- |
| | | 7-yl)-1-((1s,3s)-3-(piperazin- |
| | | 1-ylmethyl)cyclobutyl)-1H-pyrazole-4-carboxamide |
| 16 | C016 | 5-amino-3-(2-phenylquinolin- |
| | | 7-yl)-1-((1r,3r)-3-(piperazin- |
| | | 1-ylmethyl)cyclobutyl)-1H-pyrazole-4-carboxamide |
| 17 | C017 | (1s,3s)-3-(5-amino-4-carbamoyl- |
| | | 3-(2-phenylquinolin-7-yl)- |
| | | 1H-pyrazol-1-yl)cyclobutane-1-carboxylic acid |
| 18 | C018 | 5-amino-1-((1s,3s)-3-(azetidin- |
| | | 1-ylmethyl)cyclobutyl)-3-(2- |
| | | phenylquinolin-7-yl)-1H-pyrazole-4-carboxamide |
| 19 | C019 | 5-amino-1-((1s,3s)-3-((4-methylpiperazin-1- |
| | | yl)methyl)cyclobutyl)-3-(2-phenylquinolin-7-yl)-1H- |
| | | pyrazole-4-carboxamide |
| 20 | C020 | ethyl (1r,3r)-3-(5-amino-4- |
| | | carbamoyl-3-(2-phenylquinolin- |
| | | 7-yl)-1H-pyrazol-1-yl)-1- |
| | | methylcyclobutane-1-carboxylate |
| 21 | C021 | 5-amino-1-((1r,3r)-3-((4-methylpiperazin-1- |
| | | yl)methyl)cyclobutyl)-3-(2-phenylquinolin-7-yl)-1H- |
| | | pyrazole-4-carboxamide |
| 22 | C022 | (1r,3r)-3-(5-amino-4-carbamoyl- |
| | | 3-(2-phenylquinolin-7-yl)- |
| | | 1H-pyrazol-1-yl)-1-methylcyclobutane- |
| | | 1-carboxylic acid |
| 23 | C023 | 5-amino-1-(3-morpholinocyclobutyl)- |
| | | 3-(2-phenylquinolin- |
| | | 7-yl)-1H-pyrazole-4-carboxamide |
| 24 | C024 | 5-amino-1-isopropyl-3-(2-phenylquinolin-7-yl)-1H- |
| | | pyrazole-4-carboxamide |
| 25 | C025 | 5-amino-3-(2-phenylquinolin-7-yl)-1-(3-(piperazin-1- |
| | | yl)cyclobutyl)-1H-pyrazole-4-carboxamide |
| 26 | C026 | 5-amino-1-(3-(4-methylpiperazin- |
| | | 1-yl)cyclobutyl)-3-(2-phenylquinolin- |
| | | 7-yl)-1H-pyrazole-4-carboxamide |
| 27 | C027 | 5-amino-1-(3-(azetidin-1-yl)cyclobutyl)-3-(2- |
| | | phenylquinolin-7-yl)-1H-pyrazole-4-carboxamide |
| 28 | C028 | 5-amino-3-(8-fluoro-2-phenylquinolin- |
| | | 7-yl)-1-((1s,3s)-3-hydroxy-3- |
| | | methylcyclobutyl)-1H-pyrazole-4-carboxamide |
| 29 | C029 | 5-amino-1-(oxetan-3-yl)-3-(2-phenylquinolin- |
| | | 7-yl)-1H-pyrazole-4-carboxamide |
| 30 | C030 | 5-amino-1-(3,3-difluorocyclobutyl)- |
| | | 3-(2-phenylquinolin-7- |
| | | yl)-1H-pyrazole-4-carboxamide |
| 31 | C031 | 2-(2-phenylquinolin-7-yl)-4,5,6,7- |
| | | tetrahydropyrazolo[1,5- |
| | | alpyrimidine-3,6-dicarboxamide |
| 32 | C032 | 5-amino-1-((1s,3s)-3-hydroxy- |
| | | 3-methylcyclobutyl)-3-(4- |
| | | methoxy-2-phenylquinolin-7- |
| | | yl)-1H-pyrazole-4-carboxamide |
| 33 | C033 | 5-amino-3-(4-ethoxy-8-fluoro-2- |
| | | phenylquinolin-7-yl)-1-((1s,3s)- |
| | | 3-hydroxy-3-methylcyclobutyl)- |
| | | 1H-pyrazole-4-carboxamide |
| 34 | C034 | 5-amino-3-(8-fluoro-4-methoxy- |
| | | 2-phenylquinolin-7-yl)-1- |
| | | ((1s,3s)-3-hydroxy-3- |
| | | methylcyclobutyl)-1H-pyrazole-4- |
| | | carboxamide |
| 35 | C035 | 5-amino-3-(8-fluoro-2-(2- |
| | | fluorophenyl)-4-methoxyquinolin-7-yl)- |
| | | 1-((1s,3s)-3-hydroxy-3- |
| | | methylcyclobutyl)-1H-pyrazole-4- |
| | | carboxamide |
| 36 | C036 | 5-amino-3-(4-ethoxy-2-phenylquinolin- |
| | | 7-yl)-1-((1s,3s)-3-hydroxy-3- |
| | | methylcyclobutyl)-1H-pyrazole-4-carboxamide |
| 37 | C037 | 5-amino-3-(2-(2-fluorophenyl)quinolin- |
| | | 7-yl)-1-((1s,3s)-3-hydroxy-3- |
| | | methylcyclobutyl)-1H-pyrazole-4-carboxamide |
| 38 | C038 | 5-amino-3-(2-(2-bromophenyl)quinolin- |
| | | 7-yl)-1-((ls,3s)-3-hydroxy-3- |
| | | methylcyclobutyl)-1H-pyrazole-4-carboxamide |
| 39 | C039 | 5-amino-3-(2-(4-fluorophenyl)quinolin- |
| | | 7-yl)-1-((1s,3s)-3-hydroxy-3- |
| | | methylcyclobutyl)-1H-pyrazole-4-carboxamide |
| 40 | C040 | 5-amino-3-(2-(2-fluorophenyl)quinolin- |
| | | 7-yl)-1-((1r,3r)-3-hydroxy-3- |
| | | methylcyclobutyl)-1H-pyrazole-4-carboxamide |
| 41 | C041 | 5-amino-3-(2-(3-fluorophenyl)quinolin- |
| | | 7-yl)-1-((1s,3s)-3-hydroxy-3- |
| | | methylcyclobutyl)-1H-pyrazole-4-carboxamide |
| 42 | C042 | 5-amino-3-(8-fluoro-4-hydroxy- |
| | | 2-phenylquinolin-7-yl)-1- |
| | | ((1s,3s)-3-hydroxy-3- |
| | | methylcyclobutyl)-1H-pyrazole-4- |
| | | carboxamide |
| 43 | C043 | 5-amino-3-(4-ethoxy-2-phenylquinolin- |
| | | 7-yl)-1-(3-oxocyclobutyl)- |
| | | 1H-pyrazole-4-carboxamide |
| 44 | C044 | 5-amino-3-(4-(difluoromethoxy)- |
| | | 8-fluoro-2-phenylquinolin-7-yl)- |
| | | 1-((1s,3s)-3-hydroxy-3-methylcyclobutyl)- |
| | | 1H-pyrazole-4-carboxamide |
| 45 | C045 | 5-amino-3-(8-fluoro-4-methoxy- |
| | | 2-phenylquinolin-7-yl)-1- |
| | | ((lr,3r)-3-hydroxy-3-methylcyclobutyl)-1H- |
| | | pyrazole-4-carboxamide |
| 46 | C046 | 5-amino-3-(2-(2-fluorophenyl)- |
| | | 4-methoxyquinolin-7-yl)-1- |
| | | ((1s,3s)-3-hydroxy-3-methylcyclobutyl)-1H- |
| | | pyrazole-4-carboxamide |
| 47 | C047 | 5-amino-3-(2-(2-fluorophenyl)- |
| | | 4-methoxyquinolin-7-yl)-1- |
| | | ((1r,3r)-3-hydroxy-3-methylcyclobutyl)-1H- |
| | | pyrazole-4-carboxamide |
| 48 | C048 | 5-amino-3-(2-(3-fluorophenyl)- |
| | | 4-methoxyquinolin-7-yl)-1- |
| | | ((1s,3s)-3-hydroxy-3-methylcyclobutyl)-1H- |
| | | pyrazole-4-carboxamide |
| 49 | C049 | 5-amino-3-(2-(3-fluorophenyl)- |
| | | 4-methoxyquinolin-7-yl)-1- |
| | | ((1r,3r)-3-hydroxy-3-methylcyclobutyl)-1H- |
| | | pyrazole-4-carboxamide |
| 50 | C050 | 5-amino-3-(8-fluoro-2-(2- |
| | | fluorophenyl)-4-methoxyquinolin-7-yl)- |
| | | 1-((1r,3r)-3-hydroxy-3- |
| | | methylcyclobutyl)-1H-pyrazole-4- |
| | | carboxamide |
| 51 | C051 | 5-amino-3-(6-fluoro-4-methoxy- |
| | | 2-phenylquinolin-7-yl)-1- |
| | | ((1s,3s)-3-hydroxy-3-methylcyclobutyl)- |
| | | 1H-pyrazole-4-carboxamide |
| 52 | C052 | 5-amino-3-(6-fluoro-4-methoxy- |
| | | 2-phenylquinolin-7-yl)-1- |
| | | ((lr,3r)-3-hydroxy-3-methylcyclobutyl)- |
| | | 1H-pyrazole-4-carboxamide |
| 53 | C053 | 5-amino-3-(4-(difluoromethoxy)- |
| | | 2-phenylquinolin-7-yl)-1- |
| | | ((1s,3s)-3-hydroxy-3-methylcyclobutyl)-1H- |
| | | pyrazole-4-carboxamide |
| 54 | C054 | 5-amino-1-((1s,3s)-3-hydroxy- |
| | | 3-methylcyclobutyl)-3-(2- |
| | | phenylquinazolin-7-yl)-1H-pyrazole-4-carboxamide |
| 55 | C055 | 5-amino-3-(5-fluoro-4-methoxy- |
| | | 2-phenylquinolin-7-yl)-1- |
| | | ((1s,3s)-3-hydroxy-3-methylcyclobutyl)-1H- |
| | | pyrazole-4-carboxamide |
| 56 | C056 | 5-amino-3-(8-fluoro-2-(2- |
| | | fluorophenyl)quinolin-7-yl)-1-((1s,3s)- |
| | | 3-hydroxy-3-methylcyclobutyl)- |
| | | 1H-pyrazole-4-carboxamide |
| 57 | C057 | 5-amino-3-(8-fluoro-2-(pyridin- |
| | | 2-yl)quinolin-7-yl)-1-((1s,3s)-3- |
| | | hydroxy-3-methylcyclobutyl)- |
| | | 1H-pyrazole-4-carboxamide |
| 58 | C058 | 5-amino-3-(3,8-difluoro-2-(2- |
| | | fluorophenyl)quinolin-7-yl)-1- |
| | | ((1s,3s)-3-hydroxy-3-methylcyclobutyl)-1H- |
| | | pyrazole-4-carboxamide |
| 59 | C059 | 5-amino-3-(3,8-difluoro-2- |
| | | phenylquinolin-7-yl)-1-((1s,3s)-3- |
| | | hydroxy-3-methylcyclobutyl)-1H- |
| | | pyrazole-4-carboxamide |
| 60 | C060 | 5-amino-3-(3-fluoro-4-methoxy- |
| | | 2-phenylquinolin-7-yl)-1- |
| | | ((1s,3s)-3-hydroxy-3-methylcyclobutyl)-1H- |
| | | pyrazole-4-carboxamide |
| 61 | C061 | 5-amino-3-(3,8-difluoro-2-(2- |
| | | fluorophenyl)-4-methoxyquinolin- |
| | | 7-yl)-1-((1s,3s)-3-hydroxy-3- |
| | | methylcyclobutyl)-1H-pyrazole-4- |
| | | carboxamide |
| 62 | C062 | 5-amino-3-(3,8-difluoro-4- |
| | | methoxy-2-phenylquinolin-7-yl)-1- |
| | | ((1s,3s)-3-hydroxy-3-methylcyclobutyl)-1H- |
| | | pyrazole-4-carboxamide |
| 63 | C063 | 5-amino-3-(3-fluoro-2-(2- |
| | | fluorophenyl)-4-methoxyquinolin-7-yl)- |
| | | 1-((1s,3s)-3-hydroxy-3-methylcyclobutyl)-1H- |
| | | pyrazole-4-carboxamide |
| 64 | C064 | 5-amino-1-((1s,3s)-3-hydroxy-3-methylcyclobutyl)- |
| | | 3-(4-methoxy-2-phenylquinazolin-7- |
| | | yl)-1H-pyrazole-4-carboxamide |

### Example 1. Biochemical Assay

The inhibitory activity against IGF-1R was measured using ADP-Glo assay. The percent (%) inhibition at each concentration of compound is calculated based on and relative to the luminescence signal in the Max and Min control wells contained within each assay plate. The Max control wells contain enzyme and substrate as 0% inhibition, and the Min control wells only contain substrate without enzyme as 100% inhibition. The concentrations and % inhibition values for tested compounds are plotted and the concentration of compound required for 50% inhibition (IC₅₀) is determined with a four-parameter logistic dose response equation.

Table 3 provides IC₅₀ data of representative compounds against IGF-1R using ADP-Glo assay. The IC₅₀ data are designated within the following ranges: A: ≤0.10 μM, B: >0.10 μM to ≤1 μM, C: >1 μM to ≤20 μM

TABLE 3

| Compound | IGF1R IC₅₀ |
|---|---|
| C005, C012, C013, C014, C015, C016, C021, | A |
| C023, C025, C026, C028, C032, C033, C034, | |
| C035, C036, C037, C038, C040, C041, C043, | |
| C044, C045, C046, C047, C048, C049, C050, | |
| C051, C052, C053, C056, C060, C061, C062, | |
| C063 | |
| C002, C006, C008, C011, C019, C020, C024, | B |
| C027, C030, C039, C055, C057, C058, C059, | |
| C064 | |
| C001, C003, C004, C007, C009, C010, C017, | C |
| C022, C029, C031, C042, C054 | |

### Example 2. Cellular Assay

The CellTiter-Glo luminescent cell viability assay was used to determine the inhibitory activity of the compounds against IGF-1R in Ba/F3-TEL-IGF-1R cells. Table 4 shows cellular IC₅₀ data of representative compounds. The IC₅₀ data are designated within the following ranges: A: ≤1 μM, B: >1 μM to ≤10 μM, C: >10 μM to ≤20 μM

TABLE 4

| Compound | IC₅₀ |
|---|---|
| C005, C012, C013, C032, C033, | A |
| C034, C035, C036, C045, C046, | |
| C048, C050, C051, C056, C060, | |
| C061, C062, C063 | |

### III. Preparation of Pharmaceutical Dosage Forms

### Example 1: Oral Capsule

The active ingredient is a compound of Table 1A or 1B, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1A or 1B, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

We claim:

1. A compound, or pharmaceutically acceptable salt or solvate thereof, having the structure of Formula (I): 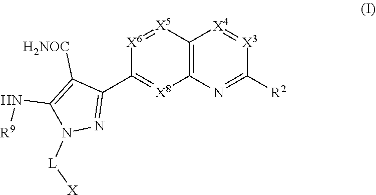
wherein,
X is optionally substituted alkyl, optionally substituted carbocyclyl, optionally substituted carbocyclylalkyl, optionally substituted aryl, optionally substituted aralkyl, optionally substituted heteroaryl, optionally substituted heteroaralkyl, optionally substituted heterocyclyl, or optionally substituted heterocyclylalkyl;
L is a bond, or optionally substituted carbocyclyl, optionally substituted carbocyclylalkyl, optionally substituted heterocyclyl, or optionally substituted heterocyclylalkyl;
R² is optionally substituted carbocyclyl, optionally substituted aryl, optionally substituted heteroaryl, or optionally substituted heterocyclyl; wherein the optional substitution of the optionally substituted carbocyclyl, optionally substituted aryl, optionally substituted heteroaryl, or optionally substituted heterocyclyl is selected from the group consisting of cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted aryloxy, optionally substituted heteroaryloxy, optionally substituted (heterocyclyl)-O—, optionally substituted C1-C6 alkyl, optionally substituted C2-C6 alkynyl, optionally substituted carbocyclyl, optionally substituted C2-C6 alkenyl, optionally substituted aryl, optionally substituted heteroaryl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—N(R¹⁰)₂, or —NR¹¹SO₂—N(R¹⁰)₂;
X³ is N or C—R³;
X⁴ is N or C—R⁴;
X⁵ is N or C—R⁵;
X⁶ is N or C—R⁶;
X⁸ is N or C—R⁸;
R³, R⁴, R⁵, R⁶, and R⁸ are independently selected from the group consisting of hydrogen, cyano, halo, hydroxy, azido, amino, nitro, —CO₂H, —S(O)—R¹⁰, —S—R¹⁰, —S(O)₂—R¹⁰, optionally substituted C1-C6 alkoxy, optionally substituted C1-C6 alkyl, optionally substituted C2-C6 alkynyl, optionally substituted carbocyclyl, optionally substituted C2-C6 alkenyl, optionally substituted heterocyclyl, —N(R¹¹)₂, —CO—R¹⁰, —CO₂—R¹⁰, —CON(R¹¹)₂, —NR¹¹CO—R¹⁰, —NR¹¹CO₂—R¹⁰, —SO₂N(R¹¹)₂, —C(═NR¹²)—N(R¹¹)₂, —NR¹¹CO—N(R¹⁰)₂, and —NR¹¹SO₂—N(R¹⁰)₂;
each R¹⁰ is independently selected from the group consisting of optionally substituted C1-C6 alkyl, optionally substituted C3-C8 carbocyclyl, optionally substituted heterocyclyl, optionally substituted aryl, and optionally substituted heteroaryl;
each R¹¹ is independently selected from the group consisting of hydrogen, optionally substituted C1-C6 alkyl, optionally substituted C3-C8 carbocyclyl, optionally substituted heterocyclyl, optionally substituted aryl, and optionally substituted heteroaryl;
R¹² is H or optionally substituted C1-C6 alkyl;
R⁹ is selected from hydrogen, optionally substituted C1-C6 alkyl, optionally substituted C3-C8 carbocyclyl, or optionally substituted C4-C10 carbocyclylalkyl; wherein either R⁹ and L, or R⁹ and X, may be joined together with any intervening atoms to form an optionally substituted heterocyclyl ring.

2. The compound of claim 1, or pharmaceutically acceptable salt or solvate thereof, wherein X³ is N, and X⁴ is C—R⁴.

3. The compound of claim 1, or pharmaceutically acceptable salt or solvate thereof, wherein X³ is C—R³, and X⁴ is N.

4. The compound of claim 1, or pharmaceutically acceptable salt or solvate thereof, wherein X³ is C—R³, and X⁴ is C—R⁴.

5. The compound of any one of claims 1-4, or pharmaceutically acceptable salt or solvate thereof, wherein L is a bond.

6. The compound of any one of claims 1-5, or pharmaceutically acceptable salt or solvate thereof, wherein X is optionally substituted C3-C7 cycloalkyl.

7. The compound of any one of claims 1-5, or pharmaceutically acceptable salt or solvate thereof, wherein X is optionally substituted C4 cycloalkyl.

8. The compound of any one of claims 1-5, or pharmaceutically acceptable salt or solvate thereof, wherein X is optionally substituted heterocyclyl.

9. The compound of any one of claims 1-5, or pharmaceutically acceptable salt or solvate thereof, wherein X is optionally substituted piperidine or pyrrolidine.

10. The compound of any one of claims 1-5, or pharmaceutically acceptable salt or solvate thereof, wherein X is optionally substituted piperidin-4-yl.

11. The compound of any one of claims 1-5, or pharmaceutically acceptable salt or solvate thereof, wherein X is optionally substituted C1-C8 alkyl.

12. The compound of any one of claims 1-4, or pharmaceutically acceptable salt or solvate thereof, wherein L is an optionally substituted cycloalkyl.

13. The compound of claim 12, or pharmaceutically acceptable salt or solvate thereof, wherein X is optionally substituted C3-C7 cycloalkyl.

14. The compound of claim 12, or pharmaceutically acceptable salt or solvate thereof, wherein X is optionally substituted heterocyclyl.

15. The compound of claim 12, or pharmaceutically acceptable salt or solvate thereof, wherein X is optionally substituted C1-C8 alkyl.

16. The compound of claim 12, or pharmaceutically acceptable salt or solvate thereof, wherein X is optionally substituted C4-C10 cycloalkylalkyl.

17. The compound of claim 12, or pharmaceutically acceptable salt or solvate thereof, wherein X is optionally substituted heterocyclylalkyl.

18. The compound of any one of claim 1, 3, 4, or 5-17, or pharmaceutically acceptable salt or solvate thereof, wherein R³ is H.

19. The compound of any one of claim 1, 2, 4, or 5-17, or pharmaceutically acceptable salt or solvate thereof, wherein R⁴ is H.

20. The compound of any one of claim 1, 2, 4, or 5-17, or pharmaceutically acceptable salt or solvate thereof, wherein R⁴ is optionally substituted C1-C4 alkoxy.

21. The compound of any one of the preceding claims, or pharmaceutically acceptable salt or solvate thereof, wherein X⁸ is N.

22. The compound of any one of claims 1-20, or pharmaceutically acceptable salt or solvate thereof, wherein X⁸ is C—R⁸.

23. The compound of claim 22, or pharmaceutically acceptable salt or solvate thereof, wherein R⁸ is H.

24. The compound of claim 22, or pharmaceutically acceptable salt or solvate thereof, wherein R⁸ is halogen.

25. The compound of claim 24, or pharmaceutically acceptable salt or solvate thereof, wherein R⁸ is F.

26. The compound of any one of the preceding claims, or pharmaceutically acceptable salt or solvate thereof, wherein R⁹ is H.

27. The compound of any one of the preceding claims, or pharmaceutically acceptable salt or solvate thereof, wherein R² is optionally substituted aryl.

28. The compound of claim 27, or pharmaceutically acceptable salt or solvate thereof, wherein R² is optionally substituted phenyl.

29. The compound of claim 27, or pharmaceutically acceptable salt or solvate thereof, wherein R² is phenyl substituted with at least one halogen.

30. The compound of any one of claims 1-26, or pharmaceutically acceptable salt or solvate thereof, wherein R² is optionally substituted heteroaryl.

31. The compound of claim 30, or pharmaceutically acceptable salt or solvate thereof, wherein R² is optionally substituted pyridine.

32. The compound of any one of the preceding claims, or pharmaceutically acceptable salt or solvate thereof, wherein X⁶ is N.

33. The compound of any one of claims 1-31, or pharmaceutically acceptable salt or solvate thereof, wherein X⁶ is C—R⁶.

34. The compound of claim 33, or pharmaceutically acceptable salt or solvate thereof, wherein R⁶ is H.

35. The compound of any one of the preceding claims, or pharmaceutically acceptable salt or solvate thereof, wherein X⁵ is N.

36. The compound of any one of claims 1-34, or pharmaceutically acceptable salt or solvate thereof, wherein X⁵ is C—R⁵.

37. The compound of claim 36, or pharmaceutically acceptable salt or solvate thereof, wherein R⁵ is H.

38. A compound, or pharmaceutically acceptable salt or solvate thereof, having the structure of a compound provided in Table 1A or Table 1B.

39. A pharmaceutical composition comprising a compound, or pharmaceutically acceptable salt or solvate thereof, as described in any one of claims 1-38 and a pharmaceutically acceptable excipient.

40. A method of preparing a pharmaceutical composition comprising mixing a compound, or pharmaceutically acceptable salt or solvate thereof, of any one of claims 1-38, and a pharmaceutically acceptable carrier.

41. A compound of any one of claims 1-38, or pharmaceutically acceptable salt or solvate thereof, for use in a method of treatment of the human or animal body.

42. A compound of any one of claims 1-38, or pharmaceutically acceptable salt or solvate thereof, for use in a method of treatment of cancer or neoplastic disease.

43. Use of a compound of any one of claims 1-38, or pharmaceutically acceptable salt or solvate thereof, in the manufacture of a medicament for the treatment of cancer or neoplastic disease.

44. A method of treating cancer in a patient in need thereof, comprising administering to the patient a compound as described in any one of claims 1-38, or pharmaceutically acceptable salt or solvate thereof.

45. A method of treating cancer in a patient in need thereof, comprising administering to the patient a pharmaceutical composition comprising a compound as described in any one of claims 1-38, or pharmaceutically acceptable salt or solvate thereof, and a pharmaceutically acceptable excipient.

46. A compound of any one of claims 1-38, or pharmaceutically acceptable salt or solvate thereof, for use in a method of treatment of autoimmune disease.

47. Use of a compound of any one of claims 1-38, or pharmaceutically acceptable salt or solvate thereof, in the manufacture of a medicament for the treatment of autoimmune disease.

48. A method of treating autoimmune disease in a patient in need thereof, comprising administering to the patient a compound as described in any one of claims 1-38, or pharmaceutically acceptable salt or solvate thereof.

49. A method of treating autoimmune disease in a patient in need thereof, comprising administering to the patient a pharmaceutical composition comprising a compound as described in any one of claims 1-38, or pharmaceutically acceptable salt or solvate thereof, and a pharmaceutically acceptable excipient.

50. A compound of any one of claims 1-38, or pharmaceutically acceptable salt or solvate thereof, for use in a method of treatment of thyroid eye disease.

51. Use of a compound of any one of claims 1-38, or pharmaceutically acceptable salt or solvate thereof, in the manufacture of a medicament for the treatment of thyroid eye disease.

52. A method of treating thyroid eye disease in a patient in need thereof, comprising administering to the patient a compound as described in any one of claims 1-38, or pharmaceutically acceptable salt or solvate thereof.

53. A method of treating thyroid eye disease in a patient in need thereof, comprising administering to the patient a pharmaceutical composition comprising a compound as described in any one of claims 1-38, or pharmaceutically acceptable salt or solvate thereof, and a pharmaceutically acceptable excipient.
